(19) United States
(12) Patent Application Publication
(10) Pub. No.: US 2020/0383331 A1
(43) Pub. Date: Dec. 10, 2020

(54) COMPOSITIONS AND METHODS FOR LARGE-SCALE IN VITRO PLANT BIOCULTURE

(71) Applicant: BOOSHOOT, LLC, Hailey, ID (US)

(72) Inventor: Jackie HEINRICHER, Anacortes, WA (US)

(21) Appl. No.: 16/728,478

(22) Filed: Dec. 27, 2019

Related U.S. Application Data
(63) Continuation of application No. PCT/US2018/040637, filed on Jul. 2, 2018.
Application No. PCT/US2018/040637 is a continuation of application No. PCT/US2018/040646, filed on Jul. 2, 2018.
(60) Provisional application No. 62/527,946, filed on Jun. 30, 2017.
(60) Provisional application No. 62/611,858, filed on Dec. 29, 2017.
(60) Provisional application No. 62/527,862, filed on Jun. 30, 2017.

(51) Int. Cl. A01N 43/90; A01N 59/00; A01N 59/08; A01N 59/20; A01N 59/16; A01N 59/14; A01N 31/06; A01N 43/78; A01N 37/10; A01N 43/82; A01N 59/12; A01N 37/44; A01N 43/40; A01N 43/08; A01N 37/52; A01N 47/30; A01G 22/15

(52) U.S. Cl. CPC A01N 43/90; A01G 31/00; A01N 59/08; A01N 59/20; A01N 59/16; A01N 59/14; A01N 31/06; A01N 43/78; A01N 37/10; A01N 43/82; A01N 59/12; A01N 37/44; A01N 43/40; A01N 43/08; A01N 37/52; A01N 47/30; A01G 22/15; A01N 59/00

(57) ABSTRACT

The present invention provides media, kits, systems, and methods for achieving large scale pistachio production within a short time via bioculture, large scale yam production within a short time via bioculture, high multiplication rate of plants including cannabis via in vitro micropropagation, high induction rates of somatic embryos from later buds in bamboo, reduced production of phenolic compounds in plants, high production of virus-free plants, including potato, and large scale hemp production via culturing. The present invention for pistachio and yam production results in shorter tuber development phase and higher yield. In some embodiments, the present invention provides compositions, methods, and systems for the micropropagation and mass production of perennials, grasses, bamboos, cannabis and phyto-pharmaceutical plants as well as hemp plants. In some embodiments, the present invention provides compositions, methods, and systems for reducing the production of a phenolic by a plant, such as bamboo.

### CROSS-REFERENCE TO RELATED APPLICATIONS

This application is the Continuation of International Patent Application No. PCT/US2018/040637, filed Jul. 2, 2018, which claims the benefit of priority to U.S. Provisional Application Nos. 62/527,946, filed Jun. 30, 2017; and 62/611,858, filed Dec. 29, 2017; and also a Continuation of PCT/US2018/040646, filed Jul. 2, 2018, which claims the benefit of priority to U.S. Provisional Application No. 62/527,862, filed Jun. 30, 2017; each of which is herein incorporated by reference in its entirety.

### TECHNICAL FIELD

This invention provides compositions, systems, and methods for efficient, rapid and large scale production of plants using bioculture in vitro. In some embodiments, the present invention provides compositions, methods, and systems for the micropropagation and mass production of perennials, grasses, monocots, dicots, and phyto-pharmaceutical plants. In some embodiments, the present invention provides compositions, methods, and systems for the production of virus-free plants.

### BACKGROUND

This invention generally relates to a method of rapid regeneration/proliferation of plant tissues. More particularly, it relates to an improved method of plant tissue proliferation which comprises culturing a tissue or an organ of a plant, a part of the same or cultured cells to proliferate the tissue, organ or cultured cells, thereby regenerating a plant body or producing a useful substance formed by that plant, for the purposes of rapidly generating plants.

The state of the art is such that the demand for plants and plant products far outweight the availability possible with the techniques known in the art. There exists a clear nead in the art for the rapid proliferation of viable plants, and the present application seeks to meet the demand.

### SUMMARY OF THE INVENTION

The present invention provides compositions, methods, kits, bioreactors, and systems for efficient and rapid propagation of pistachio plants at a large scale via bioculture.

In some embodiments, the present invention describes an automated, or semi-automated, low-cost system for the production of pistachio plants, which significantly increases the quantity and quality of pistachio plants, the number and size of the resulting plants, reduces the cost and shortens the cultivation time.

This invention provides novel compositions and an efficient and rapid system for mass propagation of pistachio plants in vitro.

In one embodiment, the present invention provides media for plant micropropagation. In some further embodiments, the media are used for micropropagation of pistachio plants.

In some embodiments, the media are initiation media, multiplication media, and rooting media, such as the BOO3, BOO4, BOO5, BOO6, BOO7, BOO8, BOO9, BOO11, BOO10, BOO13, BOO14, BOO15, BOO16, combination thereof, or functional equivalents thereof (e.g., by reducing or increasing one or more component concentration, or by adding or removing one or more component, wherein the media maintain the same function). As used herein, the media named “BOO” is equivalent to “BOOS.” For example, the media of the present invention are referred to herein as a “BOO3, BOO4, BOO5, BOO6, BOO7, BOO8, BOO9, BOO11, BOO10, BOO13, BOO14, BOO15, BOO16, etc.”, which are also known as (a.k.a) “BOOS3, BOOS4, BOOS5, BOOS6, BOOS7, BOOS8, BOOS9, BOOS11, BOOS10, BOOS13, BOOS14, BOOS15, BOOS16, etc.”, respectively. Therefore, the media designated as “BOO” herein is interchangeably used as “BOOS” in the present invention.

In some embodiments, the initiation media comprise Murashige & Skoog (MS) salts, Woody Plant (WPM) tissue culture salts, and/or Driver Kuniyuki Walnut (DKW) tissue culture salts, and sucrose. In some embodiments, the concentration of one or more components in the MS, WPM, or DKW salts is modified. In some embodiments, the sucrose concentration is about 25 to 35 g/L, for example, about 30 g/L.

In some embodiments, the initiation media comprises at least one cytokinin. In some embodiments, the initiation media further comprises at least one auxin.

In some embodiments, the cytokinin is meta-topolin (mT) or functional derivatives thereof. In some embodiments, the mT concentration in the initiation media is about 0.1 to 30 mg/L, for example, about 1-3 mg/L.

In some embodiments, the auxin is Naphthaleneacetic acid or functional derivatives thereof. In some embodiments, the NAA concentration in the initiation media is about 0.01 to 1 mg/L, for example, about 0.1 mg/L.

In some embodiments, the auxin is IBA or functional derivatives thereof. In some embodiments, the IBA concentration in the initiation media is about 0.01 to 1 mg/L, for example, about 0.1 mg/L.

In some embodiments, the media further comprises a gibberellin acid. In some embodiments, the gibberellin acid is GA3 or functional derivatives thereof. In some embodiments, the gibberellin acid concentration in the initiation media is about 0.2 to 20 mg/L, for example, about 2 mg/L.

In some embodiments, the initiation media are liquid, semi-liquid, solid, or semi-solid media. In some embodiments, the initiation media comprise about 4 to about 10 grams gelling agent, such as agar.

In some embodiments, the initiation media has a pH of about 5.0 to 6.0, for example, about 5.7.

In some embodiments, the multiplication media are similar to, or as the same as the initiation media.

In some embodiments, the rooting media comprise Murashige & Skoog (MS) salts, Woody Plant (WPM) tissue culture salts, and/or Driver Kuniyuki Walnut (DKW) tissue culture salts, and sucrose. In some embodiments, the sucrose concentration is about 25 to 35 g/L, for example, about 30 g/L. In some embodiments, the sucrose concentration is much higher, for example, at least 60 g/L, such as about 60 g/L to about 120 g/L. In some embodiments, the rooting media comprises at least one auxin. In some embodiments, the rooting media do not comprise any cytokinin.

In some embodiments, the auxin is Indole-3-butyric acid (IBA) or functional derivatives thereof. In some embodiments, the IBA concentration in the rooting media is about 0.5 to 5 mg/L, for example, about 1-3 mg/L. In some embodiments, the IBA concentration is much higher, for example, about 100 mg/L to about 1500 mg/L, such as about 250 mg/L to about 1000 mg/L.

In some embodiments, the rooting media comprises charcoal. In some embodiments, the charcoal has a concentration of about 500 mg/L to 1500 mg/L, such as about 1000 mg/L.

In some embodiments, the rooting media are liquid, semi-liquid, solid, or semi-solid media. In some embodiments, the rooting media comprise at least two types of gelling agents. In some embodiments, at least one gelling agent is agar or carrageenan. In some embodiments, the total concentration of gelling agents is about 5 to 10 g/L, for example, about 5 g/L. In some embodiments, the media comprising much higher concentration of auxin is a liquid or semi-liquid media.

In some embodiments, the rooting media has a pH of about 5.0 to 6.0, for example, about 5.7.

In some embodiments, the initiation medium and/or the multiplication medium comprise MS medium containing double concentration of meso elements (e.g., one or more of CaCl2.2H2O, MgSO4.7H2O, and KH2PO4), double iron, and one or more Gamborg's vitamins (e.g., one or more of myo-inositol, Nictotinic acid, pyridoxine salts, and thiamine salts).

The present invention also provides kits for producing plants. In some embodiments, the kits are used for producing pistachio plants. In some embodiments, the kits comprise one or more initiation medium described herein, one or more multiplication medium as described herein, and/or one or more rooting medium as described herein.

The present invention further provides methods for producing plants. In some embodiments, the methods are used for producing pistachio plants.

In some embodiments, the methods of the present invention comprise (a) obtaining pistachio explant. In some embodiments, the pistachio explant is selected from the group consisting of single-node explants, shoot tips, basal (bottom) parts of plants with multiple buds.

In some embodiments, the methods further comprise (b) initiating shoot from the explant obtained in step (a) on an initiation medium. In some embodiments, the step is done in a container, e.g., culture vessels (such as baby food jars) with ventilated lids wherein the container contains an initiation medium. In some embodiments, the initiation medium is a BOO3, BOO4, BOOS, BOO6, or BOO7 media. The pistachio explant is cultured in the container until the explant forms multiple meristematic buds, and the first axillary shoots appear.

In some embodiments, the methods further comprise (c) multiplying the shoot initiated from step (b) on a multiplication medium. In some embodiments, the step comprises transferring the materials obtained from step (b) to a new container comprising a multiplication medium of the present invention. In some embodiments, the multiplication medium is a BOO3, BOO4, BOO5, BOO6, or BOO7 medium. In some embodiments, the multiplication medium is a solid medium. In some embodiments, the multiplication medium is a liquid medium. In some embodiments, cultures are maintained under standard growth condition, such as about 20 to 28° C. (for example, about 22-24° C.), under a day/nigh photoperiod (for example, a 16/8 day/night cycle). In some embodiments, the cultures are maintained on the multiplication medium for 1, 2, 3, 4, 5, 6, 7, 8, or more cycles. In some embodiments, each cycle lasts about a couple of days to a couple of weeks, such as about 3 days to about one week. In some embodiments, the cultures are maintained on the multiplication medium for an additional 30 days, wherein the tissue multiplies by about 2 to about 7 times.

In some embodiments, the methods further optionally comprise (c′) dividing the multiplied plant tissues into clumps. In some embodiments, each clump contains about 3 to 6 shoots. 1.

In some embodiments, the methods further comprise (d) transferring the multiplied shoots of step (c) or step (c′) on a rooting medium to produce a pistachio plant. In some embodiments, the rooting medium is a BOO8, BOO9, BOO11, BOO10, BOO13, BOO14, BOO15, or BOO16 medium. In some embodiments, the rooting medium is a solid medium. In some embodiments, the rooting medium is a liquid medium. In some embodiments, clumps are kept on the medium until individual plants with roots are developed. In some embodiments, the shoot is about 3-4 weeks old.

In some embodiments, the multiplied shoots are kept on a first rooting medium having a higher sugar concentration and then transferred to a second rooting medium having a lower sugar concentration. In some embodiments, the first rooting medium has at least about 60 g/L sugar first and second rooting medium has about 30 g/L sugar. In some embodiments, the first rooting medium and the second rooting medium have about the same sugar concentration. In some embodiments, the shoot is kept on the first media having higher sugar concentration for about 1-3 weeks, and then cultured on the second media having lower sugar concentration for about 2-4 weeks until root develops.

In some embodiments, the first rooting medium and the second rooting medium has an auxin concentration of about 0.5 to 5 mg/L. In some embodiments, the multiplied shoots are kept on a first rooting medium having a higher auxin concentration and then transferred to a second rooting medium having a lower auxin concentration. In some embodiments, the first rooting medium has at least about 30 mg/L auxin and second rooting medium has about 0.5 to 5 mg/L auxin. In some embodiments, the first rooting medium and the second rooting medium have about the same sugar concentration. In some embodiments, the shoot is kept on the first media having higher auxin concentration for about 1-24 hours, and then cultured on the second media having lower auxin concentration for about 2-4 weeks until root develops.

In some embodiments of the present invention, in order to verify pathogen-free plants are produced, the methods further comprise testing for the presence or absence of one or more pistachio pathogen species after one or more cycles. In some embodiments, the pathogen species tested for is a bacteria species, fungal species and/or virus species.

In some embodiments, one or more steps described above are performed in a bioreactor, for example, a temporary immersion bioreactor. In some embodiments, the temporary immersion bioreactor is an ebb and flow bioreactor. In some embodiments, the multiplication step and/or rooting step is performed in the bioreactor. In some embodiments, when a bioreactor is used, one or more of the media mentioned above is in liquid or semi-liquid form. The size of the bioreactor can be any suitable size based on production requirements. For example, the bioreactor can be about 0.1 to about 20 L. The bioreactor can be placed under standard growth conditions, such as about 20 to 26° C. (for example, about 22-24° C.), and a day/nigh photoperiod (for example, a 16/8 day/night cycle). In some embodiments, the medium in the bioreactor is refreshed regularly or when needed. In some embodiments, the medium is refreshed about every one to about every four weeks (with each round called a “cycle”). In some embodiments, after each cycle the amount of biomass increases for about 1 to about 5 times. In some embodiments, the pistachio shoots multiplied in the bioreactor are transferred to rooting medium to perform step (d). In some embodiments, the rooting medium is a liquid medium.

In some embodiments of the present invention, the methods further comprise propagating the pistachio plants obtained to produce pistachio plants in vitro or in vivo.

The present invention also provides methods to produce important chemicals derived from pistachio plants. In some embodiments, the methods comprise producing pistachio plants by the tissue culture methods of the present invention, and then extracting the chemicals from the pistachio plants.

In some embodiments, the initiation medium and/or the multiplication medium comprise MS medium containing double concentration of meso elements (one or more of CaCl2.2H2O, MgSO4.7H2O, and KH2PO4), double iron, and one or more Gamborg's vitamins (one or more of myo-inositol, Nictotinic acid, pyridoxine salts, and thiamine salts).

The present invention also provides methods for inducing root from a pistachio explant in vitro. In some embodiments, the methods comprise growing a pistachio explant on a first rooting medium, and then transferring the pistachio explant to a second rooting medium, wherein the first rooting medium has a higher sugar concentration compared to the second rooting medium. In some embodiments, the first rooting medium comprises about 60 g/L to about 120 g/L sugar, and wherein the second rooting medium comprises about 30 g/L sugar. In some embodiment, the first rooting medium comprises about the same amount of auxin. In some embodiments, the pistachio explant is pre-rooted on the first rooting medium for about 1 to about 3 weeks before being transferred to the second rooting medium. In some embodiments, the pistachio explant is grown on the second rooting medium for about 2 to 4 weeks until roots develop.

Alternatively, the methods comprise growing a pistachio explant on a first rooting medium, and then transferring the pistachio explant to a second rooting medium, wherein the first rooting medium has a higher auxin concentration compared to the second rooting medium. In some embodiments, the first rooting medium comprises about 100 mg/L to 1500 mg/L IBA, and wherein the second rooting medium comprises about 0.1 to 10 mg/L IBA. In some embodiments, the first rooting medium comprises about the same amount of sugar. In some embodiments, the pistachio explant is pre-rooted on the first rooting medium for about 1 to 24 hours before being transferred to the second rooting medium. In some embodiments, the pistachio explant is grown on the second rooting medium for about 2 to 4 weeks until roots develop.

The present invention provides compositions, methods, kits, bioreactors, and systems for efficient and rapid propagation of yam microtubers at a large scale via bioculture.

In some embodiments, the present invention describes an automated, or semi-automated, low-cost system for the production of microtubers, which significantly increases the quality of yam plants, the number and size of the resulting tubers, and shortens the tuberization stage (i.e., time between tuber induction and harvesting).

This invention provides novel compositions and an efficient and rapid system for mass propagation of yam microtubers in vitro, with more than 2 times higher yield of microtubers than using presently-available conventional protocols.

The present invention provides small microtubers (e.g., from about 2 mm to about 3 mm in length) produced using the micropropagation media, systems and methods of the present invention, wherein such microtubers produce as much or more total yield of tubers from yam plants produced from such microtubers as the total yield of tubers from yam plants produced from larger microtubers (e.g., from about 5 mm to about 7 mm in length).

In one embodiment, the present invention provides media for plant micropropagation. In some further embodiments, the media are used for micropropagation of yam microtubers.

In some embodiments, the media are propagation and multiplication media.

In some embodiments, the media are pre-tuberization media.

In some embodiments, the media are tuberization media.

In some embodiments, the propagation and multiplication media comprise Murashige & Skoog (MS) salts and sucrose. In some embodiments, the sucrose concentration is about 15 g/L to 25 g/L, for example, about 20 g/L. In some embodiments, the propagation and multiplication medium does not contain any plant hormones or plant growth regulators.

In some embodiments, the pre-tuberization media comprise sucrose and

(i) at least one cytokinin and at least one auxin;
(ii) at least one growth retardant; or
(iii) at least one cytokinin, at least one auxin, and at least one growth retardant.

In some other embodiments, the sucrose concentration in the pre-tuberization media is about 25 g/L to 35 g/L.

In some embodiments, the at least one cytokinin is 2ip. In some embodiments, the 2ip concentration in the pre-tuberization media is about 1 mg/L to 10 mg/L.

In some embodiments, the at least one auxin is IAA. In some embodiments, the IAA concentration in the pre-tuberization media is about 0.1 mg/L to 10 mg/L.

In some embodiments, the tuberization media comprise sucrose and

(i) at least one auxin;
(ii) at least one growth retardant; or
(iii) at least one auxin and at least one growth retardant.

In some embodiments, the sucrose concentration in the tuberization media is about 50 g/L to 100 g/L.

In some other embodiments, the tuberization media do not comprise any cytokinin.

In some embodiments, the at least one auxin is NAA. In some embodiments, the NAA concentration in the tuberization media is about 0.01 mg/L to about 0.1 mg/L.

In some embodiments of the pre-tuberization media and tuberization media of the present invention, the growth retardant is a gibberellin acid antagonist. In some further embodiments, the gibberellin acid antagonist is ancymidol. In some additional embodiments, the ancymidol concentration in the pre-tuberization media is about 0.1 mg/L to 10 mg/L.

In some embodiments of the pre-tuberization media and tuberization media of the present invention, the media are solid, semi-solid, liquid, or semi-liquid.

In some embodiments of the pre-tuberization media and tuberization media of the present invention, the media have a pH of about 5.5 to 6.2.

The present invention also provides sets (i.e., combinations, collections, etc.) of media for producing plants. In some embodiments, the sets of media are used for producing yam microtubers. In some embodiments, each set of media comprises:

(1) one or more propagation and multiplication medium as described herein;
(2) one or more pre-tuberization medium as described herein; and
(3) one or more tuberization medium as described herein.

In some embodiments, the pre-tuberization medium in the set of media comprises sucrose at concentration S1 and the tuberization medium in the set of media comprises sucrose at concentration S2, wherein S1 is smaller than S2. In some embodiments, S1 is about 25 g/L to 35 g/L and S2 is about 50 g/L to 100 g/L.

The present invention also provides kits for producing plants. In some embodiments, the kits are used for producing yam microtubers. In some embodiments, the kits comprise one or more pre-tuberization medium described herein and one or more tuberization medium as described herein. In some embodiments, the kits further comprise one or more propagation and multiplication medium as described herein. In some embodiments, the kits comprise one or more sets of media as described herein.

The present invention further provides methods for producing plants. In some embodiments, the methods are used for producing yam microtubers.

In some embodiments, the methods of the present invention comprise obtaining pathogen-free yam sprouts. In some embodiments, such methods comprise breaking field tuber dormancy to induce buds. In some further embodiments, the field tuber dormancy is broken naturally, or by treatment with GA3, ethanol, temperature, thiourea, ethylene chlorohydrins, rindite, carbon disulphide, and/or bromoethane.

In some embodiments of the present invention, the induced buds are further grown into sprouts. In some further embodiments, the sprouts are further sterilized. In some additional embodiments, the sprouts are sterilized in sodium dichloroisocyanurate (NaDCC), such as 0.5% solution of NaDCC. In some further embodiments, the sterilized sprouts are cultivated in vitro for one or more cycles until pathogen-free sprouts are produced.

In some embodiments of the present invention, sterilized sprouts are cultivated on a first medium for one or more cycle, and then cultivated on a second medium for one or more cycles until pathogen-free yam plants are produced. In some embodiments, the first medium is a solid, semi-solid, liquid, or semi-liquid medium comprising MS salts, IAA, 2ip, and sucrose. In some further embodiments, the concentration of IAA is about 0.1 mg/L to 1 mg/L; the concentration of 2ip is about 1 mg/L to 10 mg/L; and the concentration of sucrose is about 10 g/L to 40 g/L.

In some further embodiments, the first medium is a BOO18 medium.

In some embodiments of the present invention, the second medium comprises MS salts and sucrose without any hormones or growth regulators. In some embodiments, the sucrose concentration is about 10 g/L to 40 g/L, such as about 20 g/L.

In some embodiments, the sterilized sprouts are grown under about 20° C. to 28° C., such as about 24° C., until pathogen-free yam plants are produced.

In some embodiments of the present invention, the sterilized sprouts are grown under day/night light cycle, with a day time for about 12 hours to 20 hours, such as about 16 hours.

In some embodiments, the sterilized sprouts are grown under a photon flux density of about 50 μmol/m²/s to 120 μmol/m²/s, such as about 85-100 μmol/m²/s.

In some embodiments of the present invention, in order to verify pathogen-free plants are produced, the methods further comprise testing for the presence or absence of one or more yam pathogen species after one or more cycles. In some embodiments, the pathogen species tested for is a bacteria species, fungal species and/or virus species.

In some embodiments of the present invention, the methods further comprise propagating the pathogen-free yam sprouts obtained to produce yam plants in vitro or in vivo. In some embodiments, this involves propagating the pathogen free yam sprouts in a culture tube or a bioreactor. In some further embodiments, the bioreactor is a temporary immersion bioreactor. In some embodiments, the temporary immersion bioreactor is an ebb and flow bioreactor, such as the ones described herein. In some embodiments, the bioreactor comprises a solid, semi-solid, liquid, or semi-liquid medium comprising MS salts and sucrose.

In some embodiments, the medium used in the bioreactors does not have any hormones or growth regulators. In some further embodiments, the sucrose concentration of the media used in the bioreactors is about 10 g/L to 40 g/L, such as about 20 g/L. In some embodiments, use of the bioreactors produces pathogen-free yam sprouts wherein each plant has about 4-7 axillary buds (e.g., embryonic shoots that lie at the junction of the stem and petiole of the plant). In some embodiments, each bud would develop into a single plant. In some further embodiments, the multiplication factor of using such bioreactors is about 3× to 10×, such as about 5× to 6×. In some embodiments, the bioreactor use includes about 4-6 weeks on a solid medium, or about 2 weeks to about 3 weeks or, in some other embodiments about 2.5 weeks to about 3 weeks, on a liquid medium depending on yam variety. In some embodiments, the yam plants are incubated in a PV1 system. In some embodiments, the yam plants are incubated in a PV2 system.

In some embodiments of the present invention using bioreactors, the sprouts are multiplied under about 20° C. to 28° C., such as about 24° C.

In some further embodiments using bioreactors, the sterilized sprouts are grown under day/night light cycle, with a day time for about 12 hours to 20 hours, such as about 16 hours.

In some embodiments, the photon flux density is about 50 μmol/m²/s to 120 μmol/m²/s, such as about 85-100 μmol/m²/s when the sprouts are grown in a cultivation tube, or about 20-100 μmol/m²/s, such as 30-80 μmol/m²/s when the sprouts are grown in a bioreactor.

In some embodiments of the present invention, the methods further comprise pretreating the yam plants in a bioreactor. In some such embodiments, the bioreactor is a temporary immersion bioreactor, such as an ebb and flow bioreactor described herein. In some embodiments, the yam plants are pretreated in a pre-tuberization medium described herein, such as the BOO18, BOO23, BOO19, BOO20, BOO24, or combination thereof.

In some embodiments, the duration of such pretreatment is about 1 to 3 weeks. In some embodiments, the duration can be shorter or long than 1 to 3 weeks whenever it is appropriate or necessary.

In some embodiments of pretreatment, yam plants are grown under about 20° C. to 28° C., such as about 24° C. In some embodiments of pretreatment, the sterilized sprouts are grown under day/night light cycle, with a day time for about 12 hours to 20 hours, such as about 16 hours. In some embodiments, yam plants in the pretreatment step are grown under a photon flux density of about 20 μmol/m²/s to 100 μmol/m²/s, such as about 30-80 μmol/m²/s.

In some embodiments, the methods further comprise initiating yam microtubers in a bioreactor. In some such embodiments, the bioreactor is a temporary immersion bioreactor, such as an ebb and flow bioreactor described herein. In some embodiments of the initiating process, the yam plants are grown in a tuberization medium described herein, such as the BOO21, BOO25, BOO26, BOO27, BOO22, or combination thereof. In some embodiments of the initiating process, the sucrose concentration in the tuberization medium is higher than the sucrose concentration in the pre-tuberization medium described above. In some embodiments of the initiating process, the tuberization medium has a sugar concentration of about 60 g/L to 80 g/L.

In some embodiments, the duration of the initiation process is about 3 weeks to 6 weeks or less. In some embodiments of the initiation process, the duration can be shorter or long than 3 weeks to 6 weeks whenever it is appropriate or necessary, for example, so long as enough yam microtubers are produced.

In some embodiments of the initiating process, the yam plants are grown under a temperature lower than the temperature used in the pretreatment process, and/or a photon flux density lower than the photon flux density used in the pretreatment process. In some embodiments of the initiation process, the plants are grown under a temperature of about 15° C. to 25° C., such as 20° C. to 24° C.

In some embodiments of the present invention, the yam plants in the tuberization stage are grown under continuous darkness.

In some embodiments of the present invention, the methods for producing yam microtubers comprise utilizing one or more sets of medium or one or more kits as described herein.

The present invention further provides bioreactors for plant propagation. In some embodiments, the bioreactors are temporary immersion bioreactors, such as ebb and flow bioreactors.

In some embodiments, the temporary immersion bioreactors comprise:

a growth vessel for incubating plant tissue in a sterile or substantially sterile environment;

a first media container having a first fluid port and a second fluid port, the first fluid port fluidically coupleable to the growth vessel;

a second media container having a first fluid port and a second fluid port, the first fluid port fluidically coupleable to the growth vessel;

a gas source fluidically coupleable to the second fluid port of the first media container and second fluid port of the second media container; and

a controller operable in a first operating mode in which pressurized gas is delivered from the gas source to the first media container to displace a first volume of liquid contained therein to the growth vessel, and a second operating mode in which pressurized gas is delivered from the gas source to the second media container to displace a second volume of liquid contained therein to the growth vessel.

In some embodiments of the present invention, the controller is operable in a third operating mode in which liquid contained in the growth vessel is allowed to flow from the growth vessel into at least one of the first media container and the second media container. In some further embodiments, the controller is operable in a first incubation sequence in which the third operating mode is executed subsequent to the first operating mode. In some additional embodiments, the controller is operable in a second incubation sequence in which the third operating mode is executed subsequent to the second operating mode. In some other embodiments, the controller is further operable in a plant propagation mode in which the first incubation sequence and the second incubation sequence are executed. In some additional embodiments, the growth vessel is elevated above the first and second media containers to allow liquid to flow from the growth vessel into at least one of the first media container and the second media container in the third operating mode.

In some embodiments of the present invention, the bioreactors further comprise a manifold fluidically coupleable to the growth vessel, the first media container, and the second media container. In some further embodiments, he manifold is operable to control liquid flow between the growth vessel and the first media container and between the growth vessel and the second media container. In some additional embodiments, the growth vessel includes a fluid conduit configured to siphon liquid from the growth vessel to at least one of the first media container and the second media container. In some other embodiments, the growth vessel is an ebb and flow bioreactor growth vessel.

In some embodiments of the present invention, the controller is operable to control fluid communication between the growth vessel and the first media container, between the growth vessel and the second media container, between the gas source and the first media container, and between the gas source and the second media container.

The present invention further provides methods for producing plants comprising utilizing the temporary immersion bioreactors as described herein. In some further embodiments, the temporary immersion bioreactors are used for production of yam microtubers.

The present invention further provides systems for plant propagation. In some embodiments, the systems are for production of yam microtubers. In some further embodiments, the systems comprise a temporary immersion bioreactor, a yam explant, a pre-tuberization medium, and a tuberization medium. In some additional embodiments, the temporary immersion bioreactor is the ebb and flow bioreactor described herein. In some further embodiments, the yam explant is a pathogen-free yam plants or plant part, such as a plant seedling. In some other embodiments, the yam seeding comprises about 4 to 7 axillary buds.

In some embodiments of the present invention, the pre-tuberization medium comprises sucrose and

(i) at least one cytokinin and at least one auxin;
(ii) at least one growth retardant; or
(iii) at least one cytokinin, at least one auxin, and at least one growth retardant.

In some embodiments, the tuberization medium comprises sucrose and

(i) at least one auxin;
(ii) at least one growth retardant; or
(iii) at least one auxin and at least one growth retardant.

The present invention also provides methods of producing yam tubers comprising obtaining pathogen-free microtubers via in vitro propagation, wherein the microtubers are produced by using any one of the methods set forth herein; planting the microtubers; and obtaining yam tubers. In some embodiments of the present invention, such microtubers are about 2 mm-about 3 mm long. In other embodiments, the microtubers are about 5 mm-about 7 mm long.

The present invention also provides methods of increasing yam tuber production, comprising obtaining pathogen-free microtubers via in vitro propagation; planting the microtubers; and obtaining yam tubers, wherein the microtubers are less than about 5 mm long, and wherein the weight of yam tuber produced by using the microtubers less than about 5 mm long is higher than the weight of yam tuber produced by using microtubers more than 5 mm long. In some embodiments of the present invention, the microtubers are about 2 mm-about 3 mm long.

The present invention provides compositions, methods and systems for plant tissue culture, for example, compositions, methods and systems for plant micropropagation. In some embodiments, the compositions, methods and systems are used for plant in vitro micropropagation. In some embodiments, the present invention provides compositions, methods, and systems for the micropropagation and mass production of perennials, grasses, and phyto-pharmaceutical plants.

One aspect of the present invention is a method for micropropagating a plant comprising: (a) incubating an explant of the plant on a solid media; (b) transferring a microshoot from the explant of (a) to a bioreactor with liquid media; (c) exposing the microshoot to a pulsing media; (d) harvesting the microshoot at maturity; and (e) transferring the mature microshoot to a media for rooting, wherein the plant is a perennial, grass, or phyto-pharmaceutical plant. In some embodiments, the method can further comprise exposing the microshoot to medium comprising meta-topoline after step (c).

In some embodiments, the solid media is selected from FIG. 26A or 26B. In some embodiments, the liquid media is a media selected from FIG. 26A or 26B and without agar. In some embodiments, the solid media and liquid media are the same, except the liquid media does not comprise agar.

The pulsing media can be selected from FIG. 26B, such that pulsing media is Pulsing media 1 or Pulsing media 2 of FIG. 26B. In some embodiments, the pulsing media is Pulsing media 1 of FIG. 26B, wherein TDZ is substituted with a different cytokinin, such as BAP, Zeatin, CPPU, or DPU.

In some embodiments, the media for rooting is selected from FIG. 27. The media for rooting can be BOO68, BOO69, BOO70, or BOO71 from FIG. 27.

Another aspect of the present invention is media for micropropagating a plant, a perennial, grass, or phyto-pharmaceutical plant and kits comprising the same. In one embodiment, the media is selected from FIG. 26A or 26B. In one embodiment, the media is Pulsing media 1 or Pulsing media 2.

A kit of the present invention can comprise a media is selected from FIG. 26A or 26B, such as Pulsing media 1 or Pulsing media 2. In one embodiment, the pulsing media is Pulsing media 1, wherein TDZ is substituted with a different cytokinin, such as BAP, Zeatin, CPPU, or DPU. The kit can comprise different combinations of media, such that the media can be used sequentially. Thus, in some embodiments, the kit can further comprise a rooting media, such as selected from FIG. 27. In some embodiments, the kit comprises a BOO54, BOO55, BOO56, BOO57, BOO58, BOO59, BOO60, BOO61, BOO62, BOO63, BOO64, BOO65, BOO66, or BOO67 media from FIG. 26A or 26B.

In some embodiments, the present invention provides compositions, methods, and systems for induction of somatic embryos from immature lateral buds in bamboo.

Accordingly, in one aspect of the present invention, a method for inducing a somatic embryo from an immature lateral bud of bamboo comprises: (a) incubating the immature lateral bud in BOO72, BOO73, or BOO74 media; (b) subculturing material from the lateral bud from (a) to fresh media until a pro-embryo structure is induced; (c) transferring the pro-embryo from (b) to BOO77 or BOO78 media for the pro-embryo to develop into an embryo; and (d) transferring the embryo from (c) to BOO79 or BOO80 media for embryo maturation.

In one embodiment, the method further comprises desicatting or germinating the embryo from (d) on BOO72 media. In another embodiment, the BOO72 media in step (d) does not contain a growth regulator.

In another embodiment, in step (a), the immature lateral bud is in BOO72, BOO73, or BOO74 media for one to three days. In another embodiment, in step (a), the immature lateral bud is in BOO72, BOO73, or BOO74 media for up to seven days.

In some embodiments, in step (a) the immature lateral bud is pulsed with BOO75 and BOO76 media. In one embodiment, the pulsing with BOO75 and BOO76 media is for one to three days. In another embodiment, the pulsing with BOO75 and BOO76 media is for up to seven days.

In some embodiments, in step (b), subculturing to fresh media is performed every 28 days. In one embodiment, the subculturing is performed every 28 days for a period of about 6 months.

The bamboo can be Phyllostachys edulisi ‘Moso’, Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, Guadua Angustifolia, Phylostachys Nigra, Fargesia rufa, Fargesia nitida, Borinda Boliana, Fargesia murielae, Pleioblastus fortune, Fargesia robusta, or Bambusa Oldhamii.

Another aspect of the present invention is a media for inducing a somatic embryo from an immature lateral bud of bamboo, wherein the media is BOO72, BOO73, BOO74, BOO75, BOO76, BOO77, BOO78, BOO79, or BOO80. In some embodiments, a combination of different media is used sequentially.

Also provided herein is a kit comprising a media, wherein the media is BOO72, BOO73, BOO74, BOO75, BOO76, BOO77, BOO78, BOO79, or BOO80. The kit can comprise different combinations of media, such that the media can be used sequentially.

In some embodiments, provided are media for initiating bamboo somatic embryogenesis. In some embodiments, the media comprise at least one plant growth regulator. In some embodiments, the media comprise at least one cytokinin. In some embodiments, the media comprise at least one auxin and at least two cytokinins.

In some embodiments, the cytokinins are isolated or synthetic. In some embodiments, the cytokinins are selected from the group consisting of N⁶-benzylaminopurine (BAP), thidiazuron (TDZ), 2-isopentenyladenine (2-ip), kinetin, meta-topolin (mT), kinetin, dicamba, 2,4-D, picloram, derivatives thereof, analogs thereof, and any combinations thereof. In some embodiments, the concentration of each of the cytokinins ranges from about 0.01 μM to 100 μM, or about 0.1 to 10 mg/L, such as about 1 to 5 mg/L.

In some embodiments, the media for initiating bamboo somatic embryogenesis further comprise one or more carbon sources. In some embodiments, the carbon sources are selected from the group consisting of sucrose, glucose, maltose, lactose, or a combination thereof.

In some embodiments, the media for initiating bamboo somatic embryogenesis are liquid media, semi-liquid media, solid, or semi-solid media.

In some embodiments, the media for initiating bamboo somatic embryogenesis are solidified by one or more gelling agents. In some embodiments, the gelling agents are selected from the group consisting of agar, carrageenan, gellan gum, alginic acid and its salts, agarose, and any combinations thereof. In some embodiments, the media are solidified by 5 g/L agar.

In some embodiments, the media for initiating bamboo somatic embryogenesis further comprise one or more macronutrients, one or more micronutrients, and/or one or more vitamins.

In some embodiments, the macronutrients, the micronutrients, and the vitamins are those found in the standard MS media. In some embodiments, amount of one ore more components in the MS media can be doubled.

Also provided are methods for in vitro propagation of bamboo. In some embodiments, the methods comprise propagating bamboo through somatic embryogenesis. In some embodiments, the bamboo is moso bamboo.

In some embodiments, the methods comprise (a) culturing an explant obtained from a bamboo plant on a first medium to produce one or more embryos. In some embodiments, the first medium is the medium for initiating bamboo somatic embryogenesis as described herein. Non-limiting examples of first media are BOO72, BOO73, and BOO74.

In some embodiments, the explant is obtained from a juvenile or a mature bamboo plant. In some embodiments, the explant is selected from the group consisting of node segments, immature leaves, immature embryos, and mature seeds. In some embodiments, the explant comprises meristematic cells located in axillary or lateral buds.

In some embodiments, optionally the methods further comprise (a′) culturing the embryo obtained from step (a) in a pulsing media. Non-limiting examples of pulsing media are BOO75 and BOO76. In some embodiments, the cells are kept on pulsing media for about 1 to 3 days or for up to seven days. In some embodiments, the pulsing media is essentially as the same as a first media, except for that the concentration of one or more components is doubled or tripled, or more compared to that of a first media.

In some embodiments, the methods further comprise (b) culturing the embryo obtained from step (a) or (a′) in second medium to propagate embryonic cells.

In some embodiments, the second medium is a liquid nutrient medium. In some embodiments, the second medium is a solid nutrient medium. In some embodiments, the solid nutrient media of the present invention comprise only mT, but not TDZ. In some embodiments, the second nutrient media comprise one or more amino acids. In some embodiments, the amino acids are selected from the group consisting of glutamine, adenine, derivatives thereof, analogs thereof, and any combinations thereof. In some embodiments, the amino acids are proline and serine. In some embodiments, the concentration of amino acid is about 0.5 to 2 g/L. Non-limiting examples of second media are BOO77 and BOO78.

In some embodiments, the second nutrient media further comprise one or more vitamins. In some embodiments, the vitamins are selected from the group consisting of vitamin A (e.g., retinol, pro-vitamin A carotenoids), vitamin B1 (e.g., thiamine), vitamin B2 (e.g., riboflavin), vitamin B3 (e.g., niacin), vitamin B5 (e.g., pantothenic acid), vitamin B6 (e.g., pyridoxine), vitamin B8 (e.g., biotin), vitamin B9 (e.g., folic acid), vitamin B12 (e.g., cobalamin), vitamin C (e.g., ascorbic acid), vitamin K (e.g., phylloquinone, menaquinone), and any combinations thereof.

In some embodiments, the second nutrient media comprises casein.

In some embodiments, the second nutrient media further comprise one or more carbon source. In some embodiments, the carbon source is selected from the group consisting of sucrose, glucose, maltose, lactose, or a combination thereof.

In some embodiments, the methods further comprise (c) transferring and culturing the embryogenic suspension obtained from step (b) onto one or more third media to produce mature somatic embryos. In some embodiments, the third media are solid media. In some embodiments, the third media comprise MS salts. In some embodiments, amount of one ore more components in the MS media can be doubled. In some embodiments, the third media comprise abscisic acid (ABA). In some embodiments, the concentration of ABA in the third media is about 1.0 to about 100 μM. Non-limiting examples of third media are BOO79 and BOO80.

In some embodiments, the third media further comprise charcoal (e.g., active charcoal). In some embodiments, the concentration of the charcoal is about 0.01% to 10% by weight.

In some embodiments, the methods further comprise (d) germinating the mature somatic embryos obtained from step (c) to produce bamboo plants. In some embodiments, the mature somatic embryos are germinated on one or more fourth media. In some embodiments, the third media are solid media. In some embodiments, the fourth media comprise MS salts, LV salts, or a combination thereof. In some embodiments, the fourth media further comprise one or more amino acids. In some embodiments, the amino acids are selected from the group consisting of glutamine, asparagines, arginine, proline, analogs thereof, and combinations thereof. In some embodiments, the concentration of each amino acid is about 0.01 to 100 mM. In some embodiments, the fourth media does not comprise any plant growth regulator.

In some embodiments, one or more or all of the media used in the methods described herein are liquid and/or semi-liquid media. In some embodiments, some of the media used herein are liquid and/or semi-liquid media, while the others are solid and/or semi-solid media.

In some embodiments, one or more or all steps in the methods described herein are performed in a bioreactor. In some embodiments, the bioreactor is a temporary immersion bioreactor, such as an ebb and flow bioreactor.

Also provided are kits comprising the initiated embryogenic tissue or the mature somatic embryos obtained by the methods described herein. In some embodiments, the kits comprise one or more media of the present invention.

In some embodiments, the present invention provides compositions, methods, and systems for the reduction of a phenolic in plant, such as bamboo. The bamboo may be in in vitro cultures.

Accordingly, in one aspect of the present invention, a method for reducing the production of a phenolic in bamboo comprises incubating a bamboo tissue culture, explant or seed in BOO32, BOO33 or BOO34 media, wherein the bamboo tissue culture, explant, or seed produces less of the phenolic as compared to a bamboo tissue culture, explant or seed incubated in media that is not BOO32, BOO33 or BOO34. In one embodiment, the phenolic is a polyphenol. In some embodiments, the phenolic is a luteolin derivative, flavone, flavone glycoside or phenolic acid.

The bamboo can be Phyllostachys edulisi ‘Moso’, Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, Guadua Angustifolia, Phylostachys Nigra, Fargesia rufa, Fargesia nitida, Borinda Boliana, Fargesia murielae, Pleioblastus fortune, Fargesia robusta, or Bambusa Oldhamii.

Another aspect of the present invention is a media for reducing phenolic production by bamboo, wherein the media is BOO32, BOO33, or BOO34. In some embodiments, a combination of different media is used sequentially.

Also provided herein is a kit comprising a media, wherein the media is BOO32, BOO33, or BOO34. The kit can comprise different combinations of media, such that the media can be used sequentially.

In some embodiments, the present invention provides compositions, methods, and systems for the production of virus-free plants, such as agricultural plants. The plant can be a plantlet.

Accordingly, in one aspect of the present invention, a method for producing a virus-free plant comprises (a) incubating a plant culture with BOO81 medium; (b) subjecting an explant of the plant culture of (a) to thermotherapy, wherein the explant grows into a plantlet; (c) excising an apical meristem from the plantlet of (b); (d) placing the apical meristem of (c) into a regeneration media; wherein a virus-free plantlet is produced from the apical meristem of (e).

In one embodiment, the culture is incubated under a 16 h light photoperiod at 80-100 μmol/m²/x light intensity at 24° C. in step (a). The incubation can be for one to two weeks.

In another embodiment, thermotherapy comprises incubating the explant under a 16 h light photoperiod at 30-40 μmol/m²/s light intensity at 37° C. The thermotherapy can be for one week.

In some embodiments, the method comprises excising the apical meristem in step (c) using a method as depicted as in FIG. 33.

In some embodiments, step (d) comprises incubating the apical meristem in regeneration media under a 16 h light photoperiod at 80-100 μmol/m²/x light intensity at 24° C.

In some embodiments, the regeneration media of step (d) is selected from FIG. 32. In other embodiments, the regeneration media of step (d) comprises an antiviral, such as Ribavirain (also known as Virazole).

The method for producing a virus-free plant can also further comprise: (e) subculturing the apical meristem of step (d), wherein the subculturing can be performed every two to three weeks.

In some embodiments, the method for producing a virus-free plant further comprises: (f) transferring the apical meristem to a regeneration media. In some embodiments, the regeneration media of step (f) is different than the regeneration media of step (d). In other embodiments, the regeneration media of step (f) is the same as the regeneration media of step (d).

The method for producing a virus-free plant can also further comprise: (g) subculturing the apical meristem, wherein the subculturing can be every two or three weeks.

The plantlet produced by a method disclosed herein can be subcultured or tested for viruses, such as by ELISA. The plantlet can be an agricultural plant, such as potato, tomato, yam, sugar beet, cassava, cucumber or cauliflower.

Another aspect of the present invention is the media for producing a virus-free plantlet and kits comprising the same. In one embodiment, the media is selected from FIG. 32. In one embodiment, the media is BOO81.

A kit of the present invention can comprise BOO81 media. The kit can comprise different combinations of media, such that the media can be used sequentially. Thus, in some embodiments, the kit comprises comprise BOO81 media and one or more regeneration media, wherein the regeneration media can be selected from FIG. 32.

This present invention provides compositions, methods, and systems for the production of virus-free potato plants. Accordingly, in one aspect of the present invention, a method for producing a virus-free potato plant comprises (a) incubating a potato explant in a plant culture with BOO81 medium; (b) subjecting an explant of the plant culture of (a) to thermotherapy, wherein the explant grows into a plantlet; (c) excising an apical meristem from the plantlet of (b); (d) placing the apical meristem of (c) into a regeneration media; wherein a virus-free plantlet is produced from the apical meristem of (e).

In one embodiment, the culture is incubated under a 16 h light photoperiod at 80-100 μmol/m²/x light intensity at 24° C. in step (a). The incubation can be for one to two weeks.

In another embodiment, thermotherapy comprises incubating the explant under a 16 h light photoperiod at 30-40 μmol/m²/s light intensity at 37° C. The thermotherapy can be for one week.

In some embodiments, the method comprises excising the apical meristem in step (c) using a method as depicted as in FIG. 33.

In some embodiments, step (d) comprises incubating the apical meristem in regeneration media under a 16 h light photoperiod at 80-100 μmol/m²/x light intensity at 24° C.

In some embodiments, the regeneration media of step (d) is selected from FIG. 20. In other embodiments, the regeneration media of step (d) comprises an antiviral, such as Ribavirain (also known as Virazole).

The method for producing a virus-free plant can also further comprise: (e) subculturing the apical meristem of step (d), wherein the subculturing can be performed every two to three weeks.

In some embodiments, the method for producing a virus-free plant further comprises: (f) transferring the apical meristem to a regeneration media. In some embodiments, the regeneration media of step (f) is different than the regeneration media of step (d). In other embodiments, the regeneration media of step (f) is the same as the regeneration media of step (d).

The method for producing a virus-free plant can also further comprise: (g) subculturing the apical meristem, wherein the subculturing can be every two or three weeks.

The plantlet produced by a method disclosed herein can be subcultured or tested for viruses, such as by ELISA.

Another aspect of the present invention is the media for producing a virus-free potato plant and kits comprising the same. In one embodiment, the media is selected from FIG. 32. In one embodiment, the media is BOO81.

A kit of the present invention can comprise BOO81 media. The kit can comprise different combinations of media, such that the media can be used sequentially. Thus, in some embodiments, the kit comprises comprise BOO81 media and one or more regeneration media, wherein the regeneration media can be selected from FIG. 32

In some embodiments of the disclosure, the media/medium is selected from on or more of BOO1-BOO91.

The present invention provides compositions, methods, kits, bioreactors, and systems for efficient and rapid propagation of hemp plants at a large scale via bioculture.

In some embodiments, the present invention describes an automated, or semi-automated, low-cost system for the production of hemp plants, which significantly increases the quantity and quality of hemp plants, the number and size of the resulting plants, reduces the cost and shortens the cultivation time.

This invention provides novel compositions and an efficient and rapid system for mass propagation of hemp plants in vitro.

In one embodiment, the present invention provides media for plant micropropagation. In some further embodiments, the media are used for micropropagation of hemp plants.

In some embodiments, the media are initiation media, multiplication media, and rooting media, such as the BOO3, BOO4, BOO5, BOO6, BOO7, BOO8, BOO9, BOO11, BOO10, BOO13, BOO14, BOO15, BOO16, combination thereof, or functional equivalents thereof (e.g., by reducing or increasing one or more component concentration, or by adding or removing one or more component, wherein the media maintain the same function).

In some embodiments, the initiation, multiplication, and/or rooting media comprise Murashige & Skoog (MS) salts, Woody Plant (WPM) tissue culture salts, and/or Driver Kuniyuki Walnut (DKW) tissue culture salts, and sucrose. In some embodiments, the concentration of one or more components in the MS, WPM, or DKW salts is modified. In some embodiments, the sucrose concentration is about 25 to 35 g/L, for example, about 30 g/L. In some embodiments, the sucrose concentration is much higher, for example, at least 60 g/L, such as about 60 g/L to about 120 g/L.

In some embodiments, the initiation, multiplication, and/or rooting media comprises at least one cytokinin. In some embodiments, the cytokinin is is meta-topolin (mT) or any functional derivative. In some embodiments, the mT concentration in the initiation media is about 0.5 to 5 mg/L, for example, about 1-3 mg/L.

In some embodiments, the media further comprises a gibberellin acid. In some embodiments, the gibberellin acid is GA3 or functional derivatives thereof. In some embodiments, the gibberellin acid concentration in the rooting media is about 0.2 to 20 mg/L, for example, about 0.5-5 mg/L.

In some embodiments, the initiation, multiplication, and/or rooting media are liquid, semi-liquid, solid, or semi-solid media. In some embodiments, the initiation media comprise about 4 to about 10 grams gelling agent, such as agar.

In some embodiments, the initiation multiplication, and/or rooting media has a pH of about 5.0 to 6.0, for example, about 5.7.

In some embodiments, the initiation, multiplication, and/or rooting medium comprise MS medium containing double concentration of meso elements (e.g., one or more of CaCl2.2H2O, MgSO4.7H2O, and KH2PO4), double iron, and one or more Gamborg's vitamins (e.g., one or more of myo-inositol, Nictotinic acid, pyridoxine salts, and thiamine salts).

The present invention also provides kits for producing plants. In some embodiments, the kits are used for producing hemp plants or plant part. In some embodiments, the kits comprise one or more initiation medium described herein, one or more multiplication medium as described herein, and/or one or more rooting medium as described herein.

The present invention further provides methods for producing plants. In some embodiments, the methods are used for producing hemp plants or plant part in vitro.

In some embodiments, the methods of the present invention comprise (a) obtaining a hemp explant for hemp culture; In some embodiments, the hemp explant is selected from apical and/or lateral buds of shoots of the hemp plants.

In some embodiments, apical and lateral buds of plants are washed with a mild detergent and surface sterilized under aseptic conditions. Surface sterilization can be achieved by immersing the shoot buds in solutions of bleach. In some embodiments, sterilization process is performed for a period of time ranging from about a few minutes to about a couple of hours. In some embodiments, the shoots were placed in a sterile surface such as the laminar flow hood. In further embodiments, dead tissues can be removed using a sharp scalpel after surface sterilization.

In some embodiments, the methods further comprise (b) initiating the hemp culture with the explant from step (a) on an initiation medium. In some embodiments, the step is done in a test tubes and/or a container such as culture vessels (such as baby food jars) with ventilated lids wherein the container contains an initiation medium. In some embodiments, the initiation medium is a BOO3, BOO4, BOO5, BOO6, or BOO7 media. In some embodiments, the initiation medium is a solid medium. In some embodiments, the initiation medium is a liquid medium. In some embodiments, the initiation step (b) further comprises inoculating the explant on the initiation medium in vitro.

In some embodiments, the methods further comprise (c) multiplying the initiated hemp culture from step (b) on a multiplication medium. In some embodiments, the step comprises transferring the materials obtained from step (b) to a new container comprising a multiplication medium of the present invention. In some embodiments, the multiplication medium is a BOO3, BOO4, BOO5, BOO6, or BOO7 medium. In some embodiments, the initiation medicume is the same as multiplication medium. In some embodiments, the multiplication medium is a solid medium. In some embodiments, the multiplication medium is a liquid medium. In some embodiments, cultures are maintained under standard growth condition, such as about 20 to 28° C. (for example, about 23-27° C.), under a day/nigh photoperiod (for example, a 16/8 day/night cycle). In some embodiments, the cultures are maintained on the multiplication medium for 1, 2, 3, 4, 5, 6, 7, 8, or more cycles. In some embodiments, each cycle lasts about a couple of days to a couple of weeks, such as about four weeks to about eight weeks. In some embodiments, about 5 plants up to about 15,000 plants per the culture vessel are produced for each cycle. In some embodiments, size and volume of a culture vessel for multiplying initiated hemp culture step range from about 5 ml to about 50 gallons, for example, about 100 ml to about 10 gallans.

In some embodiments, the methods further comprise (d) transferring the multiplied hemp culture of step (c) on a rooting medium to produce a hemp plant with root. In some embodiments, the rooting medium is a BOO3, BOO4, BOOS, BOO6, or BOO7 medium.

The present invention provides compositions, methods and systems for plant tissue culture, for example, compositions, methods and systems for plant micropropagation. In some embodiments, the compositions, methods and systems are used for plant in vitro micropropagation. In some embodiments, the present invention provides compositions, methods, and systems for the micropropagation and mass production of cannabis plants.

One aspect of the present invention is a method for micropropagating a plant comprising: (a) incubating an explant of the plant on a solid media; (b) transferring a microshoot from the explant of (a) to a bioreactor with liquid media; (c) exposing the microshoot to a pulsing media; (d) harvesting the microshoot at maturity; and (e) transferring the mature microshoot to a media for rooting, wherein the plant is a cannabis plant and the plant part is a cannabis plant part. In some embodiments, the method can further comprise exposing the microshoot to medium comprising meta-topoline after step (c). In some embodiments of the disclosure, the media/medium is selected from on or more of BOO1-BOO91.

In some embodiments, the solid media is selected from FIG. 26A or 26B. In some embodiments, the solid media is BOO54, BOO55, BOO56, BOO57, BOO58, BOO59, BOO60, BOO61, BOO62, BOO63, BOO64, BOO65, BOO66 or BOO67 media. In some embodiments, the liquid media is a media selected from FIG. 26A or 26B and without agar. In some embodiments, the solid media and liquid media are the same, except the liquid media does not comprise agar.

The pulsing media can be selected from FIG. 26B, such that pulsing media is Pulsing media 1 or Pulsing media 2 of FIG. 26B. In some embodiments, the pulsing media is Pulsing media 1 of FIG. 26B, wherein TDZ is substituted with a different cytokinin, such as BAP, Zeatin, CPPU, or DPU.

In some embodiments, the media for rooting is selected from FIG. 27. The media for rooting can be BOO68, BOO69, BOO70, or BOO71 from FIG. 27.

Another aspect of the present invention is media for micropropagating a cannabis plant and kits comprising the same.

In one embodiment, the media is selected from FIG. 26A or 26B. In one embodiment, the media is Pulsing media 1 or Pulsing media 2 of FIG. 26B. In one embodiment, the pulsing media is Pulsing media 1 of FIG. 26B, wherein TDZ is substituted with a different cytokinin, such as BAP, Zeatin, CPPU, or DPU. The media can comprise different combinations of media, such that the media can be used sequentially. Thus, in some embodiments, the media can further comprise a rooting media, such as selected from FIG. 27. In some embodiments, the media comprises a BOO54, BOO55, BOO56, BOO57, BOO58, BOO59, BOO60, BOO61, BOO62, BOO63, BOO64, BOO65, BOO66, or BOO67 media from FIG. 26A or 26B.

A kit of the present invention can comprise a media is selected from FIG. 26A or 26B, such as Pulsing media 1 or Pulsing media 2 of FIG. 26B. In one embodiment, the pulsing media is Pulsing media 1 of FIG. 26B, wherein TDZ is substituted with a different cytokinin, such as BAP, Zeatin, CPPU, or DPU. The kit can comprise different combinations of media, such that the media can be used sequentially. Thus, in some embodiments, the kit can further comprise a rooting media, such as selected from FIG. 27. In some embodiments, the kit comprises a BOO54, BOO55, BOO56, BOO57, BOO58, BOO59, BOO60, BOO61, BOO62, BOO63, BOO64, BOO65, BOO66, or BOO67 media from FIG. 26A or 26B.

In some embodiments of the disclosure, the media/medium is selected from on or more of BOO1-BOO91.

### BRIEF DESCRIPTION OF THE DRAWINGS

FIG. 1 depicts shoot tip necrosis in pistachio plant cultured in vitro on BOO12 medium.

FIG. 2 depicts that new shoots developed from axillary buds of single-node explants of pistachio after 3 days of culture on BOO3 medium.

FIG. 3 depicts root development in pistachio (Pistacia atlantia x Pistacia intergerrina) plants cultures on rooting medium BOO10 for 2 weeks. The image on the right is a close-up of the left image. The arrows point to the new root.

FIG. 4 depicts well-developed, healthy pistachio plants growing in vitro on BOO3 medium.

FIG. 5 is a block diagram of an example of one system of the invention.

FIG. 6 is a schematic illustration of a non-limiting embodiment of the system of FIG. 5.

FIG. 7 is a schematic illustration of a media container of the system of FIG. 6.

FIG. 8 is a schematic illustration of a manifold of the system of FIG. 6.

FIG. 9A is a front view of a growth vessel of the system of FIG. 6.

FIG. 9B is a side view of the growth vessel of FIG. 9A.

FIG. 9C is a top view of the growth vessel of FIG. 9A.

FIG. 10 is a flowchart of a plant propagation sequence of the invention.

FIG. 11 is a table depicting illustrative compositions and media for in vitro propagation and tuberization of yams and other plants.

FIG. 12 is a table depicting illustrative compositions of additional media for in vitro tuberization of yams.

FIG. 13 is a table showing medium recipes of BOO31, BOO48, BOO49, DKW, BOO51, BOO50, BOO52, and BOO53. All components are represented in milligrams per/L except sugar which is represented in grams per/L.

FIG. 14 is a front perspective view of an oscillating rack, according to an embodiment.

FIG. 15 is a side perspective view of the oscillating rack of FIG. 14.

FIG. 16 is an enlarged exploded view of a portion of the oscillating rack labeled as Region Z in FIG. 15.

FIG. 17 is a cross-sectional view of an upright included in the oscillating rack of FIG. 8, taken along line 4-4 in FIG. 14.

FIG. 18 is a perspective view of a shelf assembly included in the oscillating rack of FIG. 14.

FIG. 19 is a perspective view of a portion of the shelf assembly of FIG. 18.

FIG. 20 is a cross-sectional view of a platform included in the portion of the shelf assembly taken along line 7-7 in FIG. 19.

FIG. 21 is a perspective view of bushings included in the shelf assembly of FIG. 17.

FIG. 22 is an exploded view of a drive assembly included in the oscillating rack of FIG. 14.

FIG. 23 is a side view of a portion of the oscillating rack of FIG. 14, in a first configuration.

FIG. 24 is a side view of the portion of the oscillating rack of FIG. 14, in a second configuration.

FIG. 25 is a side view of the portion of the oscillating rack of FIG. 14, in a third configuration.

FIG. 26A and FIG. 26B are tables showing media formulations of BOO54, BOO55, BOO56, BOO57, BOO58, BOO59, BOO66, and BOO61 (FIG. 26A), and BOO62, BOO63, BOO64, BOO65, BOO66, and BOO67; and pulsing media (FIG. 26B); which may be used for micropropagation of aloe vera, ginger, grape, cannabis, garlic, onion, Echinacea, miscanthus, arundo donax, switch grass, rice, sugar cane, echinacea, geranium, and other plants disclosed herein.

FIG. 27 is a table showing rooting media for micropropagation of plants.

FIG. 28 is a photograph of Echinacea plants ready to be planted.

FIG. 29 is a photograph of established Hakonechloa plant in the greenhouse.

FIG. 30 is a table showing medium recipes for induction, establishment and maturation of Moso somatic embryos. DT-200=Thidiazuron; St-10=meta-topolin

FIG. 31 is a table showing medium recipes for BOO32, BOO33, and BOO34, which are media that reduce phenolic production by bamboo tissue cultures.

FIG. 32 is a table showing medium recipes for producing virus-free plants. BOO81 medium contains an antiviral ribavirin (Virazole), while the remaining media, referred to as “regeneration media” can be used for culturing meristems, such as apical meristems from plantlets that were treated with antiviral chemicals and/or thermotherapy. BOO82 and BOO83 can be used for potato, while the other media are named according to the plant in which the media can be used for.

FIG. 33 is schematic of meristem excision from young potato shoots in vitro.

FIG. 34A, FIG. 34B, FIG. 34C, and FIG. 34D are photographs of meristemic potato clones from Russet Burbank FIG. 34A-34B, Mazama (FIG. 34C), and Yukon Gold (FIG. 34D) potatoes.

FIG. 35 is a photograph of plant regeneration from Mazama meristemic clones.

FIG. 36 is a photograph of plant regeneration from Yukon Gold meristemic clones.

FIG. 37 is a photograph of plant regeneration from Russet Burbank 2 meristemic clones.

FIG. 38 is a table showing composition of media for propagation and rooting of plant culture in vitro, such as pistachio, hemp and other plants disclosed herein.

### DETAILED DESCRIPTION

Pistachio is a member of the Anacardiaceae family in the Pistacia genus. It is referred to as the “green gold tree” due to its high economic value.

The plant material used in this study was interspecific hybrid UCB I (P. atlantica x P. intergerrina) from California, which is the result of a closed pollination of the single P. atlantica tree with P. integerrima pollen. This P. atlantica x P. intergerrina hybrid has increased vigor, as compared with P. atlantica, and generally equal or greater vigor than P. intergerrina. In addition, the UCB I is more resistant to Verticillium vilt, which is a common soil-borne fungal disease of pistachio rootstocks in California.

The information on micropropagation of pistachio plants in vitro is very limited, and refers only to P. vera L. Moreover, those few published protocols for growing pistachio plants in vitro failed to provide satisfactory growth of our cultivar. Thus, it is virtually new and unexplored area. In addition, rooting of pistachio presents a major obstacle for the pistachio industry, as well as in vitro shoot tip necrosis.

Here, we describe a highly efficient system for propagation of pistachio plants in vitro, which provided easy scale-up of high quality, healthy plants within short period of time.

Yams are generally grown from seed yams—these are tubers specifically grown to be disease free and provide consistent and healthy plants. To be disease free, the areas where seed yams are grown are selected with care. In the USA this restricts production of seed yams to only 15 states out of the 50 states that grow yams. These locations are selected for their cold hard winters that kill pests and long sunshine hours in the summer for optimum growth.

Virus and viroid diseases are among the most significant diseases in yam seed production and certification. They include Dioscorea bacilliform virus (DB V, genus Badnavirus), Yam mosaic virus (YMV, genus Potyvirus), and Yam mild mosaic virus (YMMV, genus Potyvirus). Viral disease is an important reason attributed to lower yield of yam varieties. Other diseases, such as bacterial and fungal diseases also present a problem for yam production.

Meristem tissue culture has been successfully applied in yams for development of virus free plants and virus free yam tuber plants.

Yam microtubers offer several advantages over seed yams and meristem tissue culture for the seed production of yams, including relatively shorter field times necessary to supply commercial growers (3 or 4 years compared with 7 or more years), and greatly improved seed tuber quality (fewer viral, bacterial, fungal problems) (see Donelly et al., 2003). In contrast to in vitro propagated plants, microtubers can be stored and transplanted directly to the field without an acclimatization stage. Because of relatively small size, their handling, storage and transport are more practical than that of regular tubers or in vitro plants. Many countries lacking isolated and pathogen-free growing areas that permit the production of yam seed tubers use microtuber propagation as a vital component for yam production.

Potatoes are generally grown from seed potatoes—these are tubers specifically grown to be disease free and provide consistent and healthy plants. To be disease free, the areas where seed potatoes are grown are selected with care. In the USA this restricts production of seed potatoes to only 15 states out of the 50 states that grow potatoes. These locations are selected for their cold hard winters that kill pests and long sunshine hours in the summer for optimum growth.

Virus and viroid diseases are among the most significant diseases in potato seed production and certification. They include Potato leaf roll virus (PLRV), Potato virus A (PVA), Potato virus M (PVM), Potato virus S (PVS), Potato virus X (PVX), Potato virus Potato virus S (PVS), Potato virus X (PVX), Potato virus Y (PVY) and Potato spindle tuber viroid (PSTVd). Viral disease is an important reason attributed to lower yield of potato varieties. Other diseases, such as bacterial and fungal diseases also present a problem for potato production.

Though meristem tissue culture has been successfully applied in potato for development of virus free plants and virus free potato tuber plants, there is continuing need for improvement in methods of producing virus-free potato plants. The present invention meets this need and provides related advantages.

The main problems associated with microtuber production are low yield of tubers (usually less than 1 tuber per plant), small tuber size (usually less than 1 cm in diameter), long production time (usually 4-6 months), and higher labor costs. Thus, there is a continuing need for improvement of microtuber production, which the present invention addresses.

With increasing burdens on land to produce food and biomass for energy and materials additional attention is being placed on identifying and utilizing faster growing and more productive plants. Although many plants are suitable for such purposes, there is still a great need to develop compositions, methods, and systems for fast, economical plant propagation.

### Definition

As used herein, the verb “comprise” as is used in this description and in the claims and its conjugations are used in its non-limiting sense to mean that items following the word are included, but items not specifically mentioned are not excluded.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “plant” refers to any living organism belonging to the kingdom Plantae (i.e., any genus/species in the Plant Kingdom). to trees, herbs, bushes, grasses, vines, ferns, mosses and green algae. The term refers to both monocotyledonous plants, also called monocots, and dicotyledonous plants, also called dicots. Examples of particular plants include but are not limited to bamboo, corn, potatoes, roses, apple trees, sunflowers, wheat, rice, bananas, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g. broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g. garlic, leek, onion (dry bulb, green, and Welch), shallot, and other bulb vegetable crops), citrus fruits (e.g. grapefruit, lemon, lime, orange, tangerine, citrus hybrids, pummelo, and other citrus fruit crops), cucurbit vegetables (e.g. cucumber, citron melon, edible gourds, gherkin, muskmelons (including hybrids and/or cultivars of cucumis melons), water-melon, cantaloupe, and other cucurbit vegetable crops), fruiting vegetables (including eggplant, ground cherry, pepino, pepper, tomato, tomatillo, and other fruiting vegetable crops), grape, leafy vegetables (e.g. romaine), root/tuber and corm vegetables (e.g. potato), and tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, lackberries, cloudberries, loganberries, raspberries, salmonberries, thimbleberries, and wineberries), cereal crops (e.g., corn, rice, wheat, barley, sorghum, millets, oats, ryes, triticales, buckwheats, fonio, and quinoa), pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fiber crops (e.g. hemp, cotton), ornamentals, and the like. For example, the plant is a species in the tribe of Camelineae, such as C. alyssum, C. anomala, C. grandiflora, C. hispida, C. laxa, C. microcarpa, C. microphylla, C. per sistens, C. rumelica, C. sativa, C. Stiefelhagenii, or any hybrid thereof. For example, in some embodiments, the plant is a species in the Pistachioa genus. In some embodiments, the plant is W. japonica. In some embodiments, the plant is a species in the Solanum genus, such as S. tuberosum S. stenotomum, S. phureja, S. goniocalyx, S. ajanhuiri. S. chaucha, S. juzepczukii, and S. curtilobum. In some embodiments, the plant is a yam variety of the S. tuberosum species.

In some embodiments, the compositions, methods, and systems are useful for crop plant in vitro propagation. In some embodiments, a crop plant is an agricultural plant. As used herein, the term “crop plant” refers to any plant grown for any commercial purpose, including, but not limited to the following purposes: seed production, hay production, ornamental use, fruit production, berry production, vegetable production, oil production, protein production, forage production, animal grazing, golf courses, lawns, flower production, landscaping, erosion control, green manure, improving soil tilth/health, producing pharmaceutical products/drugs, producing food or food additives, smoking products, pulp production and wood production. For example, an agricultural plant can be potato, tomato, yam, sugar beet, cassava, cucumber, or cauliflower.

In some embodiments, the compositions, methods, and systems are useful for monocotyledon plants propagation. As used herein, the term “monocotyledon” or “monocot” refer to any of a subclass (Monocotyledoneae) of flowering plants having an embryo containing only one seed leaf and usually having parallel-veined leaves, flower parts in multiples of three, and no secondary growth in stems and roots. Examples include lilies; orchids; rice; corn, grasses, such as tall fescue, goat grass, and Kentucky bluegrass; grains, such as wheat, oats and barley; irises; onions and palms.

In some embodiments, the compositions, methods, and systems are useful for propagation of perennials. The perennial can be an evergreen, deciduous, monocarpic, woody, or herbaceous perennial. In some embodiments, the perennial is Begonia, banana, goldenrod, mint, agave, maple tree, pine tree, apple tree, alfalfa or red clover.

In some embodiments, the compositions, methods, and systems are useful for propagation of grasses. The grass can be of the Poaceae (or Gramineae), Cyperaceae or Juncaceae family. The grass can be a perennial grass or a cereal grass. The grass can be switchgrass, big bluestem, miscanthus, alfalfa, orchard grass, or reed canarygrass. The grass can be bamboo, tall fescue, goat grass, or Kentucky bluegrass. Other types of grasses include wheat, rye, oat, barley, soy, and hemp, as well as straws derived therefrom.

In some embodiments, the compositions, methods, and systems are useful for propagation of phyto-pharmaceutical plants. A phyto-pharmaceutical plant is a plant that can be used for a plant-based medicament. In one embodiment, one or more active ingredients in a phyto-pharmaceutical is derived from a plant disclosed herein. In some embodiments, the active ingredient is a plant disclosed herein.

In some embodiments, the compositions, methods, and systems are useful for propagation of Aloe vera, Ginger, Grape, Cannabis, Garlic, Onion, Echinacea, Geranium, Hakonechloa, Miscanthus, Arundo donax, Switch grass, Rice, or Sugar cane.

In some embodiments, the compositions, methods, and systems are useful for bamboo plant in vitro propagation. As used herein, the term “bamboo” refers to plants in the subfamily of Bambusoideae. Representative genera of bamboo are described in International Patent Application Publication No. WO2011100762, which is incorporated herein by reference in its entirety.

As used herein, the terms “herb”, “herbs” and “herbal” all refer to an annual, biennial, or perennial plant that does not develop persistent woody tissue but dies down at the end of a growing season. Herbal plants typically are capable of flowering and producing seeds. In some contexts the terms refer to a plant or plant part valued for its medicinal, savory, or aromatic qualities. Examples of herbs include, but are not limited to, sage, rosemary, parsley, basil, catnip and marijuana.

As used herein, “herbal medicine” or “herbal medicinal” refer to herbs, herbal materials, herbal preparations, and finished herbal products that contain parts of plants, other plant materials, or combinations thereof as active ingredients. Herbs include crude plant material, for example, leaves, flowers, fruit, seed, and stems. Herbal materials include, in addition to herbs, fresh juices, gums, fixed oils, essential oils, resins, and dry powders of herbs. Herbal preparations are the basis for finished herbal products and may include comminuted or powdered herbal materials, or extracts, tinctures, and fatty oils of herbal materials. Finished herbal products consist of herbal preparations made from one or more herbs. See, e.g., Perspectives in Clinical Research, April-June 2016, 7(2):59-61.

As used herein, the term “phytopharmaceutical” (aka “phyto-pharmaceutical”) refers to a pharmaceutical of plant origin.

As used herein, the term “plant part” refers to any part of a plant including but not limited to the shoot, root, stem, axillary buds, seeds, stipules, leaves, petals, flowers, ovules, bracts, branches, petioles, node, internodes, bark, pubescence, tillers, rhizomes, fronds, blades, pollen, stamen, microtubers, and the like. The two main parts of plants grown in some sort of media, such as soil, are often referred to as the “above-ground” part, also often referred to as the “shoots”, and the “below-ground” part, also often referred to as the “roots”.

As used herein, the term “germplasm” refers to the genetic material with its specific molecular and chemical makeup that comprises the physical foundation of the hereditary qualities of an organism.

As used herein, the phrase “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the term “genotype” refers to the genetic makeup of an individual cell, cell culture, tissue, organism (e.g., a plant), or group of organisms.

As used herein, the term “clone” refers to a cell, group of cells, a part, tissue, organism (e.g., a plant), or group of organisms that is descended or derived from and genetically identical or substantially identical to a single precursor. In some embodiments, the clone is produced in a process comprising at least one asexual step.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “population” means a genetically homogeneous or heterogeneous collection of plants sharing a common genetic derivation.

As used herein, the term “bioreactor” refers to any vessel, device or system capable of holding, supporting and/or growing viable tissue. In other words, the term “bioreactor” as used herein may refer to a growth vessel that holds viable plant tissue, various other components internal or external to the growth vessel that are required for or aid the holding, supporting and/or growing of viable plant tissue, and any subsystem thereof.

As used herein, the phrase “temporary immersion bioreactor” refers to any bioreactor designed to temporarily wet a part or entire culture or plant tissue with nutrient medium (e.g., liquid or semi-liquid) followed by draining a part or all of the excess nutrient medium.

As used herein, a “plant propagation system” is a bioreactor for growing viable plant tissue.

As used herein, the media named “BOO” is equivalent to “BOOS.” The media designated as “BOO” herein is interchangeably used as “BOOS” in the present invention. For example, the media of the present invention are referred to herein as a “BOO3, BOO4, BOO5, BOO6, BOO7, BOO8, BOO9, BOO11, BOO10, BOO13, BOO14, BOO15, BOO16, etc.”, which are also known as (a.k.a) “BOOS3, BOOS4, BOOS5, BOOS6, BOOS7, BOOS8, BOOS9, BOOS11, BOOS10, BOOS13, BOOS14, BOOS15, BOOS16, etc.”, respectively.

More rapidly growing plants are available for cultivation and industrial uses, including plants in the subfamily Bambusoidea. The subfamily Bambusoideae (of the family Poaceae), comprises both woody and herbaceous bamboos. At present roughly 120 genera of temperate and tropical woody bamboos are recognized. Bamboos are versatile plants with many different applications. It has been estimated that approximately 2.2 billion people worldwide use bamboo to some extent, and in 1985 the global revenue attributable to bamboo was estimated around U.S. $4.5 billion. The market for bamboo is also expanding. Bamboo shoots are a staple of Asian cuisine, and bamboo is found in a number of products including toothpicks, brooms, poles for viticulture and arboriculture, landscaping materials, parquet flooring, laminate materials, furniture, handicrafts and other household items. In addition, bamboo is becoming an important source of textile material as a component of paper production and as a source of structural timber.

Plant species such as bamboo are considered environmentally friendly “green” products, which have extremely rapid growth rates. Despite rapid growth rates, other characteristics of these plants make it difficult to rapidly propagate these plants at an industrial scale. For example, many commercially important bamboos only flower at intervals of as long as 60-130 years. Compounding the difficulties of this long flowering cycle is the fact that many bamboos exhibit mass (or gregarious) flowering, with all plants in the population flowering simultaneously. For example, Phyllostachys bambusoides flowers at an interval of 130 years, and in this species all plants of the same stock flower at the same time, regardless of differences in geographic locations or climatic conditions. After flowering, the bamboo dies. Bamboo's lengthy flowering interval and propensity for mass flowering makes it very difficult to obtain seeds for propagation. Compounding this problem is the fact that bamboo seeds, even when they are available, remain viable for no more than 3-6 months.

As a result of these difficulties with the propagation of bamboo and other fast growing plant species using traditional sexual reproduction, these plants are often propagated by asexual techniques such as clump division and cutting. These asexual propagation techniques, however, are insufficient to meet projected world demand because both their capacity to produce mass scale production, and their practical efficiency, are too low. In addition many asexual propagation methods have the downside of failing to eliminate pathogens present in the parent plants. Therefore, compositions, methods, and systems to achieve large scale production of plants are highly desirable. Micropropagation (also known as tissue culturing with the terms used interchangeably herein), is an excellent potential method that could be used to achieve this aim.

Micropropagation is not unlike growing plants from cuttings. However, unlike plants grown from cuttings, micropropagated plants are grown in vitro in sterile media. Typically, the growth media comprises a solid or semi-solid material such as agar, with the addition of various compounds such as nutrients, inorganic salts, growth regulators, sugars, vitamins and other compounds.

A benefit of tissue culturing plants is that the plants can be grown in a sterile environment so that they may more likely remain disease free. Other benefits include the ability to grow very large numbers of plants in a small space, the reduced water and nutrient needs of micropropagated plants, and the rapid multiplication of tissues that can in turn be used to yield more tissue culture material. Moreover micropropagation is very flexible and rapid upscaling is possible (within 1 year nearly one million plants can be produced from a single genotype). Such short time frames and large numbers cannot be rivaled by any conventional method. Tissue culturing also provides for the production of high quality plants which are easy to transport and deliver.

Some papers have published which address tissue culturing of plants. In practice, however (i.e., for large or mass scale propagation of bamboos), the compositions, methods, and systems described in these papers do not translate into commercially viable propagation systems.

The difficulties encountered in tissue culturing of plant species include high incidences of endogenous or surface contaminations and browning, factors related to dormancy or topophysis and hyperhydricity. The present disclosure provides compositions, methods, and systems that overcome these difficulties allowing the commercial-scale asexual production of plants.

Micropropagation in liquid culture media increases nutrient uptake and promotes growth. However, the advantages of in vitro culture in a liquid media are often counterbalanced by technical problems such as asphyxia, hyperhydricity, shear forces and the need for complex equipment.

International Patent Application Publication No. WO/2011/100762, which is incorporated herein by reference in its entirety, describes compositions and methods that are useful for bamboo in vitro propagation.

The present application discloses novel compositions, methods, and systems for the rapid in vitro propagation of plants. The present application also discloses novel compositions, methods, and systems for the reduction of phenolic production by plants, such as bamboo.

The present invention provides compositions and methods that can significantly increase plant tissue culture multiplication rate within a shorter time. In some embodiments, a strong cytokinin such as thidiazuron is utilized for a very brief period of time in either a solid or liquid induction medium to induce multiple shoot bud formation in explants of plant species. This bud induction treatment utilizing a media containing a strong cytokinin such as thidiazuron is followed by a shoot elongation and maintenance treatment whereby a relatively weaker cytokinin such as BAP, meta-topolin, 2ip, zeatin and or zeatin riboside is used to accomplish the shoot elongation and maintenance of the culture. This process, when alternated methodically resulted in culture multiplication rates between 2× and 28× within a 3-week culture cycle.

### Pistachio

The pistachio, Pistacia vera in the Anacardiaceae family, is a small tree originally from Greater Iran which now can also be found in regions of Syria, Lebanon, Turkey, Greece, Tunisia, Kyrgyzstan, Tajikistan, Turkmenistan, India, Pakistan, Egypt, Italy (Sicily), Uzbekistan, Afghanistan, and the United States. The tree produces an important culinary nut.

Pistacia vera often is confused with other species in the genus Pistacia that are also known as pistachio. These species can be distinguished from P. vera by their geographic distributions (in the wild) and their nuts which are much smaller, have a strong flavor of turpentine, and have a shell that is not hard.

As used herein, the term pistachio refers to all species in the genus Pistacia. In some embodiments, the pistachio is the hybrid species produced from cross of Pistacia atlantica and Pistacia intergerrina. In some embodiments, the pistachio plant is the Kerman variety.

Pistachio is a desert plant, and is highly tolerant of saline soil. It has been reported to grow well when irrigated with water having 3,000-4,000 ppm of soluble salts. Pistachio trees are fairly hardy in the right conditions, and can survive temperatures ranging between −10° C. (14° F.) in winter and 48° C. (118° F.) in summer. They need a sunny position and well-drained soil. Pistachio trees do poorly in conditions of high humidity, and are susceptible to root rot in winter if they get too much water and the soil is not sufficiently free-draining. Long, hot summers are required for proper ripening of the fruit.

Iran, the United States and Turkey are the major producers of pistachios. The trees are planted in orchards, and take approximately seven to ten years to reach significant production. Production is alternate bearing or biennial bearing, meaning the harvest is heavier in alternate years. Peak production is reached at approximately 20 years. Trees are usually pruned to size to make the harvest easier. Harvesting in the United States and in Greece is often accomplished by using shaking equipment to shake the nuts off the tree. After hulling and drying, pistachios are sorted according to open mouth and closed mouth shell. Sun drying has been found to be the best method of drying. Then they are roasted or processed by special machines to produce pistachio kernels.

Pistachio trees are vulnerable to a wide variety of diseases, including but not limited to, Alternaria late blight, Armillaria root rot, Aspergillus fruit rot, Blossom and shoot blight, Camarosporium shoot and panicle blight, Cotton root rot, Eutypa dieback, Gum canker, Leaf spot, Panicle and shoot blight, Phomopsis shoot blight, Powdery mildew, Phytophthora root and crown rot, Rust, Sclerotinia shoot blight, Seedling blight, Septoria leaf spot, Stigmatomycosis, Thread blight, Phytophthora trunk and bark canker, Verticillium wilt. Among these is infection by the fungus Botryosphaeria, which causes panicle and shoot blight (i.e., kills flowers and young shoots), and can damage entire pistachio orchards.

In California, almost all female pistachio trees are the cultivar “Kerman”. A scion from a mature female Kerman is grafted onto a one-year-old rootstock. Male pistachios may be a different variety.

Pistachios in particular significantly reduced levels of low-density lipoprotein (LDL cholesterol) while increasing antioxidant levels in the serum of volunteers. Other health benefits of pistachio include, but are not limited to, reducing LDL (“bad”) cholesterol and increase the good HDL cholesterol after only a short period of regular consumption; high in antioxidants such as vitamins A and E; fighting inflammation, protecting blood vessels and reducing risk of heart disease. preventing Type 2 diabetes, rich source of vitamin B6, having wide-ranging effects on the nervous system, having lutein and zeaxanthin (function as protective antioxidants, defending tissues from damage from free radicals); helping the body make healthy red blood cells, and helping maintain the health of lymphoid glands, such as the thymus, spleen and lymph nodes, ensuring the production of white blood cells that defend the body from infections.

### Yams

Yam is the common name for some plant species in the genus Dioscorea (family dioscoreaceae) which produce tubers, bulbils, or rhizomes having medicinal and economic importance. These are perennial herbaceous vines cultivated for the consumption of their starchy tubers in Africa, Asia, Latin America, the Caribbean, and Oceania.

True yams are botanically distinct from the sweet potato, but moist-fleshed varieties of sweet potato are often called yams in the United States. D. bulbifera, the air-potato yam, is one of the few true yams cultivated for food in the United States. Yams have thick tubers (generally a development of the base of the stem), from which protrude long, slender, annual, climbing stems bearing leaves, which are either alternate or opposite and either entire or lobed and unisexual flowers in long clusters. The flowers are generally small and individually inconspicuous, though collectively showy. Each consists of a greenish, bell-shaped or flat perianth of six pieces, enclosing six or fewer stamens in the male flowers and surmounting a three-celled, three-winged ovary in the female flowers. The ovary ripens into a membranous capsule, bursting by three valves to liberate numerous flattish or globose seeds. Exemplary yam varieties for which the present invention applies include, but are not limited to, white yam, yellow yam, Kokoro yam, water yam, winged yam, purple yam, Chinese yam, air potato, lesser yam, bitter yam, and cush-cush yam.

Depending on the species, yam grows for six to ten months and is dormant for two to four months, these two phases usually corresponding to the wet season and the dry season. For maximum yield the yam requires an annual rainfall of over 1,500 mm distributed uniformly throughout the growing season. White, yellow and water yams normally produce annually a single large tuber, often weighing 5-10 kg.

Yams yield abundantly with little effort, and adapt readily to diverse climates as long as the climate is cool and moist enough for the plants to gather sufficient water from the soil to form the starchy tubers. However, yams do not keep very well in storage and are vulnerable to molds that feed on the stored tubers, quickly turning them rotten. The major problems presently facing yam production are its high labor requirement, its low yield compared to crops such as cassava or sweet potato, the relatively large amount of planting material that is required and its long growing season.

### Potato

There are about five thousand potato varieties worldwide. Three thousand of them are found in the Andes alone, mainly in Peru, Bolivia, Ecuador, Chile, and Colombia. They belong to eight or nine species, depending on the taxonomic school. Apart from the five thousand cultivated varieties, there are about 200 wild species and subspecies, many of which can be cross-bred with cultivated varieties, which has been done repeatedly to transfer resistances to certain pests and diseases from the gene pool of wild species to the gene pool of cultivated potato species.

The major species grown worldwide is Solanum tuberosum (a tetraploid with 48 chromosomes), and modern varieties of this species are the most widely cultivated. There are also four diploid species (with 24 chromosomes): S. stenotomum, S. phureja, S. goniocalyx, and S. ajanhuiri. There are two triploid species (with 36 chromosomes): S. chaucha and S. juzepczukii. There is one pentaploid cultivated species (with 60 chromosomes): S. curtilobum. There are two major subspecies of Solanum tuberosum: andigena, or Andean; and tuberosum, or Chilean. The Andean potato is adapted to the short-day conditions prevalent in the mountainous equatorial and tropical regions where it originated. The Chilean potato, native to the Chiloé Archipelago, is adapted to the long-day conditions prevalent in the higher latitude region of southern Chile.

Most modern potatoes grown in North America arrived through European settlement and not independently from the South American sources. However, at least one wild potato species, Solanum fendleri, is found as far north as Texas and used in breeding for resistance to a nematode species that attacks cultivated potatoes. A secondary center of genetic variability of the potato is Mexico, where important wild species that have been used extensively in modern breeding are found, such as the hexaploid Solanum demissum, as a source of resistance to the devastating late blight disease. Another relative native to this region, Solanum bulbocastanum, has been used to genetically engineer the potato to resist potato blight.

Potatoes yield abundantly with little effort, and adapt readily to diverse climates as long as the climate is cool and moist enough for the plants to gather sufficient water from the soil to form the starchy tubers. Potatoes do not keep very well in storage and are vulnerable to molds that feed on the stored tubers, quickly turning them rotten. By contrast, grain can be stored for several years without much risk of rotting.

Potato contains vitamins and minerals, as well as an assortment of phytochemicals, such as carotenoids and natural phenols. Chlorogenic acid constitutes up to 90% of the potato tuber natural phenols. Others found in potatoes are 4-O-caffeoylquinic acid (crypto-chlorogenic acid), 5-O-caffeoylquinic (neo-chlorogenic acid), 3,4-dicaffeoylquinic and 3,5-dicaffeoylquinic acids.[58] A medium-size 150 g (5.3 oz) potato with the skin provides 27 mg of vitamin C (45% of the Daily Value (DV)), 620 mg of potassium (18% of DV), 0.2 mg vitamin B6 (10% of DV) and trace amounts of thiamin, riboflavin, folate, niacin, magnesium, phosphorus, iron, and zinc. The fiber content of a potato with skin (2 g) is equivalent to that of many whole grain breads, pastas, and cereals.

In terms of nutrition, the potato is best known for its carbohydrate content (approximately 26 grams in a medium potato). The predominant form of this carbohydrate is starch. A small but significant portion of this starch is resistant to digestion by enzymes in the stomach and small intestine, and so reaches the large intestine essentially intact. This resistant starch is considered to have similar physiological effects and health benefits as fiber: It provides bulk, offers protection against colon cancer, improves glucose tolerance and insulin sensitivity, lowers plasma cholesterol and triglyceride concentrations, increases satiety, and possibly even reduces fat storage. The amount of resistant starch in potatoes depends much on preparation methods. Cooking and then cooling potatoes significantly increases resistant starch. For example, cooked potato starch contains about 7% resistant starch, which increases to about 13% upon cooling.

Potato has been bred into many standard or well-known varieties, each of which has particular agricultural or culinary attributes. In general, varieties are categorized into a few main groups, such as russets, reds, whites, yellows (also called Yukons) and purples—based on common characteristics. For culinary purposes, varieties are often described in terms of their waxiness. Floury, or mealy (baking) potatoes have more starch (20-22%) than waxy (boiling) potatoes (16-18%). The distinction may also arise from variation in the comparative ratio of amylose and amylopectin. In some embodiments, the potato variety of the present invention is a White Rounds potato variety, a Red Rounds potato variety, or a Russet potato variety.

In some embodiments, the potato is a variety deposited in the International Potato Center based in Lima, Peru, which holds an ISO-accredited collection of potato germplasm. The international Potato Genome Sequencing Consortium announced in 2009 that they had achieved a draft sequence of the potato genome. The potato genome contains 12 chromosomes and 860 million base pairs making it a medium-sized plant genome. More than 99 percent of all current varieties of potatoes currently grown are direct descendants of a subspecies that once grew in the lowlands of south-central Chile. In some other embodiments, the potato is a variety included in the European Cultivated Potato Databased (ECPD), the Potato Association of America, the Cornell Potato Varieties List, the Canadian Registry of Potato Varieties, the UPOV potato varieties collection, etc.

Exemplary potato varieties for which the present invention applies include, but are not limited to, Adirondack Blue, Adirondack Red, Agata, Almond, Apline, Alturas, Amandine, Annabelle, Anya, Arran Victory, Atlantic, Avalanche, Bamberg, Bannock Russet, Belle de Fontenay, BF-15, Bildtstar, Bintje, Blazer, Busset, Blue Congo, Bonnotte, British Queens, Cabritas, Camota, Canela Russet, Cara, Carola, Chelina, Chiloé, Cielo, Clavela Blanca, Desiree, Estima, Fianna, Fingerling, Flava, German Butterball, Golden Wonder, Goldrush, Home Guard, Innovator, Irish Cobbler, Jersey Royal, Kennebec, Kerr's Pink, Kestrel, Keuka Gold, King Edward, Kipfler, Lady Balfour, Langlade, Linda, Marcy, Marfona, Maris Piper, Marquis, Megachip, Monalisa, Nicola, Pachacoña, Pike, Pink Eye, Pink, Fir Apple, Primura, Ranger Russet, Ratte, Record, Red LaSoda, Red Norland, Red Pontiac, Rooster, Russet Burbank, Russet Norkotah, Selma, Shepody, Sieglinde, Silverton, Russet, Sirco, Snowden, Spunta, Stobrawa, Superior, Vivaldi, Vitelotte, Yellow Finn, Yukon Gold, blue potato varieties (e.g., Cream of the Crop), Igorota, Solibao, Ganza, Eliane, BelRus, Centennial Russet, Century Russet, Frontier Russet, Hilite Russet, Krantz, Lemhi Russet, Nooksack, Norgold Russet, Norking Russet, Ranger Russet, Russet Burbank, Russet Norkotah, Russet Nugget, Allegany, Atlantic, Beacon Chipper, CalWhite, Cascade, Castile, Chipeta, Gemchip, Irish Cobbler, Itasca, Ivory Crisp, Kanona, Katandin, Kennebec, Kennebec Story, La Chipper, Lamoka, Monona, Monticello, Norchip, Norwis, Onaway, Chieftain, La Rouge, NorDonna, Norland, Red La Soda, Red Pontiac, Red Ruby, Sangre, Viking, Ontario, Pike, Sebago, Shepody, Snowden, Superior, Waneta, White Pearl, White Roseand, Mazama, and all genetically modified varieties. More potato varieties are described in Clough et al., Hort Technology, 2010, 20(1):250-256; Potato Variety Handbook, National Institute of Agricultural Botany, 2000; Chase et al., North American Potato Variety Inventory, Potato Association of America, 1988, each of which is incorporated by reference in its entirety.

Traditional potato growth has been divided into five phases. During the first phase, sprouts emerge from the seed potatoes and root growth begins. During the second, photosynthesis begins as the plant develops leaves and branches. In the third phase stolons develop from lower leaf axils on the stem and grow downwards into the ground and on these stolons new tubers develop as swellings of the stolon. This phase is often (but not always) associated with flowering. Tuber formation halts when soil temperatures reach 80° F. (26.7° C.); hence potatoes are considered a cool-season crop. Tuber bulking occurs during the fourth phase, when the plant begins investing the majority of its resources in its newly formed tubers. At this stage, several factors are critical to yield: optimal soil moisture and temperature, soil nutrient availability and balance, and resistance to pest attacks. The final phase is maturation: The plant canopy dies back, the tuber skins harden, and their sugars convert to starches.

Potato can be used to produce alcoholic beverages, food for human and domestic animals. The potato starch can be used in the food industry as thickeners and binders of soups and sauces, in the textile industry as adhesives, and for the manufacturing of papers and boards. Waste potatoes can be used to produce polylactic acid for plastic products, or used as a base for biodegradable packaging. Potato skins, along with honey, are a folk remedy for burns.

### Cannabis

Cannabis, more commonly known as marijuana, is a genus of flowering plants that includes at least three species, Cannabis sativa, Cannabis indica, and Cannabis ruderalis as determined by plant phenotypes and secondary metabolite profiles. In practice however, cannabis nomenclature is often used incorrectly or interchangeably. Cannabis literature can be found referring to all cannabis varieties as “sativas” or all cannabinoid producing plants as “indicas”. Indeed the promiscuous crosses of indoor cannabis breeding programs have made it difficult to distinguish varieties, with most cannabis being sold in the United States having features of both sativa and indica species.

Cannabis is one of the world's oldest and most useful cultivated genus of plants. Humans have used hemp varieties of cannabis for the production of industrial materials, including food, paper, textiles, plastics, detergents, and biofuels. Humans also have a long history of using psychoactive varieties of cannabis for medical and recreational applications. Cannabis has long been used for drug and industrial purposes, fiber (hemp), for seed and seed oils, for medicinal purposes, and as a recreational drug. Industrial hemp products are made from Cannabis plants selected to produce an abundance of fiber. Some Cannabis strains have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Interest in psychoactive varieties of cannabis has recently exploded following the relaxation drug laws within the United States, and with the discovery of previously unrecognized applications for cannabis in the treatment of human diseases such as diabetes, epilepsy, schizophrenia, and cancer.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

All known strains of Cannabis are wind-pollinated and the fruit is an achene. Most strains of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). http://en.wikipedia.org/wiki/Cannabis—cite_note-schultes2001a-21 Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the cannabis plant (Rudolf Brenneisen, 2007, Chemistry and Analysis of Phytocannabinoids (cannabinoids produced by cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). http://en.wikipedia.org/wiki/Cannabis—cite_note-26 The two cannabinoids usually produced in greatest abundance are cannabidiol (CBD) and/or Δ⁹-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

The cannabinoids in cannabis plants include, but are not limited to, Δ⁹-Tetrahydrocannabinol (Δ⁹-THC), Δ⁸-Tetrahydrocannabinol (Δ⁸-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ⁹-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

In addition to cannabinoids, cannabis also produces over 120 different terpenes (Russo 2011, Taming THC: potential cannabis synergy and phytocannabinoid-terpenoid entourage effects, British Journal of Pharmacology, 163:1344-1364). Within the context and verbiage of this document the terms ‘terpenoid’ and ‘terpene’ are used interchangeably. Examples of representative terpines include, but are not limited to, terpinolene, alpha phelladrene, beta ocimene, carene, limonene, gamma terpinene, alpha pinene, alpha terpinene, beta pinene, fenchol, camphene, alpha terpineol, alpha humulene, beta caryophyllene, linalool, cary oxide, and myrcene.

Cannabinoids are odorless, so terpenoids are responsible for the unique odor of cannabis, and each variety has a slightly different profile that can potentially be used as a tool for identification of different varieties or geographical origins of samples (Hillig 2004. “A chemotaxonomic analysis of terpenoid variation in Cannabis” Biochem System and Ecology 875-891). It also provides a unique and complex flavor smell, and effect profile for each variety that is appreciated by both novice users and connoisseurs. In addition to many circulatory and muscular effects, some terpenes interact with neurological receptors. A few terpenes produced by cannabis plants also bind weakly to Cannabinoid receptors. Some terpenes can alter the permeability of cell membranes and allow in either more or less THC, while other terpenes can affect serotonin and dopamine chemistry as neurotransmitters. Terpenoids are lipophilic, and can interact with lipid membranes, ion channels, a variety of different receptors (including both G-protein coupled odorant and neurotransmitter receptors), and enzymes. Some are capable of absorption through human skin and passing the blood brain barrier.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in cannabis) bear staminate and pistillate structures on individual flowers, whereas monoecious plants bear male and female flowers at different locations on the same plant.

The life cycle of cannabis varies with each variety but can be generally summarized into germination (or rooting/recovery after asexual propagation), vegetative growth, and reproductive stages. Because of heavy breeding and selection by humans, most cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a cannabis plant contain a single leaflet, with subsequent leaves developing in opposite formation. In some embodiments, subsequent leaves develop with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, cannabis plants can grow up to 2.5 inches a day, and are capable of reaching heights of up to 20 feet. Indoor growth pruning techniques tend to limit cannabis size through careful pruning of apical or side shoots.

Although, some cannabis varieties will flower without the need for external stimuli, most varieties have an absolute requirement for inductive photoperiods in the form of short days or long nights to induce fertile flowering. The first sign of flowering in cannabis is the appearance of undifferentiated flower primordial along the main stem of the nodes. At this stage, the sex of the plants are still not distinguishable. As the flower primordia continue to develop, female (pistillate), and male (staminate) flowers can be distinguished.

For most cannabinoid producing purposes, only female plants are desired. The presence of male flowers is considered undesirable as pollination is known to reduce the cannabinoid yield, and potentially ruin a crop. For this reason, most cannabis is grown “sinsemilla” through vegetative (i.e., asexual) propagation. In this way, only female plants are produced and no space is wasted on male plants.

Commercial production of these medicinal and recreational cannabis varieties however, has been slowed down by the lack of true-breeding psychoactive genetics. Indeed, most popular cannabis strains in the market do not have fixed genetics, and are unable to produce uniform progeny when propagated through seeds. Modern cannabis production techniques thus rely on asexual cuttings of single cannabis “mother” plants to produce uniform crops of genetically identical plants. Current asexual reproduction techniques however, still represent a major bottleneck in cannabis production yields. Improper techniques and incorrect hormones and nutrient formulations result in low propagation yields, and slow rooting and recovery of successful clones. Although asexual reproduction of cannabis is somewhat easily performed, its inherent constraints of time, space and resources severely limits the total number of plants that can be produced in large scale commercial operations. Furthermore, asexual reproduction of cannabis is constantly plagued by a host of other problems, including, but not limited to, abiotic disorders (e.g., nutrition, light quality and quantity, water availability, etc.); pathogens (e.g., Powdery Mildew and Pythium root rots); mites (e.g., two spotted spider mites and hemp russet mite); aphids (e.g., rice root aphid and hop aphid); white flies; viruses (e.g., Tobacco Mosaic Virus) and fungus gnats.

The present disclosure generally relates to compositions, systems, and methods for cannabis tissue culture and the Cannabis cells, calli, tissues, plant parts and whole plants produced and/or regenerated from such tissue culture. The disclosures of the present invention circumvent many of the problems associated with the asexual reproduction of cannabis.

This disclosure describes, inter alia, compositions, systems and methods for producing and maintaining Cannabis cell explants, Cannabis tissue explants, Cannabis cell cultures, isolated Cannabis cell cultures, Cannabis tissue cultures, isolated Cannabis tissue cultures, Cannabis callus, and isolated Cannabis callus. In some embodiments, the compositions, systems and methods of the present invention are used to produce clones of Cannabis plants, genotypes, strains, and/or varieties. This can be accomplished, e.g., via regeneration of whole plants from the Cannabis tissue cultures produced according to the present invention.

The compositions, systems and methods of the present invention can be used for the tissue culturing and plant regeneration of any Cannabis germplasm. With twenty-six states and the District of Columbia in the United States legalizing marijuana in some form (i.e., for medical and/or recreational use), Cannabis germplasms, strains, varieties and/or lines are publicly and commercially available. U.S. Pat. No. 6,630,507 issued on Oct. 7, 2003 and assigned on the patent face to The United States of America, is directed to methods of treating diseases caused by oxidative stress by administering therapeutically effective amounts of a cannabidiol (CBD) cannabinoid from cannabis plants that has substantially no binding to the N-methyl-D-aspartate (NMDA) receptor, wherein the CBD acts as an antioxidant and neuroprotectant. A search of the U.S.P.T.O Patent Application Information Retrieval (PAIR) system also reveals the existence of thousands of cannabis-related applications and issued patents including U.S. Pat. No. 8,034,843 (use of cannabinoids for treating nausea, vomiting, emesis, motion sickness), U.S. Pat. No. 7,698,594 (cannabinoid compositions for treatment of pain), and U.S. Pat. No. 8,632,825 (anti-tumoural effects of cannabinoid combinations) among many others. Some examples of publicly-disclosed Cannabis germplasms, strains, varieties and/or lines each of which produce different amounts and/or ratios of cannabis metabolites can be found, e.g., in U.S. Pat. Nos. 9,095,554; 9,370,164; and 9,642,317; and U.S. Published Patent Application Nos. 20110098348; 20140287068; 20160324091; and 20160360721, each of which is specifically incorporated by reference herein in its entireties, including all of the tables and figures. Specific strains of cannabis are disclosed in U.S. Published Patent Application Nos. 20140245494 and 20160073567 (‘Cannabis Plant Named Erez’); 20140245495 20160073568 (‘Cannabis Plant Named Midnight’); 20140259228 and 20160073566 (‘Cannabis Plant Named Avidekel’); 20160000843 (‘High Cannabinol Cannabis Strains’); 20160345477 (‘Cannabis Plant Named Ecuadorian Sativa’); and 20170172040 (‘Cannabis Plant Named Katelyn Faith’).

In some embodiments, any medium, or combinations thereof, of the present disclosure may be utilized in cannabis cultivation.

Also, the present invention teaches any medium, or combinations thereof, of the present disclosure may be utilized in hemp culturing and/or cultivation. Hemp, or industrial hemp (from Old English hcenep), typically found in the northern hemisphere, is a variety of the Cannabis sativa plant species that is grown specifically for the industrial uses of its derived products. It is one of the fastest growing plants and was one of the first plants to be spun into usable fiber 10,000 years ago. It can be refined into a variety of commercial items including paper, textiles, clothing, biodegradable plastics, paint, insulation, biofuel, food, and animal feed.

Modern attempts at classifying cannabis now focus on each individual plant's morphologies and intended use. At the broadest level, cannabis plants can be categorized as either hemp or ‘drug type’ cannabis (“Marijuana”) strains. Hemp strains generally refer to fiber-producing cannabis plants that exhibit tall unbranched (sativa-like) morphologies. These plants have been bred to focus their energies on producing long fibrous stalks and generally only accumulate low levels of cannabinoid drug compounds, which can be used for recreational or medicinal applications. “Drug type” cannabis (“Marijuana”) strains on the other hand, refer to plants that are designed for human recreational or medicinal consumption. These plants focus their energies on producing large numbers of resinous flowers, and thus typically exhibit shorter, highly branched morphologies adapted to indoor grows.

Although marijuana cannabis stains as a drug and industrial hemp both derive from the Cannabis family and contain the psychoactive component tetrahydrocannabinol (THC), they are distinct strains with unique phytochemical compositions and uses. Hemp has lower concentrations of THC and higher concentrations of cannabidiol (CBD), which decreases or eliminates its psychoactive effects. The legality of industrial hemp varies widely between countries. Some governments regulate the concentration of THC and permit only hemp that is bred with an especially low THC content.

In contrast to cannabis for medical use, varieties grown for fiber and seed have less than 0.3% THC and are unsuitable for producing hashish and marijuana (Sawler J et al, 2015, PLOS One. 10(8): e0133292). Present in industrial hemp, cannabidiolis a major constituent among some 560 compounds found in hemp. The major differences between the two types of plants are the appearance, and the amount of Δ⁹-tetrahydrocannabinol (THC) secreted in a resinous mixture by epidermal hairs called glandular trichomes, although they can also be distinguished genetically. Oilseed and fiber varieties of Cannabis approved for industrial hemp production produce only minute amounts of this psychoactive drug, not enough for any physical or psychological effects. Typically, hemp contains below 0.3% THC, while cultivars of Cannabis grown for medicinal or recreational use can contain anywhere from 2% to over 20%.

Furthermore, President Obama signed the Agricultural Act of 2014, or the 2014 Farm Bill, which included Section 7606 allowing for universities and state departments of agriculture to begin cultivating industrial hemp for limited purposes. Specifically, the law allows universities and state departments of agriculture to grow or cultivate industrial hemp if: “(1) the industrial hemp is grown or cultivated for purposes of research conducted under an agricultural pilot program or other agricultural or academic research; and (2) the growing or cultivating of industrial hemp is allowed under the laws of the state in which such institution of higher education or state department of agriculture is located and such research occurs.” For purposes of the Farm Bill, industrial hemp is defined as Cannabis sativa L., having a THC concentration ≤0.3%.

The law also requires that the grow sites be certified by—and registered with—their state. A bipartisan group of U.S. senators introduced the Industrial Hemp Farming Act of 2015 that would allow American farmers to produce and cultivate industrial hemp. The bill would remove hemp from the controlled substances list as long as it contained no more than 0.3 percent THC. The U.S. Department of Agriculture, in consultation with the U.S. Drug Enforcement Agency (DEA) and the U.S. Food and Drug Administration, released a Statement of Principles on Industrial Hemp in the Federal Register on Aug. 12, 2016, on the applicable activities related to hemp in the 2014 Farm Bill.

Hemp is used to make a variety of commercial and industrial products including rope, clothes, food, paper, textiles, plastics, insulation and biofuel. The bast fibers can be used to make textiles that are 100% hemp, but they are commonly blended with other organic fibers such as flax, cotton or silk, to make woven fabrics for apparel and furnishings. The inner two fibers of the plant are more woody and typically have industrial applications, such as mulch, animal bedding and litter. When oxidized (often erroneously referred to as “drying”), hemp oil from the seeds becomes solid and can be used in the manufacture of oil-based paints, in creams as a moisturizing agent, for cooking, and in plastics. Hemp seeds have been used in bird feed mix as well. Also, more than 95% of hemp seed sold in the European Union was used in animal and bird feed according to the 2013 research data. Thus, the hemp seed can be used for animal and bird feed.

In some embodiments, any medium, or combinations thereof, of the present disclosure may be utilized in hemp culture and/or hemp cultivation.

### Media

The present invention provides media comprising compounds with unique types, concentrations, and combinations. In some embodiments, the medium is a liquid, semi-liquid, solid or semi-solid medium.

In some embodiments, liquid cultures offer several advantages. The liquid cultivation saves time, because it enables replacement of the full medium in the vessel containing multiple explants be made at once, instead of individual transfers of single plant. In addition, a liquid culture results in increased shoot length because a larger area of the explant can get in contact with the medium.

The present invention provides media used for in vitro micropropagation of plants, such as bamboo plants and agricultural plants. Media useful for the production of perennials, grasses and phyto-pharmaceutical plants, is also provided herein.

Medium and methods used for plant micropropagation have been described at least in M. R. Ahuja, Micropropagation of woody plants, Springer, 1993, ISBN 0792318072, 9780792318071; Narayanaswamy, Plant cell and tissue culture, Tata McGraw-Hill Education, 1994, ISBN 0074602772, 9780074602775; Singh and Kumar, Plant Tissue Culture, APH Publishing, 2009, ISBN 8131304396, 9788131304396; Y. P. S. Bajaj High-tech and micropropagation V, Springer, 1997, ISBN 3540616063, 9783540616061; Trigiano and Gray, Plant Tissue Culture, Development and Biotechnology, CRC Press, 2010, ISBN 1420083260, 9781420083262; Gupta and Ibaraki, Plant tissue culture engineering Volume 6 of Focus on biotechnology, Springer, 2006, ISBN 1402035942, 9781402035944; Jain and Ishii, Micropropagation ofwoody trees andfruits Volume 75 of Forestry sciences, Springer, 2003, ISBN 1402011350, 9781402011351; and Aitken-Christie et al., Automation and environmental control in plant tissue culture, Springer, 1995, ISBN 0792328418, 9780792328414, each of which is incorporated herein by reference in its entirety.

Medium and methods for bamboo micropropagation have been described in International Patent Application Publication No. WO2011100762, which is incorporated herein by reference in its entirety.

The physical state of the media can vary by the incorporation of one or more gelling agents. Any gelling agent known in the art that is suitable for use in plant tissue culture media can be used. Agar is most commonly used for this purpose. Examples of such agars include Agar Type A, E or M and Bacto™ Agar. Other exemplary gelling agents include carrageenan, gellan gum (commercially available as PhytaGel™, Gelrite® and Gelzan™), alginic acid and its salts, and agarose. Blends of these agents, such as two or more of agar, carrageenan, gellan gum, agarose and alginic acid or a salt thereof also can be used. In some embodiments, no gelling agent or very little gelling agent is used for a liquid medium.

In some embodiments, the media comprise one or more minimum nutrition necessary for plant growth, such as amino acids, macroelements, microelements, aluminum, boron, chlorine (chloride), chromium, cobalt, copper, iodine, iron, lead, magnesium, manganese, molybdenum, nitrogen (nitrates), potassium, phosphorous (phosphates), silicon, sodium, sulphur (sulphates), titanium, vanadium, zinc, inositol and undefined media components such as casein hydrolysates or yeast extracts. For example, the media can include any combination of NH₄NO₃; KNO₃; Ca(NO₃)₂; K₂SO₄; MgSO₄; MnSO₄; ZnSO₄; K₂SO₅; CuSO₄; CaCl₂; KI; CoCl₂; H₃BO₃; Na₂MoO₄; KH₂PO₄; FeSO₄; Na₂EDTA; Na₂H₂PO₄; inositol (e.g., myo-inositol); thiamine; pyridoxine; nicotinic acid; glycine; and riboflavin. It is known to those in the art that one or more components mentioned above can be omitted without affecting the function of the media.

The media can comprise one or more carbon source, such as a sugar. Non-limiting exemplary sugars include sucrose, glucose, maltose, galactose and sorbitol or combinations thereof.

In some embodiments, the media can comprise one inorganic salts, growth regulators, carbon source, and/or vitamins. In some embodiments, at least one of the vitamins is provided by the Murashige and Skoog medium salts (Murashige and Skoog, 1962), Woody Plant (WPM) tissue culture salts, Driver Kuniyuki Walnut (DKW) tissue culture, and/or functional variations thereof.

The media further comprise one or more effective amount of plant growth regulators (PGRs). Examples of plant growth regulators include plant hormones, such as auxins and compounds with auxin-like activity, cytokinins and compounds with cytokinin-like activity. The term “cytokinin” refers to a class of plant growth regulators that are characterized by their ability to stimulate cell division and shoot organogenesis in tissue culture. Non-limiting examples of cytokinins include, N⁶-benzylaminopurine (BAP) (a.k.a. N⁶-benzyladenine (BA)), meta-topolin, zeatin, kinetin, thiadiazuron (TDZ), meta-topolin, 2-isopentenyladenine (a.k.a., 6-γ-γ-(dimethylallylamino)-purine or 2ip), adenine hemisulfate, dimethylallyladenine, 4-CPPU (N-(2-chloro-4-pyridyl)-N′-phenylurea)), and analogs thereof. The term “auxin” refers to a class of plant growth regulators that are characterized principally by their capacity to stimulate cell division in excised plant tissues. In addition to their role in cell division and cell elongation, auxins influence other developmental processes, including root initiation. Non-limiting examples of f3-naphthoxyacetic acid (NAA), 2,4-Dichlorophenoxy acetic acid (2,4-D), indole-3-butyric acid (IBA), indole-3-acetic acid (IAA), picloram, and analogs thereof. More cytokinins and auxins are described in WO2011100762, U.S. Pat. No. 5,211,738, US20100240537, US20060084577, US20030158043, and Aremu et al., 2011, which are incorporated by reference in their entireties. In some embodiments, the cytokinin is BAP or any functional variant thereof. In some embodiments, the auxin is IAA or any functional variant thereof.

In some embodiments, other plant growth regulators can be added in the media to improve cell growth and development. In some embodiments, growth inhibitors and/or growth retardants are used.

Non-limiting examples of growth inhibitors include, abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, and 2,3,5-tri-iodobenzoic acid, derivatives thereof, or combinations thereof.

Non-limiting examples of growth retardant include, ancymidol (e.g., A-Rest®, Abide®), chlormequat chloride (e.g., Chlormequat E-Pro®, Citadel®, Cycocel®), daminozide (e.g., B-Nine®, Dazide®), ethephon (e.g., Florel®), flurprimidol (e.g., Topflor®), paclobutrazol (e.g., Bonzi®, Downsize®, Paczol®, Piccolo®), mefluidide, paclobutrazol, tetcyclacis and uniconazole (e.g., Concise®, Sumagic®). In some embodiments, the growth retardant is an gibberellic acid (GA3) antagonist which can inhibit GA3 pathway, for example, ancymidol, tannins, paclobutrazol (PBZ), (2-Chloroethyl) trimethylammonium chloride, abscisin, exogenous ABA, derivatives thereof, or combinations thereof.

Exemplary concentrations of the components described above are shown in Table 1. The concentrations of these components can be adjusted based on plant species, tissue type, and purposes, etc, without substantially affecting the media function. The exemplary concentrations are by no means limiting, and merely encompass some of the embodiments.

TABLE 1
Exemplary Concentrations

| | Concentrations |
|---|---|
| | (mg/L in all unless |
| Component | otherwise noted) |
| NH₄NO₃ | about 800-about 2500 |
| KNO₃ | about 900-about 3000 |
| Ca(NO₃)₂ | 0-about 800 |
| K₂SO₄ | 0-about 800 |
| MgSO₄ | about 150-about 550 |
| MnSO₄ | about 8.0-about 26.0 |
| ZnSO₄ | about 4.0-about 12.0 |
| CuSO₄ | about 0.010-about 0.4 |
| CaCl₂ | about 200-about 660 |
| KI | about 0.4-about 1.5 |
| CoCl₂ | about 0.010-about 0.4 |
| H₃BO₃ | about 3.0-about 9.0 |
| Na₂MoO₄ | about 0.10-about 0.4 |
| KH₂PO₄ | about 80-about 250 |
| FeSO₄ | about 25-about 90 |
| Na₂EDTA | about 35-about 120 |
| Na₂H₂PO₄ | about 0-250/about 80-250 |
| myo-Inositol | about 50-about 150 |
| Thiamine | about 0.2-about 0.6 |
| Pyridoxine | about 0.1-about 10 |
| Nicotinic acid | about 0.1-about 10 |
| Sugar | about 10 g/L-about 100 g/L |
| Glycine | about 0-about 5 |
| Riboflavin | about 0-about 5 |
| Ascorbic Acid | about 0-about 5 |
| Gelling agent* | about 2.5 g/L-about 8.0 g/L |

*The amount of gelling agent may vary depending on the type of the agent, and the type of the media (e.g., semi-solid or solid media)

As used herein and in the claims, where the term “about” is used with a numerical value, the numerical value may vary from the explicit number; the variation will be ±10%.

Optionally, the media further comprise one or more buffering agent. The buffering agent can buffer the salt concentration and/or the pH in the medium. For example, the buffering agent can maintain the pH of the liquid mixture so the pH is kept around about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5 about 6.6, about 6.7, about 6.8, about 6.9, or about 7.0. For example, the pH of the liquid mixture is in a range between about 5.0 to about 7.0. In some embodiments, the buffering agent is 2-(N-morpholino)ethanesulfonic acid (MES), Adenosine deaminase (ADA), piperazine-N,N′-bis(2-ethanesulfonic acid) (PIPES), N-(2-Acetamido)-2-aminoethanesulfonic acid (ACES), Cholamine chloride, etc. In some embodiments, the buffering agent is IViES, and its concentration is about 500-1200 mg/L. In some embodiments, the pH of the medium is maintained at about 5.5 to 6.5, for example, about 5.8. In some embodiments, the pH of the medium is maintained at about 5.0 to 6.0, for example, about 5.7.

If present in a media, each cytokinin can be present in an amount from about 0.001 mg/L-about 10 mg/L and all amounts in between. For example, the concentration of a cytokinin is about 0.001, 0.01, 0.1, 1, 2, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99. or about 100 mg/L. In some embodiment, at least one cytokinin is meta-topolin, and its concentration is about 0.2 to 20 mg/L, for example, about 2 mg/L. In some embodiment, at least one cytokinin is 2ip, and its concentration is about 1 to 10 mg/L, for example, about 4-5 mg/L.

In some embodiments, the media comprise meta-topolin and/or its analogues. In some embodiments, meta-topolin is present in an amount equal to or greater than 1.5 mg/L, equal to or greater than 2.0 mg/L, equal to or greater than 2.5 mg/L, equal to or greater than 3.0 mg/L, equal to or greater than 3.5 mg/L, equal to or greater than 4.5 mg/L or equal to or greater than 5.0 mg/L. In other embodiments, meta-topolin is present in an amount of 3.2 mg/L or 5.36 mg/L. In another embodiment, the amount of meta-topolin cannot be less than 1.5 mg/L, cannot be less than 2.0 mg/L, cannot be less than 2.5 mg/L, cannot be less than 3.0 mg/L, cannot be less than 3.5 mg/L, cannot be less than 4.5 mg/L or cannot be less than 5.0 mg/L. In some embodiments, meta-topolin and/or its analogues can be included in any amount up to 200 mg/L. In some embodiments, the media is used for bamboo micropropagation. In some embodiments, the bamboo plant is selected from the species consisting of Arundinaria, Bambusa, Borinda, chusquea, Dendrocalamus, fargesia, Guadua, Phyllostachys, Pleioblastus and Thamnocalamus.

In some embodiments, the media comprise thidiazuron and/or its analogues. In some embodiments, thidiazuron and/or its analogues can be present at 0.001 mg/L, 0.01, 0.025, 0.05, 0.075, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.50, 1.75, 2.0, 2.25, 2.5, 2.75, 3.5, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, thidiazuron and/or its analogues can also be included in any amount up to 200 mg/L.

If present in a media, each auxin can be present in an amount from about 0.01 mg/L-about 100 mg/L and all amounts in between. For example, the concentration of an auxin is about 0.001, 0.01, 0.02, 0.03, 0.04, 0.05, 0.06, 0.07, 0.08, 0.09, 0.1, 0.5, 1, 2, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or about 100 mg/L. In some embodiments, at least one auxin is IAA, and the concentration is about 0.1 to 10 mg/L, for example, about 1 mg/L. In some embodiments, at least one auxin is NAA, and the concentration is about 0.001 to 1 mg/L, for example, about 0.02 mg/L.

In some embodiments, at least one auxin is NAA, IBA or combination thereof. In some embodiments, at least one auxin is IBA. In some embodiments, NAA or IBA is presented in an initiation medium or a multiplication medium, and the concentration is about 0.01-10 mg/L, for example, about 0.02-1 mg/L. In some embodiments, NAA or IBA is presented in a rooting medium. In some embodiments, the NAA/MA concentration in a rooting medium is about 1 to 10 mg/L, for example, about 1-3 mg/L. In some embodiments, the NAA/IBA concentration in a rooting medium is about 100-1500 mg/L, for example, about 250-1000 mg/L.

In some embodiments, the present invention provides several types of media that are used in in vitro micropropagation of plants of the present disclosure.

The first type of media, referred herein as the initiation media, is similar to or essentially the same as the Murashige and Skoog medium (Murashige and Skoog, 1962), Woody Plant (WPM) tissue culture salts, Driver Kuniyuki Walnut (DKW), but comprise at least one auxin and/or at least one cytokinin. In some embodiments, the sucrose concentration in the propagation and multiplication media is about 20-40 g/L or higher. In some embodiments, at least one auxin is mT. In some embodiments, at least one cytokinin is NAA. In some embodiments, the media comprise at least one gelling agent such as agar. In some embodiments, the concentration of the gelling agent is about 4 to about 10 grams, for example, about 7 grams. In some embodiments, the initiation media do not contain Na2H2PO4. In some embodiments, the initiation media do not contain pyridoxine, nicotinic acid, and/or riboflavin.

The second type of media, referred herein as the micropropagation media or multiplication media, are as the same as, or similar to the initiation media. In some embodiments, the sucrose concentration in the propagation and multiplication media is about 20-40 g/L or higher.

In some embodiments, at least one auxin is mT. In some embodiments, at least one cytokinin is IBA. In some embodiments, the media comprise at least one gelling agent such as agar. In some embodiments, the media comprise at least one gelling agent such as agar. In some embodiments, the concentration of the gelling agent is about 4 to about 10 grams, for example, about 5 grams.

In some embodiments, the initiation medium and/or the multiplication medium comprise MS medium containing double concentration of meso elements (e.g., one or more of CaCl2.2H2O, MgSO4.7H2O, and KH2PO4), double iron, and one or more Gamborg's vitamins (e.g., one or more of myo-inositol, Nictotinic acid, pyridoxine salts, and thiamine salts).

The third type of media, referred herein as the rooting media, are similar to or essentially the same as the Murashige and Skoog medium, Woody Plant (WPM) tissue culture salts, Driver Kuniyuki Walnut (DKW), but comprise at least one auxin. In some embodiments, the auxin is IBA. In some embodiments, the media do not comprise any cytokinin. In some embodiments, the sucrose concentration in the propagation and multiplication media is about 20-40 g/L or at least 60 g/L. In some embodiments, the concentration of IBA is about 0.1 to 10 mg/L. In some embodiments, the concentration of IBA is about 100 to 1500 mg/L. In some embodiments, the concentration of the gelling agent is about 4 to about 10 grams, for example, about 6 grams.

In some embodiments, the micropropagation and multiplication media (or elongation and multiplication media), is similar to or essentially the same as the Murashige and Skoog medium (Murashige and Skoog, 1962), but without any hormone or growth regulator. In some embodiments, the sucrose concentration in the propagation and multiplication media is about 10-20 g/L.

In some embodiments, the pre-tuberization media, comprises one or more cytokinins and one or more auxin. In some embodiments, the cytokinin is 2ip and the auxin is IAA. Alternatively, instead of cytokinin and auxin, the pre-tuberization media comprises one or more plant retardant in low amount, such as ancymidol or analog thereof. In some embodiments, the pre-tuberization media comprise one or more cytokinin, one or more auxin, and one or more growth retardant. In some embodiments, the concentration of 2ip is about 1 to 10 mg/L, for example, about 4-5 mg/L. In some embodiments, the concentration of IAA is about 0.1 to 10 mg/L, for example, about 1 mg/L. In some embodiments, the concentration of ancymidol is about 0.1 to 10 mg/L. In any case, the sucrose concentration is about 20 g/L to about 40 g/L, for example, about 30 g/L.

In some embodiments, the tuberization media, comprises one or more auxin, but does not comprise any cytokinin. Alternatively, instead of auxin, the tuberization media comprises one or more plant retardant, such as ancymidol or analog thereof. In some embodiments, the tuberization media comprise one or more auxin and one or more growth retardant. In some embodiments, the auxin is NAA. In some embodiments, the NAA concentration is about 0.01 to about 0.05 mg/L. In some embodiments, the concentration of ancymidol is about 0.1 to 10 mg/L. In any case, the sucrose concentration in the media is higher compared to the sucrose concentration in the pre-tuberization media. For examples, the sucrose concentration in the tuberization media is about 50 g/L to about 100 g/L or more.

In some embodiments, the media comprise NAA and/or its analogues. In some embodiments, NAA and/or its analogues can be present at 0.001 mg/L, 0.01, 0.0125, 0.015, 0.0175, 0.02, 0.0225, 0.025, 0.0275, 0.03, 0.0325, 0.035, 0.0375, 0.04, 0.0425, 0.045, 0.0475, 0.05, 0.0525, 0.055, 0.0575, 0.06, 0.0625, 0.065, 0.0675, 0.07, 0.0725, 0.075, 0.0775, 0.08, 0.0825, 0.085, 0.0875, 0.09, 0.0925, 0.095, 0.0957, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.50, 1.75, 2.0, 2.25, 2.5, 2.75, 3.5, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, NAA and/or its analogues can be included any amount up to 200 mg/L.

In some embodiments, the media comprise IBA and/or its analogues. In some embodiments, IBA and/or its analogues can be present at 0.001 mg/L, 0.01, 0.025, 0.05, 0.075, 0.08, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1.0, 1.25, 1.50, 1.75, 2.0, 2.25, 2.5, 2.75, 3.0, 3.25, 3.5, 3.75, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, IBA and/or its analogues can be included any amount up to 200 mg/L.

In some embodiments, the media comprise BAP and/or its analogues. In some embodiments, BAP and/or its analogues can be present at 0.001 mg/L, 0.01, 0.025, 0.05, 0.06, 0.07, 0.0725, 0.075, 0.0775, 0.08, 0.0825, 0.085, 0.0875, 0.09, 0.0925, 0.095, 0.0975, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.75, 0.8, 0.85, 0.9, 0.95, 1.0, 1.25, 1.50, 1.75, 2.0, 2.25, 2.5, 2.75, 3.5, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, BAP and/or its analogues can be included any amount up to 200 mg/L.

In some embodiments, the media comprise 2ip and/or its analogues. In some embodiments, 2ip and/or its analogues can be present at 0.001 mg/L, 0.01, 0.025, 0.05, 0.075, 0.08, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.50, 1.75, 2.0, 2.25, 2.5, 2.75, 3.0, 3.5, 4, 0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, 2ip and/or its analogues can be included any amount up to 200 mg/L.

In some embodiments, the media comprise DPU and/or its analogues. In some embodiments, DPU and/or its analogues can be present at 0.001 mg/L, 0.01, 0.025, 0.05, 0.075, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.50, 1.75, 2.0, 2.25, 2.5, 2.75, 3.5, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, DPU and/or its analogues can be included any amount up to 200 mg/L.

In some embodiments, the media comprise CPPU and/or its analogues. In some embodiments, CPPU and/or its analogues can be present at 0.001 mg/L, 0.01, 0.025, 0.05, 0.075, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.50, 1.75, 2.0, 2.25, 2.5, 2.75, 3.5, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/L. In some embodiments, CPPU and/or its analogues can be included any amount up to 200 mg/L.

In some embodiments, one or more cytokinins in combination with one or more other cytokinins or auxins, and auxins in combination with other auxins or cytokinins can also be utilized in ratios. For example, in some embodiments, any two cytokinins and/or auxins in pairs disclosed herein can be included in the following exemplary ratios: 100:1, 95:1, 90:1, 85:1, 80:1, 75:1, 70:1, 65:1, 60:1, 55:1, 50:1, 45:1, 40:1, 35:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1; 9:1, 8:1, 7:1, 6.9:1, 6.8:1, 6.7:1, 6.6:1, 6.5:1, 6.4:1, 6.3:1, 6.2:1, 6.1:1, 6:1, 5.9:1, 5.8:1, 5.7:1, 5.6:1, 5.5:1, 5.4:1, 5.3:1, 5.2:1, 5.1:1, 5:1; 4:1, 3:1, 2:1, 1:1, 0.75:1, 0.5:1, 0.25:1, 0.1:1, 0.075:1, 0.05:1, 0.025:1 or 0.001:1. These ratios can also be utilized between meta-topolin (and analogues) with thidiazuron (and analogues), with NAA (and analogues), with BAP (and analogues), with IBA (and analogues), with 2ip (and analogues), with DPU (and analogues) and/or with CPPU (and analogues). Similarly, the ratios can be utilized between thidiazuron (and analogues) with NAA (and analogues), with BAP (and analogues), with IBA (and analogues), with 2ip (and analogues), with DPU (and analogues) and/or with CPPU (and analogues). The ratios can also be utilized between NAA (and analogues) with BAP (and analogues), with IBA (and analogues), with 2ip (and analogues), with DPU (and analogues) and/or with CPPU (and analogues). The ratios can also be utilized between BAP (and analogues) with IBA (and analogues), with 2ip (and analogues), with DPU (and analogues) and/or with CPPU (and analogues). In short, each of the cytokinins and/or auxins or its analogues can be included with a second cytokinin and/or auxin disclosed herein according to any of the disclosed ratios.

In some embodiments, the present invention provides different types of media that are useful in the production of perennials, grasses and phyto-pharmaceutical plants. In some embodiments, the medium useful for producing perennials, grasses and phyto-pharmaceutical plants is a liquid medium. In some embodiments, the medium is a solid medium.

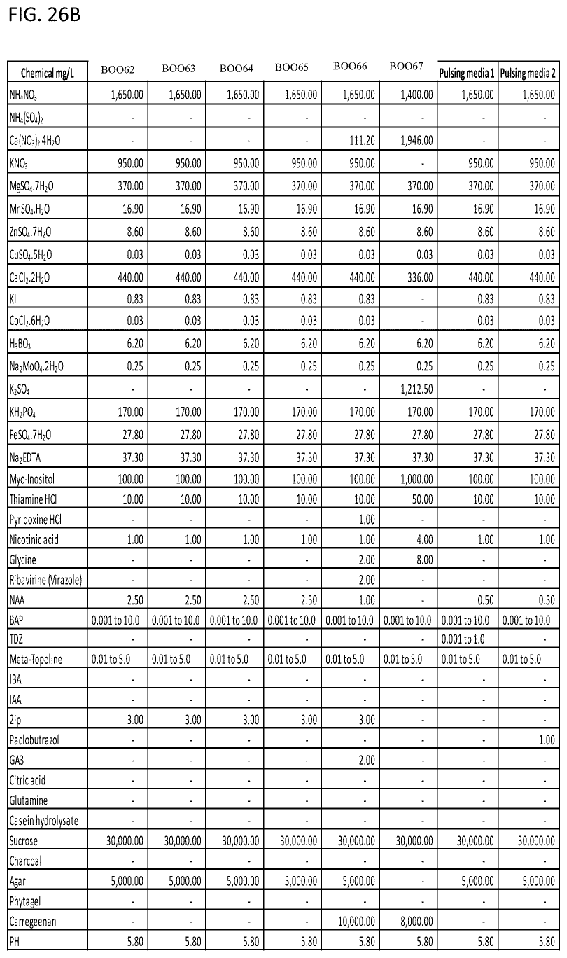

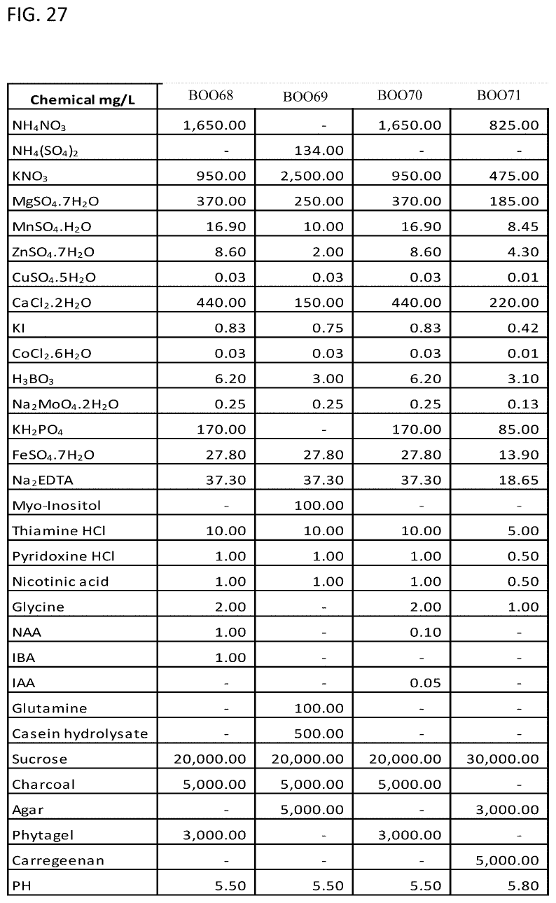

The media useful for the production of perennials, grasses, and phtyo-pharmaceutical plants can be any one of the media described in FIGS. 26A, 26B, and 27. In one embodiment, the media is selected from FIGS. 26A and 26B. In one embodiment, the media is Pulsing media 1 or Pulsing media 2 from FIGS. 26A and 26B. In another embodiment, the media is Pulsing media 1, wherein TDZ is substituted with a different cytokinin, such as BAP, Zeatin, CPPU, or DPU. In another embodiment, the media is BOO68, BOO69, BOO70, or BOO71 from FIG. 27. In some embodiments, BOO68, BOO69, BOO70, BOO71 are used as rooting media. In yet another embodiment, the media is BOO54, BOO55, BOO56, BOO57, BOO58, BOO59, BOO60, BOO61, BOO62, BOO63, BOO64, BOO65, BOO66, or BOO67 media from FIGS. 26A and 26B.

In some embodiments, a combination of the media described in FIGS. 26A and 26B is useful for the production of perennials, grasses, and phyto-pharmaceutical plants. The combination of media can be used sequentially. In one embodiment, the combination comprises Pulsing media 1, Pulsing media 2 or both; and BOO54, BOO55, BOO56, BOO57, BOO58, BOO59, BOO60, BOO61, BOO62, BOO63, BOO64, BOO65, BOO66, or BOO67 media from FIGS. 26A and 26B. In some embodiments, the combination comprises Pulsing media 1, Pulsing media 2 or both; and BOO68, BOO69, BOO70, or BOO71 from FIG. 27. In some embodiments, the combination comprises Pulsing media 1, Pulsing media 2 or both; BOO54, BOO55, BOO56, BOO57, BOO58, BOO59, BOO60, BOO61, BOO62, BOO63, BOO64, BOO65, BOO66, or BOO67 media from FIG. 27; and BOO68, BOO69, BOO70, or BOO71 from FIG. 27. In some embodiments, the Pulsing media 1 in the combination is Pulsing media 1, wherein TDZ is substituted with a different cytokinin, such as BAP, Zeatin, CPPU, or DPU.

In some embodiments, wasabi is grown in BOO51, lily is grown in BOO52 (light) and/or BOO53 (dark), aloe vera is grown in BOO54, ginger is grown in BOO55, grape is grown in BOO56, cannabis is grown in BOO57, garlic is grown in BOO58, onion is grown in BOO59 hakonechloa is grown in BOO60, miscanthus is grown in BOO61, Arundo donax is grown in BOO62, switch grass is grown in BOO63, rice is grown in BOO64, sugar cane is grown in BOO65, echinacea is grown in BOO66, and/or geranium is grown in BOO67.

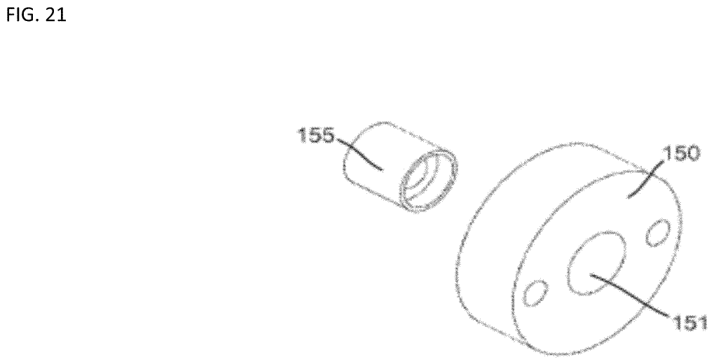

In some embodiments, the present invention provides different types of media that are useful in the reduction of phenolic production in plants, such as from bamboos. The media useful for induction of somatic embryos can be any one of the media described in FIG. 20 (i.e. BOO32, BOO33, BOO34). The media can be useful in reducing the production of a phenolic, such as a polyphenol, in bamboo. In some embodiments, the phenolic is a luteolin derivative, flavone, flavone glycoside or phenolic acid. The reduction of phenolic production can be determined by comparing the phenolic production of a bamboo tissue culture, explants or seed incubated in BOO32, BOO33, or BOO34 media to the phenolic production of a bamboo tissue culture, explants or seed incubated in media that is not BOO32, BOO33, or BOO34. Phenolic production can be determined by HPLC-DAD (diode array detector), LC-MS/MS, or any other method known in the arts. In some embodiments, the medium useful for reducing phenolic production in bamboo is a liquid medium. In some embodiments, the medium is a solid medium.

In some embodiments, the present invention provides different types of media that are useful in the production of virus-free plants, such as agricultural plants. The media useful for the production of virus-free plants can comprise an antiviral. The antiviral can be acemannan, acyclovir, adefovir, alovudine, alvircept, amantadine, aranotin, arildone, atevirdine, pyridine, cidofovir, cipamfylline, cytarabine, desciclovir, disoxaril, edoxudine, enviradene, enviroxime, famdclovir, famotine, fiacitabine, fialuridine, floxuridine, fosarilate, fosfonet, ganciclovir, idoxuridine, kethoxal, lobucavir, memotine, methisazone, penciclovir, pirodavir, somantadine, sorivudine, tilorone, trifluridine, valaciclovir, vidarabine, viroxime, zinviroxime, moroxydine, podophyllotoxin, ribavirine, rimantadine, stallimycine, statolon, tromantadine and xenazoic acid, and their pharmaceutically acceptable salts. In one embodiment, the antiviral is ribavirine (also known as Virazole) or derivatives thereof, such as viramidine (also known as Taribavirin). In some embodiments, a media useful for the production of virus-free plants comprises one or more antivirals.

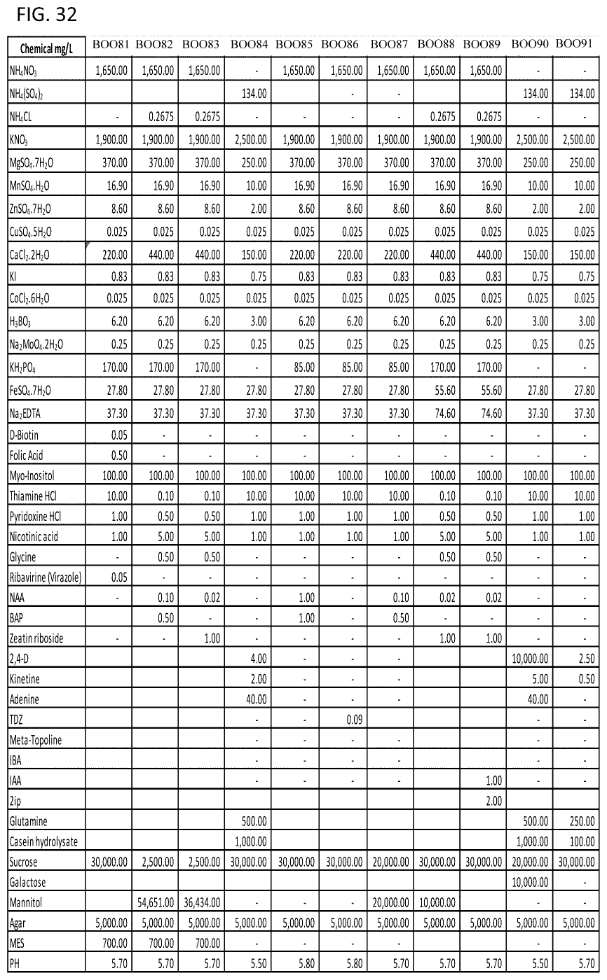

The media useful for the production of virus-free plants can be any one of the media described in FIG. 32. In one embodiment, the media is BOO81. In some embodiments, a combination of the media described in FIG. 32 is useful for the production of virus-free plants. The combination of media can be used sequentially. In one embodiment, the combination comprises BOO81 and a regeneration media. In some embodiments, the combination comprises BOO81 and at least two or more regeneration media.

A regeneration media can promote the growth of explants (such as explants incubated in a medium with an antiviral, such as BOO81) and apical meristems (such as apical meristems from explants incubated in a medium with an antiviral). In some embodiments, the regeneration media comprises an antiviral. In other embodiments, the regeneration media does not comprise an antiviral. The regeneration can be BOO82, BOO83, BOO84, BOO85, BOO86, BOO87, BOO88, BOO89, BOO90, or BOO91, as depicted in FIG. 32.

In one embodiment, the combination of media useful for the production of virus-free plants comprises BOO81, BOO82 and BOO83. In another embodiment, the combination comprises BOO81 and BOO84. In another embodiment, the combination comprises BOO81, BOO85, and BOO86. In yet another embodiment, the combination comprises BOO81, BOO87 and BOO88. In yet another embodiment, the combination comprises BOO81 and BOO89. In yet another embodiment, the combination comprises BOO81 and Sugar Beeet. In yet another embodiment, the combination comprises BOO81 and BOO91. In some embodiments BOO82 and/or BOO83 may be used with potato, BOO84 may be used with tomato, BOO85 and/or BOO86 may be used with cucumber, BOO87 and/or BOO88 may be used with yam, BOO89 may be used with cauliflower, BOO90 may be used with sugar beet, BOO91 may be used with cassava.

In some embodiments, the medium useful for producing virus-free plants is a liquid medium. In some embodiments, the medium is a solid medium.

In other embodiments, the present invention provides at least two types of media that are used in in vitro micropropagation. The first media, referred herein as the “bud induction media (B001)”, comprises at least one strong cytokinin, such as a thidiazuron, or analogs thereof. The second media, referenced herein as the “shoot elongation/maintenance media (BOO2)”, comprises one or more cytokinins other than the cytokinin in the bud induction media (BOO1). For example, the cytokinins are selected from meta-topolin, kinetin, isopentenyl adenine (iP), zeatin, trans-zeatin, zeatin riboside, dihydrozeatin, benzyleadenin (BAP), benzyladenosine ([9R]BAP), analogs thereof, or combination thereof. Other cytokinins available for use in tissue culture can also be substituted for the above cytokinins to achieve similar effects.

In some embodiments, the bud induction medium (BOO1) is a liquid medium. In some embodiments, the bud induction medium (BOO1) is a solid medium. In some embodiments, the shoot elongation/maintenance medium (BOO2) is a liquid medium. In some embodiments, the shoot elongation/maintenance media (BOO2) is a solid media.

The bud induction media (BOO1), the shoot elongation/maintenance media (BOO2), and media useful for producing perennials, grasses and phyto-pharmaceutical plants comprise components of a minimum media for plant tissue culture, such as carbon source and salts. In some embodiments, the media can comprise one or more components selected from NH₄NO₃, KNO₃, Ca(NO₃)₂, K₂SO₄, MgSO₄, MnSO₄, ZnSO₄, CuSO₄, K₂SO₅, CaCl₂, Kl, CoCl₂, H₃BO₃, Na₂MoO₄, KH₂PO₄, FeSO₄, Na₂EDTA, Na₂H₂PO₄, Glycine, myo-Inositol, Thiamine, Pyridoxine, Nicotinic acid, and Riboflavin.

In some embodiments, the media useful for producing perennials, grasses and phyto-pharmaceutical plants comprises only one strong cytokinin, for example, thidiazuron (TDZ), or an analog thereof. In some embodiments, the media comprises one additional cytokinin. In some embodiments, the media further comprises one or more auxin, such as NAA, 2,4-D, IBA, IAA, picloram, or analogs thereof. In other embodiments, the media useful for producing perennials, grasses and phyto-pharmaceutical plants does not comprise a cytokinin or auxin. In yet other embodiments, the media useful for producing perennials, grasses and phyto-pharmaceutical plants does not comprise a plant hormone.

In some embodiments, the concentration of the strong cytokinin (e.g., TDZ) in the media useful for producing perennials, grasses and phyto-pharmaceutical plants, is about 0.25 mg/L (±10%) to about 100 mg/L (±10%), for example, is about 0.2 mg/L (±10%), about 0.5 mg/L (±10%), about 1.0 mg/L (±10%), about 5 mg/L (±10%), about 10 mg/L (±10%), about 20 mg/L (±10%), about 30 mg/L (±10%), about 40 mg/L (±10%), about 50 mg/L (±10%), about 60 mg/L (±10%), about 70 mg/L (±10%), about 80 mg/L (±10%), about 90 mg/L (±10%), or about 100 mg/L (±10%). For example, the concentration of TDZ is about 0.2 (±10%) to about 20 (±10%) mg/L, about 0.4 (±10%) to about 10 (±10%) mg/L, or about 0.5 (±10%) to about 2 (±10%) mg/L.

In some embodiments, the concentration of TDZ or analog thereof in the media useful for producing perennials, grasses and phyto-pharmaceutical plants, is about 0.25 mg/L to about 100 mg/L, for example, about 0.5 mg/L to about 2 mg/L. Thus, the concentration of TDZ or analog thereof can, for example, be about 0.25 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about out 1.5 mg/L, about 2.0 mg/L, about 2.5 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L or about 100 mg/L.

In some embodiments, the bud induction media (BOO1) comprises only one strong cytokinin, for example, thidiazuron (TDZ), or an analog thereof. In some embodiments, the embryo induction media or bud induction media (BOO1) comprises one additional cytokinin. In some embodiments, the embryo induction media or bud induction media (BOO1) further comprises one or more auxin, such as NAA, 2,4-D, IBA, IAA, picloram, or analogs thereof.

In some embodiments, the concentration of the strong cytokinin (e.g., TDZ) in the bud induction media (BOO1) is about 0.25 mg/L (±10%) to about 100 mg/L (±10%), for example, is about 0.2 mg/L (±10%), about 0.5 mg/L (±10%), about 1.0 mg/L (±10%), about 5 mg/L (±10%), about 10 mg/L (±10%), about 20 mg/L (±10%), about 30 mg/L (±10%), about 40 mg/L (±10%), about 50 mg/L (±10%), about 60 mg/L (±10%), about 70 mg/L (±10%), about 80 mg/L (±10%), about 90 mg/L (±10%), or about 100 mg/L (±10%). For example, the concentration of TDZ is about 0.2 (±10%) to about 20 (±10%) mg/L, about 0.4 (±10%) to about 10 (±10%) mg/L, or about 0.5 (±10%) to about 2 (±10%) mg/L.

In some embodiments, the concentration of the strong cytokinin (e.g., TDZ) in the media useful for producing virus-free plants, such as in agricultural plants, is about 0.25 mg/L (±10%) to about 100 mg/L (±10%), for example, is about 0.2 mg/L (±10%), about 0.5 mg/L (±10%), about 1.0 mg/L (±10%), about 5 mg/L (±10%), about 10 mg/L (±10%), about 20 mg/L (±10%), about 30 mg/L (±10%), about 40 mg/L (±10%), about 50 mg/L (±10%), about 60 mg/L (±10%), about 70 mg/L (±10%), about 80 mg/L (±10%), about 90 mg/L (±10%), or about 100 mg/L (±10%). For example, the concentration of TDZ is about 0.2 (±10%) to about 20 (±10%) mg/L, about 0.4 (±10%) to about 10 (±10%) mg/L, or about 0.5 (±10%) to about 2 (±10%) mg/L.

In some embodiments, the concentration of TDZ or analog thereof in the media useful for producing virus-free plants, such as agricultural plants, is about 0.25 mg/L to about 100 mg/L, for example, about 0.5 mg/L to about 2 mg/L. Thus, the concentration of TDZ or analog thereof can, for example, be about 0.25 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about out 1.5 mg/L, about 2.0 mg/L, about 2.5 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L or about 100 mg/L.

In some embodiments, the concentration of TDZ or analog thereof in the bud induction medium (BOO1) is effective to induce shoot buds. In some embodiments, the concentration of TDZ or analog in the bud induction media (BOO1) thereof is about 0.25 mg/L to about 100 mg/L, for example, about 0.5 mg/L to about 2 mg/L. Thus, the concentration of TDZ or analog thereof in the bud induction media (BOO1) can, for example, be about 0.25 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about out 1.5 mg/L, about 2.0 mg/L, about 2.5 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L or about 100 mg/L.

In some embodiments, the bud induction medium (BOO1) and/or the shoot elongation/maintenance medium (BOO2) further comprise one or more auxins. In some embodiments, the auxins are selected from the group consisting of β-naphthoxyacetic acid (NAA), 2,4-Dichlorophenoxy acetic acid (2,4-D), indole-3-butyric acid (IBA), indole-3-acetic acid (IAA), picloram, and analogs of each thereof. For example, the auxin is NAA or analogs thereof.

In some embodiments, the concentration of the auxin in the bud induction media (BOO1) is about 0.01 mg/L (±10%) to about 10 mg/L (±10%), for example, is about 0.01 mg/L (±10%), about 0.05 mg/L (±10%), about 0.1 mg/L (±10%), about 0.5 mg/L (±10%), about 1 mg/L (±10%), about 5 mg/L (±10%), or about 10 mg/L (±10%).

In some embodiments, the shoot elongation/maintenance media (BOO2) comprises one or more cytokinins other than TDZ, such as meta-topolin, kinetin, isopentenyladenine (ip, e.g., 2ip), zeatin, trans-zeatin, zeatin riboside, dihydrozeatin, benzyleadenin (BAP), benzyladenosine ([9R]BAP), analogs thereof. In some embodiments, the shoot elongation/maintenance media (BOO2) further comprise one or more auxin, such as NAA, 2,4-D, IBA, IAA, picloram, or analogs thereof.

In some embodiments, the concentration of cytokinin in the shoot elongation/maintenance media (BOO2) is about 0.01 mg/L (±10%) to about 100 mg/L (±10%), for example, is about 0.01 mg/L (±10%), about 0.05 mg/L (±10%), about 0.1 mg/L (±10%), about 0.5 mg/L (±10%), about 1 mg/L (±10%), about 5 mg/L (±10%), about 10 mg/L (±10%), about 20 mg/L (±10%), about 30 mg/L (±10%), about 40 mg/L (±10%), about 50 mg/L (±10%), about 60 mg/L (±10%), about 70 mg/L (±10%), about 80 mg/L (±10%), about 90 mg/L (±10%), or about 100 mg/L (±10%). In some embodiments, the concentration of the cytokinin in the shoot elongation/maintenance media (BOO2) is about 0.01 (±10%) to about 20 (±10%) mg/L, about 0.1 (±10%) to about 10 (±10%) mg/L, or about 0.25 (±10%) to about 5 (±10%) mg/L.

In some embodiments, the concentration of the one or more cytokinins other than TDZ or an analog thereof in the shoot elongation/maintenance medium (BOO2) is effective to elongate shoots. In some embodiments, the concentration of the one or more cytokinins other than TDZ or an analog thereof in the shoot elongation/maintenance medium (BOO2) from about 0.01 mg/L to about 100 mg/L, for example, from about 0.25 mg/L to about 5 mg/L. Thus, the concentration of one or more cytokinins other than TDZ or analog thereof in the shoot elongation/maintenance media (BOO2) can, for example, be about 0.01 mg/L, about 0.02 mg/L, about 0.03 mg/L, about 0.04 mg/L, about 0.05 mg/L, about 0.06 mg/L, about 0.07 mg/L, about 0.08 mg/L, about 0.09 mg/L, about 0.10 mg/L, about 0.15 mg/L, about 0.20 mg/L, about 0.25 mg/L, about 0.3 mg/L, about 0.35 mg/L, about 0.4 mg/L, about 0.45 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3 mg/L, about 4 mg/L, about 5 mg/L, about 6 mg/L, about 7 mg/L, about 8 mg/L, about 9 mg/L, about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L or about 100 mg/L.

In some embodiments, the concentration of the auxin in the bud induction media (BOO1) is about 0.01 mg/L (±10%) to about 50 mg/L (±10%), for example, is about 0.01 mg/L (±10%), about 0.05 mg/L (±10%), about 0.1 mg/L (±10%), about 0.5 mg/L (±10%), about 1 mg/L (±10%), about 5 mg/L (±10%), about 10 mg/L (±10%), about 20 mg/L (±10%), about 30 mg/L (±10%), about 40 mg/L (±10%), or about 50 mg/L (±10%). In some embodiments, the concentration of the auxin in the shoot elongation/maintenance media (BOO2) is about 0.01 (±10%) to about 20 (±10%) mg/L, about 0.02 (±10%) to about 10 (±10%) mg/L, or about 0.05 (±10%) to about 5 (±10%) mg/L.

Non-limiting concentrations of the components in the bud induction media (BOO1) and shoot elongation/maintenance media (BOO2) are shown in Table 2. One or more components in table 2 can be omitted or replaced without affecting the function of the media. The concentration of each component can be adjusted without affecting the function of the media.

TABLE 2
Exemplary concentrations of bud induction media (BOO1)
and shoot elongation and maintenance media (BOO2).

| | | Shoot elongation |
|---|---|---|
| | Bud Induction media | & maintenance |
| Media | (BOO1) (mg/L) | media (BOO2) (mg/L) |
| NH₄NO₃ | about 800-about 2500, | about 800-about 2500, |
| | e.g., 1650 | e.g., 1650 |
| KNO₃ | about 900-about 3000, | about 900-about 3000, |
| | e.g., 1900 | e.g., 1900 |
| Ca(NO₃)₂ | 0-about 800, | 0-about 800, |
| | e.g., 0 | e.g., 0 |
| K₂SO₄ | 0-about 800, | 0-about 800, |
| | e.g., 0 | e.g., 0 |
| MgSO₄ | about 150-about 550, | about 150-about 550, |
| | e.g., 370 | e.g., 370 |
| MnSO₄ | about 8.0-about 26.0, | about 8.0-about 26.0, |
| | e.g., 16.9 | e.g., 16.9 |
| ZnSO₄ | about 4.0-about 12.0, | about 4.0-about 12.0, |
| | e.g., 8.6 | e.g., 8.6 |
| CuSO₄ | about 0.010-about 0.4, | about 0.010-about 0.4, |
| | e.g., 0.025 | e.g., 0.025 |
| CaCl₂ | about 200-about 660, | about 200-about 660, |
| | e.g., 440 | e.g., 440 |
| KI | about 0.4-about 1.5, | about 0.4-about 1.5, |
| | e.g., 0.83 | e.g., 0.83 |
| CoCl₂ | about 0.010-about 0.4, | about 0.010-about 0.4, |
| | e.g., 0.025 | e.g., 0.025 |
| H₃BO₃ | about 3.0-about 9.0, | about 3.0-about 9.0, |
| | e.g., 6.2 | e.g., 6.2 |
| Na₂MoO₄ | about 0.10-about 0.4, | about 0.10-about 0.4, |
| | e.g., 0.25 | e.g., 0.25 |
| KH₂PO₄ | about 80-about 250, | about 80-about 250, |
| | e.g., 170 | e.g., 170 |
| K₂SO₅ | as necessary | as necessary |
| FeSO₄ | about 25-about 90, | about 25-about 90, |
| | e.g., 55.7 | e.g., 55.7 |
| Na₂EDTA | about 35-about 120, | about 35-about 120, |
| | e.g., 74.6 | e.g., 74.6 |
| Na₂H₂PO₄ | about 80-250, | about 80-250, |
| | e.g., 170 | e.g., 170 |
| myo-Inositol | about 50-about 150, | about 50-about 150, |
| | e.g., 100 | e.g., 100 |
| Thiamine | about 0.2-about 0.6, | about 0.2-about 0.6, |
| | e.g, 0.4 | e.g, 0.4 |
| Sugar | about 15 gr-about | about 15 gr-about |
| | 45 gr, e.g. 30 gr. | 45 gr, e.g. 30 gr. |
| agar (solid) | about 2.5 gr-about | about 2.5 gr-about |
| | 8.0 gr, e.g., 4.5 gr | 8.0 gr, e.g., 4.5 gr |
| PH | about 5.5-6.5, | about 5.5-6.5, |
| | e.g., 5.7 | e.g., 5.7 |
| HORMONES | | |
| NAA | about 0.05 | about 0.01-50 |
| BAP | about 1 | about 0.01-50 |
| TDZ | about 0.25-about 100 | 0 |
| ST-10 | about 0.01-about 50 | about 0.01-about 50 |
| 2ip | 0 | about 6 |

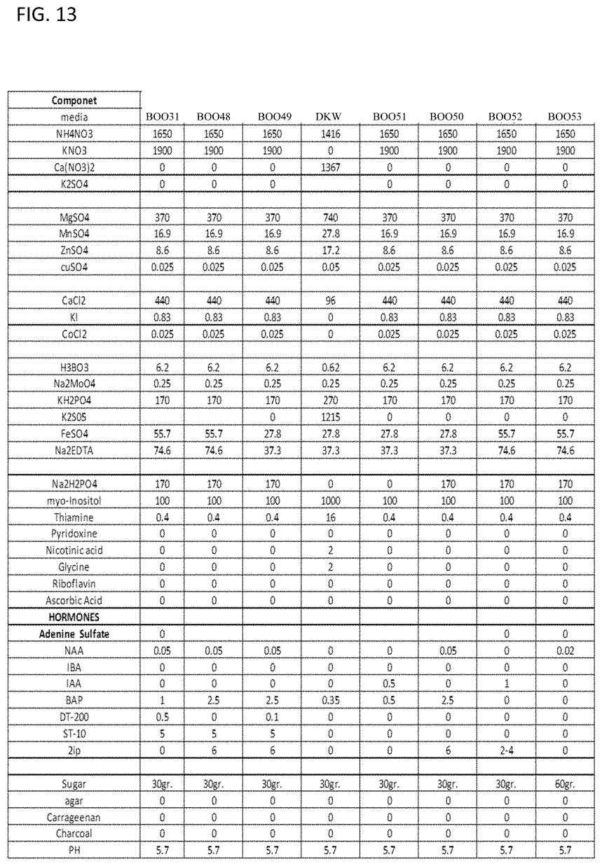

In some embodiments, the media can be selected from the ones listed in the table shown in FIG. 13, or any medium equivalent thereof.

In some embodiments, the bud induction media (BOO1) comprise thidiazuron (TDZ) or analog thereof, and the elongation and maintenance media comprise one or more cytokinins other than TDZ or an analog thereof. In some embodiments, the cytokinins other than TDZ are selected from the group consisting of N6-benzylaminopurine (BAP), meta-topolin (mT), zeatin, zeatin riboside, dihydrozeatin, kinetin, isopentenyladenine (ip, e.g., 2ip), adenine hemi sulfate, dimethylallyladenine, N-(2-chloro-4-pyridyl)-N′-phenylurea) (4-CPPU), and analogs thereof. In some embodiments, the media can be used for plants in vitro micropropagation of monocots or dicots. In some embodiments, the media can be used for bamboo plants in vitro micropropagation.

In some embodiments, the bud induction medium (BOO1) comprises an effective amount of thidiazuron (TDZ) or analog thereof, and wherein the shoot elongation/maintenance medium (BOO2) comprises an effective amount of one or more cytokinins other than TDZ or an analog thereof. In some embodiments, the one or more cytokinins other than TDZ or an analog thereof in the shoot elongation/maintenance medium (BOO2) is selected from the group consisting of N6-benzylaminopurine (BAP), meta-topolin (mT), zeatin, zeatin riboside, dihydrozeatin, kinetin, 2-isopentenyladenine (2ip), adenine hemi sulfate, dimethylallyladenine, N-(2-chloro-4-pyridyl)-N′-phenylurea) (4-CPPU), and analogs thereof.

In addition to the “bud induction media (BOO1)” and “shoot elongation/maintenance media (BOO2)” combination described above, the present invention provides other alternative media combinations for plant propagation. For example, provided are media combinations comprising at least one Stage 1 medium and at least one Stage 2 medium. In some embodiments, the number assigned to a media within a given process is maintained when a certain media is used more than one time. For example, certain embodiments disclosed herein include cycling explants or shoots in a rotation of media. For example, an explant may be placed in a Stage 1 media followed by a Stage 2 media and then returned back to its Stage 1 media. In this context, when exposure to a media is repeated, it retains its lowest Stage number within the particular process. In some embodiments, the alternative media are selected from Stage 1 media, Stage 2 media, Stage 3 media, Stage 4 media, Stage 5 media, Stage 6 media, Stage 7 media, etc. as described herein.

In some embodiments, the alternative media comprise meta-topolin or an analogue thereof. In some embodiments, the alternative media comprise at least two other cytokinins. In some embodiments, the alternative media comprise at least three cytokinins. In some embodiments, said alternative media comprise at least one auxin and at least two cytokinins. In some embodiments, said alternative media comprise at least two auxins and at least two cytokinins. In some embodiments, said alternative media comprise at least two auxins and at least three cytokinins. In some embodiments, the media supports multiplication cycles for a predetermined period of time. In some embodiments, the media support multiplication cycles for at least six months.

In some embodiments, to begin the process, a Stage 1 media can be obtained or prepared. Stage 1 media include a pH that is generally hospitable to plants (typically from 4.0-7.0 or 4.5-6.5). Stage 1 media disclosed herein can include (i) meta-topolin; (2) at least three cytokinins; (3) the cytokinin meta-topolin or an analogue thereof in combination with at least two other cytokinins; (4) at least one auxin and at least two cytokinins; (5) at least two auxins and at least two cytokinins or (6) at least two auxins and at least three cytokinins. In certain embodiments, Stage 1 media must include more than 1 auxin. In other embodiments, Stage 1 media must include more than 1 cytokinin. In further embodiments, Stage 1 media must include more than 1 auxin and more than 1 cytokinin.

In some embodiments, the cytokinins and auxins are chosen from one or more of meta-topolin, thidiazuron, NAA, IBA, BAP, 2ip, CCPU and DPU. In additional embodiments, the cytokinins and auxins are chosen from one or more of meta-topolin or analogues thereof, thidiazuron or analogues thereof, NAA or analogues thereof, IBA or analogues thereof, BAP or analogues thereof, 2ip or analogues thereof, CCPU or analogues thereof and DPU or analogues thereof.

In some embodiments, the media and at least two other cytokinins. In some embodiments, the media supports multiplication cycles for at least six months.

In some embodiments, the media comprise at least three cytokinins. In some embodiments, the media can support multiplication cycles for at least six months. In some embodiments, provided are media comprising the cytokinin meta-topolin or an analogue thereof in combination with at least two other cytokinins.

In some embodiments, the media comprise at least one auxin and at least two cytokinins. In some embodiments, the media can support multiplication cycles for at least six months. In some embodiments, at least one cytokinin is meta-topolin or an analogue thereof.

In some embodiments, the media comprise at least two auxins and at least two cytokinins. In some embodiments, the media can support multiplication cycles for at least six months.

In some embodiments, the media comprise at least two auxins and at least three cytokinins. In some embodiments, the media can support multiplication cycles for at least six months.

In some embodiments, the micropropagated plants are grown in vitro in sterile media.

In some embodiments, the media can be liquid, semi-solid, or solid, and the physical state of the media can be varied by the incorporation or removal of one or more gelling agents. Any gelling agent known in the art that is suitable for use in plant tissue culture media can be used. Agar is most commonly used for this purpose. Examples of such agars include Agar Type A, E or M and Bacto® Agar (Becton Dickinson & Co.). Other exemplary gelling agents include carrageenan, gellan gum (commercially available as PhytaGel™ (Sigma-Aldrich), Gelrite® (Sigma-Aldrich) and Gelzan™ (Caisson Labs)), alginic acid and its salts, and agarose. Blends of these agents, such as two or more of agar, carrageenan, gellan gum, agarose and alginic acid or a salt thereof also can be used.

Examples of plant growth regulators include abscisic acid (ABA), triacontanol, phloroglucinol, auxins and compounds with auxin-like activity, cytokinins and compounds with cytokinin-like activity. Exemplary auxins include 4-fluorophenoxyacetic acid (FA), 2,4,5-trichlorophenoxyacetic acid (2,4,5-T), 3-bromooxindole-3-aceitc acid, 4-bromophenoxyacetic acid, dicamba, p-chlorophenoxyacetic acid (CPA) indole-3-propinoic acid (IPA), 2,4-dichlorophenoxyacetic acid (2,4-D), indole-3-butyric acid (IBA), indole-3-acetic acid (IAA), picloram and combinations thereof. Exemplary cytokinins include meta-topolin, thidiazuron, N-(2-chloro-4-pyridyl)-N-phenylurea (CCPU), 1,3-diphenylurea (DPU), adenine hemi sulfate, benzyladenine, dimethylallyladenine, kinetin, zeatin, riboside, adenosine, meta-topolin riboside, meta-topolin-9-glucoside, ortho-topolin, ortho-topolin riboside, ortho-topolin-9-glucoside, para-topolin, para-topolin riboside, para-topolin-9-glucoside, ortho-methoxytopolin, ortho-methoxytopolin riboside, meta-methoxytopolin, meta-methoxytopolin riboside, meta-methoxytopolin-9-glucoside and combinations thereof as well as plant extracts having cytokinin-like activity, such as coconut water, banana powder, malt extract, pineapple powder or tomato powder.

Gibberellic acid also can be included in the media. A sugar or combination of sugars can be included in the media and can serve as a carbon source. Such sugars are known to those of ordinary skill in the art. Exemplary sugars include fructose, sucrose, glucose, maltose, galactose mannitol and sorbitol or combinations thereof. Other exemplary additives (with suggested but non-limiting functions) include polyamines (regeneration enhancer); citric acid, polyvinylpyrodine (PVP) and sodium thiosulfate (anti-browning agents); CaNO3 or calcium gluconate (hyperhydricity reducer); paclobutrazol or ancymidol (multiplication enhancer); acetyl salicylic acid (ethylene inhibitor) and p-chlorophenoxyisobutyric acid (PCIB) and triiodobenzoic acid (TIBA) (anti-auxins).

In some embodiments, basal media can be Murashige and Skoog (MS). Suitable nutrient salts also include, without limitation, Anderson's Rhododendron, Chu's N-6, DKW, Gamborg's B-5, Hoaglands No. 2, Kao & Michayluk, Nitsch & Nitsch, Schenk and Hildebrant, Vacin and Went, Whites and WPM, available from commercial sources such as Caisson Laboratories, Inc or Phytotechnology Laboratories.

One example of a Stage 1 media includes meta-topolin. Another non-limiting example includes meta-topolin, thidiazuron, NAA and BAP. Another non-limiting example includes meta-topolin, NAA and BAP. Another non-limiting example includes meta-topolin, NAA, BAP and IBA. Another non-limiting example includes meta-topolin, thidiazuron, NAA, BAP and IBA. Another non-limiting example includes thidiazuron, NAA, BAP and 2ip. Another non-limiting example includes thidiazuron, NAA and 2ip. Another non-limiting example includes meta-topolin, thidiazuron, NAA, BAP, IBA and 2ip. Another non-limiting example includes meta-topolin, IBA, 2ip and BAP. Another non-limiting example includes meta-topolin, thidiazuron, CPPU, NAA and BAP. Another non-limiting example includes meta-topolin, thidiazuron, DPU, NAA and BAP. Another non-limiting example includes thidiazuron, CPPU, BAP, IBA and 2ip. Another non-limiting example includes CPPU, DPU, NAA and BAP. Another non-limiting example includes meta-topolin, thidiazuron, CPPU, DPU, NAA, BAP, IBA and 2ip. Each of these non-limiting examples can also include analogues of meta-topolin, thidiazuron, NAA, BAP, DPU, CPPU, IBA and/or 2ip.

The Stage 1 media is then placed into test tubes or other appropriate containers (including jars, boxes, jugs, cups, sterile bag technology, bioreactors, temporary immersion vessels, etc. wherein when not specified are collectively referred to as “tubes”). These tubes can be capped or covered and autoclaved to sterilize the tubes and media. In another embodiment, sterilization is achieved by autoclaving at 5-25 pounds pressure psi at a temperature of 200° F.-for 200° F. 10-25 minutes. In another embodiment, sterilization is achieved by autoclaving at 15 pounds pressure psi at a temperature of 250° F. for 15-18 minutes. Liquid media can be subjected to filter sterilization.

After the explants are allowed to establish themselves on Stage 1 media, the cell cultures grown from the explants are transferred into a Stage 2 media. Stage 2 media disclosed herein can include (i) meta-topolin; (2) at least three cytokinins; (3) the cytokinin meta-topolin or an analogue thereof in combination with at least two other cytokinins; (4) at least one auxin and at least two cytokinins; (5) at least two auxins and at least two cytokinins or (6) at least two auxins and at least three cytokinins. In certain embodiments, Stage 2 media must include more than 1 auxin. In other embodiments, Stage 2 media must include more than 1 cytokinin. In further embodiments, Stage 2 media must include more than 1 auxin and more than 1 cytokinin.

In some embodiments, the cytokinins and auxins are chosen from one or more of meta-topolin, thidiazuron, NAA, IBA, BAP, 2ip, CCPU and DPU. In additional embodiments, the cytokinins and auxins are chosen from one or more of meta-topolin or analogues thereof, thidiazuron or analogues thereof, NAA or analogues thereof, IBA or analogues thereof, BAP or analogues thereof, 2ip or analogues thereof, CCPU or analogues thereof and DPU or analogues thereof.

In some embodiments, one example of a Stage 2 media includes meta-topolin. Another non-limiting example includes meta-topolin, thidiazuron, NAA and BAP. Another non-limiting example includes meta-topolin, NAA and BAP. Another non-limiting example includes meta-topolin, NAA, BAP and IBA. Another non-limiting example includes meta-topolin, thidiazuron, NAA, BAP and IBA. Another non-limiting example includes thidiazuron, NAA, BAP and 2ip. Another non-limiting example includes thidiazuron, NAA and 2ip. Another non-limiting example includes meta-topolin, thidiazuron, NAA, BAP, IBA and 2ip. Another non-limiting example includes meta-topolin, IBA, 2ip and BAP. Another non-limiting example includes meta-topolin, thidiazuron, CPPU, NAA and BAP. Another non-limiting example includes meta-topolin, thidiazuron, DPU, NAA and BAP. Another non-limiting example includes thidiazuron, CPPU, BAP, IBA and 2ip. Another non-limiting example includes CPPU, DPU, NAA and BAP. Another non-limiting example includes meta-topolin, thidiazuron, CPPU, DPU, NAA, BAP, IBA and 2ip. Each of these non-limiting examples can also include analogues of meta-topolin, thidiazuron, NAA, BAP, DPU, CPPU, IBA and/or 2ip.

In particular embodiments disclosed herein, both Stage 1 and Stage 2 media include meta-topolin. In another non-limiting embodiment, both Stage 1 and Stage 2 media include meta-topolin, thidiazuron, NAA and BAP. In another non-limiting embodiment, both Stage 1 and Stage 2 media include meta-topolin, NAA and BAP. In another non-limiting embodiment, both Stage 1 and Stage 2 media include meta-topolin, NAA, BAP and IBA. In another non-limiting embodiment, both Stage 1 and Stage 2 media include meta-topolin, thidiazuron, NAA, BAP and IBA. In another non-limiting embodiment, both Stage 1 and Stage 2 media include thidiazuron, NAA, BAP and 2ip. In another non-limiting embodiment, both Stage 1 and Stage 2 media include thidiazuron, NAA and 2ip. In another non-limiting embodiment, both Stage 1 and Stage 2 media include meta-topolin, thidiazuron, NAA, BAP, IBA and 2ip. In another non-limiting embodiment, both Stage 1 and Stage 2 media include meta-topolin, IBA, 2ip and BAP. In another non-limiting embodiment, both Stage 1 and Stage 2 media include meta-topolin, thidiazuron, CPPU, NAA and BAP. In another non-limiting embodiment, both Stage 1 and Stage 2 media include meta-topolin, thidiazuron, DPU, NAA and BAP. In another non-limiting embodiment, both Stage 1 and Stage 2 media include thidiazuron, CPPU, BAP, IBA and 2ip. In another non-limiting embodiment, both Stage 1 and Stage 2 media include CPPU, DPU, NAA and BAP. In another non-limiting embodiment, both Stage 1 and Stage 2 media include meta-topolin, thidiazuron, CPPU, DPU, NAA, BAP, IBA and 2ip. Each of these non-limiting examples can also include analogues of meta-topolin, thidiazuron, NAA, BAP, DPU, CPPU, IBA and/or 2ip.

In some embodiments, examples of Stage 1 and Stage 2 media are described in WO/2011/100762, which is incorporated herein by references in its entirety. For example, Stage 1 and Stage 2 media can be selected from the group consisting of Media BOO38(i-v); Media BOO40 (i-v); Media BOO41(i-v); Media BOO36 (i-v); Media BOO42(i-v); Media BOO39(i-v); Media BOO43(i-v); Media BOO44(i-v); Media BOO37 (i-v); Media BOO31 (i-v); Media BOO28 (i-v); Media BOO29 (i-v); Media BOO30 (i-v).

In some embodiments, examples of Stage 1 and Stage 2 media are selected from the ones described below:

### Media BOO38(i-v):

| | Standard | Standard | Standard | Standard | Standard |
|---|---|---|---|---|---|
| Component | BOO38-i | BOO38-ii | BOO38-iii | BOO38-iv | BOO38-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| Ca(NO₃)₂ | 225-775 | 410-690 | 495-605 | 550 | 550 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.020-0.030 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | l ± 0.2 |
| Thidiazuron | 0.36-1.12 | 0.56-0.94 | 0.67-.083 | 0.75 | 0.75 ± .02 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

Media BOO40 (i-v):

| | Standard | Standard | Standard | Standard | Standard |
|---|---|---|---|---|---|
| Component | BOO40-i | BOO40-ii | BOO40-iii | BOO40-iv | BOO40-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| Ca(NO₃)₂ | 225-775 | 410-690 | 495-605 | 550 | 550 ± 2 |
| MgSO₄ | 185-555 | 275-465 | 330-410 | 370 | 370 ± 2 |
| MnSO₄ | 8.0-26.0 | 12.0-22.0 | 15.0-19.0 | 16.9 | 16.9 ± 0.2 |
| ZnSO₄ | 4.0-12.0 | 6.0-10.0 | 8.0-9.0 | 8.6 | 8.6 ± 0.2 |
| CuSO₄ | 0.012-0.37 | 0.02-0.03 | 0.02-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.02-0.028 | 0.025 | 0.025 ± .002 |
| H₃BO₃ | 3.0-9.0 | 4.0-8.0 | 5.0-7.0 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | | | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | 1 ± 0.2 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

### Media BOO41(i-v):

| | Standard | Standard | Standard | Standard | Standard |
|---|---|---|---|---|---|
| Component | BOO41-i | BOO41-ii | BOO41-iii | BOO41-iv | BOO41-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| Ca(NO₃)₂ | 225-775 | 410-690 | 495-605 | 550 | 550 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.02-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.05-0.15 | 0.07-0.12 | 0.09-0.11 | 0.1 | 0.1 ± 0.02 |
| BAP | 0.5-1.5 | 0.7-1.3 | 0.9-1.1 | 1 | 1 ± 0.2 |
| IBA | 0.1-0.3 | 0.15-0.25 | 0.17-0.22 | 0.2 | 0.2 ± 0.1 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

Media BOO36 (i-v):

| | Standard | Standard | Standard | Standard | Standard |
|---|---|---|---|---|---|
| Component | BOO36-i | BOO36-ii | BOO36-iii | BOO36-iv | BOO36-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8.0-26.0 | 12.0-22.0 | 15.0-19.0 | 16.9 | 16.9 ± 0.2 |
| ZnSO₄ | 4.0-12.0 | 6.0-10.0 | 8.0-9.0 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.4-1.25 | 0.6-1.05 | 0.7-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | l ± 0.2 |
| Thidiazuron | 0.12-0.36 | 0.18-0.31 | .22-.28 | 0.25 | 0.25 ± .02 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

### Media BOO42(i-v):

| | Standard | Standard | Standard | Standard | Standard |
|---|---|---|---|---|---|
| Component | BOO42-i | BOO42-ii | BOO42-iii | BOO42-iv | BOO42-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.05-0.15 | 0.07-0.12 | 0.09-0.11 | 0.1 | 0.1 ± 0.02 |
| BAP | 0.5-1.5 | 0.7-1.3 | 0.9-1.1 | 1 | 1 ± 0.2 |
| IBA | 0.1-0.3 | 0.15-0.25 | 0.17-0.22 | 0.2 | 0.2 ± 0.1 |
| Thidiazuron | 0.12-0.36 | 0.18-0.31 | .22-.28 | 0.25 | 0.25 ± .02 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

### Media BOO39(i-v):

| | Standard | Standard | Standard | Standard | Standard |
|---|---|---|---|---|---|
| Component | BOO39-i | BOO39-ii | BOO39-iii | BOO39-iv | BOO39-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | 1 ± 0.2 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |
| Silicon | 0.5-1.5 | 0.7-1.3 | 0.9-1.1 | 1 | 1 ± 0.2 |
| Solution mL | | | | | |

### Media BOO43(i-v):

| | Standard | Standard | Standard | Standard | Standard |
|---|---|---|---|---|---|
| Component | BOO43-i | BOO43-ii | BOO43-iii | BOO43-iv | BOO43-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | 1 ± 0.2 |
| IBA | 0.1-0.3 | 0.15-0.25 | 0.17-0.22 | 0.2 | 0.2 ± 0.1 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |
| Silicon | 0.5-1.5 | 0.7-1.3 | 0.9-1.1 | 1 | 1 ± 0.2 |
| Solution mL | | | | | |

### Media BOO44(i-v):

| | Standard | Standard | Standard | Standard | Standard |
|---|---|---|---|---|---|
| Component | BOO44-i | BOO44-ii | BOO44-iii | BOO44-iv | BOO44-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.4-1.2 | 0.6-1.0 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| K₂SO₅ | 181.8-545.6 | 272.8-454.6 | 327.4-400.0 | 363.75 | 363.75 ± .02 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.05-0.15 | 0.07-0.12 | 0.09-0.11 | 0.1 | 0.1 ± 0.02 |
| BAP | 0.5-1.5 | 0.7-1.3 | 0.9-1.1 | 1 | 1 ± 0.2 |
| IBA | 0.1-0.3 | 0.15-0.25 | 0.17-0.22 | 0.2 | 0.2 ± 0.1 |
| Meta-topolin | 2.5-7.5 | 3.7-6.2 | 4.5-5.5 | 5 | 5 ± 2 |
| Thidiazuron | 0.12-0.36 | 0.18-0.31 | .22-.28 | 0.25 | 0.25 ± .02 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

Media BOO37 (i-v):

| | Standard | Standard | Standard | Standard | Standard |
|---|---|---|---|---|---|
| Component | BOO37-i | BOO37-ii | BOO37-iii | BOO37-iv | BOO37-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4.0-12.0 | 6.0-10.0 | 8.0-9.0 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37.-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | 1 ± 0.2 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.5-7.5 | 3.7-6.2 | 4.5-5.5 | 5 | 5 ± 2 |

Media BOO31 (i-v):

| | Standard | Standard | Standard | Standard | Standard |
|---|---|---|---|---|---|
| Component | BOO31-i | BOO31-ii | BOO31-iii | BOO31-iv | BOO31-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | 1 ± 0.2 |
| Thidiazuron | 0.2-0.8 | 0.3-0.7 | 0.4-0.6 | 0.5 | 0.5 ± 0.2 |
| Meta-topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

Media BOO28 (i-v):

| | Standard | Standard | Standard | Standard | Standard |
|---|---|---|---|---|---|
| Component | BOO28-i | BOO28-ii | BOO28-iii | BOO28-iv | BOO28-v |
| NH₄NO₃ | 600-1800 | 900-1500 | 1080-1320 | 1200 | 1200 ± 2 |
| Ca(NO₃)₂ | 838-2515 | 1257-2096 | 1510-1844 | 1677 | 1677 ± 2 |
| K₂SO₄ | 121-363 | 181-302 | 218-266 | 242 | 242 ± 2 |
| MgSO₄ | 270-830 | 410-690 | 500-610 | 555 | 555 ± 2 |
| MnSO₄ | 12.6-38.0 | 19.0-31.7 | 22.8-27.8 | 25.35 | 25.35 ± .02 |
| ZnSO₄ | 6.4-19.5 | 9.6-16.2 | 11.5-14.0 | 12.9 | 12.9 ± 0.2 |
| CuSO₄ | 0.01-0.05 | 0.02-0.04 | 0.033-0.041 | 0.037 | 0.037 ± 0.002 |
| CaCl₂ | 48-144 | 72-120 | 85-105 | 96 | 96 ± 2 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27.0-84.0 | 40.0-70.0 | 50.0-60.0 | 55.7 | 55.7 ± 0.2 |
| Na₂EDTA | 37.0-112.0 | 55.0-94.0 | 67.0-82.0 | 74.6 | 74.6 ± 0.2 |
| Na₂H₂PO₄ | 42-128 | 63-106 | 75-95 | 85 | 85 ± 2 |
| myo-Inositol | 100-300 | 150-250 | 180-220 | 200 | 200 ± 2 |
| Thiamine | 0.4-1.4 | 0.6-1.1 | 0.8-1.0 | 0.9 | 0.9 ± 0.2 |
| Pyridoxine | 0.2-0.8 | 0.3-0.7 | 0.4-0.6 | 0.5 | 0.5 ± 0.2 |
| Nicotinic acid | 0.2-0.8 | 0.3-0.7 | 0.4-0.6 | 0.5 | 0.5 ± 0.2 |
| Glycine | 1-3 | 1.5-2.5 | 1.75-2.25 | 2 | 2 ± 1 |
| Riboflavin | 10-30 | 15-25 | 18-22 | 20 | 20 ± 2 |
| BAP | 0.1-0.3 | 0.15-0.25 | 0.17-0.22 | 0.2 | 0.2 ± 0.1 |
| NAA | 0.2-0.8 | 0.3-0.7 | 0.4-0.6 | 0.5 | 0.5 ± 0.2 |
| Thidiazuron | 0.36-1.12 | 0.56-0.94 | 0.67-.083 | 0.75 | 0.75 ± .02 |
| 2ip | 7-23 | 11-19 | 13-17 | 15 | 15 ± 2 |
| Sugar g/L | 15-45 | 22-37 | 27-33 | 30 | 30 ± 2 |
| Carrageenan g/L | 3-11 | 4-10 | 5-8 | 7 | 7 ± 2 |

Media BOO29 (i-v):

| | Standard | Standard | Standard | Standard | Standard |
|---|---|---|---|---|---|
| Component | BOO29-i | BOO29-ii | BOO29-iii | BOO29-iv | BOO29-v |
| NH₄NO₃ | 600-1800 | 900-1500 | 1080-1320 | 1200 | 1200 ± 2 |
| Ca(NO₃)₂ | 838-2515 | 1257-2096 | 1510-1844 | 1677 | 1677 ± 2 |
| K₂SO₄ | 121-363 | 181-302 | 218-266 | 242 | 242 ± 2 |
| MgSO₄ | 270-830 | 410-690 | 500-610 | 555 | 555 ± 2 |
| MnSO₄ | 12.6-38.0 | 19.0-31.7 | 22.8-27.8 | 25.35 | 25.35 ± .02 |
| ZnSO₄ | 6.4-19.5 | 9.6-16.2 | 11.5-14.0 | 12.9 | 12.9 ± 0.2 |
| CuSO₄ | 0.01-0.05 | 0.02-0.04 | 0.03-0.04 | 0.037 | 0.037 ± .002 |
| CaCl₂ | 48-144 | 72-120 | 85-105 | 96 | 96 ± 2 |
| H₃BO₃ | 3.0-9.0 | 4.0-8.0 | 5.0-7.0 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 42-128 | 63-106 | 75-95 | 85 | 85 ± 2 |
| myo-Inositol | 100-300 | 150-250 | 180-220 | 200 | 200 ± 2 |
| Thiamine | 0.4-1.4 | 0.6-1.1 | 0.8-1.0 | 0.9 | 0.9 ± 0.2 |
| Pyridoxine | 0.2-0.8 | 0.3-0.7 | 0.4-0.6 | 0.5 | 0.5 ± 0.2 |
| Nicotinic acid | 0.2-0.8 | 0.3-0.7 | 0.4-0.6 | 0.5 | 0.5 ± 0.2 |
| Glycine | 1-3 | 1.5-2.5 | 1.75-2.25 | 2 | 2 ± 1 |
| Riboflavin | 10-30 | 15-25 | 18-22 | 20 | 20 ± 2 |
| BAP | 2.5-7.5 | 3.7-6.2 | 4.5-5.5 | 5 | 5 ± 2 |
| NAA | 0.5-1.5 | 0.7-1.3 | 0.9-1.1 | 1 | 1 ± 0.2 |
| Thidiazuron | 0.36-1.12 | 0.56-0.94 | 0.67-.083 | 0.75 | 0.75 ± .02 |
| 2ip | 10-30 | 15-25 | 18-22 | 20 | 20 ± 2 |
| Sugar g/L | 15-45 | 22-37 | 27-33 | 30 | 30 ± 2 |
| Carrageenan g/L | 3-11 | 4-10 | 5-8 | 7 | 7 ± 2 |

Media BOO30 (i-v):

| | Standard | Standard | Standard | Standard | Standard |
|---|---|---|---|---|---|
| Component | BOO30-i | BOO30-ii | BOO30-iii | BOO30-iv | BOO30-v |
| NH₄NO₃ | 600-1800 | 900-1500 | 1080-1320 | 1200 | 1200 ± 2 |
| Ca(NO₃)₂ | 838-2515 | 1257-2096 | 1510-1844 | 1677 | 1677 ± 2 |
| K₂SO₄ | 121-363 | 181-302 | 218-266 | 242 | 242 ± 2 |
| MgSO₄ | 270-830 | 410-690 | 500-610 | 555 | 555 ± 2 |
| MnSO₄ | 12.6-38.0 | 19.0-31.7 | 22.8-27.8 | 25.35 | 25.35 ± .02 |
| ZnSO₄ | 6.4-19.5 | 9.6-16.2 | 11.5-14.0 | 12.9 | 12.9 ± 0.2 |
| CuSO₄ | 0.01-0.05 | 0.02-0.04 | 0.033-0.041 | 0.037 | 0.037 ± .002 |
| CaCl₂ | 48-144 | 72-120 | 85-105 | 96 | 96 ± 2 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 42-128 | 63-106 | 75-95 | 85 | 85 ± 2 |
| myo-Inositol | 100-300 | 150-250 | 180-220 | 200 | 200 ± 2 |
| Thiamine | 0.4-1.4 | 0.6-1.1 | 0.8-1.0 | 0.9 | 0.9 ± 0.2 |
| Pyridoxine | 0.2-0.8 | 0.3-0.7 | 0.4-0.6 | 0.5 | 0.5 ± 0.2 |
| Nicotinic acid | 0.2-0.8 | 0.3-0.7 | 0.4-0.6 | 0.5 | 0.5 ± 0.2 |
| Glycine | 1-3 | 1.5-2.5 | 1.75-2.25 | 2 | 2 ± 1 |
| Riboflavin | 10-30 | 15-25 | 18-22 | 20 | 20 ± 2 |
| NAA | 0.5-1.5 | 0.7-1.3 | 0.9-1.1 | 1 | 1 ± 0.2 |
| Thidiazuron | 0.12-0.36 | 0.18-0.31 | .22-.28 | 0.25 | 0.25 ± .02 |
| 2ip | 2.5-7.5 | 3.7-6.2 | 4.5-5.5 | 5 | 5 ± 2 |
| Sugar g/L | 12-37 | 15-35 | 20-30 | 25 | 25 ± 2 |
| Agar g/L | 2.7-8.2 | 4.1-6.8 | 4.9-6.1 | 5.5 | 5.5 ± 0.2 |
| Carrageenan g/L | 1-3 | 1.5-2.5 | 1.75-2.25 | 2 | 2 ± 1 |

### Media BOO35

| | Standard | Standard | Standard | Standard | Standard |
|---|---|---|---|---|---|
| Component | BOO35-i | BOO35-ii | BOO35-iii | BOO35-iv | BOO35-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| K₂SO₄ | 242-726 | 363-605 | 436-532 | 484 | 484 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.02-0.028 | 0.025 | 0.025 ± .002 |
| CaCl₂ | 220-660 | 330-350 | 400-480 | 440 | 440 ± 2 |
| KI | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3.0-9.0 | 4.0-8.0 | 5.0-7.0 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 100-300 | 150-250 | 180-220 | 200 | 200 ± 2 |
| Thiamine | 0.45-1.35 | 0.67-1.13 | 0.8-1.0 | 0.9 | 0.9 ± 0.2 |
| NAA | 0.15-0.45 | 0.22-0.38 | 0.27-0.33 | 0.3 | 0.3 ± 0.2 |
| BAP | 1-3 | 1.25-2.75 | 1.5-2.5 | 2 | 2 ± 0.5 |
| Thidiazuron | 0.25-0.75 | 0.37-0.63 | 0.45-0.55 | 0.5 | 0.5 ± .2 |
| Meta-Topolin | 2.5-7.5 | 3.7-6.2 | 4.5-5.5 | 5 | 5 ± 2 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

### Media BOO38 CPPU

| | Standard | Standard | Standard | Standard | Standard |
|---|---|---|---|---|---|
| | BOO3 | BOO38 | BOO38 | BOO38 | BOO38 |
| Component | CPPU-i | CPPU-ii | CPPU-iii | CPPU-iv | CPPU-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| Ca(NO₃)₂ | 225-775 | 410-690 | 495-605 | 550 | 550 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| KI | 0.40-1.25 | 0.60-1.05 | 0.75-0.90 | 0.83 | 0.83 ± .02 |
| CoCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| Casein | 1-3 | 1.5-2.5 | 1.75-2.25 | 2 | 2 ± 1 |
| Hydroxylate g/L | | | | | |
| NAA | 0.02-0.08 | 0.03-0.07 | 0.04-0.06 | 0.05 | 0.05 ± .02 |
| BAP | 0.5-1.5 | 0.7-1.3 | 0.9-1.1 | 1 | 1 ± 0.2 |
| Thidiazuron | 0.36-1.12 | 0.56-0.94 | 0.67-.083 | 0.75 | 0.75 ± .02 |
| Meta-Topolin | 2.5-7.5 | 3.7-6.2 | 4.5-5.5 | 5 | 5 ± 2 |
| CPPU | 0.36-1.12 | 0.56-0.94 | 0.67-.083 | 0.75 | 0.75 ± .02 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

### Media BOO38 DPU

| | Standard | Standard | Standard | Standard | Standard |
|---|---|---|---|---|---|
| | BOO38 | BOO38 | BOO38 | BOO38 | BOO38 |
| Component | CPU-i | CPU-ii | CPU-iii | CPU-iv | CPU-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| Ca(NO₃)₂ | 225-775 | 410-690 | 495-605 | 550 | 550 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| KI | 0.40-1.25 | 0.60-1.05 | 0.75-0.90 | 0.83 | 0.83 ± .02 |
| COCl₂ | 0.01-0.37 | 0.02-0.03 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 27-84 | 40-70 | 50-60 | 55.7 | 55.7 ± 0.2 |
| Na₂EDTA | 37-112 | 55-94 | 67-82 | 74.6 | 74.6 ± 0.2 |
| Na₂H₂PO₄ | 85-255 | 120-210 | 150-190 | 170 | 170 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± 0.2 |
| Casein | 1-3 | 1.5-2.5 | 1.75-2.25 | 2 | 2 ± 1 |
| Hydroxylate | | | | | |
| g/L | | | | | |
| NAA | 0.02-0.08 | 0.03-0.07 | 0.04-0.06 | 0.05 | 0.05 ± .02 |
| BAP | 0.5-1.5 | 0.7- 1.3 | 0.9-1.1 | 1 | 1 ± 0.2 |
| Thidiazuron | 0.36-1.12 | 0.56-0.94 | 0.67-.083 | 0.75 | 0.75 ± .02 |
| Meta-Topolin | 2.5-7.5 | 3.7-6.2 | 4.5-5.5 | 5 | 5 ± 2 |
| DPU | 0.36-1.12 | 0.56-0.94 | 0.67-.083 | 0.75 | 0.75 ± .02 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

In some embodiments, the non-cytokinin components are those found in Anderson's Rhododendron, Chu's N-6, DKW, Gamborg's B-5, Hoaglands No. 2, Kao & Michayluk, Nitsch & Nitsch, Schenk and Hildebrant, Vacin and Went, Whites and WPM, available from commercial sources. Particular media can have higher or lower levels of macronutrients than those provided in the preceding tables and others will lack nitrates. In some embodiments, the media have higher or lower levels of macronutrients and lack nitrates. In some embodiments, the media have higher levels of macronutrients and lack nitrates.

Media disclosed herein also include spiked media. Spiked media are those in which the concentration of at least one cytokinin and/or auxin in the media described above is increased by, for example and without limitation, 1%, 3%, 5%, 7%, 10%, 15%, 20%, 25%, 30%, 35%, 40%, 45%, 50%, 55%, 60%, 65%, 70%, 75%, 80%, 85%, 90% 95%, 100%, 105%, 110% or 200%. In other embodiments, the concentration of at least one cytokinin and/or auxin in the media described above is increased by, for example and without limitation, 1-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-100%, 95-105%, 100-110%, 105-115%, 110-120%, 115-125%, 120-130%, 125-135%, 130-140%, 135-145%, 140-150%, 145-155%, 150-160%, 155-165%, 160-170%, 165-175%, 170-180%, 175-185%, 180-190%, 185-195%, 190-200%, 195-205%, 3-6%, 7-17%, 12-22%, 17-27%, 22-32%, 27-37%, 32-42%, 37-47%, 42-52%, 47-57%, 52-62%, 57-67%, 62-72%, 67-77%, 72-82%, 77-87%, 82-92%, 87-97%, 92-102%, 97-107%, 102-112%, 107-117%, 112-122%, 117-127%, 122-132%, 127-137%, 132-142%, 137-147%, 142-152%, 147-157%, 152-162%, 157-167%, 162-172%, 167-177%, 172-182%, 177-187%, 162-172%, 167-177%, 182-192%, 187-197%, 192-202%, 197-207%, or 200-210%. When more than one cytokinin and/or auxin is spiked, the concentrations of each raised cytokinin and/or auxin can be raised by the same amount or a different amount than other cytokinins and/or auxins in the media.

The following tables provide non-limiting examples of spiked media disclosed herein. Each media includes the components described in its respective table above or adjusted as described in the next paragraph with the following adjustments to cytokinin levels.

### Media BOO38(i-v):

| | Spiked | Spiked | Spiked | Spiked | Spiked |
|---|---|---|---|---|---|
| Component | BOO38-i | BOO38-ii | BOO38-iii | BOO38-iv | BOO38-v |
| NAA | 0.1 | 0.1 | 0.05 | 0.05 | 0.5 |
| BAP | 2 | 1 | 1.25 | 1 | 1.5 |
| Thidiazuron | 0.75 | 0.7 | 1 | 0.75 | 1.25 |
| Meta-topolin | 10 | 5 | 5 | 6 | 7.5 |

Media BOO40 (i-v):

| | Spiked | Spiked | Spiked | Spiked | Spiked |
|---|---|---|---|---|---|
| Component | BOO40-i | BOO40-ii | BOO40-iii | BOO40-iv | BOO40-v |
| NAA | 0.05 | 0.05 | 0.2 | 0.1 | 0.05 |
| BAP | 1 | 2 | 1 | 2 | 5 |
| Meta-topolin | 20 | 10 | 7.5 | 10 | 5 |

### Media BOO41(i-v):

| | Spiked | Spiked | Spiked | Spiked | Spiked |
|---|---|---|---|---|---|
| Component | BOO41-i | BOO41-ii | BOO41-iii | BOO41-iv | BOO41-v |
| NAA | 0.1 | 0.1 | 0.4 | 0.1 | 0.5 |
| BAP | 1 | 1 | 3 | 1 | 2 |
| IBA | 0.2 | 0.75 | 0.2 | 0.5 | 1 |
| Meta-topolin | 8 | 16 | 9 | 30 | 5 |

Media BOO36 (i-v):

| | Spiked | Spiked | Spiked | Spiked | Spiked |
|---|---|---|---|---|---|
| Component | BOO36-i | BOO36-ii | BOO36-iii | BOO36-iv | BOO36-v |
| NAA | 1 | 2 | 0.5 | 0.25 | 0.05 |
| BAP | 11 | 5 | 50 | 10 | 1 |
| Thidiazuron | 0.5 | 1 | 1.5 | 1.75 | 0.25 |
| Meta-topolin | 9 | 8 | 7 | 6 | 500 |

### Media BOO42(i-v):

| | Spiked | Spiked | Spiked | Spiked | Spiked |
|---|---|---|---|---|---|
| Component | BOO42-i | BOO42-ii | BOO42-iii | BOO42-iv | BOO42-v |
| NAA | 0.1 | 0.1 | 0.1 | 0.1 | 0.1 |
| BAP | 1 | 1 | 2 | 3 | 4 |
| IBA | 0.4 | 0.2 | 1 | 0.5 | 0.2 |
| Thidiazuron | 0.25 | 0.5 | 1 | 5 | 0.3 |
| Meta-topolin | 50 | 40 | 5 | 15 | 10 |

### Media BOO39(i-v):

| | Spiked | Spiked | Spiked | Spiked | Spiked |
|---|---|---|---|---|---|
| Component | BOO39-i | BOO39-ii | BOO39-iii | BOO39-iv | BOO39-v |
| NAA | 2 | 0.05 | 0.05 | 10 | 0.15 |
| BAP | 1 | 100 | 150 | 80 | 5 |
| Meta-topolin | 85 | 300 | 100 | 5 | 25 |

### Media B0043(i-v):

| | Spiked | Spiked | Spiked | Spiked | Spiked |
|---|---|---|---|---|---|
| Component | BOO43-i | BOO43-ii | BOO43-iii | BOO43-iv | BOO43-v |
| NAA | 0.05 | 0.15 | 0.05 | 0.1 | 3 |
| BAP | 1 | 1 | 5 | 1 | 4 |
| IBA | 0.2 | 0.4 | 0.2 | 1 | 5 |
| Meta-topolin | 1000 | 890 | 75 | 30 | 5 |

### Media BOO44(i-v):

| | Spiked | Spiked | Spiked | Spiked | Spiked |
|---|---|---|---|---|---|
| Component | BOO44-i | BOO44-ii | BOO44-iii | BOO44-iv | BOO44-v |
| NAA | 0.1 | 0.1 | 2 | 0.4 | 0.5 |
| BAP | 3 | 1 | 4 | 1.5 | 2 |
| IBA | 0.5 | 0.2 | 0.4 | 0.3 | 1 |
| Thidiazuron | 0.25 | 0.25 | 0.5 | 10 | 3 |
| Meta-topolin | 450 | 20 | 10 | 5 | 7.5 |

Media BOO37 (i-v):

| | Spiked | Spiked | Spiked | Spiked | Spiked |
|---|---|---|---|---|---|
| Component | BOO37-i | BOO37-ii | BOO37-iii | BOO37-iv | BOO37-v |
| NAA | 0.05 | 0.05 | 1 | 0.25 | 0.05 |
| BAP | 1.3 | 3 | 1.6 | 2 | 1 |
| Meta-topolin | 65 | 70 | 75 | 80 | 20 |

Media BOO31 (i-v):

| | Spiked | Spiked | Spiked | Spiked | Spiked |
|---|---|---|---|---|---|
| Component | BOO31-i | BOO31-ii | BOO31-iii | BOO31-iv | BOO31-v |
| NAA | 0.05 | 0.5 | 1 | 1.75 | 0.05 |
| BAP | 4 | 3 | 2 | 1 | 1 |
| Thidiazuron | 5 | 10 | 15 | 20 | 0.5 |
| Meta-topolin | 750 | 43 | 12 | 300 | 125 |

Media BOO28 (i-v):

| | Spiked | Spiked | Spiked | Spiked | Spiked |
|---|---|---|---|---|---|
| Component | BOO28-i | BOO28-ii | BOO28-iii | BOO28-iv | BOO28-v |
| BAP | 0.2 | 0.4 | 0.6 | 0.8 | 0.3 |
| NAA | 3 | 0.75 | 0.5 | 1 | 0.5 |
| Thidiazuron | 7.5 | 75 | 9 | 150 | 3.5 |
| 2ip | 80 | 60 | 45 | 30 | 20 |

Media BOO29 (i-v):

| | Spiked | Spiked | Spiked | Spiked | Spiked |
|---|---|---|---|---|---|
| Component | BOO29-i | BOO29-ii | BOO29-iii | BOO29-iv | BOO29-v |
| BAP | 30 | 10 | 7 | 5 | 5 |
| NAA | 4 | 3 | 2 | 1 | 1 |
| Thidiazuron | 0.75 | 75 | 7.5 | 5 | 0.75 |
| 2ip | 60 | 20 | 80 | 40 | 25 |

Media BOO30 (i-v):

| | Spiked | Spiked | Spiked | Spiked | Spiked |
|---|---|---|---|---|---|
| Component | BOO30-i | BOO30-ii | BOO30-iii | BOO30-iv | BOO30-v |
| NAA | 1 | 1 | 4 | 10 | 1 |
| Thidiazuron | 1 | 2 | 5 | 10 | 0.25 |
| 2ip | 15 | 7.5 | 5 | 5 | 5 |

### Media BOO35

| | Spiked | Spiked | Spiked | Spiked | Spiked |
|---|---|---|---|---|---|
| Component | BOO35-i | BOO35-ii | BOO35-iii | BOO35-iv | BOO35-v |
| NAA | 1 | 0.6 | 0.3 | 0.4 | 0.30 |
| BAP | 4 | 3 | 1.5 | 2 | 1 |
| Thidiazuron | 0.75 | 0.25 | 0.5 | 0.5 | 0.25 |
| Meta-Topolin | 10 | 5 | 5 | 6 | 5 |

### Media BOO38 CPPU

| | Spiked | Spiked | Spiked | Spiked | Spiked |
|---|---|---|---|---|---|
| | BOO38 | BOO38 | BOO38 | BOO38 | BOO38 |
| Component | CPPU-i | CPPU-ii | CPPU-iii | CPPU-iv | CPPU-v |
| NAA | 0.75 | 1 | 0.05 | 1 | 0.05 |
| BAP | 1.25 | 2 | 1 | 1 | 2 |
| Thidiazuron | 1 | 1.5 | 1 | 0.75 | 1.5 |
| Meta-Topolin | 6 | 10 | 8 | 5 | 5 |
| CPPU | 0.8 | 1.5 | 1.5 | 0.75 | 0.75 |

### Media BOO38 DPU

| | Spiked | Spiked | Spiked | Spiked | Spiked |
|---|---|---|---|---|---|
| | BOO38 | BOO38 | BOO38 | BOO38 | BOO38 |
| Component | CPU-i | CPU-ii | CPU-iii | CPU-iv | CPU-v |
| NAA | 0.1 | 0.1 | 1.8 | 3 | 0.3 |
| BAP | 1.5 | 2 | 1.8 | 2 | 1.5 |
| Thidiazuron | 1.5 | 1.5 | 1.8 | 17 | 3 |
| Meta-Topolin | 10 | 9 | 5 | 6 | 5 |
| DPU | 4 | 3 | 1.8 | 0.75 | 0.75 |

Additional spiked media can include any standard media described above with the addition or adjustment to the following cytokinin and/or auxin concentrations:

### Media AA

| Component | AA-i | AA-ii | AA-iii |
|---|---|---|---|
| BAP | 1 | 1 | 1 |
| Thidiazuron | 2.5 | 5 | 10 |

### Media AB

| Component | AB-i | AB-ii | AB-iii | AB-iv |
|---|---|---|---|---|
| BAP | 5 | 10 | 20 | 40 |

### Media AC

| Component | AC-i | AC-ii | AC-iii | AC-iv | AC-v |
|---|---|---|---|---|---|
| BAP | 1 | 1 | 1 | 1 | 1 |
| CPPU | 2.5 | 7.5 | 10 | 25 | 50 |

As explained more fully below, when a spiked media is utilized, explants or shoots generally (but not necessarily) remain on the spiked media for a shorter period of time than those kept on non-spiked media and following culture on a spiked media, the explants or shoots are transferred to a media containing standard, reduced or no levels of cytokinins and/or auxins (those containing reduced or no cytokinins and/or auxins are both referred to as “reduced” media herein).

One or more compositions of the media disclosed herein can also be adjusted based on the plant species.

In some embodiments, the media disclosed herein can be used for bamboo tissue culture. Representative genera of bamboo are described in WO/2011/100762, which is incorporated herein by reference in its entirety.

In some embodiments, Stage 1 media are selected from the group consisting of BOO38-v media, BOO38-CPPU-v media, BOO38 DPU-v, BOO35-v media or BOO37-v media or spiked versions thereof.

In some embodiments, Stage 2 media are selected from the group consisting of BOO39-v media; BOO40-v media; BOO41-v media; BOO42-v media; BOO43-v media; BOO44-v media, BOO38-CPPU-v media, BOO38 DPU-v, BOO35-v media or spiked and reduced/standard versions thereof for 10-120 day cycles (as modified for spiked media as described more fully below).

In some embodiments, one or more Stage 3 media can be included. In some embodiments, Stage 3 media are selected from the group consisting of BOO46-v media; BOO45-v media or BOO47-v media or spiked and reduced/standard versions thereof (as modified for spiked media as described more fully below).

In some embodiments, non-limiting examples of Stage 2, Stage 3, Stage 4, Stage 5, Stage 6, Stage 7 media include:

Media BOO45 (i-v):

| | Standard | Standard | Standard | Standard | Standard |
|---|---|---|---|---|---|
| Component | BOO45-i | BOO45-ii | BOO45-iii | BOO45- iv | BOO45-v |
| NH₄NO₃ | 825-2475 | 1237-2063 | 1485-1815 | 1650 | 1650 ± 2 |
| KNO₃ | 950-2850 | 1425-2375 | 1710-2090 | 1900 | 1900 ± 2 |
| MgSO₄ | 185-555 | 275-465 | 330-410 | 370 | 370 ± 2 |
| MnSO₄ | 8-26 | 12-22 | 15-19 | 16.9 | 16.9 ± 0.2 |
| ZnSO₄ | 4-12 | 6-10 | 8-9 | 8.6 | 8.6 ± 0.2 |
| CuSO₄ | 0.01-0.37 | 0.02-0.03 | 0.022-0.028 | 0.025 | 0.025 ± .002 |
| CaCl₂ | 220-660 | 330-350 | 400-480 | 440 | 440 ± 2 |
| Kl | 0.40-1.25 | 0.60-1.05 | 0.75-0.90 | 0.83 | 0.83 ± .02 |
| CoCl₂ | 0.012-0.378 | 0.020-0.030 | 0.022-0.028 | 0.025 | 0.025 ± .002 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 85-255 | 120-210 | 150-190 | 170 | 170 ± 2 |
| FeSO₄ | 13-42 | 20-34 | 25-30 | 27.8 | 27.8 ± 0.2 |
| Na₂EDTA | 18-56 | 28-46 | 33-41 | 37.3 | 37.3 ± 0.2 |
| myo-Inositol | 50-150 | 75-125 | 90-110 | 100 | 100 ± 2 |
| Thiamine | 0.2-0.6 | 0.3-0.5 | 0.35-0.45 | 0.4 | 0.4 ± 0.2 |
| Pyridoxine | 0.2-0.8 | 0.3-0.7 | 0.4-0.6 | 0.5 | 0.5 ± 0.2 |
| Nicotinic acid | 0.2-0.8 | 0.3-0.7 | 0.4-0.6 | 0.5 | 0.5 ± 0.2 |
| Glycine | 1-3 | 1.5-2.5 | 1.75-2.25 | 2 | 2 ± 1 |
| NAA | 0.05-0.15 | 0.07-0.12 | 0.09-0.11 | 0.1 | 0.1 ± 0.02 |
| IAA | 0.02-0.08 | 0.03-0.07 | 0.04-0.06 | 0.05 | 0.05 ± .02 |
| Sugar g/L | 15-45 | 22-37 | 27-33 | 30 | 30 ± 2 |
| Agar g/L | 2.7-8.2 | 4.1-6.8 | 4.9-6.1 | 5.5 | 5.5 ± 0.2 |

Media BOO46 (i-v):

| | Standard | Standard | Standard | Standard | Standard |
|---|---|---|---|---|---|
| Component | BOO46-i | BOO46-ii | BOO46-iii | BOO46-iv | BOO46-v |
| NH₄NO₃ | 700-2100 | 1050-1750 | 1260-1540 | 1400 | 1400 ± 2 |
| Ca(NO₃)₂ | 973-2919 | 1459-2433 | 1752-2140 | 1946 | 1946 ± 2 |
| K₂SO₄ | 606-1818 | 909-1515 | 1091-1333 | 1212.5 | 1212.5 ± 0.2 |
| MgSO₄ | 370-1110 | 555-925 | 665-815 | 740 | 740 ± 2 |
| MnSO₄ | 16.9-50.7 | 25.4-42.3 | 30.5-37.1 | 33.8 | 33.8 ± 0.2 |
| ZnSO₄ | 8.6-25.8 | 12.9-21.5 | 15.5-18.0 | 17.2 | 17.2 ± 0.2 |
| CuSO₄ | 0.02-0.08 | 0.03-0.07 | 0.04-0.06 | 0.05 | 0.05 ± .02 |
| CaCl₂ | 72-216 | 108-180 | 130-158 | 144 | 144 ± 2 |
| H₃BO₃ | 3-9 | 4-8 | 5-7 | 6.2 | 6.2 ± 0.2 |
| Na₂MoO₄ | 0.12-0.36 | 0.18-0.31 | .22-.28 | 0.25 | 0.25 ± .02 |
| KH₂PO₄ | 72-342 | 202-338 | 243-297 | 270 | 270 ± 2 |
| FeSO₄ | 16.68-50.04 | 25.02-41.70 | 30.06-36.66 | 33.36 | 33.36 ± .02 |
| Na₂EDTA | 22.38-67.14 | 33.57-55.95 | 40.36-49.16 | 44.76 | 44.76 ± .02 |
| myo-Inositol | 100-300 | 150-250 | 180-220 | 200 | 200 ± 2 |
| Thiamine | 0.4-1.4 | 0.6-1.2 | 0.8-1.0 | 0.9 | 0.9 ± 0.2 |
| Pyridoxine | 0.2-0.8 | 0.3-0.7 | 0.4-0.6 | 0.5 | 0.5 ± 0.2 |
| Nicotinic acid | 0.2-0.8 | 0.3-0.7 | 0.4-0.6 | 0.5 | 0.5 ± 0.2 |
| Glycine | 1-3 | 1.5-2.5 | 1.75-2.25 | 2 | 2 ± 1 |
| Riboflavin | 10-30 | 15-25 | 18-22 | 20 | 20 ± 2 |
| Ascorbic Acid | 50-150 | 75-125 | 90-110 | 100 | 100 ± 2 |
| NAA | 0.2-0.8 | 0.3-0.7 | 0.4-0.6 | 0.5 | 0.5 ± 0.2 |
| Sugar g/L | 15-45 | 22-37 | 27-33 | 30 | 30 ± 2 |
| Carrageenan g/L | 4-12 | 6-10 | 7-9 | 8 | 8 ± 2 |
| Charcoal g/L | 150-450 | 220-370 | 270-330 | 300 | 300 ± 2 |

Media BOO47 (i-v):

| | Standard | Standard | Standard | Standard | Standard |
|---|---|---|---|---|---|
| Component | BOO47-i | BOO47-ii | BOO47-iii | BOO47-iv | BOO47-v |
| NH₄NO₃ | 410-1240 | 620-1030 | 740-910 | 825 | 825 ± 2 |
| Ca(NO₃)₂ | 475-1425 | 710-1190 | 855-1045 | 950 | 950 ± 2 |
| MgSO₄ | 90-280 | 140-230 | 160-200 | 185 | 185 ± 2 |
| MnSO₄ | 4.20-12.70 | 6.30-10.60 | 7.65-9.25 | 8.45 | 8.45 ± .02 |
| ZnSO₄ | 2.0-6.5 | 3.0-5.5 | 3.5-5.0 | 4.3 | 4.3 ± 0.2 |
| CuSO₄ | .0062-0188 | .0094-.0156 | .0115-.0135 | 0.0125 | 0.0125 ± .0002 |
| CaCl₂ | 110-330 | 165-285 | 195-240 | 220 | 220 ± 2 |
| Kl | .20-.62 | .31-.52 | .37-.45 | 0.415 | 0.415 ± .002 |
| H₃BO₃ | 1.5-4.6 | 2.3-4.0 | 2.8-3.4 | 3.1 | 3.1 ± 0.2 |
| CaCl₂ | .006-.018 | .009-.015 | .011-013 | 0.0125 | 0.0125 ± .0002 |
| Na₂MoO₄ | .06-.18 | .09-.15 | .11-13 | 0.125 | 0.125 ± .002 |
| KH₂PO₄ | 40-130 | 60-110 | 75-95 | 85 | 85 ± 2 |
| FeSO₄ | 6.8-20.9 | 10.4-17.5 | 12.5-15.5 | 13.9 | 13.9 ± 0.2 |
| Na₂EDTA | 9.3-27.9 | 13.9-23.3 | 16.8-20.4 | 18.65 | 18.65 ± .02 |
| Na₂H₂PO₄ | 40-130 | 60-110 | 75-95 | 85 | 85 ± 2 |
| myo-Inositol | 50-150 | 75-125 | 90-110 | 100 | 100 ± 2 |
| Thiamine | 0.2-0.6 | 0.3-0.5 | 0.36-0.44 | 0.4 | 0.4 ± .2 |
| Pyridoxine | 0.2-0.8 | 0.3-0.7 | 0.4-0.6 | 0.5 | 0.5 ± 0.2 |
| Nicotinic acid | 1-3 | 1.5-2.5 | 1.75-2.25 | 2 | 2 ± 1 |
| Riboflavin | 2.5-7.5 | 3.7-6.2 | 4.5-5.5 | 5 | 5 ± 2 |
| NAA | 0.2-0.8 | 0.3-0.7 | 0.4-0.6 | 0.5 | 0.5 ± 0.2 |
| IBA | 0.5-1.5 | 0.7-1.3 | 0.9-1.1 | 1 | l ± 0.5 |
| Sugar g/L | 15-45 | 22-37 | 27-33 | 30 | 30 ± 2 |
| Agar g/L | 1.5-4.5 | 2.0-4.0 | 2.5-3.5 | 3 | 3 ± 2 |
| Carrageenan g/L | 2.5-7.5 | 3.7-6.2 | 4.5-5.5 | 5 | 5 ± 2 |
| Charcoal g/L | 2.5-7.5 | 3.7-6.2 | 4.5-5.5 | 5 | 5 ± 2 |

In some embodiments, non-limiting examples of spiked versions of these media include:

Media BOO45 (i-v):

| | Spiked | Spiked | Spiked | Spiked | Spiked |
|---|---|---|---|---|---|
| Component | BOO45-i | BOO45-ii | BOO45-iii | BOO45-iv | BOO45-v |
| NAA | 0.2 | 0.1 | 0.1 | 0.3 | 2 |
| IAA | 0.5 | 0.5 | 1 | 0.05 | 0.75 |

Media BOO46 (i-v):

| | Spiked | Spiked | Spiked | Spiked | Spiked |
|---|---|---|---|---|---|
| Component | BOO46-i | BOO46-ii | BOO46-iii | BOO46-iv | BOO46-v |
| NAA | 1 | 1.5 | 2 | 50 | 5 |

Media BOO47 (i-v):

| | Spiked | Spiked | Spiked | Spiked | Spiked |
|---|---|---|---|---|---|
| Component | BOO47-i | BOO47-ii | BOO47-iii | BOO47-iv | BOO47-v |
| NAA | 1 | 1.5 | 0.5 | 0.5 | 3 |
| IBA | 12 | 10 | 5 | 2 | 1 |

### Cytokinins and Analogs

Compounds useful according to the present disclosure include meta-topolin analogues having a general formula

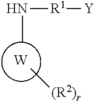

wherein W is an aryl or heteroaryl;
R1 is substituted or unsubstituted alkyl wherein any C in the alkyl can be substituted with 0, N or S;
each R2 is independently H, OH, C1-C6 alkyl, C1-C6 alkylene, C1-C6 alkynyl, halogen, cyano, C1-C6 alkyloxy, aryl or heteroaryl each optionally substituted with a C1-C6 alkyl, SH, NHR3, CO2R3 or halogen;
R3 is H, OH, C1-C6 alkyl, C1-C6 alkylene, C1-C6 alkynyl, halogen, carboxylic group, ester group, aldehyde or cyano;
r is 0 to 8.

In some embodiments, W is

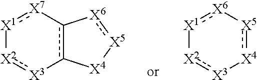

wherein a dashed line represents the presence or absence of a bond;

X1-X⁷ is each independently selected from C, N, O, S with the proviso that the X linking the ring to N is C.

In some embodiments, the compounds have a structure,

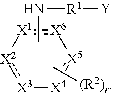

wherein a dashed line represents the presence or absence of a bond.

In some embodiments, the compounds have a structure,

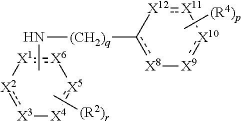

wherein a dashed line represents the presence or absence of a bond;

X8-X12 is each independently selected from C, N, O, S;
each R4 is independently H, OH, C1-C6 alkyl, C1-C6 alkylene, C1-C6 alkynyl, halogen, cyano, C1-C6 alkyloxy, aryl or heteroaryl each optionally substituted with a C1-C6 alkyl, SH, NHR3, CO2R3 or halogen;
R3 is H, OH, C1-C6 alkyl, C1-C6 alkylene, C1-C6 alkynyl, halogen, carboxylic group, ester group, aldehyde or cyano; p is 0 to 5; and q is 0 to 6.

In some embodiments, the compounds have a structure,

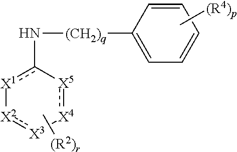

In some embodiments, the compounds have a structure,

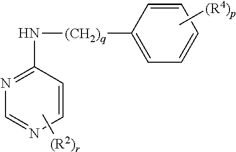

Further still, compounds can have structures selected from

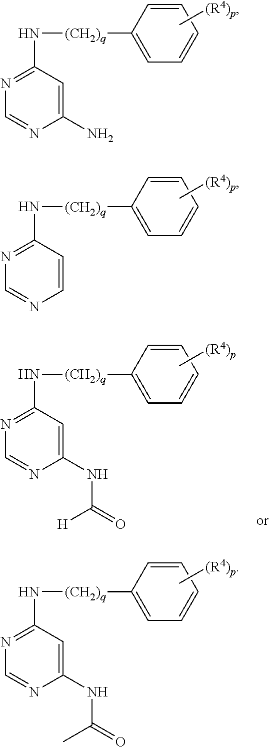

In one embodiments, R4 is OH.

In some embodiments, compounds have a structure selected from

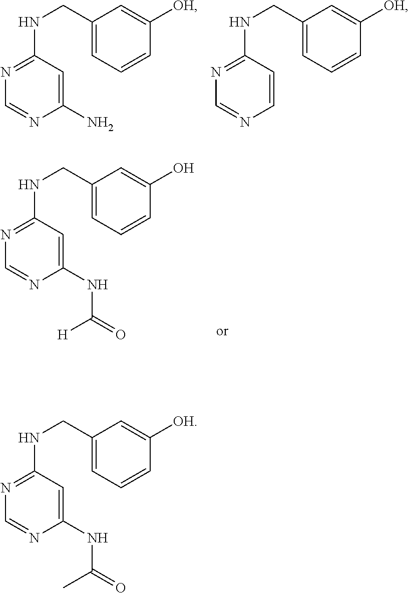 [SMILES: Nc1cc(NCc2cccc(O)c2)ncn1 ; Oc1cccc(CNc2ccncn2)c1 ; O=CNc1cc(NCc2cccc(O)c2)ncn1 ; CC(=O)Nc1cc(NCc2cccc(O)c2)ncn1]

In some embodiments, the compounds have a structure,

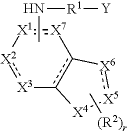

wherein a dashed line represents the presence or absence of a bond.

In another embodiment, the compounds have a structure

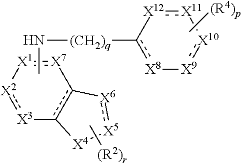

wherein a dashed line represents the presence or absence of a bond;

X8-X12 is each independently selected from C, N, O, S;
each R4 is independently H, OH, C1-C6 alkyl, C1-C6 alkylene, C1-C6 alkynyl, halogen, cyano, C1-C6 alkyloxy, aryl or heteroaryl each optionally substituted with a C1-C6 alkyl, SH, NHR3, CO2R3 or halogen;
R3 is H, OH, C1-C6 alkyl, C1-C6 alkylene, C1-C6 alkynyl, halogen, carboxylic group, ester group, aldehyde or cyano;
p is 0 to 5; and
q is 0 to 6.

In some embodiments, the compounds have a structure

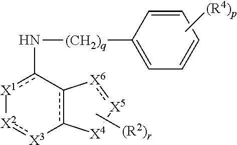

In still some embodiments, the compounds have a structure

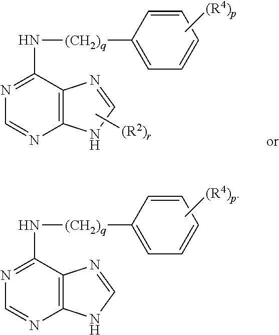

In some embodiments, the compound is meta-topolin, also known as 6-(3-hydroxybenzylamino)-purine, and by the abbreviation mT, having a empirical formula of C12H10N5OH, a molecular weight of 241.25, and the following structural formula:

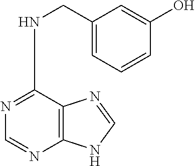 [SMILES: Oc1cccc(CNc2ncnc3[nH]cnc23)c1]

wherein said meta-topolin is a derivative of a willow tree or a poplar tree.

Meta-topolin analogues particularly include, without limitation, meta-topolin riboside, meta-topolin-9-glucoside, ortho-topolin, ortho-topolin riboside, ortho-topolin-9-glucoside, para-topolin, para-topolin riboside, para-topolin-9-glucoside, ortho-methoxytopolin, ortho-methoxytopolin riboside, meta-methoxytopolin, meta-methoxytopolin riboside and meta-methoxytopolin-9-glucoside. In particular embodiments, referred to herein as “mT limited embodiments”, 6-(3-fluorobenzylamino)purine (FmT), 6-(3-flurobenzylamino)purine-9-riboside (FmTR) and/or 6-(3-methoxybenzylamino)purine-9-riboside (memTR) can be excluded from the class of meta-topolin analogs.

Compounds useful according to the present disclosure include thidiazuron analogues having a general formula

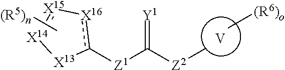

wherein V is an aryl or heteroaryl;
each R5 and R6 is each independently H, OH, C1-C6 alkyl, C1-C6 alkylene, C1-C6 alkynyl, halogen, cyano, C1-C6 alkyloxy, aryl or heteroaryl each optionally substituted with a C1-C6 alkyl or halogen;
n is 0 to 4;
o is 0 to 5
X13-X16 is each independently selected from C, N, O, S;
Z1 and Z2 are each independently NH, 0, SH or CH or Z1 and Z2 can be combined to form a substituted or unsubstituted aryl or heteroaryl; and

### Y1 is O or S.

In another embodiment, compounds have a structure

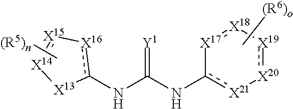

wherein X17-X21 is each independently selected from C, N, O, S.

In other embodiments, compounds include

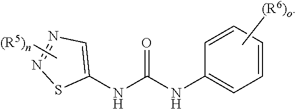

In one embodiment, the compound is thidiazuron, also known as 1-phenyl-3-(1,2,3-thiadiazol-5-yl)urea and 5-phenylcarbamoylamino-1,2,3-thiadiazole, has the empirical formula of C9H8N4OS, a molecular weight of 220.25 and the following structural formula

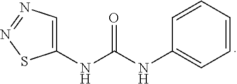 [SMILES: O=C(Nc1ccccc1)Nc1cnns1]

Compounds useful according to the present disclosure include B-naphthoxyacetic analogues having a general formula:

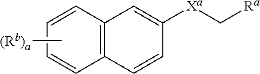

or a salt thereof;
wherein Ra is COR3, CO2R3, CONR3R4, or CN;
each Rb is independently R3; OR3; F; Cl; Br; I; CN; NO2; OCF3; CF3; NR2R3; SR3, SOR3, SO2R3, CO2R3, COR3, CONR3R4, CSNR4R5; or optionally substituted aryl or optionally substituted heteroaryl, wherein each substituent of aryl or heteroaryl is independently C1-C6 alkyl, F, Cl, Br, or I;
a is 1, 2, 3, 4, 5, 6, or 7;

### Xa is NH, S or O;

each R3 is independently H, C1-C6 alkyl, C2-C6 alkenyl, or C2-C6 alkynyl; and
each R4 is independently R3 or optionally substituted phenyl, wherein each substituent of phenyl is independently C1-C6 alkyl, F, Cl, Br, or I.

In another embodiment, compounds have a structure:

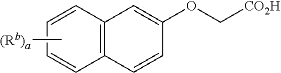

In one embodiment, the compound is B-naphthoxyacetic acid (NAA), also known as acetic acid, (2-naphthalenoxy)-(9CI) and has a CAS Number of 120-23-0, has the empirical formula of C12H10O3, a molecular weight of 202.21 and the following structural formula:

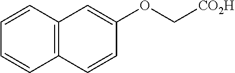 [SMILES: O=C(O)COc1ccc2ccccc2c1]

Other examples of NAA analogues may include, but are not limited to:

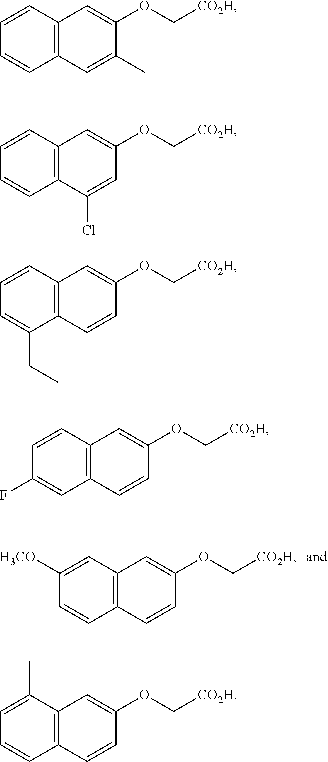

Compounds useful according to the present disclosure include indole butyric acid (IBA) analogues having a general formula:

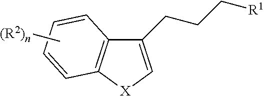

or a salt thereof;
wherein R1 is COR3, CO2R3, CONR3R4, or CN;
each R2 is independently R3; OR3; F; Cl; Br; I; CN; NO2; OCF3; CF3; NR2R3; SR3, SOR3, SO2R3, CO2R3, COR3, CONR3R4, CSNR4R5; or optionally substituted aryl or optionally substituted heteroaryl, wherein each substituent of aryl or heteroaryl is independently C1-C6 alkyl, F, Cl, Br, or I;
n is 1, 2, 3, or 4;

### X is NH, S or O;

each R3 is independently H, C1-C6 alkyl, C2-C6 alkenyl, or C2-C6 alkynyl; and
each R4 is independently R3 or optionally substituted phenyl, wherein each substituent of phenyl is independently C1-C6 alkyl, F, Cl, Br, or I.

In another embodiment, compounds have a structure:

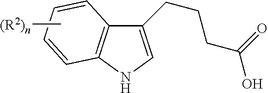

In one embodiment, the compound is indole butyric acid (IBA), also known as 1-Indole-3-butanoic acid, and has a CAS Number of 133-32-4, has the empirical formula of C12H13NO2, a molecular weight of 203.24, and the following structural formula:

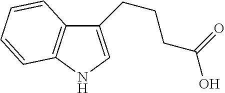 [SMILES: O=C(O)CCCc1c[nH]c2ccccc12]

Other examples of IBA analogues may include, but are not limited to:

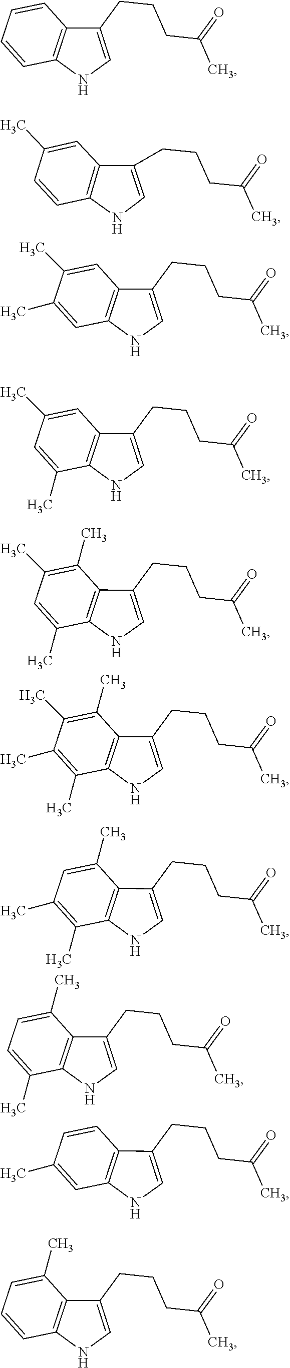

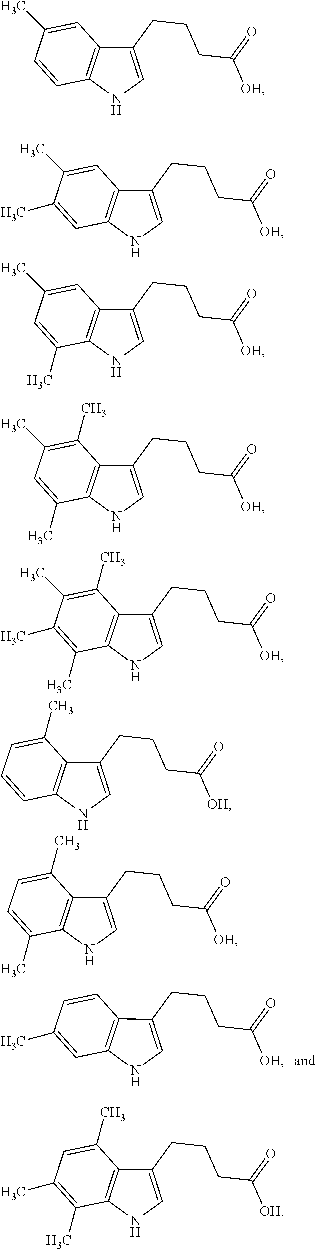

Compounds useful according to the present disclosure include benzylaminopurine (BAP) analogues having a general formula:

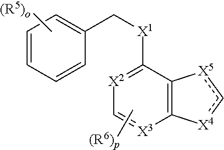

or a salt thereof;
wherein a dashed line represents the presence or absence of a bond;
each R5 and each R6 is independently R3; OR3; F; Cl; Br; I; CN; NO2; OCF3; CF3; NR2R3; SR3, SOR3, SO2R3, CO2R3, COR3, CONR3R4, CSNR4R5; or optionally substituted aryl or optionally substituted heteroaryl, wherein each substituent of aryl or heteroaryl is independently C1-C6 alkyl, F, Cl, Br, or I;
o is 0, 1, 2, 3, 4, or 5;
p is 0, 1, or 2;

### X1 is NH, S or O;

### X4 is —N═ and X5 is —NH—, —S—, or —O—; or X5 is —N═ and X4 is —NH—, —S—, or —O—;

X2 and X3 and are independently N or CR6;
each R3 is independently H, C1-C6 alkyl, C2-C6 alkenyl, or C2-C6 alkynyl; and
each R4 is independently R3 or optionally substituted phenyl, wherein each substituent of phenyl is independently C1-C6 alkyl, F, Cl, Br, or I.

In some embodiments, X4 is —N═ and X5 is —NH—, —S—, or —O—, and the dashed line represents the presence or absence of a bond. Thus, compounds of according to the formula below are contemplated.

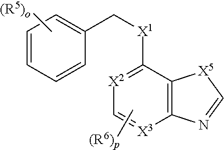

In other embodiments, X5 is —N═ and X4 is —NH—, —S—, or —O—. Thus, compounds of the formula below are contemplated.

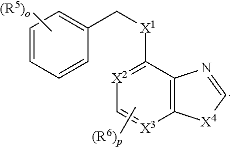

In another embodiment, the compounds have a structure:

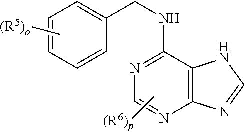

In another embodiment, the compounds have a structure:

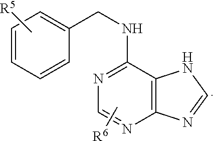

In one embodiment, the compound is benzylaminopurine (BAP), also known as 9H-Purin-6-amine, N-(phenylmethyl)-, which has a CAS Number of 1214-39-7, an empirical formula of C12H11N5, a molecular weight of 225.25, and the following structural formula:

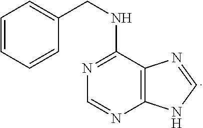 [SMILES: c1ccc(CNc2ncnc3[nH]cnc23)cc1]

Other examples of BAP analogues may include, but are not limited to:

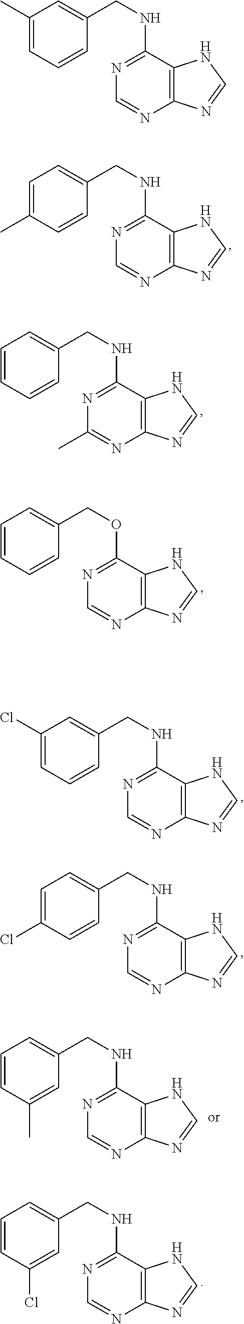

Compounds useful according to the present disclosure include 6-y-y-(dimethylallylamino)-purine (2ip) analogues having a general formula:

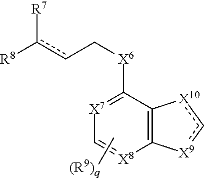

or a salt thereof;
wherein a dashed line represents the presence or absence of a bond;
wherein R7, R8, and each R9 are independently R3; OR3; F; Cl; Br; I; CN; NO2; OCF3; CF3; NR2R3; SR3, SOR3, SO2R3, CO2R3, COR3, CONR3R4, CSNR4R5; or optionally substituted aryl or optionally substituted heteroaryl, wherein each substituent of aryl or heteroaryl is independently C1-C6 alkyl, F, Cl, Br, or I;
q is 0, 1, or 2;

### X6 is NH, S or O;

### X9 is —N═ and X10 is —NH—, —S—, or —O—; or X10 is —N═ and X9 is —NH—, —S—, or —O—;

X7 and X8 and are independently N or CR9; and
each R3 is independently H, C1-C6 alkyl, C2-C6 alkenyl, or C2-C6 alkynyl; and
each R4 is independently R3 or optionally substituted phenyl, wherein each substituent of phenyl is independently C1-C6 alkyl, F, Cl, Br, or I.

In some embodiments, the dashed line represents the presence or absence of a bond. Thus, compounds of according to the formulas below are contemplated.

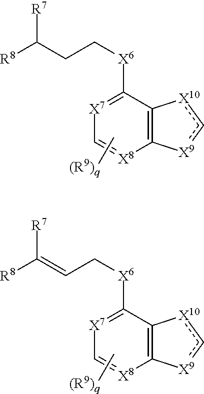

In some embodiments X9 is —N═ and X10 is —NH—, —S—, or —O—. Thus, compounds according the formula below are contemplated.

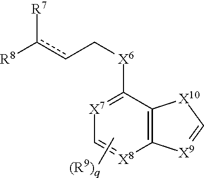

In other embodiments, X10 is —N═ and X9 is —NH—, —S—, or —O—. Thus, compounds having the structure shown below are contemplated.

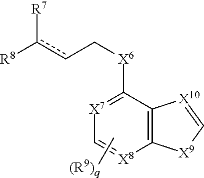

In another embodiment, compounds have a structure:

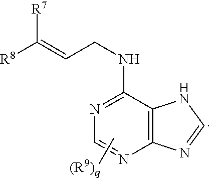

In one embodiment, the compound is 6-y,y,-(dimethylallylamino)-purine (2ip) or DAP, also known as 9H-purin-6-amine, N-(3-methyl-2-butene-1-yl)-, having a CAS No. 2365-40-4, an empirical formula of C10H13N5, a molecular weight of 203.24, and the following structural formula:

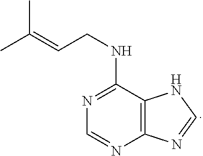

Other examples of Zip analogues may include, but are not limited to:

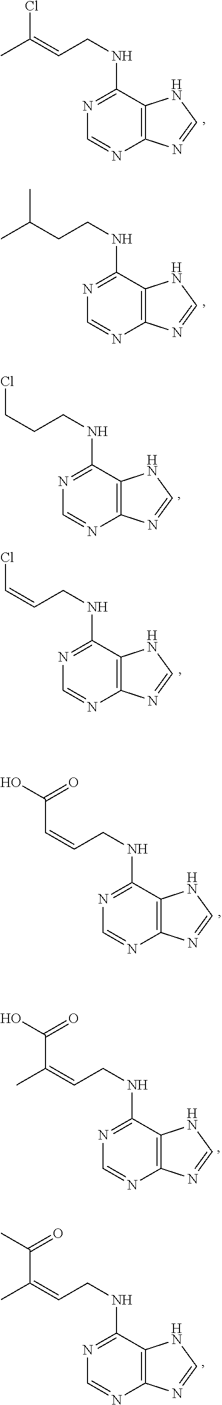

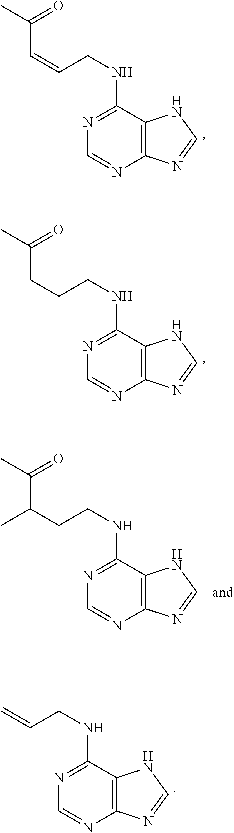

Compounds useful according to the present disclosure include N,N-diphenylurea (DPU) analogues having a general formula:

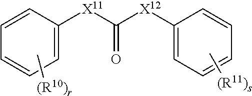

or a salt thereof;
wherein each R10 and each R11 is independently R3; OR3; F; Cl; Br; I; CN; NO2; OCF3; CF3; NR2R3; SR3, SOR3, SO2R3, CO2R3, COR3, CONR3R4, CSNR4R5; or optionally substituted aryl or optionally substituted heteroaryl, wherein each substituent of aryl or heteroaryl is independently C1-C6 alkyl, F, Cl, Br, or I;
r and s are independently 0, 1, 2, 3, 4, or 5;
X11 and X12 are independently NR10, S, or O;
each R3 is independently H, C1-C6 alkyl, C2-C6 alkenyl, or C2-C6 alkynyl; and
each R4 is independently R3 or optionally substituted phenyl, wherein each substituent of phenyl is independently C1-C6 alkyl, F, Cl, Br, or I.

In another embodiment, compounds have a structure:

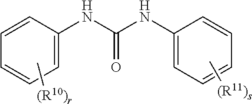

In one embodiment, the compound is N,N-diphenylurea (DPU), which is represented by a formula:

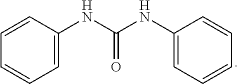 [SMILES: O=C(Nc1ccccc1)Nc1ccccc1]

Other examples of DPU analogues may include, but are not limited to:

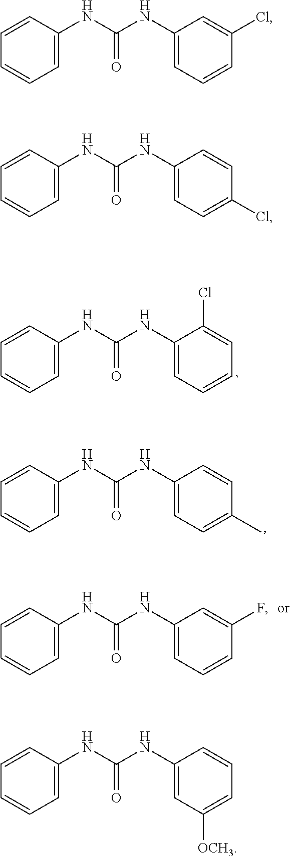 [SMILES: O=C(Nc1ccccc1)Nc1cccc(Cl)c1 ; O=C(Nc1ccccc1)Nc1ccc(Cl)cc1 ; O=C(Nc1ccccc1)Nc1ccccc1Cl ; Cc1ccc(NC(=O)Nc2ccccc2)cc1 ; O=C(Nc1ccccc1)Nc1cccc(F)c1 ; COc1cccc(NC(=O)Nc2ccccc2)c1]

Compounds useful according to the present disclosure include N-pyridinyl-N′-phenylurea (PPU used interchangeably with CPPU herein) analogues having a general formula:

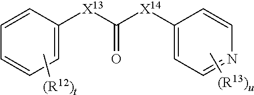

or a salt thereof;
wherein each R12 and each R13 is independently R3; OR3; F; Cl; Br; I; CN; NO2; OCF3; CF3; NR2R3; SR3, SOR3, SO2R3, CO2R3, COR3, CONR3R4, CSNR4R5; or optionally substituted aryl or optionally substituted heteroaryl, wherein each substituent of aryl or heteroaryl is independently C1-C6 alkyl, F, Cl, Br, or I;
t and u are independently 0, 1, 2, 3, 4, or 5;
X13 and X14 are independently NR12, S, or O;
each R3 is independently H, C1-C6 alkyl, C2-C6 alkenyl, or C2-C6 alkynyl; and
each R4 is independently R3 or optionally substituted phenyl, wherein each substituent of phenyl is independently C1-C6 alkyl, F, Cl, Br, or I.

In one embodiment, compounds have a structure:

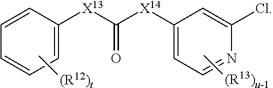

In another embodiment, compounds have a structure:

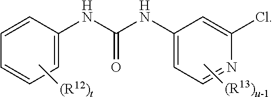

In one embodiment, the compound is N-(2-chloropyridin-4-yl)-N′-phenylurea, which is represented by a formula:

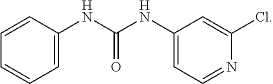 [SMILES: O=C(Nc1ccccc1)Nc1ccnc(Cl)c1]

Other examples of PPU analogues may include, but are not limited to:

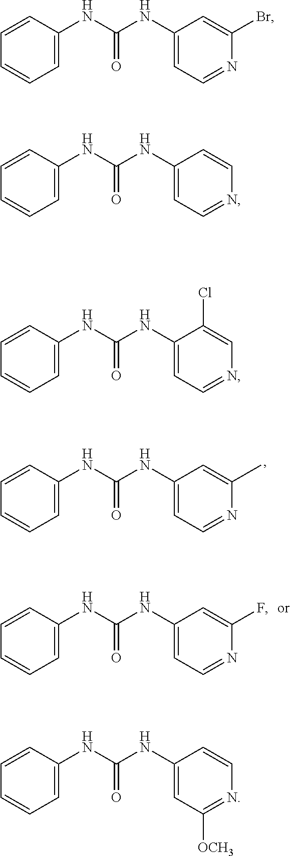 [SMILES: O=C(Nc1ccccc1)Nc1ccnc(Br)c1 ; O=C(Nc1ccccc1)Nc1ccncc1 ; O=C(Nc1ccccc1)Nc1ccncc1Cl ; Cc1cc(NC(=O)Nc2ccccc2)ccn1 ; O=C(Nc1ccccc1)Nc1ccnc(F)c1 ; COc1cc(NC(=O)Nc2ccccc2)ccn1]

### Propagation/Micropropagation

Micropropagation is the practice of rapidly multiplying stock plant material to produce a large number of progeny plants, using modern plant tissue culture methods. Micropropagation is used to multiply novel plants, such as those that have been genetically modified or bred through conventional plant breeding methods. It is also used to provide a sufficient number of plantlets for planting from a stock plant which does not produce seeds, or does not respond well to vegetative reproduction.

Micropropagation can first begin with the selection of plant material to be propagated. Clean stock materials that are free of viruses and fungi are important in the production of the healthiest plants.

Once the plant material is chosen for culture, the collection of explant(s) begins and is dependent on the type of tissue to be used, including stem tips, anthers, petals, pollen and others plant tissues. The explant material is then surface sterilized, usually in multiple courses of bleach and alcohol washes and finally rinsed in sterilized water. This small portion of plant tissue, sometimes only a single cell, is placed on a growth medium, typically containing sucrose as an energy source and one or more plant growth regulators (plant hormones). Usually the medium is thickened with agar to create a gel which supports the explant during growth. Some plants are easily grown on simple media but others require more complicated media for successful growth; the plant tissue grows and differentiates into new tissues depending on the medium. For example, media containing cytokinins are used to create branched shoots from plant buds.

Multiplication is the taking of tissue samples produced during the first stage and increasing their number. Following the successful introduction and growth of plant tissue, the establishment stage is followed by multiplication. Through repeated cycles of this process, a single explant sample may be increased from one to hundreds or thousands of plants. Depending on the type of tissue grown, multiplication can involve different methods and media. If the plant material grown is callus tissue, it can be placed in a blender and cut into smaller pieces and recultured on the same type of culture medium to grow more callus tissue. If the tissue is grown as small plants called plantlets, hormones are often added that cause the plantlets to produce many small offshoots that can be removed and recultured.

The next stage (“pretransplant” stage) involves treating the plantlets/shoots produced to encourage root growth and “hardening.” It is performed in vitro, or in a sterile or substantially sterile environment. “Hardening” refers to the preparation of the plants for a natural growth environment. Until this stage, the plantlets have been grown in “ideal” conditions, designed to encourage rapid growth. Due to lack of necessity, the plants are likely to be highly susceptible to disease and often do not have fully functional dermal coverings and will be inefficient in their use of water and energy. In vitro conditions are high in humidity and plants grown under these conditions do not form a working cuticle and stomata that keep the plant from drying out, when taken out of culture the plantlets need time to adjust to more natural environmental conditions. Hardening typically involves slowly weaning the plantlets from a high-humidity, low light, warm environment to what would be considered a normal growth environment for the species in question.

In the final stage of plant micropropagation, the plantlets are removed from the plant media and transferred to soil or (more commonly) potting compost for continued growth by conventional methods. This stage is often combined with the “pretransplant” stage.

Modern plant tissue culture is performed under aseptic conditions under filtered air. Living plant materials from the environment are naturally contaminated on their surfaces (and sometimes interiors) with microorganisms, so surface sterilization of starting materials (explants) in chemical solutions (usually alcohol or bleach) is required. Explants are then usually placed on the surface of a solid culture medium, but are sometimes placed directly into a liquid medium, particularly when cell suspension cultures are desired. Solid and liquid media are generally composed of inorganic salts plus a few organic nutrients, vitamins and plant hormones. Solid media are prepared from liquid media with the addition of a gelling agent, usually purified agar.

The composition of the medium, particularly the plant hormones and the nitrogen source (nitrate versus ammonium salts or amino acids) have profound effects on the morphology of the tissues that grow from the initial explant. For example, an excess of auxin will often result in a proliferation of roots, while an excess of cytokinin may yield shoots. A balance of both auxin and cytokinin will often produce an unorganized growth of cells, or callus, but the morphology of the outgrowth will depend on the plant species as well as the medium composition. As cultures grow, pieces are typically sliced off and transferred to new media (subcultured) to allow for growth or to alter the morphology of the culture. The skill and experience of the tissue culturist are important in judging which pieces to culture and which to discard. As shoots emerge from a culture, they may be sliced off and rooted with auxin to produce plantlets which, when mature, can be transferred to potting soil for further growth in the greenhouse as normal plants.

The tissue obtained from the plant to culture is called an explant. Based on work with certain model systems, particularly tobacco, it has often been claimed that a totipotent explant can be grown from any part of the plant. However, this concept has been vitiated in practice. In many species explants of various organs vary in their rates of growth and regeneration, while some do not grow at all. The choice of explant material also determines if the plantlets developed via tissue culture are haploid or diploid. Also the risk of microbial contamination is increased with inappropriate explants. Thus it is very important that an appropriate choice of explant be made prior to tissue culture.

The specific differences in the regeneration potential of different organs and explants have various explanations. The significant factors include differences in the stage of the cells in the cell cycle, the availability of or ability to transport endogenous growth regulators, and the metabolic capabilities of the cells. The most commonly used tissue explants are the meristematic ends of the plants like the stem tip, auxiliary bud tip and root tip. These tissues have high rates of cell division and either concentrate or produce required growth regulating substances including auxins and cytokinins. Some explants, like the root tip, are hard to isolate and are contaminated with soil microflora that become problematic during the tissue culture process. Certain soil microflora can form tight associations with the root systems, or even grow within the root. Soil particles bound to roots are difficult to remove without injury to the roots that then allows microbial attack. These associated microflora will generally overgrow the tissue culture medium before there is significant growth of plant tissue. Aerial (above soil) explants are also rich in undesirable microflora. However, they are more easily removed from the explant by gentle rinsing, and the remainder usually can be killed by surface sterilization. Most of the surface microflora do not form tight associations with the plant tissue. Such associations can usually be found by visual inspection as a mosaic, de-colorization or localized necrosis on the surface of the explant.

An alternative for obtaining uncontaminated explants is to take explants from seedlings which are aseptically grown from surface-sterilized seeds. The hard surface of the seed is less permeable to penetration of harsh surface sterilizing agents, such as hypochlorite, so the acceptable conditions of sterilization used for seeds can be much more stringent than for vegetative tissues.

Tissue cultured plants are clones, if the original mother plant used to produce the first explants is susceptible to a pathogen or environmental condition, the entire crop would be susceptible to the same problem, and conversely any positive traits would remain within the line also. Plant tissue culture is used widely in plant science; it also has a number of commercial applications. Applications include:

1. Micropropagation is widely used in forestry and in floriculture. Micropropagation can also be used to conserve rare or endangered plant species.

2. A plant breeder may use tissue culture to screen cells rather than plants for advantageous characters, e.g. pathogen resistance/tolerance.

3. Large-scale growth of plant cells in liquid culture inside bioreactors as a source of secondary products, like recombinant proteins used as biopharmaceuticals.

4. To cross distantly related species by protoplast fusion and regeneration of the novel hybrid.

5. To cross-pollinate distantly related species and then tissue culture the resulting embryo, which would otherwise normally die (Embryo Rescue).

6. For production of doubled monoploid (dihaploid) plants from haploid cultures to achieve homozygous lines more rapidly in breeding programs, usually by treatment with colchicine which causes doubling of the chromosome number.

7. As a tissue for transformation, followed by either short-term testing of genetic constructs or regeneration of transgenic plants.

8. Certain techniques such as meristem tip culture can be used to produce clean plant material from infected stock, such as potatoes and many species of soft fruit.

9. Micropropagation using meristem and shoot culture to produce large numbers of identical individuals.

Micropropagated plants can begin from a selected piece of plant tissue, called an “explant” or “mother plant.” This explant is the source of cells to be developed during the tissue culturing process. For example, the explant can be any segment or collection of cells from apical meristems, terminal buds, axillary buds, adventitious buds, accessory buds, pseudo-terminal buds, cambium, lateral meristem, lateral bud, vegetative buds, reproductive buds, mixed buds, shoot segments, shoot apices, stem segments, immature nodal sections from stems, lateral shoots, seedlings, seeds, shoots starting to rise from the ground, immature flower buds, inflorescences, crown segments, leaf segments, or any part thereof. In one embodiment, the explant is taken from a plant of about 1 week old, about 2 weeks old, about 3 weeks old, about 1 month old, about 2 month old, about 3 months old, about half year old, about 1 year old, about 2 years old plant, about 3 years old, about 5 years old, or more. The plant from which the explant is obtained can be grown in any suitable conditions, including but not limited to growing in a growth chamber, growing in a greenhouse, growing in a field, or growing in a tissue culture container (petri dish, margenta box, etc.). In some embodiments, the explant is tissue culture obtained from shoot clumps maintained as stock on growth media. In some embodiments, the explant is a nodal section having one or more axillary bud, which can be dormant or active. In some other embodiments, the explant is a seed or a part thereof.

In some embodiments, the tissue culture is obtained from shoot clumps maintained on growth media as stock. In some embodiments, the explant is a segment of bamboo cane. In some embodiments, the segment of bamboo cane comprises an internode. In some embodiments, the segment of bamboo cane comprises a nodal section. In some embodiments, the nodal section comprises a single bud. In some embodiments, the bud is dormant or active. In some embodiments, the explant is taken from a plant of about 1 week old, about 2 weeks old, about 3 weeks old, about 1 month old, about 2 month old, about 3 months old, about half year old, about 1 year old, about 2 years old plant, about 3 years old, about 5 years old, or more. In some embodiments, a bamboo seed or a part thereof is used.

Availability of virus free starting material is desirable for an agricultural seed production program. Thus, in some embodiments, the virus-free micropropagated plants begin from an explant that is subjected to one or more antiviral treatments, such as a chemical antiviral, thermotherapy, and/or meristem-tip culture. Meristem culture is one procedure used to produce a virus-free plant. In this method, apical or axillary growing tips (0.1-0.3 mm) are dissected and allowed to grow into plantlets on special culture medium under controlled conditions. The meristem culture for virus elimination is based on the principle that many viruses are unable to infect the apical/axillary meristem of a growing plant and that a virus-free plant can be produced if a small (e.g. 0.1-0.3 mm) piece of meristemic tissue is propagated. Excision of very small meristems typically requires a high degree of expertise and the development of plants from these small meristems (mericlones) can be lengthy (i.e. 4 to 8 months). To increase the percentage of virus freedom in regenerated mericlones, meristem culture can be combined with other antiviral treatments, such as thermotherapy (high temperature treatment) or chemotherapy (treatment with antiviral compounds) to increase the production of virus-free plants.

Thus, in some embodiments, the method comprises using meristem culture, thermotherapy, chemotherapy, or any combination thereof to produce a virus-free plant. In one embodiment, meristem culture, thermotherapy, and chemotherapy, are used to produce a virus-free plant. In some embodiments, the use of an antiviral can increase the success in producing a virus-free plant by at least two or three times. In one embodiment, chemotherapy comprises using an antiviral in a medium to culture the explant. In another embodiment, thermotherapy comprises incubating an explant under a 16 h light photoperiod at 30-40 μmol/^{m2}/s light intensity at 37° C. In some embodiments, the thermotherapy is for one week.

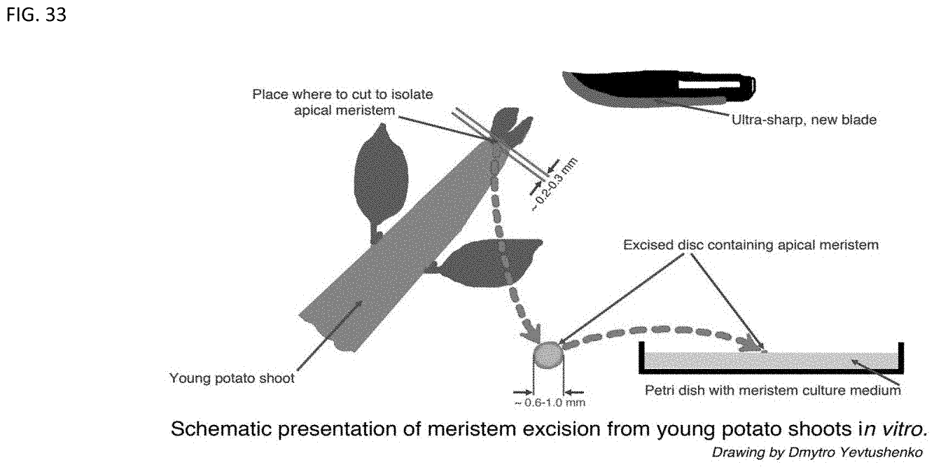

Accordingly, in one aspect of the present invention, a method for producing a virus-free plant comprises incubating an explant with medium, optionally comprising an antiviral; optionally, subjecting an explant of the plant culture to thermotherapy, wherein the explant grows into a plantlet; excising an apical meristem from the plantlet; and placing the apical meristem into a regeneration media; wherein a virus-free plantlet is produced. The excision is of a very small piece of meristem, and can be performed as depicted in FIG. 33.

In some embodiments, the regeneration media is selected from FIG. 32. In other embodiments, the regeneration media comprises an antiviral, such as Ribavirain (also known as Virazole). The method for producing a virus-free plant can also comprise culturing or subculturing, using conditions such as disclosed in PCT Publication No. WO2013016198, which is incorporated by reference in its entirety. For example, culturing or subculturing can be of the explant, apical meristem, the plantlet, or any combination thereof. The culturing or subculturing can be performed every one, two to three weeks. In one embodiment, culturing comprises incubating the explant under a 16 h light photoperiod at 80-100 μmol/m²/x light intensity at 24° C. In some embodiments, the method for producing a virus-free plant uses one or more different regeneration media, such as depicted in FIG. 32.

The plantlet produced by a method disclosed herein can be subcultured or tested for viruses. Any method known for testing for the presence of a virus can be used, such as by enzyme-linked immunosorbent assay (ELISA). The plantlet can be multiplied and subcultured, and used for further propagation. The pssent invention is applicable to a whole range of agricultural crops where a protocol for isolation and culture of meristematic cells or meristematic zones in vitro are available. Plantlets could be further induced and regenerated from the above cultures using either organogenesis or somatic embryogenesis.

In some embodiments, the tissue culture is obtained from shoot clumps maintained on growth media as stock. In some embodiments, the explant is a segment of bamboo cane. In some embodiments, the segment of bamboo cane comprises an internode. In some embodiments, the segment of bamboo cane comprises a nodal section. In some embodiments, the nodal section comprises a single bud. In some embodiments, the bud is dormant or active. In some embodiments, the explant is taken from a plant of about 1 week old, about 2 weeks old, about 3 weeks old, about 1 month old, about 2 month old, about 3 months old, about half year old, about 1 year old, about 2 years old plant, about 3 years old, about 5 years old, or more. In some embodiments, a bamboo seed or a part thereof is used.

The present invention provides methods for in vitro micropropagation of plant, for example, gymnosperm plants, angiosperm plants, monocot plants, dicot plants, crops, agriculturally/economically/environmentally important plants, etc.

The present invention provides methods for in vitro micropropagation of plant, for example, gymnosperm plants, angiosperm plants, monocot plants, dicot plants, crops, agriculturally/economically/environmentally important plants, etc.

In some embodiments, the methods disclosed herein can be used for in vitro micropropagation of a gymnosperm plant. For example, the methods can be used for in vitro micropropagation of the plants in the family/order of Cycadaceae, Zamiaceae, Ginkgoaceae, Welwitschiaceae, Gnetaceae, Ephedraceae, Pinaceae, Araucariaceae, Podocarpaceae, Sciadopityaceae, Cupressaceae, or Taxaceae.

In some embodiments, the methods disclosed herein can be used for in vitro micropropagation of an angiosperm plant. For example, the methods can be used for in vitro micropropagation of the plants in the family/order of Ceratophyllum, Chloranthaceae, eudicots, magnoliids, or monocots.

In some embodiments, the methods disclosed herein can be used for in vitro micropropagation of a dicot plant. For example, the methods can be used for in vitro micropropagation of the plants in the family/order of Buxaceae, Didymelaceae, Sabiaceae, Trochodendraceae, Tetracentraceae, Ranunculales, Proteales, Aextoxicaceae, Berberidopsidaceae, Dilleniaceae, Gunnerales, Caryophyllales, Saxifragales, Santalales, rosids, Aphloiaceae, Geissolomataceae, Ixerbaceae, Picramniaceae, Strassburgeriaceae, Vitaceae, Crossosomatales, Geraniales, Myrtales, Zygophyllaceae, Krameriaceae, Huaceae, Celastrales, Malpighiales, Oxalidales, Fabales, Rosales, Cucurbitales, Fagales, Tapisciaceae, Brassicales, Malvales, Sapindales, asterids, Cornales, Ericales, Boraginaceae, Icacinaceae, Oncothecaceae, Vahliaceae, Garryales, Solanales, Gentianales, Lamiales, Bruniaceae, Columelliaceae, Desfontainiaceae, Eremosynaceae, Escalloniaceae, Paracryphiaceae, Polyosmaceae, Sphenostemonacae, Tribelaceae, Aquifoliales, Apiales, Dipsacales, or Asterales.

In some embodiments, the methods disclosed herein can be used for in vitro micropropagation of a monocot plant. For example, the methods can be used for in vitro micropropagation of the plants in the family/order of Acorales, Alismatales, Asparagales, Dioscoreales, Liliales, Pandanales, Petrosaviales, Dasypogonaceae, Arecales, Commelinales, Poales, or Zingiberales.

In some embodiments, the methods disclosed herein can be used for in vitro micropropagation of a bamboo species, such as Phyllostachys edulis (e.g., Phyllostachys edulisi ‘Moso’), Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, or Guadua Angustifolia. In some embodiments, the bamboo species is Phyllostachys edulis, Moso.

In some embodiments, the plant is a non-bamboo species. In some embodiments, the non-bamboo plant species is Geranium spp. (e.g., Geranium rozanne), Hakonechloa macra (e.g., Hakonechloa macra ‘Aureola’, Hakonechloa macra ‘All gold’), Helleborus (e,g., Helleborus ‘Ivory Prince’), Phormium, Wasabi (e.g., Wasabi C2), Arundinaria (e.g., Arundinaria gigantean), or Solanum (e.g., Solanum tuberosum and Solanum tuberosum).

In some embodiments, the methods are used for rapid bamboo in vitro micropropagation. High shoot multiplication rate can be achieved in the methods disclosed herein. As used herein, the phrase “multiplication rate” refers to the multiplication fold of plant shoots obtained in a micropropagation process by starting from a single explant. For example, in the situation where the explant is a nodal section comprising a single bud, and 3 shoots are obtained after a micropropagation cycle, the multiplication rate is 3×. In some embodiments, by using the bud induction media (BOO1) and the shoot elongation/maintenance media (BOO2), a multiplication rate of at least about 2× to about 30× can be achieved after micropropagation. For example, about 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, about 30×, or more can be achieved within about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or about 28 days, or more.

In some embodiments, the present invention is based on the unexpected discovery that a pulsed treatment of an explant on a first medium comprising a strong cytokinin, such as TDZ, followed by a treatment of the explant on a second medium comprising one or more cytokinins other than TDZ, e.g., cytokinins that are relatively weaker than TDZ, such as meta-topolin, kinetin, isopentenyl adenine (iP, e.g., 2ip), zeatin, trans-zeatin, zeatin riboside, dihydrozeatin, benzyleadenin (BAP), or benzyladenosine ([9R]BAP), can provide rapid in vitro micropropagation with unexpected high multiplication rate.

In some embodiments, the methods comprise using a bud induction medium (BOO1) and a shoot elongation/maintenance media (BOO2), wherein the bud induction medium (BOO1) comprises a strong cytokinin, such as TDZ, and the shoot elongation/maintenance medium (BOO2) comprises a relatively weaker cytokinin, such as meta-topolin, kinetin, isopentenyl adenine (iP, e.g., 2ip), zeatin, trans-zeatin, zeatin riboside, dihydrozeatin, benzyleadenin (BAP), or benzyladenosine ([9R]BAP).

Examples of a bud induction medium (BOO1) are described herein. In some embodiments, a bud induction medium (BOO1) comprises one or more strong cytokinin or analog thereof. In some embodiments, the bud induction medium (BOO1) comprises only one strong cytokinin, wherein the cytokinin is TDZ or analog thereof. In some embodiments, the concentration of the strong cytokinin (e.g., TDZ) in the bud induction media (BOO1) is about 0.25 mg/L to about 100 mg/L, for example, about 0.5 mg/L to about 2 mg/L.

Examples of a shoot elongation/maintenance media (BOO2) are described herein. In some embodiments, a shoot elongation/maintenance medium (BOO2) comprises one or more cytokinin that is relatively weaker cytokinin, such as a cytokinin other than TDZ. In some embodiments, the shoot elongation/maintenance medium (BOO2) comprises only one relatively weaker cytokinin, such as BAP, meta-topolin, ip (e.g., 2ip), zeatin, zeatin riboside, or combination thereof. In some embodiments, the shoot elongation/maintenance medium (BOO2) comprises more than one cytokinins. In some embodiments, the concentration of a cytokinin in a shoot elongation/maintenance medium (BOO2) is about 0.01 mg/L to about 100 mg/L, for example, 0.25 mg/L to about 5 mg/L.

In some embodiments, the bud induction medium (BOO1) and/or the shoot elongation/maintenance medium (BOO2) comprises one or more auxin, such as β-naphthoxyacetic acid (NAA), 2,4-Dichlorophenoxy acetic acid (2,4-D), indole-3-butyric acid (IBA), indole-3-acetic acid (IAA), picloram, or analogs thereof. In some embodiments, the bud induction medium (BOO1) and/or the shoot elongation/maintenance medium (BOO2) comprises NAA. In some embodiments, the concentration of an auxin in the media is 0.01 mg/L to about 50 mg/L, for example, about 0.25 mg/L to about 0.5 mg/L.

In some embodiments, the methods are used for micropropagating plants in vitro. In some embodiments, the methods comprise (a) incubating a plant tissue culture, explant or seed in a first medium, and (b) then incubating the plant tissue obtained from step (a) in a second medium. In some embodiments, the first medium is a bud induction medium (BOO1), and the second medium is a shoot elongation and maintenance medium (BOO2).

In some embodiments, the methods comprise (a) incubating a tissue culture, explant or seed/seed part in a bud induction medium (BOO1) to induce shoot bud formation; (b) incubating the shoot buds obtained in step (a) in a shoot elongation/maintenance medium (BOO2).

The methods can further comprise (c) incubating the shoots from step (b) in a bud induction medium (BOO1) to induce shoot bud formation; and (d) incubating the shoot buds obtained in step (c) in a shoot elongation/maintenance medium (BOO2).

In some embodiments, the shoot buds obtained in step (a) and/or step (c) are separated prior to incubating the shoot buds in step (b) and/or step (d). In some embodiments, the separation produces groups of 1 to 3 shoot buds per separation prior to incubating the shoot buds in step (b) and/or step (d).

In some embodiments, the methods further comprises (e) repeating the incubating steps (c) and step (d) for at least once.

In some embodiments, when a bud induction medium (BOO1) and a shoot elongation and maintenance medium (BOO2) are used, the methods further comprise: (e) repeating the incubating step (c) and step (d) for at least one, at least two, at least three, at least four, at least five, at least six, at least seven, at least eight, or more additional cycles. There is no limit to how many times the cycling of step (c) and step (d) can be repeated. Buds and/or shoots obtained in step (a) or step (c) can be separated prior to the buds and/or shoots entering step (b) or step (d), respectively, wherein such separation can result in a single bud or shoot, 2 buds and/or shoots, 3 buds and/or shoots, or 4 or more buds and/or shoots per separation. Optimum separation for maximum, rapid production of bamboo copies of a single species, genotype or clone usually involves separating the buds and/or shoots obtained in step (a) or step (c) into 1-3 buds and/or shoots prior to entering into step (b) or step (d), respectively. Where there are 2 or more buds and/or shoots per separation this is known in the art as a clumping or “clump” of buds and/or shoots. Some variation in the methodologies of the present invention may be necessary so as to fine-tune the process for specific species, genotypes or clones of bamboo and such process variations are within the disclosure of this invention.

In some embodiments, both the bud induction medium (BOO1) and the shoot elongation/maintenance medium (BOO2) are liquid media. The advantage of liquid media is that the one can replace old media with fresh media, or replace one type of media with another type of media quickly and easily, without transferring the seedlings of plant from one container to another. Therefore, in some embodiments, the whole micropropagation process is achieved in a single container, for example, in a bioreactor.

In some embodiments, the bud induction media (BOO1) and/or the shoot elongation/maintenance media (BOO2) are semi-solid or solid media. In some embodiments, liquid media and semi-solid or solid media can be used subsequently with any desired order. For example, the bud induction medium (BOO1) in step (a) and/or step (c) is liquid, semi-solid, or solid; the shoot elongation/maintenance medium (BOO2) in step (b) and/or step (d) is liquid, semi-solid, or solid. Thus, in some embodiments, the bud induction medium (BOO1) of step (a) and/or step (c) is a liquid medium. In some embodiments, the bud induction medium (BOO1) of step (a) and/or step (c) is a solid medium. In some embodiments, the shoot elongation/maintenance medium (BOO2) of step (b) and/or step (d) is a liquid medium. In some embodiments, the shoot elongation/maintenance media (BOO2) of step (b) and/or step (d) is a solid media.

In some embodiments the methods of the present invention may involve using a liquid media for one step and a solid media for the next step of a particular cycle. For example, the present invention encompasses methods whereby step (a) is accomplished using liquid media and step (b) is accomplished using solid media. Alternatively, if both steps (a) and (b) and/or steps (c) and (d) are both done using liquid media, then the present invention contemplates that the liquid media may be changed without moving the buds and/or shoots to another container (e.g., test tube, bioreactor, jar, etc.). For example, if both steps (a) and (b) are accomplished using liquid media in a hydroponic setup, then the buds and/or shoots may remain in their fixed or unfixed position while the liquid media is replaced.

In some embodiments, the incubation of step (a) and/or step (c) lasts for a period that is sufficient to produce more than one shoot bud. For example, the period is set so as to produce at least 1, at least 2, at least 3, at least 4, at least 5, at least 6, at least 7, at least 8, at least 9, at least 10, at least 15, at least 20, at least 25, at least 26, at least 27, at least 28, at least 30, or more shoot buds for each bamboo tissue culture, explant or seed placed in the bud induction medium (BOO1) of step (a) or (c).

In some embodiments, the incubation period of step (a) or step (c) lasts for about one hour to about three weeks, or more. For example, the incubation period of step (a) or step (c) lasts for about 1 hour, about 2 hours, about 3 hours, about 4 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 24 hours, about 30 hours, about 36 hours, about 42 hours, about 48 hours, about 54 hours, about 60 hours, about 1 week, about 1.5 weeks, about 2 weeks, about 2.5 weeks, about 3 weeks, about 3.5 weeks, about 4 weeks, or more. In some embodiments, the incubation of step (a) and/or step (c) lasts from about 24 hours to about 60 hours. Thus, the incubation of step (a) and/or step (c) can last for about 24 hours, about 25 hours, about 26 hours, about 27 hours, about 28 hours, about 29 hours, about 30 hours, about 35 hours, about 40 hours, about 45 hours, about 50 hours, about 55 hours, about 56 hours, about 57 hours, about 58 hours, about 59, hours, or about 60 hours. In some embodiments the incubation stage of step (a) or step (c) can last longer than 60 hours, e.g., about 1 week, about 2 weeks, about 3 weeks, about 4 weeks, or longer.

In some embodiments, the incubation of step (b) and/or step (d) lasts for any desired period. In some embodiments, the incubation of step (b) and/or step (d) lasts from about 24 hours to about four weeks, or more. For example, the incubation of step (b) and/or step (d) lasts from about three days to about five days, or more. Thus, the incubation of step (b) and/or step (d) can last for about 24 hours, about 25 hours, about 26 hours, about 27 hours, about 28 hours, about 29 hours, about 30 hours, about 35 hours, about 40 hours, about 45 hours, about 48 hours, about 50 hours, about 55 hours, about 56 hours, about 57 hours, about 58 hours, about 59, hours, about 60 hours, about 72 hours, about 96 hours or about 120 hours. In some embodiments the incubation of step (b) and/or (d) can last longer than 120 hours, e.g., about 1 week, about 2 weeks, about 3 weeks, about 4 weeks, or longer. In some embodiments, the incubation period of step (b) or step (d) lasts for about 0.5 week, about 1 week, about 1.5 weeks, about 2 weeks, about 2.5 weeks, about 3 weeks, about 3.5 weeks, about 4 weeks, about 4.5 weeks, about 5 weeks, about 5.5 weeks, about 6 weeks, about 7 weeks, about 8 weeks, or more.

The incubation periods in the steps can be adjusted depending on the species of the plant, type of the explant, a desired multiplication rate. Without wishing to be bound by any theory, in some embodiments, for a bamboo species, the incubation period in step (a) or step (c) can be about 1 hour to about 3 weeks, for example, about 24 hours to about 60 hours; and the incubation period in step (b) or step (d) can be about 24 hours to about 4 weeks, for example, about 3 days to about 5 days.

The shoot multiplication rate can be further improved by repeating step (c) and step (d). For example, the multiple shoots developed after treatment of step (a) and treatment of step (b) can be subjected to one or more round of treatment of step (c) and treatment of step (d). In some embodiments, treatment in step (c) and treatment of step (d) are conducted at least once, at least twice, at least three times, at least four times, at least five times, at least six times, at least seven times, at least eight time, or more. Since the treatments in all steps are in short periods, a very short total time is needed to reach a very high shoot multiplication rate.

In some embodiments, each step of (a) to (e) can also be repeated before conducting the next step, by replacing old media with fresh media, for once, twice, three times, or more. In some embodiments, step (e) is repeated at least once, twice, three times, or more. In some embodiments, steps (a) to (e) take approximately one week, two weeks, three weeks, four weeks, five weeks, six weeks, or more.

In some embodiments, starting from a single explant, the present methods can provide about 10× to about 30× shoot multiplication rate in approximately three weeks. In addition, at least about 500, at least about 1,000, at least about 2,000, at least about 3,000, at least about 4,000, at least about 5,000, at least about 6,000, at least about 7,000, at least about 8,000, at least about 9,000, at least about 10,000, at least about 20,000, at least about 30,000, at least about 40,000, at least about 50,000, at least about 60,000, at least about 70,000, at least about 80,000, at least about 90,000, at least about 100,000, or more plant shoots can be obtained within about 6 weeks, about 10 weeks, about 2 months, about 2.5 months, about 3 months, about 4 months, about 5 months, about 6 months.

To further improve the shoot multiplication rate, a separation step can be added during or immediately after one or more steps selected from steps (a), (b), (c), and (d). For example, multiple shoot buds produced in step (a) and/or step (c), or multiple shoots produced in step (c) and/or step (c) can be separated into individual pieces, and each of the separated pieces can be placed in an individual container comprising fresh media. For example, multiple shoot buds developed in a bud induction medium (BOO1) can be divided into individual pieces, and placed either on a fresh bud induction medium (BOO1), or on a fresh shoot elongation/maintenance medium (BOO2); multiple shoots developed in a shoot elongation/maintenance medium (BOO2) can be separated into individual pieces, and placed either on a fresh shoot elongation/maintenance medium (BOO2), or a fresh bud induction medium (BOO1). Each separated piece may comprise 1, 2, 3, 4, 5, 6, 7, 8, 9, 10, or more shoot buds or shoots.

The present invention also provides methods for plant micropropagation using a plant growth system described herein.

In some embodiments, a plant growth system of the present invention is used for plant micropropagation. In some embodiments, it is used for bamboo micropropagation. In some embodiments, it is used for micropropagation of Phyllostachys edulisi ‘Moso’, Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, or Guadua Angustifolia, Nigra Henon, Rufa, or Nigra.

In some embodiments, a plant growth system of the present invention is used for plant micropropagation, wherein the plant is a perennial, grass, or phtyo-pharmaceutical plant. In some embodiments, it is used for micropropagation of a perennial. The perennial can be an evergreen, deciduous, monocarpic, woody, or herbaceous perennial. In some embodiments, the perennial is Begonia, banana, goldenrod, mint, agave, maple tree, pine tree, apple tree, alfalfa or red clover. In some embodiments, it is used for micropropagation of a grass. The grass can be of the Poaceae (or Gramineae), Cyperaceae or Juncaceae family. The grass can be a perennial grass or a cereal grass. The grass can be switchgrass, big bluestem, miscanthus, alfalfa, orchard grass, or reed canarygrass. The grass can be bamboo, tall fescue, goat grass, or Kentucky bluegrass.

Other types of grasses include wheat, rye, oat, barley, soy, and hemp, as well as straws derived therefrom. In some embodiments, it is used for micropropagation of a phyto-pharmaceutical plant. In some embodiments, it is used for micropropagation of Aloe vera, Ginger, Grape, Cannabis, Garlic, Onion, Echinacea, Geranium, Hakonechloa, Miscanthus, Arundo donax, Switch grass, Rice, or Sugar cane.

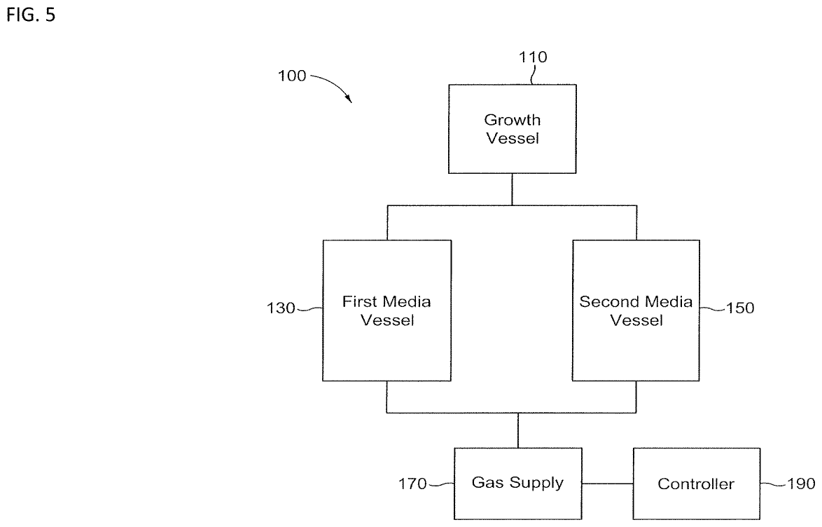

Referring again to the system 100 in FIG. 5, in use for plant micropropagation, the plant propagation sequence starts with placing an explant into the growth vessel 110. In some embodiments, the first media container 130 comprises a bud induction medium (BOO1) as described herein, and the second media container 150 comprises a shoot elongation/maintenance medium (BOO2).

In some embodiments, the bud induction medium (BOO1) comprises an effective amount of thidiazuron (TDZ) or analog thereof, and wherein the shoot elongation/maintenance medium (BOO2) comprises an effective amount of one or more cytokinins other than TDZ or an analog thereof. In some embodiments, the concentration of TDZ or analog thereof in the bud induction medium (BOO1) is about 0.25 mg/L to about 100 mg/L, e.g., from 0.5 mg/L to about 2 mg/L. In some embodiments, one or more cytokinins other than TDZ or an analog thereof in the shoot elongation/maintenance medium (BOO2) is selected from the group consisting of N⁶-benzylaminopurine (BAP), meta-topolin (mT), zeatin, kinetin, 2-isopentenyladenine (2ip), adenine hemi sulfate, dimethylallyladenine, N-(2-chloro-4-pyridyl)-N′-phenylurea) (4-CPPU), and analogs of each thereof. In some embodiments, the concentration of the one or more cytokinins other than TDZ or an analog thereof is from about 0.01 mg/L to about 100 mg/L, e.g., from about 0.25 mg/L to about 5 mg/L. In some embodiments, the bud induction medium (BOO1) and/or the shoot elongation/maintenance medium (BOO2) further comprises one or more auxins, such as β-naphthoxyacetic acid (NAA), 2,4-Dichlorophenoxy acetic acid (2,4-D), indole-3-butyric acid (IBA), indole-3-acetic acid (IAA), picloram, and analogs of each thereof.

In some embodiments, the first incubation sequence of 304 lasts for about half hour to about three weeks, e.g., for about 24 hours to about 60 hours, and the second incubation sequence of 314 lasts for about 24 hours to about four weeks, e.g., for about three days to about five days.

In some embodiments, the length the plant propagation sequence is determined by the multiplication rate reached. In some embodiments, the multiplication rate is from at least about 1,000 to at least about 100,000 within about 3 weeks to about 6 months.

As far as the inventors know, this is the first time that such a system has been used for plant micropropagation, especially for bamboo micropropagation. The methods using a bioreactor are unique at least for the following reasons:

1. The methods are suitable for both small scale (e.g., laboratory) and large scale (e.g., industrial) plant micropropagation.

2. The methods allow the pulsing micropropagation technology described herein to be used in a more efficient way (e.g., recycled medium; less labor; more accurate control; less contamination; etc.).

3. The methods enable greatly improved shoot/plant multiplication over prior methods (e.g., micropropagation using solid medium, and micropropagation using liquid medium without a bioreactor).

4. The methods enable greater plant survival rate over prior methods, particularly for certain plant species, such as Moso bamboo.

5. Bamboo releases phenolics which are harmful to the shoots/plants when they buildup in the media/environment, which has been a problem with using solid medium.

Moving from solid growth environment (e.g., plant micropropagation in tissue culture tubes/boxes) to the liquid environment and combining pulsing methods and a bioreactor system, the present invention achieves a major improvement in number of shoots/plants that are obtained, as well as improving the resultant plants' health and ability to produce full size plants. Without wishing to be bound by any theory, the inventors believe these achievements are the result of controlling/reducing the exposure of the shoots/plantlets to toxic components in the growth compositions (e.g., certain plant hormones, such as TDZ) and/or plant produced by-products (e.g., phenolics), by utilizing the bioreactor systems of the present invention.

In addition to the methods described above which are based on using “bud induction media (BOO1)” and “shoot elongation/maintenance media (BOO2)” combination, the present invention also provides alternative plant micropropagation methods based on using “Stage 1 media”, “Stage 2 media”, “Stage 3 media”, and/or more media.

In embodiments, the methods comprising using at least one “Stage 1 media” and at least one “Stage 2 media”, and an explant. In some embodiments, the Stage 1 and Stage 2 media are used sequentially during plant propagation. In some embodiments, the explants remain on the Stage 1 medium for about 1 to about 36 hours (e.g., when spiked media are used). In some embodiments, the explants remain on the Stage 1 medium for 10-120 days (e.g., when standard or reduced media are used). In some embodiments, the explants stay on Stage 1 medium for at least 1, 2, 3, 4, 5, 6, 7, 8, 9, 10 or more rounds (e.g., in each round, explants are transferred from an old Stage 1 medium to a fresh Stage 1 medium) before being transferred to the Stage 2 medium. In some embodiments, the explants remain on the Stage 2 medium for about 1 to about 36 hours (e.g., when spiked media are used). In some embodiments, the explants remain the Stage 2 medium for about 10-120 days (e.g., when standard or reduced media are used). In some embodiments, the explants stay on Stage 2 medium for at least 1, 2, 3, 4, 5, 6, 7, 8, 9, 10 or more rounds (e.g., in each round, explants are transferred from an old Stage 2 medium to a fresh Stage 2 medium).

In some embodiments, the Stage 1 and Stage 2 media are used in rotation during plant propagation. In some embodiments, the rotation is continuous for at least 1, 2, 3, 4, 5, 6, 7, 8, 9, 10 or more cycles. In some embodiments, the explants stay on Stage 1 medium for at least 1, 2, 3, 4, 5, 6, 7, 8, 9, 10 or more rounds first before being transferred to the Stage 2 medium. In some embodiments, the explants stay on Stage 2 medium for at least 1, 2, 3, 4, 5, 6, 7, 8, 9, 10 or more rounds first before being transferred back to the Stage 1 medium. In some embodiments, the explants are on the Stage 1 medium for about 1-36 hours (e.g., when spiked media are used) or more (e.g., when standard or reduced media are used) followed by being transferred to the Stage 2 medium. In some embodiments, the rotation is continuous until multiple shoots are observed. In some embodiments, the rotation takes about 1 week to about 24 months, depending on plant species and media.

Optionally, the multiplied shoots are then placed on a Stage 3 medium for further multiplication until desired number of shoots is obtained, depending on previous treatments. The explants on the Stage 3 medium can be further transferred to a Stage 4 medium. In some embodiments, the explants are on the Stage 3 medium for about 1-36 hours (e.g., when spiked media are used) followed by being transferred to the Stage 4 medium. In some embodiments, the explants are on the Stage 3 medium for about 10-120 days or more (e.g., when standard or reduced media are used) followed by being transferred to the Stage 4 medium. In some embodiments, the explants remain the Stage 4 medium for about 10-120 days.

Alternatively, the multiplied shoots obtained from a Stage 2 medium can be rotated between at least one Stage 3 medium and at least one Stage 4 medium. In some embodiments, the rotation is continuous for at least 1, 2, 3, 4, 5, 6, 7, 8, 9, 10 or more cycles. In some embodiments, the rotation is continuous until desired number of shoots. In some embodiments, the desired number of shoots is obtained by separation into new tubes and further expansion. In some embodiments, about one to ten shoots per tube are obtained per multiplication cycle.

In some embodiments, the explants are placed on a Stage 1 medium, a Stage 2 medium, and a Stage 3 medium in rotation, until desired number of shoots is obtained. In some embodiments, the multiplied shoots are then placed on a Stage 4 medium.

In some embodiments, the explants are placed on a Stage 1 medium and a Stage 2 medium in rotation, until desired number of shoots is obtained. In some embodiments, the multiplied shoots are then placed on a rotation of a Stage 3 medium and a Stage 4 medium.

In some embodiments, the explants are placed on a Stage 1 medium for about 1, 2, 3, 4, 5, 6, 7, 8, 9, 10, or more rounds, and then transferred to a Stage 2 medium for about 1, 2, 3, 4, 5, 6, 7, 8, 9, 10, or more rounds, until desired number of shoots is obtained. In some embodiments, the multiplied shoots are then placed on a rotation of a Stage 3 medium and a Stage 4 medium.

In some embodiments, the explants are placed on a Stage 1 medium first. In some embodiments, the explants are on the Stage 1 medium for about 1-36 hours (e.g., when spiked media are used). In some embodiments, the explants are on the Stage 1 medium for about 10-120 days or more (e.g., when standard or reduced media are used). In some embodiments, this step comprises 1, 2, 3, 4, 5, 6, 7, 8, 9, 10 or more rounds of fresh Stage 1 medium. Then, the explants are kept on a rotation of a Stage 2 medium and a Stage 3 medium, until desired number of shoots is obtained. In some embodiments, the multiplied shoots are then placed on a Stage 4 medium. In some embodiments, the multiplied shoots remain on the Stage 4 medium for about 10-120 days.

Still optionally, the multiplied shoots obtained from a Stage 4 medium can be transferred onto a Stage 5 medium as described herein. In some embodiments, the shoots are placed on a Stage 5 medium for about 1-24 hours or more (e.g., when spiked media are used). In some embodiments, the shoots are placed on a Stage 5 medium for about 10 to 120 days (e.g., when standard or reduced media are used). In some embodiments, the shoots are transferred to small tissue culturing boxes, such as the magenta boxes.

Still optionally, the explants are kept on the Stage 5 medium first until the desired number of shoots is obtained, then transferred to a Stage 6 medium as described herein. In some embodiments, the shoots are placed on a Stage 6 medium for about 1-24 hours or more (e.g., when spiked media are used). In some embodiments, the shoots are placed on a Stage 6 medium for about 10 to 120 days (e.g., when standard or reduced media are used).

In some embodiments, the explants obtained from the Stage 4 medium are placed on a rotation of a Stage 5 medium and a Stage 6 media, until the desired number of shoots is obtained.

Still optionally, the explants kept on the Stage 6 medium are transferred to a Stage 7 medium as described herein. In some embodiments, the shoots are placed on a Stage 7 medium for about 1-24 hours or more (e.g., when spiked media are used). In some embodiments, the shoots are placed on a Stage 7 medium for about 10 to 120 days (e.g., when standard or reduced media are used).

Still optionally, the multiplied shoots obtained from a Stage 4 medium can be transferred onto a rotation of a Stage 4 medium, a Stage 5 medium, and a Stage 6 medium as described herein.

Still optionally, the multiplied shoots obtained from a Stage 7 medium can be transferred one or more other additional media (e.g., a Stage 8, a Stage 9, etc.) for further propagation if needed.

A variety of appropriate explants can be used in accordance with the present disclosure. In certain embodiments according to the present disclosure, immature nodal sections from stems can be used as the explant material. In one embodiment, the explants can be new growth canes with the lateral shoots just breaking the sheath at nodal section(s). New growth canes include those obtained from the plant within a current season or year, wherein such new growth canes can be obtained from any node on the plant. In one particular embodiment, explant material includes or is limited to the third node from the base of a cane.

In some embodiments, the plant is a bamboo. Detailed methods for collecting and initially disinfecting bamboo explants are described in WO/2011/100762, which is incorporated herein by reference in its entirety. In some embodiments, the disinfectant such as dichloroisocyanuric acid, dichloroisanuric acid, trichlorotriazinetriona, mercuric chloride, hydrogen peroxide, FungiGone™ (bioWorld, Inc., Dublin, Ohio), plant preservatives can be used. In some embodiments, following the initial disinfection, the outer sheaths of a bamboo can be peeled off and discarded and the remaining piece can be put into an approximately 1% to about 50% solution of a commercial bleach or a similar disinfecting solution. In some embodiments, the bleach can be heated to about 20-60° C., such as 23-50° C. In some embodiments, sonication and vacuum infiltration of the tissue can also be used with the described disinfection procedures.

In some embodiments, the multiplication process can continue substantially indefinitely by continuing to separate and multiply shoots. In some embodiments, the multiplication cycles can be repeated without initiating new explants for at least 1 month, at least 3 months, at least 6 months, at least 12 months, at least 24 months, at least 36 months, or more. In some embodiments, the multiplication cycles include 1-10 days per cycle, 2-9 days per cycle, 3-6 days per cycle, 0.5-3 days per cycle, 4-5 days per cycle, 0.5-1 day per cycle, 10-120 days per cycle, etc.

The present invention has many advantages. Without wishing to be bound by any theory, the methods disclosed herein do not require the use of seeds or inflorescence to start plants, or selection of diseased starting plants, or the use of antibiotics, somatic embryogenesis, pseudospiklets, or induction and/or reversion of flowering. For successful growth following tissue culture, the produced plants do not require watering directly on the pot but remain robust with overhead watering and do not require multiple adjustments to light intensity or humidity conditions prior to transfer to a greenhouse or other growing conditions. Moreover, media can be free from polyaspartic acid(s), seaweed concentrates and/or surfactants. These improvements over prior methods provide even additional advantages related to the health of produced plants and efficiency of growth and processing.

In some embodiments, the present invention can be used for grass propagation. In some embodiments, the micropropagated plants have not been genetically modified. Other particular embodiments exclude the use of timentin and/or kanamycin in the micropropagation procedure.

In some embodiments, when the methods are used for bamboo propagation, explants from a bamboo plant between the age of 3 months and 3 years are used. In some embodiments, a node from the cane with the lateral shoot just breaking the sheath can be used as the explants. In some embodiments, each nodal section can be cut into 3-5 millimeter sections with the shoot intact. In some embodiments, the outer sheaths can be peeled off and discarded and the remaining nodal section piece put into a 10% bleach solution with a final concentration of 0.6% sodium hydrochloride. In some embodiments, the explant in bleach solution can be placed onto a Lab Rotators, Adjustable speed, Barnstead/Lab line orbital Shaker (model number KS 260) shaker table for 1 hour at 6-9 revolutions per minute. The explants can then be put into a 1% bleach solution with a final concentration of 0.06% sodium hydrochloride, and be placed back onto the shaker table for 30 minutes. This 1% bleach solution step can then be repeated.

In some embodiments, individual explants can then be placed on a Stage 1 media (15-25 mL) within a tube and the tubes can be placed into a regulated clean growth chamber at a temperature of from 65° F.-70° F. and a full spectrum light level of 36-54 μmole/m²/s². The initial Stage 1 media can be BOO38-iv at a pH of 5.7. The explants can then be transferred to fresh BOO38-iv media every 10-120 days (usually every 21 days), with contaminated tubes being discarded.

In some embodiments, if a spiked version of the BOO38-iv media is utilized, the explants can be placed in the spiked media for 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours before transition to a “standard” media disclosed herein or to a media containing substantially reduced or no cytokinins (“reduced” media as used herein) for the remainder of the 10-120 day cycle. Additional time periods for placement in a spiked media include anywhere between 0.1 and 240 hours and can include, without limitation, 0.1-0.5 hours, 0.3-2.5 hours, 2.5-6 hours, 1-10 hours, 5-15 hours, 10-20 hours, 15-25 hours, 20-30 hours, 25-35 hours, 30-40 hours, 35-45 hours, 40-50 hours, 45-55 hours, 50-60 hours, 55-65 hours, 60-70 hours, 65-75 hours, 70-80 hours, 75-85 hours, 80-90 hours, 85-95 hours, 90-100 hours, 95-105 hours, 100-110 hours, 105-115 hours, 110-120 hours, 115-125 hours, 120-130 hours, 125-135 hours, 130-140 hours, 135-145 hours, 140-150 hours, 145-155 hours, 150-160 hours, 155-165 hours, 160-170 hours, 165-175 hours, 170-180 hours, 175-185 hours, 180-190 hours, 185-195 hours, 190-200 hours, 195-205 hours, 200-210 hours, 205-215 hours, 210-220 hours, 215-225 hours, 220-230 hours, 225-235 hours, 230-240 hours, 235-245 hours, 240-250 hours, 3-6 hours, 7-17 hours, 12-22 hours, 17-27 hours, 22-32 hours, 27-37 hours, 32-42 hours, 37-47 hours, 42-52 hours, 47-57 hours, 52-62 hours, 57-67 hours, 62-72 hours, 67-77 hours, 72-82 hours, 77-87 hours, 82-92 hours, 87-97 hours, 92-102 hours, 97-107 hours, 102-112 hours, 107-117 hours, 112-122 hours, 117-127 hours, 122-132 hours, 127-137 hours, 132-142 hours, 137-147 hours, 142-152 hours, 147-157 hours, 152-162 hours, 157-167 hours, 162-172 hours, 167-177 hours, 172-182 hours, 177-187 hours, 162-172 hours, 167-177 hours, 182-192 hours, 187-197 hours, 192-202 hours, 197-207 hours, 202-212 hours, 207-217 hours, 212-222 hours, 217-227 hours, 222-232 hours, 227-237 hours, 232-242 hours, 237-247 hours or 242-252 hours. Placement in spiked media can also be 0.5 hours less than a cycle in standard or reduced media, 1 hour less than a cycle in standard or reduced media and all time periods in between 1 and 240 hours less than a cycle in standard or reduced media. Alternatively, in place of spending the remainder of the cycle in the standard or reduced media, explants can be placed on a spiked media for a period of time followed by culture on a standard or reduced media for the full cycle time (i.e. 10-120 days not reduced by time spent in the spiked media).

Media containing no cytokinins or substantially reduced cytokinins can be a reduced BOO36 media, reduced BOO40 media, reduced BOO37 media, reduced BOO31 media, reduced BOO38 media, reduced BOO28 media, reduced BOO29 media, reduced BOO30 media, reduced BOO39 media, reduced BOO41 media, reduced BOO42 media, reduced BOO43 media, reduced BOO44 media, reduced BOO35 media, reduced BOO38 CPPU media, reduced BOO38 DPU media with all cytokinins and/or auxins removed or can have at least one cytokinin and/or auxin's amount reduced by 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%, 1-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, 90-100%, 3-6%, 7-17%, 12-22%, 17-27%, 22-32%, 27-37%, 32-42%, 37-47%, 42-52%, 47-57%, 52-62%, 57-67%, 62-72%, 67-77%, 72-82%, 77-87%, 82-92% or 87-97%. Non-limiting examples of reduced media include (embodiments with no cytokinins or auxins not shown in table format):

### Media BOO35

| | Reduced | Reduced | Reduced | Reduced | Reduced |
|---|---|---|---|---|---|
| Component | BOO35-i | BOO35-ii | BOO35-iii | BOO35-iv | BOO35-v |
| NAA | 0.15 | 0.01 | 0.1 | 0.15 | 0.1 |
| BAP | 1 | 0.75 | 0.9 | 1 | 0.5 |
| Thidiazuron | 0.2 | 0.25 | 0.2 | 0.25 | 0.25 |
| Meta- | 4 | 5 | 4.5 | 5 | 5 |
| Topolin | | | | | |

### Media BOO38 CPPU

| | Reduced | Reduced | Reduced | Reduced | Reduced |
|---|---|---|---|---|---|
| | BOO38 | BOO38 | BOO38 | BOO38 | BOO38 |
| Component | CPPU-i | CPPU-ii | CPPU-iii | CPPU-iv | CPPU-v |
| NAA | 0.05 | 0.04 | 0.025 | 0.04 | 0.05 |
| BAP | 1 | 1 | 0.5 | 0.9 | 1 |
| Thidiazuron | 0.2 | 0.3 | 0.75 | 0.7 | 0.5 |
| Meta- | 3 | 3 | 5 | 4.5 | 2.5 |
| Topolin | | | | | |
| CPPU | 0.75 | 0.5 | 0.75 | 0.7 | 0.5 |

### Media BOO38 DPU

| | Reduced | Reduced | Reduced | Reduced | Reduced |
|---|---|---|---|---|---|
| | BOO38 | BOO38 | BOO38 | BOO38 | BOO38 |
| Component | CPU-i | CPU-ii | CPU-iii | CPU-iv | CPU-v |
| NAA | 0.01 | 0.05 | 0.025 | 0.05 | 0.05 |
| BAP | 0.25 | 1 | 0.75 | 1 | 0.5 |
| Thidiazuron | 0.2 | 0.6 | 0.75 | 0.75 | 0.3 |
| Meta- | 2 | 3 | 4 | 5 | 5 |
| Topolin | | | | | |
| DPU | 0.75 | 0.75 | 0.6 | 0.75 | 0.4 |

Alternatively, the cytokinins noted above can be replaced with weaker cytokinins at similar or higher levels. Exemplary weaker cytokinins include zeatin and kinetin.

Contaminated tubes can be identified by bacterial discoloration of the agar or by visible surface contamination. These explants can stay on the chosen BOO38-iv media for 3-4 10-120 day cycles (usually 21 day cycles) or as modified in the spiked procedure (spiked media for a period of 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours before transition to a standard or reduced media for the remainder of the 10-120 day cycle or for a full 10-120 day cycle). Additional time periods for placement in a spiked media include anywhere between 0.1 and 240 hours and can include, without limitation, 0.1-0.5 hours, 0.3-2.5 hours, 2.5-6 hours, 1-10 hours, 5-15 hours, 10-20 hours, 15-25 hours, 20-30 hours, 25-35 hours, 30-40 hours, 35-45 hours, 40-50 hours, 45-55 hours, 50-60 hours, 55-65 hours, 60-70 hours, 65-75 hours, 70-80 hours, 75-85 hours, 80-90 hours, 85-95 hours, 90-100 hours, 95-105 hours, 100-110 hours, 105-115 hours, 110-120 hours, 115-125 hours, 120-130 hours, 125-135 hours, 130-140 hours, 135-145 hours, 140-150 hours, 145-155 hours, 150-160 hours, 155-165 hours, 160-170 hours, 165-175 hours, 170-180 hours, 175-185 hours, 180-190 hours, 185-195 hours, 190-200 hours, 195-205 hours, 200-210 hours, 205-215 hours, 210-220 hours, 215-225 hours, 220-230 hours, 225-235 hours, 230-240 hours, 235-245 hours, 240-250 hours, 3-6 hours, 7-17 hours, 12-22 hours, 17-27 hours, 22-32 hours, 27-37 hours, 32-42 hours, 37-47 hours, 42-52 hours, 47-57 hours, 52-62 hours, 57-67 hours, 62-72 hours, 67-77 hours, 72-82 hours, 77-87 hours, 82-92 hours, 87-97 hours, 92-102 hours, 97-107 hours, 102-112 hours, 107-117 hours, 112-122 hours, 117-127 hours, 122-132 hours, 127-137 hours, 132-142 hours, 137-147 hours, 142-152 hours, 147-157 hours, 152-162 hours, 157-167 hours, 162-172 hours, 167-177 hours, 172-182 hours, 177-187 hours, 162-172 hours, 167-177 hours, 182-192 hours, 187-197 hours, 192-202 hours, 197-207 hours, 202-212 hours, 207-217 hours, 212-222 hours, 217-227 hours, 222-232 hours, 227-237 hours, 232-242 hours, 237-247 hours or 242-252 hours. Placement in spiked media can also be 0.5 hours less than a cycle in standard or reduced media, 1 hour less than a cycle in standard or reduced media and all time periods in between 1 and 240 hours less than a cycle in standard or reduced media. If an explant is cultured on a particular spiked media type (e.g. BOO38), when transferred to a standard or reduced media, the standard or reduced media can be of the same type (e.g. standard or reduced BOO38) or of a different type (e.g. standard or reduced BOO39, BOO40, BOO44, BOO30, BOO36 etc.).

In particular embodiments disclosed herein, culture periods are less than 12 weeks, less than 9 weeks, or less than 6 weeks.

Explants can be taken off the media after the third cycle if multiplication is occurring. If multiplication is not occurring or not occurring to a significant degree, explants can be left on the media for a fourth cycle.

Live shoots can next be transferred to a Stage 2 media (if standard BOO38 used in the previous step or a Stage 3 media if a basic spiked procedure was used), such as BOO36, BOO39, BOO40, BOO41, BOO42, BOO43, BOO44, BOO30, BOO35, BOO38 CPPU or BOO38 DPU at a pH of 5.7. The cultures can stay on this Stage 2 media until the desired number of shoots is obtained by separation into new tubes and further expansion. Generally, the range of time includes 10-120 day cycles (usually 14-21 day cycles) between which the cultures are assigned to go through another multiplication round or transitioned to a Stage 3 or Stage 4 media, for example, BOO37-iv or BOO31-iv at a pH of 5.7 for further multiplication.

In particular embodiments disclosed herein, culture periods are less than 12 weeks, less than 9 weeks, or less than 6 weeks.

Alternatively, live shoots can also be placed on a spiked BOO36, BOO39, BOO40, BOO41, BOO42, BOO43, BOO44, BOO30, BOO35, BOO38 CPPU, BOO38 DPU media for a period of 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours before transition to a same or different type of standard or reduced media for the remainder of the 10-120 day cycle or for a full 10-120 day cycle. Additional time periods for placement in a spiked media include anywhere between 0.1 and 240 hours and can include, without limitation, 0.1-0.5 hours, 0.3-2.5 hours, 2.5-6 hours, 1-10 hours, 5-15 hours, 10-20 hours, 15-25 hours, 20-30 hours, 25-35 hours, 30-40 hours, 35-45 hours, 40-50 hours, 45-55 hours, 50-60 hours, 55-65 hours, 60-70 hours, 65-75 hours, 70-80 hours, 75-85 hours, 80-90 hours, 85-95 hours, 90-100 hours, 95-105 hours, 100-110 hours, 105-115 hours, 110-120 hours, 115-125 hours, 120-130 hours, 125-135 hours, 130-140 hours, 135-145 hours, 140-150 hours, 145-155 hours, 150-160 hours, 155-165 hours, 160-170 hours, 165-175 hours, 170-180 hours, 175-185 hours, 180-190 hours, 185-195 hours, 190-200 hours, 195-205 hours, 200-210 hours, 205-215 hours, 210-220 hours, 215-225 hours, 220-230 hours, 225-235 hours, 230-240 hours, 235-245 hours, 240-250 hours, 3-6 hours, 7-17 hours, 12-22 hours, 17-27 hours, 22-32 hours, 27-37 hours, 32-42 hours, 37-47 hours, 42-52 hours, 47-57 hours, 52-62 hours, 57-67 hours, 62-72 hours, 67-77 hours, 72-82 hours, 77-87 hours, 82-92 hours, 87-97 hours, 92-102 hours, 97-107 hours, 102-112 hours, 107-117 hours, 112-122 hours, 117-127 hours, 122-132 hours, 127-137 hours, 132-142 hours, 137-147 hours, 142-152 hours, 147-157 hours, 152-162 hours, 157-167 hours, 162-172 hours, 167-177 hours, 172-182 hours, 177-187 hours, 162-172 hours, 167-177 hours, 182-192 hours, 187-197 hours, 192-202 hours, 197-207 hours, 202-212 hours, 207-217 hours, 212-222 hours, 217-227 hours, 222-232 hours, 227-237 hours, 232-242 hours, 237-247 hours or 242-252 hours. Placement in spiked media can also be 0.5 hours less than a cycle in standard or reduced media, 1 hour less than a cycle in standard or reduced media and all time periods in between 1 and 240 hours less than a cycle in standard or reduced media.

Generally, one-ten shoots per tube can be obtained per multiplication cycle.

Following removal from the multiplication process, the shoots can be transferred to small tissue culturing boxes (known as “magenta boxes”) for 10-120 days (usually 14-21 days) containing a Stage 3, Stage 4 or Stage 5 media, in this Example, BOO46 at a pH of 5.7 for 10-120 days (usually 14-21 days) or BOO47 at a pH of 5.7 for 10-120 days (usually 14-21 days). As above, shoots can be placed in spiked media for shorter time periods followed by placement into a standard or reduced media for the remainder of or for a full 10-120 day cycle.

In particular embodiments disclosed herein, culture periods are less than 12 weeks, less than 9 weeks, or less than 6 weeks.

As will be understood by one of ordinary skill in the art, when spiked media are used, the use of the spiked media increases the number of media stages within a particular process due the following use of a standard or reduced media. If spiked media are used at only one stage, the process generally expands by 1 media stage. If spiked media are used at two stages, the process generally expands by 2 media stages. If spiked media are used at three stages, the process generally expands by 3 media stages, etc.

Alternatively, the following procedures may also be used (Stage 1, Stage 2, Stage 3, etc, media are defined elsewhere herein):

Individual explants can then be placed on a Stage 1 media (15-25 mL) within a tube and the tubes placed into a regulated clean growth chamber at a temperature of from 65° F.-70° F. and a full spectrum light level of 36-90 μmole/m²/s². The Stage 1 media can be standard BOO38-iv at a pH of 5.7 or spiked BOO38-iv media. If placed on standard BOO38-iv, the explants can be transferred to fresh BOO38-iv media every 10-120 days (usually every 21 days), with contaminated tubes being discarded. If on spiked BOO38-iv media, the explants can remain on the spiked media for 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours and then be transferred to a media without spiked components (standard or reduced) for the remainder of the 10-120 day cycle or for a full 10-120 day cycle. Additional time periods for placement in a spiked media include anywhere between 0.1 and 240 hours and can include, without limitation, 0.1-0.5 hours, 0.3-2.5 hours, 2.5-6 hours, 1-10 hours, 5-15 hours, 10-20 hours, 15-25 hours, 20-30 hours, 25-35 hours, 30-40 hours, 35-45 hours, 40-50 hours, 45-55 hours, 50-60 hours, 55-65 hours, 60-70 hours, 65-75 hours, 70-80 hours, 75-85 hours, 80-90 hours, 85-95 hours, 90-100 hours, 95-105 hours, 100-110 hours, 105-115 hours, 110-120 hours, 115-125 hours, 120-130 hours, 125-135 hours, 130-140 hours, 135-145 hours, 140-150 hours, 145-155 hours, 150-160 hours, 155-165 hours, 160-170 hours, 165-175 hours, 170-180 hours, 175-185 hours, 180-190 hours, 185-195 hours, 190-200 hours, 195-205 hours, 200-210 hours, 205-215 hours, 210-220 hours, 215-225 hours, 220-230 hours, 225-235 hours, 230-240 hours, 235-245 hours, 240-250 hours, 3-6 hours, 7-17 hours, 12-22 hours, 17-27 hours, 22-32 hours, 27-37 hours, 32-42 hours, 37-47 hours, 42-52 hours, 47-57 hours, 52-62 hours, 57-67 hours, 62-72 hours, 67-77 hours, 72-82 hours, 77-87 hours, 82-92 hours, 87-97 hours, 92-102 hours, 97-107 hours, 102-112 hours, 107-117 hours, 112-122 hours, 117-127 hours, 122-132 hours, 127-137 hours, 132-142 hours, 137-147 hours, 142-152 hours, 147-157 hours, 152-162 hours, 157-167 hours, 162-172 hours, 167-177 hours, 172-182 hours, 177-187 hours, 162-172 hours, 167-177 hours, 182-192 hours, 187-197 hours, 192-202 hours, 197-207 hours, 202-212 hours, 207-217 hours, 212-222 hours, 217-227 hours, 222-232 hours, 227-237 hours, 232-242 hours, 237-247 hours or 242-252 hours. Placement in spiked media can also be 0.5 hours less than a cycle in standard or reduced media, 1 hour less than a cycle in standard or reduced media and all time periods in between 1 and 240 hours less than a cycle in standard or reduced media. These explants can stay on BOO38-iv media or spiked BOO38-iv media for 2 10-120 day cycles (usually 21 day cycles). Between cycles, excess sheaths can be removed. At the time of transfer to the third cycle, explants can be transitioned to a Stage 2 media or Stage 3 media (depending on whether spiked procedures are used), in this Example, standard BOO38-iv supplemented with 7 g/L carageenan rather than the 5.5 g/L provided above or a spiked BOO38-iv supplemented with 7 g/L carageenan rather than the 5.5 g/L provided above for 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours followed by transition to standard or reduced BOO38-iv. Additional time periods for placement in a spiked media include anywhere between 0.1 and 240 hours and can include, without limitation, 0.1-0.5 hours, 0.3-2.5 hours, 2.5-6 hours, 1-10 hours, 5-15 hours, 10-20 hours, 15-25 hours, 20-30 hours, 25-35 hours, 30-40 hours, 35-45 hours, 40-50 hours, 45-55 hours, 50-60 hours, 55-65 hours, 60-70 hours, 65-75 hours, 70-80 hours, 75-85 hours, 80-90 hours, 85-95 hours, 90-100 hours, 95-105 hours, 100-110 hours, 105-115 hours, 110-120 hours, 115-125 hours, 120-130 hours, 125-135 hours, 130-140 hours, 135-145 hours, 140-150 hours, 145-155 hours, 150-160 hours, 155-165 hours, 160-170 hours, 165-175 hours, 170-180 hours, 175-185 hours, 180-190 hours, 185-195 hours, 190-200 hours, 195-205 hours, 200-210 hours, 205-215 hours, 210-220 hours, 215-225 hours, 220-230 hours, 225-235 hours, 230-240 hours, 235-245 hours, 240-250 hours, 3-6 hours, 7-17 hours, 12-22 hours, 17-27 hours, 22-32 hours, 27-37 hours, 32-42 hours, 37-47 hours, 42-52 hours, 47-57 hours, 52-62 hours, 57-67 hours, 62-72 hours, 67-77 hours, 72-82 hours, 77-87 hours, 82-92 hours, 87-97 hours, 92-102 hours, 97-107 hours, 102-112 hours, 107-117 hours, 112-122 hours, 117-127 hours, 122-132 hours, 127-137 hours, 132-142 hours, 137-147 hours, 142-152 hours, 147-157 hours, 152-162 hours, 157-167 hours, 162-172 hours, 167-177 hours, 172-182 hours, 177-187 hours, 162-172 hours, 167-177 hours, 182-192 hours, 187-197 hours, 192-202 hours, 197-207 hours, 202-212 hours, 207-217 hours, 212-222 hours, 217-227 hours, 222-232 hours, 227-237 hours, 232-242 hours, 237-247 hours or 242-252 hours. Placement in spiked media can also be 0.5 hours less than a cycle in standard or reduced media, 1 hour less than a cycle in standard or reduced media and all time periods in between 1 and 240 hours less than a cycle in standard or reduced media. Following the third cycle, explants can be cleaned. The explants can be kept on BOO38-iv supplemented with 7 g/L carageenan rather than the 5.5 g/L provided above for 10-120 day cycles (usually 21 day cycles) until multiple shoots are observed. Observation of multiple shoots can occur within 3-15 months. When multiple cycles are used, explants can be cultured in standard media for all cycles, on spiked media followed by standard media for all cycles or on spiked media followed by reduced media for all cycles. Alternatively, explants can be exposed to one or more of these treatments across cycles in any combination and order.

In particular embodiments disclosed herein, culture periods are less than 12 weeks, less than 9 weeks, or less than 6 weeks.

Once an explant exhibits multiple shoots, it can be either maintained on its current media when shooting occurred (with transfer to fresh media every 10-120 days) or transferred to a subsequent media. Non-limiting subsequent media include, without limitation a BOO36 media, a BOO39 media, a BOO40 media a BOO41 media, a BOO42 media, a BOO43 media, a BOO44 media or a BOO30 media at a pH of 5.7 or spiked versions of the same followed by transition to a standard or reduced media. The cultures can stay on the current or subsequent media until the desired number of shoots is obtained by separation into new tubes and further expansion. Generally, the range of time includes 10-120 day cycles (usually 21 day cycles) between which the cultures can be assigned to go through another multiplication round or transitioned to a next stage media, such as a BOO46 media at a pH of 5.7 for 10-120 days (usually 21 days) in “magenta boxes” or a BOO47 media at a pH of 5.7 for 10-120 days (usually 14-21 days).

In particular embodiments disclosed herein, culture periods are less than 12 weeks, less than 9 weeks, or less than 6 weeks.

In even more particular non-limiting embodiments, the following species can be micropropagated in the following media (at a pH of 5.5-5.7) according to procedures described in the proceeding paragraphs [000198]-[0002-1]:

Arundinaria gigantea: BOO36-v, BOO39-v, BOO41-v, BOO42-v, BOO43-v, BOO44-v, BOO35-v, BOO38 CPPU-v, BOO38 DPU-v or spiked and reduced versions thereof;

Bambusa balcoa: BOO36-v, BOO39-v, BOO41-v, BOO42-v, BOO43-v, BOO44-v, BOO35-v, BOO38 CPPU-v, BOO38 DPU-v or spiked and reduced versions thereof;

Bambusa vulgaris: BOO36-v, BOO39-v, BOO41-v, BOO42-v, BOO43-v, BOO44-v, BOO35-v, BOO38 CPPU-v, BOO38 DPU-v spiked and reduced versions thereof;

Bambusa vulgaris ‘Vitatta’: BOO36-v, BOO39-v, BOO41-v, BOO42-v, BOO43-v, BOO44-v, BOO35-v, BOO38 CPPU-v, BOO38 DPU-v or spiked and reduced versions thereof;

Bambusa Oldhamii: BOO36-v, BOO39-v, BOO41-v, BOO42-v, BOO43-v, BOO44-v, BOO35-v, BOO38 CPPU-v, BOO38 DPU-v or spiked and reduced versions thereof;

Bambusa tulda: BOO36-v, BOO39-v, BOO41-v, BOO42-v, BOO43-v, BOO44-v, BOO35-v, BOO38 CPPU-v, BOO38 DPU-v or spiked and reduced versions thereof;

Dendrocalamus brandesii: BOO36-v, BOO39-v, BOO41-v, BOO42-v, BOO43-v, BOO44-v, BOO35-v, BOO38 CPPU-v, BOO38 DPU-v or spiked and reduced versions thereof;

Dendrocalamus asper: BOO36-v, BOO39-v, BOO41-v, BOO42-v, BOO43-v, BOO44-v, BOO35-v, BOO38 CPPU-v, BOO38 DPU-v or spiked and reduced versions thereof;

Dendrocalamus hamiltoni: BOO36-v, BOO39-v, BOO41-v, BOO42-v, BOO43-v, BOO44-v, BOO35-v, BOO38 CPPU-v, BOO38 DPU-v or spiked and reduced versions thereof;

Dendrocalamus giganteus: BOO36-v, BOO39-v, BOO41-v, BOO42-v, BOO43-v, BOO44-v, BOO35-v, BOO38 CPPU-v, BOO38 DPU-v or spiked and reduced versions thereof;

Dendrocalamus membranaceus: BOO36-v, BOO39-v, BOO41-v, BOO42-v, BOO43-v, BOO44-v, BOO35-v, BOO38 CPPU-v, BOO38 DPU-v or spiked and reduced versions thereof;

Dendrocalamus strictus: BOO36-v, BOO39-v, BOO41-v, BOO42-v, BOO43-v, BOO44-v, BOO35-v, BOO38 CPPU-v, BOO38 DPU-v or spiked and reduced versions thereof;

Gigantochloa aspera: BOO36-v, BOO39-v, BOO41-v, BOO42-v, BOO43-v, BOO44-v, BOO35-v, BOO38 CPPU-v, BOO38 DPU-v or spiked and reduced versions thereof;

Gigantochloa scortechini: BOO36-v, BOO39-v, BOO41-v, BOO42-v, BOO43-v, BOO44-v, BOO35-v, BOO38 CPPU-v, BOO38 DPU-v or spiked and reduced versions thereof;

Guadua culeata: BOO36-v, BOO39-v, BOO41-v, BOO42-v, BOO43-v, BOO44-v, BOO35-v, BOO38 CPPU-v, BOO38 DPU-v or spiked and reduced versions thereof;

Guadua aculeata ‘Nicaragua’: BOO36-v, BOO39-v, BOO41-v, BOO42-v, BOO43-v, BOO44-v, BOO35-v, BOO38 CPPU-v, BOO38 DPU-v or spiked and reduced versions thereof;

Guadua amplexifolia: BOO36-v, BOO39-v, BOO41-v, BOO42-v, BOO43-v, BOO44-v, BOO35-v, BOO38 CPPU-v, BOO38 DPU-v or spiked and reduced versions thereof;

Guadua angustifolia: BOO36-v, BOO39-v, BOO41-v, BOO42-v, BOO43-v, BOO44-v, BOO35-v, BOO38 CPPU-v, BOO38 DPU-v or spiked and reduced versions thereof;

Guadua angustofolia bi-color: BOO36-v, BOO39-v, BOO41-v, BOO42-v, BOO43-v, BOO44-v, BOO35-v, BOO38 CPPU-v, BOO38 DPU-v or spiked and reduced versions thereof;

Guadua paniculata: BOO36-v, BOO39-v, BOO41-v, BOO42-v, BOO43-v, BOO44-v, BOO35-v, BOO38 CPPU-v, BOO38 DPU-v or spiked and reduced versions thereof;

Melocanna bambusoides: BOO36-v, BOO39-v, BOO41-v, BOO42-v, BOO43-v, BOO44-v, BOO35-v, BOO38 CPPU-v, BOO38 DPU-v or spiked and reduced versions thereof;

Neohouzeaua dullooa (Teinostachyum): BOO36-v, BOO39-v, BOO41-v, BOO42-v, BOO43-v, BOO44-v, BOO35-v, BOO38 CPPU-v, BOO38 DPU-v or spiked and reduced versions thereof;

Ochlandra travancorica: BOO36-v, BOO39-v, BOO41-v, BOO42-v, BOO43-v, BOO44-v, BOO35-v, BOO38 CPPU-v, BOO38 DPU-v or spiked and reduced versions thereof;

Phyllostachys edulis ‘Moso’: BOO36-v, BOO39-v, BOO41-v, BOO42-v, BOO43-v, BOO44-v, BOO35-v, BOO38 CPPU-v, BOO38 DPU-v or spiked and reduced versions thereof;

Phyllostachys nigra: BOO36-v, BOO39-v, BOO41-v, BOO42-v, BOO43-v, BOO44-v, BOO35-v, BOO38 CPPU-v, BOO38 DPU-v or spiked and reduced versions thereof;

Phyllostachys nigra BOO36-v, BOO39-v, BOO41-v, BOO42-v, BOO43-v, BOO44-v, BOO35-v, BOO38 CPPU-v, BOO38 DPU-v or spiked and reduced versions thereof;

Schizostachyum lumampao: BOO36-v, BOO39-v, BOO41-v, BOO42-v, BOO43-v, BOO44-v, BOO35-v, BOO38 CPPU-v, BOO38 DPU-v or spiked and reduced versions thereof.

In some embodiments, the methods comprise using the bioreactors of the present invention. In some embodiments, the methods comprising using the racks of the present invention. In some embodiments, the methods comprise using the bioreactors and the racks of the present invention. In some embodiments, when two or more media described above are used in rotation during micropropagation, a bioreactor described herein can be used, for example, when a Stage 1 and a Stage 2 media are used in rotation, when a Stage 3 and a Stage 4 media are used in rotation, and/or when a Stage 1, a Stage 2, and a Stage 3 media are used in rotation, etc.

### Plant Propagation Bioreactor

Bioreactors can be used for plant micropropagation to more efficiently increase shoot mass than in stationary cultures. In some embodiments, bioreactors can be used for micropropagation to induce the formation of microtubers. In some embodiments, the induction of microtuber formation is done so more synchronously and in greater numbers in a bioreactor than in stationary cultures. Bioreactors offer a promising way of scaling-up micropropagation processes, making it possible to work in large containers with a high degree of control over culture parameters (e.g., pH, aeration, oxygen, carbon dioxide, hormones, nutrients, etc.). Bioreactors are also compatible with the automation of micropropagation procedures, utilizing artificial intelligence, which reduces production costs.

Previously bioreactors have been mostly applied to microbial technology, cell culture, and somatic embryogenesis and prior to the present invention, bioreactors for plant micropropagation were rare, complicated, and expensive.

To solve these problems, the present invention provides novel compositions, methods, and systems for the micropropagation of plants using a bioreactor. Without wishing to be bound by any theory, the present invention achieves greatly improved plant micropropagation through controlling/reducing exposure of the shoots/plantlets to toxic components that build-up in the growth compositions/environment (e.g., certain plant hormones, such as TDZ) and/or are produced as by-products of plant growth (e.g., phenolics).

The present application also provides a system for plant micropropagation. The system is also useful for the production of perennials, grasses and phyto-pharmaceutical plants. In some embodiments, the system comprises a growth vessel, two or more media containers, and a power source for driving fluid into and/or out of the growth vessel. In some embodiments, the system further comprises a controller. In some embodiments, the system further comprises a light source and/or a gas source providing Cm, 02, N2, or mixture thereof to the growth vessel.

In some embodiments, a temporary immersion bioreactor (TIB, a.k.a. temporary immersion system (TIS) or “Ebb and Flow” bioreactor) is used. Non-limiting examples of temporary immersion bioreactors include nutrient mist bioreactors, tilting and rocking vessels, twin flask system, or single containers with at least two compartments, such as Recipient for Automated Temporary Immersion (RITA®).

In some embodiments, the temporary immersion bioreactor is disposable. In some embodiments, the temporary immersion bioreactor is reusable.

In some embodiments, the temporary immersion bioreactor involves a wetting and drying cycle which occurs periodically in a predetermined period of time and hence it can also be termed as periodic, temporary immersion. In some embodiments, the temporary immersion bioreactor has a mere up-and-down motion of the nutrient medium without renewal. In some embodiments, renewal of nutrient medium in the bioreactor is involved.

Temporary immersion bioreactors provide an excellent way of using liquid medium at the same time controlling the gaseous environment. Moreover, it can provide the possible automation of the production system which facilitates low production costs. In other words, increasing the rate of growth and multiplication by using bioreactors more plants per unit area of the growth room are produced, which reduces the cost per plant per unit space of growth room. Liquid culture bioreactors are mainly suitable for the large-scale production of small size somatic embryos, growth of bulb, corms, micro tubers, compact shoot cultures etc. Major features of a temporary immersion bioreactor are:
Reduction of hyperhydricity, compared with that of permanent immersion, is the major achievement of a temporary immersion system. As plants are immersed in the medium for short time, the physiological disorders are reduced and the plants become healthier.
Plant growth and development can be controlled by manipulating the frequency and duration of immersion in liquid medium.
Plant growth is improved because during every immersion the plant is in direct contact with the medium and a thin film of liquid covers the plant throughout the interval period.
Air vents attached to the vessel prevent the cultures from contamination.
Due to the lack of agitation or aeration, the mechanical stress on plant tissues are generally low compared with the other bioreactor systems.

Temporary immersion bioreactors, which represent simple plastic vessels with medium (e.g., liquid, semi-liquid, etc) moving from one side to another every several minutes, can be used to generate microtubers. This temporary immersion system has been shown to stimulate shoot multiplication in many plant species. For example, the multiplication rates for sugarcane and pineapple were 6 and 3-4 times, respectively, higher compared with the rates obtained in liquid or solid media (Lorenzo et al., 1998; Escalona et al., 1999). In some embodiments, the bioreactor used in the present invention is a bioreactor described in International Patent Application No. PCT/US2012/047622, which is incorporated by reference in its entirety.

Other non-limiting examples of plant micropropagation systems include those described in U.S. Pat. Nos. 3,578,431; 4,320,594; 4,669,217; 4,908,315; 4,934,096; 5,049,505; 5,088,231; 5,104,527; 5,119,588; 5,139,956; 5,171,683; 5,184,420; 5,186,895; 5,212,906; 5,225,342; 5,558,984; 5,597,731; and 6,753,178. More examples of plant micropropagation systems can be found in Etienne et al. (Bioreactors in coffee micropropagation, Braz. J. Plant Physiol., 18(1):45-54, 2006); Ziv (Bioreactor Technology for Plant Micropropagation Horticultural Reviews, Volume 24, Edited by Jules Janick ISBN 0-471-33374-3); and Paek et al. (Application of bioreactors for large-scale micropropagation systems of plants, In Vitro Cell. Dev. Biol.-Plant 37:149-157, March-April 2001). It is understood that plant propagation systems that can be used in the present invention includes those derived from the ones described above by adding or reducing one or more parts/features of the systems known to one skilled in the art.

In some embodiments, the present invention provides novel compositions, methods, and systems for the induction, establishment, and maturation of embryos from plants using a bioreactor.

The present invention provides media for bamboo propagation. In some embodiments, the media can be used for in vitro propagation through somatic embryogenesis.

In some embodiments, provided are the first type of media that can initiate an embryogenic response in a plant or a plant part. Such media are called initiation bamboo nutrient media. None-limiting examples of the first type of media are BOO72, BOO73, BOO74, BOO75, and BOO76.

In some embodiments, the media can comprise nutrients selected from the group consisting of amino acids, macroelements, microelements, aluminum, boron, chlorine (chloride), chromium, cobalt, copper, iodine, iron, lead, magnesium, manganese, molybdenum, nitrogen (nitrates), potassium, phosphorous (phosphates), silicon, sodium, sulphur (sulphates), titanium, vanadium, zinc, inositol and undefined media components such as casein hydrolysates or yeast extracts. For example, the media can include any combination of NH₄NO₃; KNO₃; Ca(NO₃)₂; K₂SO₄; MgSO₄; MnSO₄; ZnSO₄; K₂SO₅; CuSO₄; CaCl₂; KI; CoCl₂; H₃BO₃; Na₂MoO₄; KH₂PO₄; FeSO₄; Na₂EDTA; Na₂H₂PO₄; inositol (e.g., myo-inositol); thiamine; pyridoxine; nicotinic acid; glycine; riboflavin; ascorbic acid; and silicon standard solution. It is known to those in the art that one or more components mentioned above can be omitted without affecting the function of the media.

In some embodiments, the media comprise macronutrients (e.g., ammonium nitrate, ammonium sulfate, calcium chloride anhydrous, magnesium sulfate anhydrous, potassium nitrate, potassium phosphate monobasic), micronutrients (e.g., boric acid, cobalt chloride anhydrous, cupric sulfate anhydrous, ferrous sulfate, manganese sulfate, molybdic acid sodium salt, Na₂-EDTA, potassium iodide, zinc sulfate), and vitamins (e.g., glycerine, myo-Inositol, nicotinic acid Pyridoxine-HCl, thiamine-HCl), or those found in the MS media (Murashige and Skoog, 1962). In some embodiments, the amount of one or more components of the MS media is doubled. In some embodiments, the amount of NH4NO3, KNO3, Ca(NO3)2, K2SO4, MgSO4, MnSO4, ZnSO4, CuSO4, and/or CaCl2 is doubled. In some embodiments, the liquid media are supplemented with one or more amino acids. In some embodiments, the amino acids can be any one of or combinations of the 20 fundamental amino acids known to one skilled in the art. In some embodiments, the amino acids can be any derivatives or analogs of the 20 fundamental amino acids. In some embodiments, the amino acids can be any synthetic amino acids, non-natural amino acids, or combinations thereof. In some embodiments, the amino acids comprise prolin and/or serine.

The present invention also provides a second type of media used for in vitro propagation of bamboo through somatic embryogenesis. In some embodiments, the second type of media are liquid or solid media. In some embodiments, the second media comprises one or more salts as described herein, e.g., the salts that can be found in MS media. In some embodiments, the amount of one or more components of the MS media is doubled. In some embodiments, the amount of NH4NO3, KNO3, Ca(NO3)2, K2SO4, MgSO4, MnSO4, ZnSO4, CuSO4, and/or CaCl2 is doubled. In some embodiments, the liquid media are supplemented with one or more amino acids. In some embodiments, the amino acids can be any one of or combinations of the 20 fundamental amino acids known to one skilled in the art. In some embodiments, the amino acids can be any derivatives or analogs of the 20 fundamental amino acids. In some embodiments, the amino acids can be any synthetic amino acids, non-natural amino acids, or combinations thereof. In some embodiments, the amino acids comprise prolin and/or serine.

The present invention also provides a third type of media used for in vitro propagation of bamboo through somatic embryogenesis. In some embodiments, a third type of medium is a solid medium. In some embodiments, the second media comprises one or more salts as described herein, e.g., the salts that can be found in MS media. In some embodiments, the amount of one or more components of the MS media is doubled. In some embodiments, the amount of NH4NO3, KNO3, Ca(NO3)2, K2SO4, MgSO4, MnSO4, ZnSO4, CuSO4, and/or CaCl2 is doubled.

In some embodiments, the third media are supplemented with abscisic acid (ABA), derivatives thereof, analogs thereof, or any combinations thereof. In some embodiments, the concentration of ABA is about 1.0 to about 100 μM. For example, the concentration of ABA is about 1, 2, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 μM or more. In some embodiments, the ABA concentration is about 40-60 mg/L, e.g., about 52.8 mg/L.

In some embodiments, the third media comprise charcoal, such as active charcoal. Surprisingly, addition of charcoal to this type of media can greatly enhance embryo production and maturation. In some embodiments, the concentration of charcoal is about 0.01% to about 10%, such as about 0.01%, about 0.02%, about 0.03%, about 0.04%, about 0.05%, about 0.06%, about 0.07%, about 0.08%, about 0.09%, about 1%, about 2%, about 3%, about 4%, about 5%, about 6%, about 7%, about 8%, about 9%, about 10%, or more, by weight. In some embodiments, the liquid media are supplemented with one or more amino acids. In some embodiments, the amino acids can be any one of or combinations of the 20 fundamental amino acids known to one skilled in the art. In some embodiments, the amino acids can be any derivatives or analogs of the 20 fundamental amino acids. In some embodiments, the amino acids can be any synthetic amino acids, non-natural amino acids, or combinations thereof. In some embodiments, the amino acids comprise proline and/or serine.

The present invention also provides a fourth type of media used for in vitro propagation of bamboo through somatic embryogenesis. In some embodiments, a fourth type of medium is a solid medium. In some embodiments, the fourth type of media is used for embryo germination. In some embodiments, the media of this type are solid media.

In some embodiments, the fourth type of media comprises one or more salts described herein. e.g., the salts that can be found in MS media. In some embodiments, the liquid media are supplemented with one or more amino acids. In some embodiments, the amino acids can be any one of or combinations of the 20 fundamental amino acids known to one skilled in the art. In some embodiments, the amino acids can be any derivatives or analogs of the 20 fundamental amino acids. In some embodiments, the amino acids can be any synthetic amino acids, non-natural amino acids, or combinations thereof. In some embodiments, the amino acids comprise prolin and/or serine.

The present application also provides a system for plant micropropagation. The system is also useful for induction, establishment, and maturation of embryos. The present application also provides a system for plant micropropagation. The system is also useful for the reduction of phenolics in plants. In some embodiments, the system comprises a growth vessel, two or more media containers, and a power source for driving fluid into and/or out of the growth vessel. In some embodiments, the system further comprises a controller. In some embodiments, the system further comprises a light source and/or a gas source providing Cm, 02, N2, or mixture thereof to the growth vessel.

In some embodiments, the system comprises:

a growth vessel for incubating plant tissue in a sterile or substantially sterile environment;

a first media container having a first fluid port and a second fluid port, the first fluid port fluidically coupleable to the growth vessel;

a second media container having a first fluid port and a second fluid port, the first fluid port fluidically coupleable to the growth vessel; and

a gas source fluidically coupleable to the second fluid port of the first media container and second fluid port of the second media container.

In some embodiments, the system further comprises a controller operable in a first operating mode in which pressurized gas is delivered from the gas source to the first media container to displace a first volume of liquid contained therein to the growth vessel, and a second operating mode in which pressurized gas is delivered from the gas source to the second media container to displace a second volume of liquid contained therein to the growth vessel.

In some embodiments, the controller is operable in a third operating mode in which liquid contained in the growth vessel is allowed to flow from the growth vessel into at least one of the first media container and the second media container.

In some embodiments, the controller is operable in a first incubation sequence in which the third operating mode is executed subsequent to the first operating mode.

In some embodiments, the controller is operable in a second incubation sequence in which the third operating mode is executed subsequent to the second operating mode.

In some embodiments, the controller is further operable in a plant propagation mode, in which the first incubation sequence and the second incubation sequence are executed.

In some embodiments, the growth vessel is elevated above the first and second media containers to allow liquid to flow from the growth vessel into at least one of the first media container and the second media container in the third operating mode.

In some embodiments, the system further comprises a manifold fluidically coupleable to the growth vessel, the first media container, and the second media container,

In some embodiments, the manifold is operable to control liquid flow between the growth vessel and the first media container and between the growth vessel and the second media container.

In some embodiments, the growth vessel includes a fluid conduit configured to siphon liquid from the growth vessel to at least one of the first media container and the second media container.

In some embodiments, the growth vessel is an ebb and flow bioreactor.

In some embodiments, the controller is operable to control fluid communication between the growth vessel and the first media container, between the growth vessel and the second media container, between the gas source and the first media container, and between the gas source and the second media container.

In some embodiments, the first media container comprises a bud induction medium (BOO1) as described herein, and the second media container comprises a shoot elongation/maintenance medium (BOO2). In some embodiments, the bud induction medium (BOO1) comprises an effective amount of thidiazuron (TDZ) or analog thereof, and the shoot elongation/maintenance medium (BOO2) comprises an effective amount of one or more cytokinins other than TDZ or an analog thereof.

The present application also provides methods for exchanging liquid media in a bioreactor/plant growth system for the micropropagation of plant or plant tissue.

In some embodiments, the bioreactor comprising a growth vessel for incubating the plant tissue, a first media container fluidically coupleable to the growth vessel, a second media container fluidically coupleable to the growth vessel, and a gas source fluid fluidically coupleable to the first media container and the second media container

In some embodiments, the methods comprise:

establishing fluid communication between the first media container and the growth vessel;

fluidically isolating the second media container from the growth vessel;

establishing fluid communication between the gas source and the first media container;

delivering compressed gas to the first media container to displace a first volume of liquid from the first media container to the growth vessel;

allowing at least a portion of the first volume of liquid to flow from the growth vessel back into the first media container;

establishing fluid communication between the second media container and the growth vessel;

fluidically isolating the first media container from the growth vessel;

establishing fluid communication between the gas source and the second media container;

delivering compressed gas to the second media container to displace a second volume of liquid from the first media container to the growth vessel; and

allowing at least a portion of the second volume of liquid to flow from the growth vessel back into the second media container.

In some embodiments, the compressed gas is delivered to the first media container for approximately one minute.

In some embodiments, the compressed gas is delivered to the second media container for approximately one minute.

In some embodiments, the liquid is allowed to flow from the growth vessel back into the first media container for approximately 8 minutes.

In some embodiments, the liquid is allowed to flow from the growth vessel back into the second media container for approximately 8 minutes.

In some embodiments, said systems are used for plant propagation comprising a bud induction medium (BOO1) and a shoot elongation and maintenance medium (BOO2). For example, the systems are used for plant propagations wherein a rotation of a bud induction medium (BOO1) and a shoot elongation and maintenance medium (BOO2) is involved.

In some embodiments, said systems are used for plant propagation comprising the alternative media as described herein. For example, the systems are used for plant propagations wherein (1) a rotation of a Stage 1 medium and a Stage 2 medium is involved; (2) a rotation of a Stage 2 medium and a Stage 3 medium is involved; (3) a rotation of a Stage 1 medium, a Stage 2 medium, and a Stage 3 medium is involved; (4) a rotation of a Stage 3 medium and a Stage 4 medium is involved; (5) a rotation of a Stage 4 medium and a Stage 5 medium is involved; (6) a rotation of a Stage 5 medium and a Stage 6 medium is involved; (7) a rotation of a Stage 4 medium, a Stage 5 medium, and a Stage 6 medium is involved; and/or (7) a rotation of a Stage 6 medium and a Stage 7 medium is involved.

In some embodiments, a temporary immersion bioreactor 100 is schematically illustrated in FIG. 5. The system 100 is configured for large scale multiplication of plants. In some embodiments, the system 100 is used for large scale multiplication of dicot plants. In some embodiments, the system 100 is used for large scale multiplication of yam. The system 100 includes a growth vessel 110, a first media container 130, a second media container 150, a gas source 170, and a controller 190.

The growth vessel 110 is configured to incubate plant tissue in a sterile or substantially sterile environment. The growth vessel 110 may be any suitable container capable of providing a sterile or substantially sterile environment for the plant tissue and nutrient media. The growth vessel 110 may further be of any suitable material and any desirable shape. For example, the growth vessel 110 may be transparent to permit visual observation and light stimulation of the plant tissue, and may be constructed to reduce shear forces on the incubated tissue.

In some embodiments, the growth vessel 110 comprises one or more type of light source suitable for plant growth. Alternatively, the growth vessel is transparent to permit light stimulation provided outside of the growth vessel 110.

In some embodiments, the growth vessel 110 is connected to a gas source. In some embodiments, the gas source provides carbon dioxide, oxygen, nitrogen, or combinations thereof. In some embodiments, the provided gas or mixture of gas is sterile or substantially sterile. The ratio of the gas mixture provided to the growth vessel 110 can be predetermined readily controlled depending on any of a variety of factors including, for example, the type of plants being grown in the plant propagation system 100.

The first media container 130 and second media container 150 are configured to contain a liquid and a gas and are each fluidically coupleable to both the growth vessel 110 and the gas source 170. Additional media containers can be includes depending on any of a variety of factors including, for example, the type of plants being grown in the plant propagation system 100. The media containers 130, 150 may contain identical liquid or semi-liquid media, or media that differs in content and/or composition. The media containers 130, 150 can be fluidically coupled to the growth vessel 110 in any suitable manner. For example, in some embodiments, the growth vessel 110 can have multiple fluid exchange ports (not shown), and each media container 130, 150 can be coupled to a separate media exchange port. Each connection may be direct and continuous, or include a controllable valve (e.g., manual or under electronic control of the controller 190). In some embodiments, the growth vessel 110 can have a single fluid exchange port (not shown) for connecting all media containers 130, 150, and a manifold (not shown) to control exchange of liquid media between the media containers 130, 150 and the growth vessel 110. The manifold can include any number of fluid communicators and valves (manual, electronically actuated, hydraulically actuated, etc.) to control liquid exchange between the media containers 130, 150 and the growth vessel 110. In some embodiments, the growth vessel 110 can have multiple fluid exchange ports (not shown) for connecting each media container.

The gas source 170 can be any device or system suitable for delivering pressurized gas to the media containers 130, 150. The gas source 170 can include one or more of, but is not limited to, compressed tanks of gas and gas pumps. Any number of gas sources 170 may be employed. For example, in some embodiments, each media container 130, 150 may be connected to a different gas source 170. In some embodiments, a manifold can be used to connect the gas source 170 to the media containers 130, 150. The manifold can include any number of fluid communicators and valves (manual, electronically actuated, hydraulically actuated, etc.) to control the pressurized gas supply to the media containers 130, 150. The manifold and valves can be coupled to the controller 190 to allow automated and/or electronic control of the gas supply to the medial containers 130, 150. The gas employed by the gas source 170 may be any gas that does not compromise the liquid media in the media containers 130, 150. Examples of such gases include any inert gas, oxygen, nitrogen, carbon dioxide, gas of an atmospheric composition, and combinations thereof.

The gas source 170 is operable to change the gas pressure in the media containers 130, 150 by delivering a mass of pressurized gas to one or both of the medial containers 130, 150. In some embodiments, the gas source 170 is configured to deliver pressurized gas to the first media container 130 or the second media container 150 to raise the gas pressure in the first media container 130 or the second media container 150 up to about 1 pound per square inch (psi), e.g., about 0.7 psi, about 0.8 psi, about 0.9 psi, about 1 psi, about 1.1 psi, or about 1.2 psi. The increased gas pressure in the first media container 130 or the second media 150 causes at least a portion of the liquid media contained in the first media container 130 to be displaced from the first media container 130 to the growth vessel 110, or at least a portion of the liquid media contained in the second media container 150 to be displaced from the second media container 150 to the growth vessel 110. The gas source 170 can be deactivated or isolated to allow the gas pressure in the first media container 130 or the second media container 150 to return to its original (e.g., atmospheric) value. As a result, the displaced portion of the liquid media in the growth vessel 110 returns back to the first or the second media container. The combination of pressurization and deactivation of the gas source 170 results in “pulsing” of the first media and/or the second media contacting the plant tissue in the growth vessel 110.

In some embodiments, during the deactivation stage, media container equalizes pressure, and automatic siphoning drainage begins, emptying media back to original media container. In some embodiments, the siphoning drainage rate is about 500 to 1000 mls/minute, such as 600-720 mls/minute.

In some embodiments, during the drainage, the plants are partially submerged in media for about 2 to about 4 minutes, such as about 2.5 to about 3 minutes.

In some embodiments, after the media is drained, and before the next media comes into the vessel, the plants inside the vessels are dried for a predetermined time. In some embodiments, the plants are dried for about 1-10 minutes, for example, about 5 minutes.

The pulsing process can be repeated for the first media container 130 or the second media container 150 for any number of cycles depending on, for example, the type of plants being grown in the plant propagation system 100. For example, the repeated cycles in total take about half hour to about six weeks, e.g., about 24 hours to about 60 hours, about 60 hours to about one week, or about one week to about six weeks., e.g., about 2 weeks to about 3 weeks. In some embodiments, the first media container and the second media container hold the same medium.

In some embodiments, the pulsing process can be alternated between the first media container 130 and the second media container 150. In some embodiments, the pulsing process can be repeated for the first media container 130 for a predetermined number of cycles and then switched to the second media container for a predetermined number of cycles. In some embodiments, the pulsing process can be repeated and alternated between the first media container 130 and the second media container 150 according to any of a variety of predetermined patterns depending on, for example, the type of plants being grown in the plant propagation system 100. For example, the pulsing process can repeated for the first media for about half hour to about six weeks, e.g., about 24 hours to about 60 hours, about 60 hours to about one week, or about one week to about six weeks, e.g., about 2 weeks to about 3 weeks.

Operation of the plant propagation system 100 is controlled either manually, or by the controller 190 which may be a processor, a computing device, or any programmable/configurable device or system as is known in the art. The controller 190 is configured for electronically control of the gas source 170, and controls at least activation and deactivation of the gas source 170. In some embodiments, the controller 190 is configured for electronic control of a manifold that connects the gas source 170 to each of the media containers 130, 150 to enable selection of one of the media containers. In some embodiments, the controller 190 is configured for electronic control of a manifold that connects the media containers 130, 150 with the growth vessel 110 to enable control of liquid flow between the media containers 130, 150 with the growth vessel 110. Furthermore, and not inconsistent with various embodiments and combinations thereof, the controller 190 may be connected to, and configured for control of, multiple gas sources, multiple manifolds, and/or multiple valves. Control of other aspects of the system 100 not illustrated herein (e.g., control of a gas exchange system, a temperature control system, etc.) are within the scope of this invention.

The controller 190 is operable in a first operating mode in which it causes the gas source 170 to deliver pressurized gas to the first media container 130 to displace a first volume of liquid contained therein to the growth vessel 110. Additionally, the controller 190 is operable in a second operating mode in which it causes the gas source 170 to deliver pressurized gas to the second media container 150 to displace a second volume of liquid contained therein to the growth vessel 110. In some embodiments, the first and second operating modes are run for a predetermined time. In some embodiments, the first and second operating modes are run for about one minute±half minute, e.g., about 30 seconds, about 40 seconds, about 50 seconds, about 60 seconds, about 70 seconds, about 80 seconds, or about 90 seconds. As described above, the first and second operating modes can be repeated and/or alternated according to a predetermined pattern.

Optionally, after the first operating mode or second operating mode has been executed (i.e., the growth vessel 110 contains liquid from one of the media containers 130, 150), the controller 190 is operable to be run in a third operating mode. In the third operating mode, the liquid in the growth vessel 110 is allowed to return to its respective media container, for example, by deactivating the gas source 170. In some embodiments, the third operating mode is run for a predetermined time. In some embodiments, the third operating mode is run for about eight minutes, e.g., about 7 minutes, 7.5 minutes, 8 minutes, 8.5 minutes, etc.

In some embodiments, the controller 190 is operable to run a first incubation sequence comprising one or more cycles of the first operating mode-third operating mode sequence. In some embodiments, the first incubation sequence is run from about 1 hour to about 3 weeks, e.g., from about 24 hours to about 60 hours. The controller 190 is also operable to run a second incubation sequence comprising one or more cycles of the second operating mode-third operating mode sequence. In some embodiments, the second incubation sequence is run from about 24 hours to about 4 weeks, and e.g., 3 days to about 5 days.

In some embodiments, the controller 190 is operable to run a plant propagation sequence comprising one or more cycles of the first incubation sequence each followed by the second incubation sequence. The number of cycles of the plant propagation sequence may range from one to eight, or more.

In some embodiments, one, or more, or all of the parts of the growth system can be sterilized by any known methods, such as autoclaving.

In some embodiments, the media is driven into or out of the growth vessel by gas pressure. In some embodiments, the media is driven into or out of the growth vessel by other forces, such as gravity, electricity, etc.

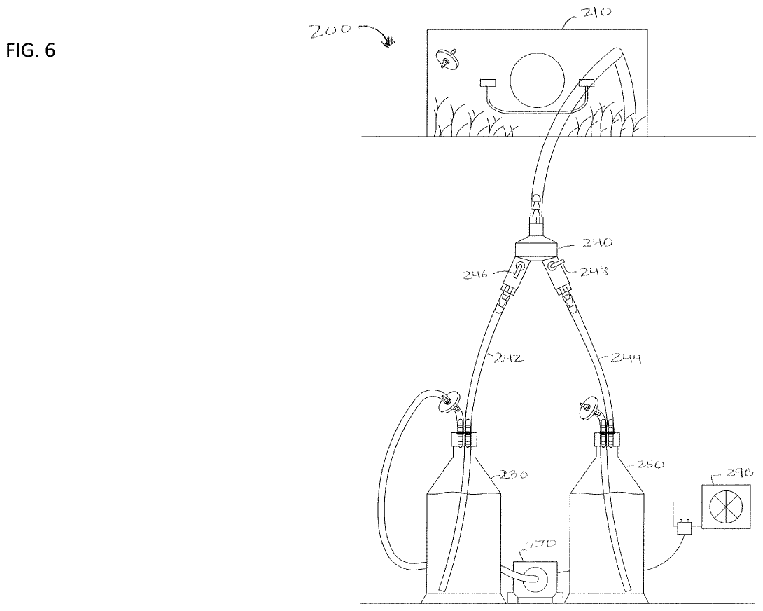

Referring now to FIG. 6-9C, an exemplary embodiment of a plant propagation system 200 is shown. The system 200 is similar in operation to the system 100 described above, thus unless stated otherwise, various components of the system 200 may be of similar design and function to that of other embodiments. For example, the growth vessel 210 may be similar to the growth vessel 110.

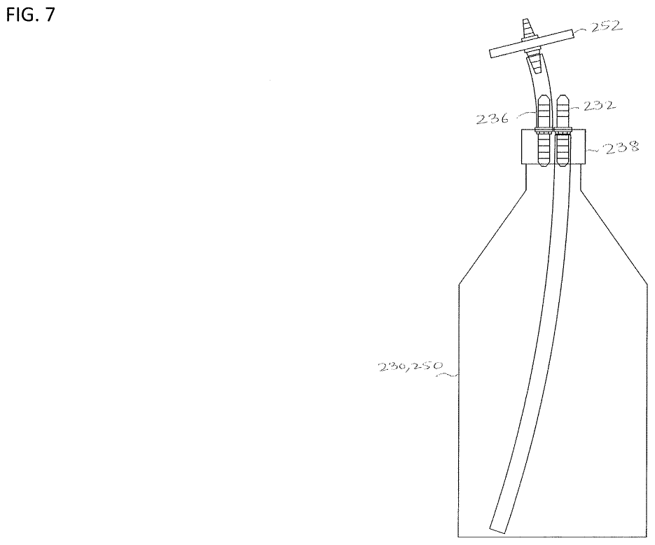

The illustrated embodiment of FIG. 6 includes a growth vessel 210, a manifold 240, a first media container 230, a second media container 250, a gas pump 270 as the gas source, and a timer-controlled circuit 290 as the controller. The single gas source 270 is attached to the first media container 230, and may be removed and reattached to the second media container 250. Advantageously, the use of a filter 252 (see FIG. 7 also) for each media container 230, 250 prevents any potential contamination of the liquid media in the media containers by the air pump 270, even upon switching. Alternatively, two gas sources can be used, with each source attached to media container 230 or 250, under the control of the timer-controlled circuit 290, so no reattachment is needed. As illustrated in FIG. 7, each media container 230, 250 has a first fluid port 232 in fluid communication or otherwise coupleable with port 222 of the growth vessel 210 to enable exchange of fluid between the media containers and the growth vessel. Each media container 230, 250 also has a second fluid port 236 in fluid communication or otherwise coupleable with the gas source 270 to change the gas pressure inside the media container. The ports 232 and 236 are formed on an adapter 238 that seals the media container to prevent contamination, a bulkhead adapter for example. As illustrated, the second fluid port 236 is additionally fitted with a filter 252 (e.g. a vent filter with stepped hose barbs) to prevent contamination of the fluid in the media container during gas exchange with the gas pump 270.

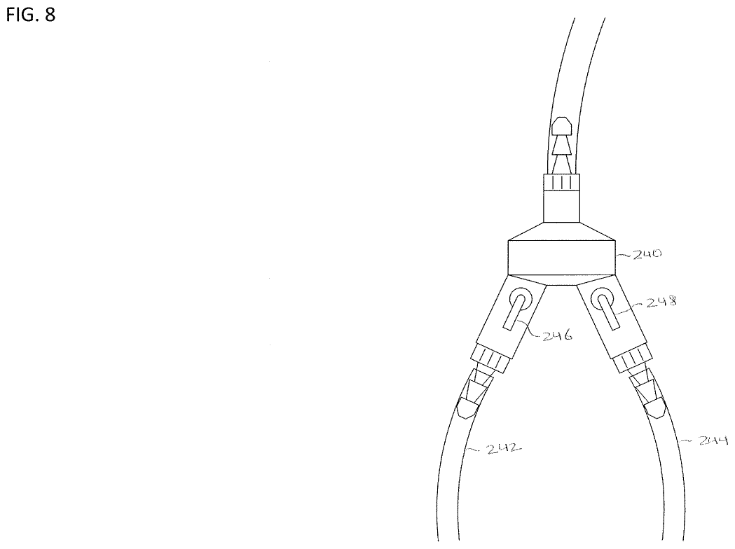

FIGS. 6 and 8 illustrate a non-limiting design of a manifold 240 that connects to tubing 242 and 244 from the media containers 230, 250, respectively. FIG. 8 illustrates valves 246 and 248 formed on manifold 240 for controlling flow from each tubing 242, 244, respectively. Any suitable 2-way valve may be employed such as, for example, ball valves, gate valves, butterfly valves, etc. Valves 246, 248 may be under electronic control of the timer 290, and/or under manual control. In the setup illustrated in FIG. 6, valve 246 is open to fluidly couple the growth vessel 210 and the first media container 230. Valve 248 is closed to fluidly isolate the second media container 250 from the growth vessel 210 as well as the first media container 230. In this manner, intermixing of fluids between the first media container 230 and the second media container 250 is prevented.

Control of the gas pump 270 may be achieved by switching on/off the power supply of the gas pump by the timer-controlled circuit 290. In an embodiment, the gas pump is a 1 psi pump which when powered by the circuit 290 (during the first or second operating mode, for example), pumps gas into the connected first media container 230 to increase the pressure to 1 psi. The gas pump 270 can be deactivated or otherwise turned off when circuit 290 shuts off the power to the gas pump 270 (during the third operating mode, for example), thereby allowing the pressure in the first media container to equalize by allowing the pumped gas to flow back into the gas pump.

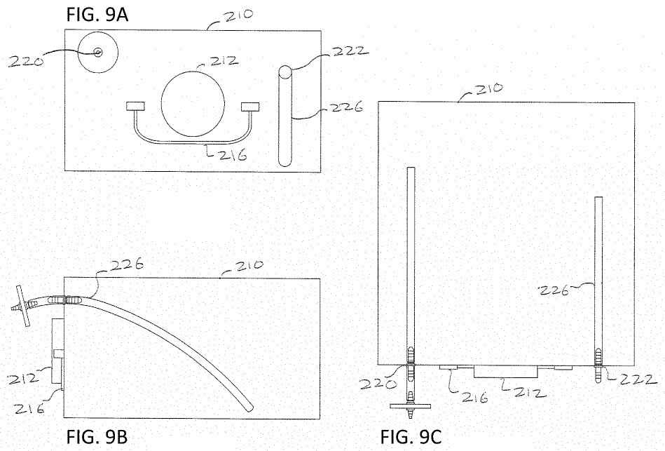

As illustrated in FIG. 9A-9C, the growth vessel 210 includes a closure 212 for accessing the interior of the growth vessel, and a handle 216 for ease of transportation. Though illustrated as formed on a front portion, the closure 212 and the handle 216 may be formed on any other part of the growth vessel 210. In an embodiment, the growth vessel 210 is an ebb and flow bioreactor.

The growth vessel 210 can also have a gas exchange port 220 and a fluid exchange port 222 formed on the growth vessel, although any number of gas exchange ports and fluid exchange ports are within the scope of the invention. The ports 220 and 222 are fitted with adapters for enabling fluid communication with the interior of the growth vessel 210, while maintaining sterility. In an embodiment, the ports 220 and 222 are fitted with bulkhead adapters. The growth vessel 210 also includes a fluid conduit 226 attached to the port 222 for exchanging fluid with the interior of the growth vessel. The conduit 226 is of sufficient length and has a lumen of appropriate cross-section to enable siphoning of fluid from the floor of the growth vessel 210 and into the selected media container 230 as is described in more detail below for the system 100 of FIG. 5.

In an embodiment, growth vessel 210 is used for large scale multiplication of plants. In some embodiments, growth vessel 210 is used for large scale multiplication of yam. In some embodiments, growth vessel further is used for pre-rooting and rooting the cultures. Referring again to the system 100 in FIG. 5, in use, the plant tissue to be incubated (e.g. yam tissue) is placed in the growth vessel 110, which is then placed at a height relative to the media containers 130, 150 to achieve the siphoning effect. First and second liquid media are placed in media containers 130, 150, respectively. In an embodiment, the first and second liquid media are different. The gas source 170 is connected to media containers 130, 150, and the controller 190 is connected to the gas source, and any other components requiring electronic control, as discussed.

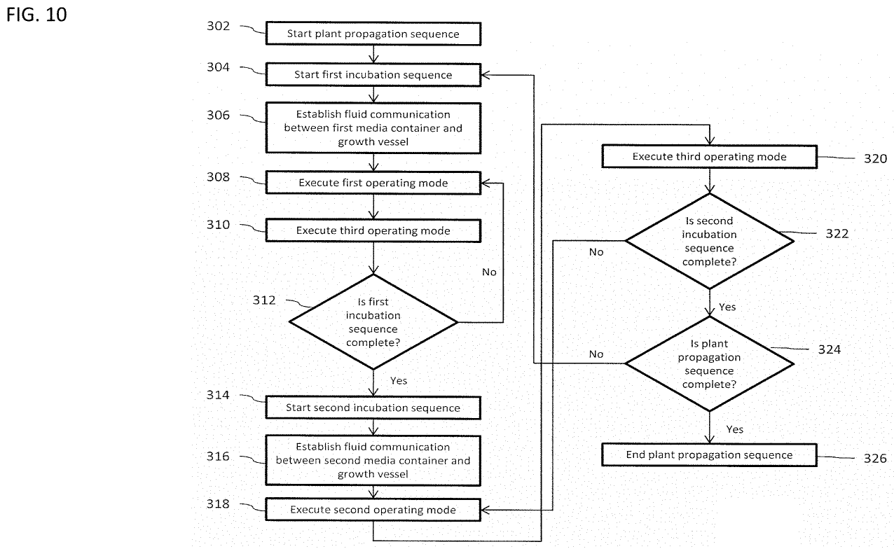

Operation of an exemplary plant propagation sequence 300 of the controller 190 is described herein as illustrated in FIG. 10. While illustrated in a stepwise manner, it is noted that the order of execution of these steps need not necessarily follow so. At 302, controller 190 starts the plant propagation sequence. At 304, controller 190 starts the first incubation sequence. At 306, the controller 190 establishes fluid communication between the first media container 130 and the growth vessel 110. The controller 190 also establishes fluid communication between the gas source 170 and the first media container 130. The controller 190 also fluidically isolates the second media container 150 from the growth vessel 110. At 308, the controller enters the first operating mode and drives the gas source 170 to deliver pressurized gas to the first media container 130 to displace a first volume of liquid contained therein to the growth vessel 110. At 310, the controller then enters the third communication mode and allows at least a portion of the first volume of liquid in the growth vessel 110 to return to the first media container 130 by deactivating the gas source 170.

At 312, if the first incubation sequence is not complete, the controller 190 returns to 308, and enters the first operating mode again. If the first incubation sequence is complete, the controller, at 314, starts the second incubation sequence. At 316, the controller establishes fluid communication between the second media container 150 and the growth vessel 110. The controller also establishes fluid communication between the gas source 170 and the second media container 150. The controller also fluidically isolates the first media container 130 from the growth vessel 110. At 318, the controller enters the second operating mode and drives the gas source 170 to deliver pressurized gas to the second media container 150 to displace a second volume of liquid contained therein to the growth vessel 110. At 320, the controller then enters the third communication mode and allows at least a portion of the second volume of liquid in the growth vessel 110 to return to the second media container 150 by deactivating the gas source 170.

At 322, if the second incubation sequence is not complete, the controller 190 returns to 318 and enters the second operating mode again. If the second incubation sequence is complete but (as determined at 324) the plant propagation sequence is not complete, the controller 190 returns to step 304 and starts the first incubation sequence again. If the plant propagation sequence is complete, the controller 190 exits the plant propagation process at 326. In an embodiment, the controller 190 includes a visual and/or audio indicator for signaling the end of the plant propagation sequence.

Aspects of the invention are hence beneficial for providing a semi-fully or fully automated, enclosed plant propagation system that is fully programmable for independently controlling, for multiple media, the pulsing time (i.e. the activation/deactivation time) and the incubation time, as well as for controlling the total number of incubation cycles for the entire set of available liquid media. Desirably, all components of the system are autoclavable, and hence reusable. Significant cost savings are realized by reduction in labor, oversight, and contamination loss.

Non-limiting examples of plant micropropagation systems include those described in U.S. Pat. Nos. 3,578,431; 4,320,594; 4,669,217; 4,908,315; 4,934,096; 5,049,505; 5,088,231; 5,104,527; 5,119,588; 5,139,956; 5,171,683; 5,184,420; 5,186,895; 5,212,906; 5,225,342; 5,558,984; 5,597,731; and 6,753,178. More examples of plant micropropagation systems can be found in Etienne et al. (Bioreactors in coffee micropropagation, Braz. J. Plant Physiol., 18(1):45-54, 2006); Ziv (Bioreactor Technology for Plant Micropropagation Horticultural Reviews, Volume 24, Edited by Jules Janick ISBN 0-471-33374-3); and Paek et al. (Application of bioreactors for large-scale micropropagation systems of plants, In Vitro Cell. Dev. Biol.-Plant 37:149-157, March-April 2001).

It is understood that plant propagation systems of the invention also include the systems derived from the exemplary systems described herein, by adding one or more parts/features of the systems known to one skilled in the art.

### Racks for Plant Micropropagation

For many plant species there are well known processes for micropropagation. In some instances, for example, it is desirable to intermittently expose cultivated plant tissue within a growth medium to a liquid nutrient solution. Some known systems designed to perform this function are inadequate and prone to breakage and/or mechanical failure. Furthermore, known systems are not sufficiently robust for a large-scale application. Thus, a need exists for improved apparatus for intermittently exposing microenvironments of tissue culture plantlets to a liquid nutrient solution. The present invention meets this need by providing devices and methods for intermittently exposing microenvironments of tissue culture plantlets to a liquid nutrient solution are described herein.

In some embodiments, an apparatus includes a frame, a shelf assembly supported on the frame, and a drive assembly coupled to the shelf assembly. In such embodiments, the drive assembly can be configured to impart an oscillating motion to the shelf assembly relative to the frame such that tissue culture plantlets in propagation vessels and supported on the shelf assembly are intermittently exposed to a liquid nutrient solution. In some embodiments, described herein relate generally to a rack and more particularly, to an oscillating rack for plant propagation vessels.

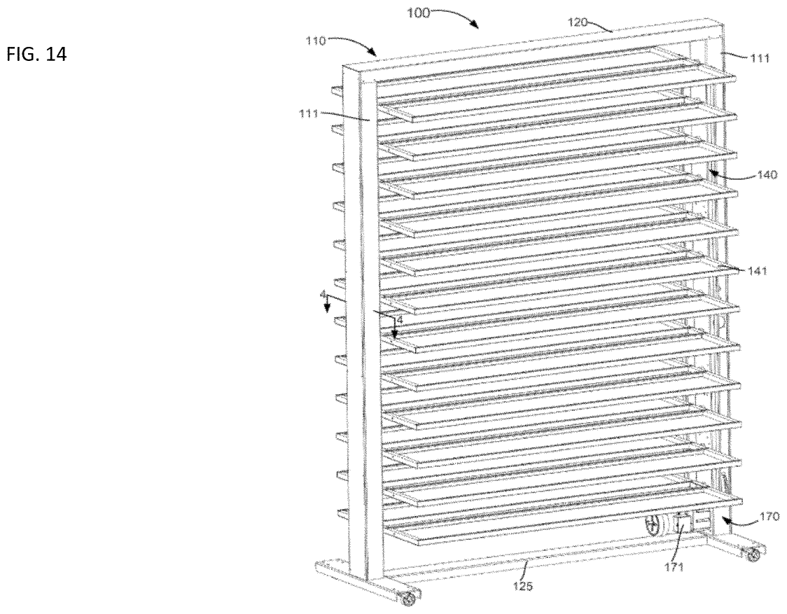

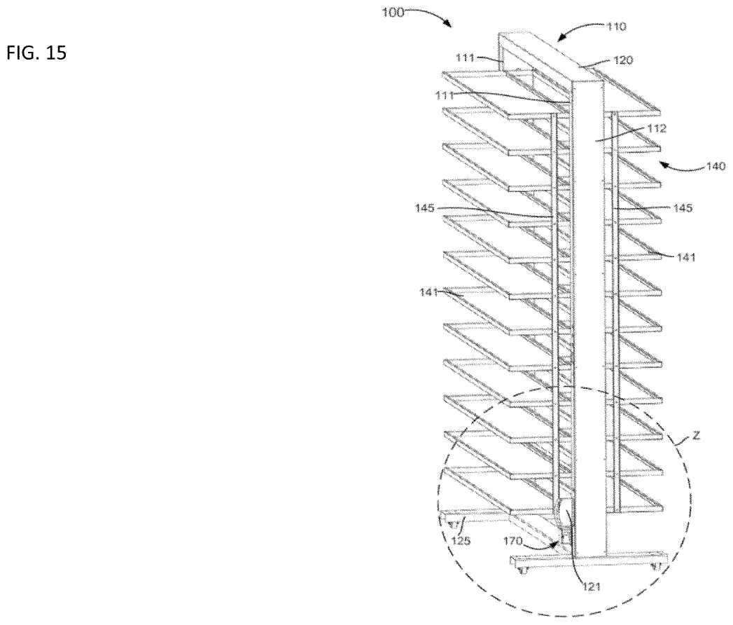

FIGS. 14 and 15 are perspective views of an oscillating rack 100, according to an embodiment. An oscillating rack 100 includes a frame 110, a shelf assembly 140, and a drive assembly 170. The frame 100 includes uprights 111, an upper cross member 120, and a base 125. The components of the frame 100 can be formed from any suitable material. For example, in some embodiments, the frame 100 can be formed from aluminum. In other embodiments, the frame 100 can be formed from an aluminum alloy, steel, and/or steel alloy and can be of any suitable gauge or thickness.

The uprights 111 can be any suitable configuration and extend upwardly from the base 125, as described in further detail herein. The upper cross member 120 can be any suitable size, shape, or configuration. For example, in some embodiments, the upper cross member 120 can be a formed (e.g., mechanically bent) or extruded C-channel. In other embodiments, the upper cross member 120 can be a substantially closed or solid structure, such as, for example, box tubing or bar stock. The upper cross member 120 is configured to be coupled to an upper portion of the uprights 111. In this manner, the upper cross member 120 can increase the rigidity and/or strength of an upper portion of the oscillating rack 100.

The base 125 can be any suitable platform or structure. For example, while shown in FIG. 14-16 as being substantially I-shaped (e.g., including a cross member coupled to two support members perpendicularly aligned to the cross member), in other embodiments, the base 125 can be any shape or configuration. In some embodiments, for example, the base 125 can be a substantially rectangular structure. In other embodiments, the base 125 can include stiffening members and/or the like. For example, in some embodiments, the base 125 can include a sheet metal portion coupled (e.g., screwed, welded, riveted, or otherwise fastened) to a top surface of the base 125 configured to increase the rigidity and/or strength of the base 125. Furthermore, in the embodiments shown in FIG. 14-16, the base 125 includes a set of caster wheels such that the oscillating rack 100 can be moved or repositioned.

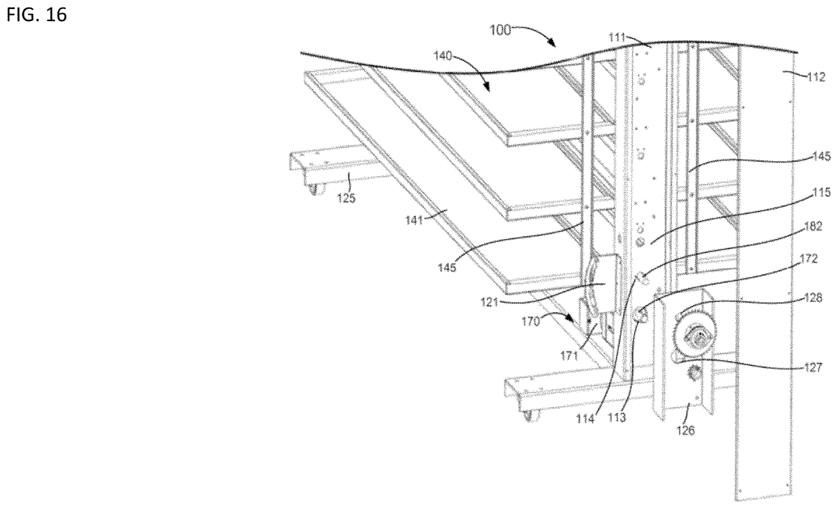

As shown in FIG. 16, the base 125 includes a support member 126 that extends from a top surface of the base 125. More specifically, the support members 126 are fixedly coupled (e.g., welded) to the top surface of the base 125 and receive at least a portion of the one of the uprights 111. Furthermore, one of the support members 126 includes a drive shaft opening 127 and a rocker shaft opening 128 configured to receive a drive shaft 172 and a rocker shaft 182 of the drive assembly 170, respectively.

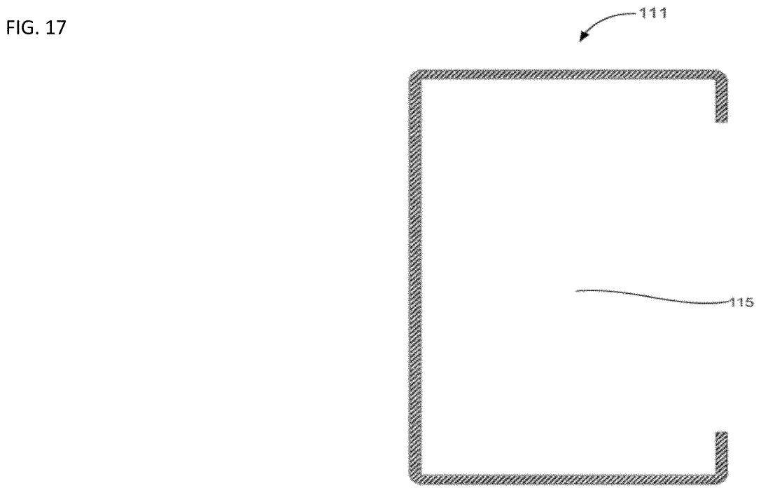

The uprights 111 include a set of walls that define a substantially C-Shaped cross-section (FIG. 17) and a cover 112 (FIG. 16). As shown in FIG. 14-16, the uprights 111 are configured to extend from the top surface of the base 125. More specifically, the uprights 111 are disposed around the support members 126 such that the uprights 111 extend away from the top surface of the base 125. Similarly stated, the support members 126 are disposed within a volume 115 defined by the set of walls that define the C-shaped cross-section. In this manner, the uprights 111 can be coupled (e.g., welded and/or fastened) to the base 125 and/or support members 126. The uprights 111 can further define any number of holes configured to receive portions of the oscillating rack 100. For example, at least one upright 111 can include a drive shaft opening 113 and a rocker shaft opening 114 configured to receive a drive shaft 172 and a rocker shaft 182, respectively, included in the drive assembly 170, respectively, and positioned to align with the drive shaft opening 127 and the rocker shaft opening 128, respectively, in the support member 126.

In some embodiments, the uprights 111 can define any number of holes and/or protrusions configured to engage a portion of a lighting system (not shown) and/or control system (not shown). In some embodiments, each cover 112 can be coupled to a respective upright 111 such that the cover 112 and the upright 111 house a set of electrical components (not shown) within the volume 115. For example, in some embodiments, the volume 115 can contain wires, switches, relays, electronic devices (e.g., a programmable logic controller (PLC) including, for example, at least a processor, a memory, and a network interface), and/or the like. In some embodiments, at least one upright 111 can include a sensor bracket 121. In such embodiments, a sensor can be disposed on the sensor bracket 121 and can indicate and/or monitor the position of the shelf assembly 140 relative to the frame 110, as further described herein.

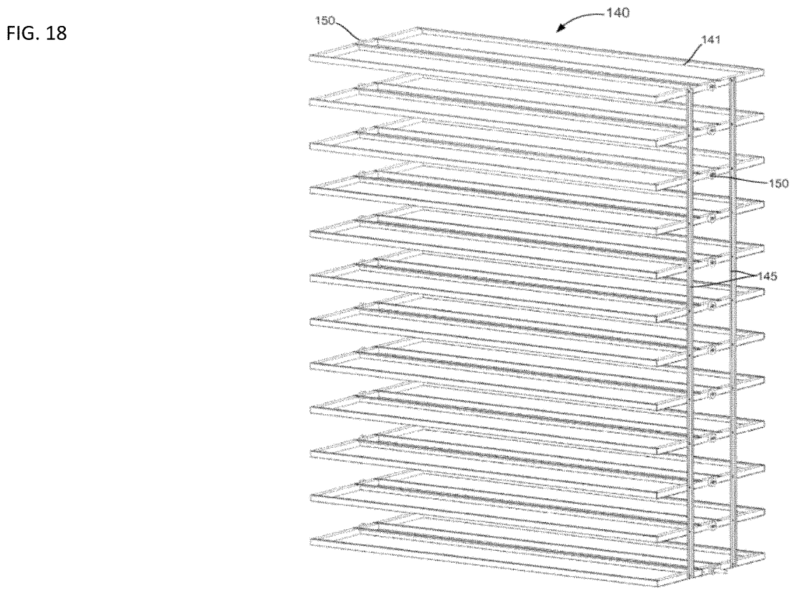

The shelf assembly 140 is rotatably coupled to the uprights 111 (see e.g., FIG. 14) and includes a set of shelves 141, a set of outer bushings 150, a set of inner bushings 155, and a set of linkages 145. For example, as shown in FIG. 18, the shelf assembly 140 can include any suitable number of shelves 141 configured to be vertically stacked. Furthermore, the shelves 141 are operatively coupled together via the linkages 145 (e.g., the linkages 145 transfer at least a portion of a force to cause each shelf 141 of the shelf assembly 140 to pivot simultaneously, as further described herein). While shown in FIG. 18 as including two linkages 145, in some embodiments, a shelf assembly can include any suitable number of linkages 141. For example, in some embodiments, a shelf assembly can include a set of four linkages 141 such that a first set of two linkages 145 are disposed on a first side of the shelves 141 and a second set of two linkages 145 are disposed on a second side of the shelves 141.

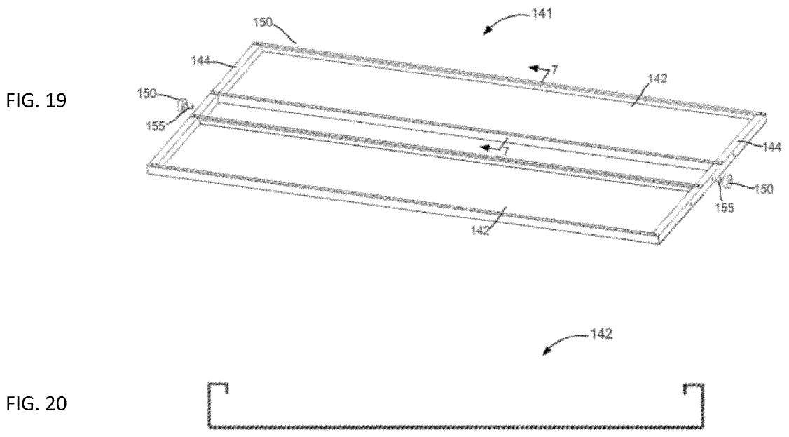

As shown in FIG. 19, each shelf 141 includes a set of platforms 142 coupled together by support tubes 144 (e.g., a first support tube 144 is disposed on a first side of a shelf 141 and a second support tube 144 is disposed on a second side of a shelf 141). As shown in FIG. 20, the platforms 142 define a cross-sectional shape defining a double return, thereby increasing the strength and rigidity of the platform 142. Furthermore, at least one of the support tubes 144 of a shelf 141 is configured to be coupled to the linkages 145.

The outer bushing 150 and the inner bushing 155 (FIG. 21) are configured to rotatably couple the shelf assembly 140 to the uprights 111. More specifically, the inner bushings 155 are rigidly coupled to the support tubes 144 of the shelves 141 and the outer bushings 150 are rigidly coupled to the uprights 111. In this manner, the inner bushings 155 can be rotatably disposed within an opening 151 defined by the outer bushings 150. Thus, the shelves 141 can pivot about the inner bushings 155, disposed within the openings 151 of the outer bushings 150, in response to at least a portion of a force exerted by the drive assembly 170.

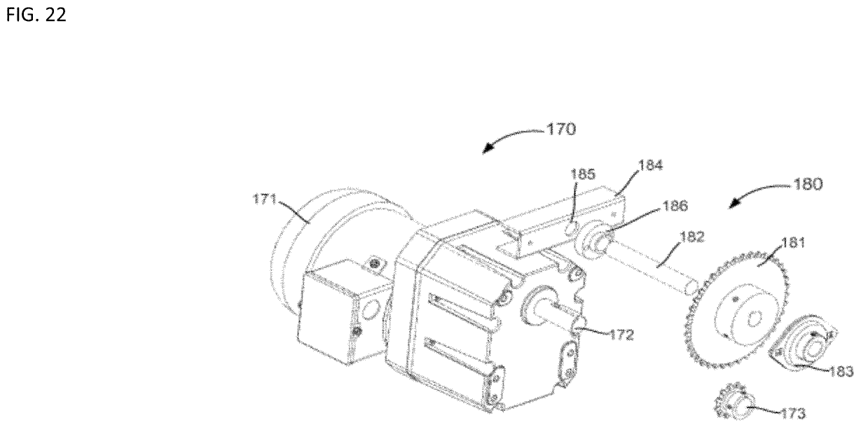

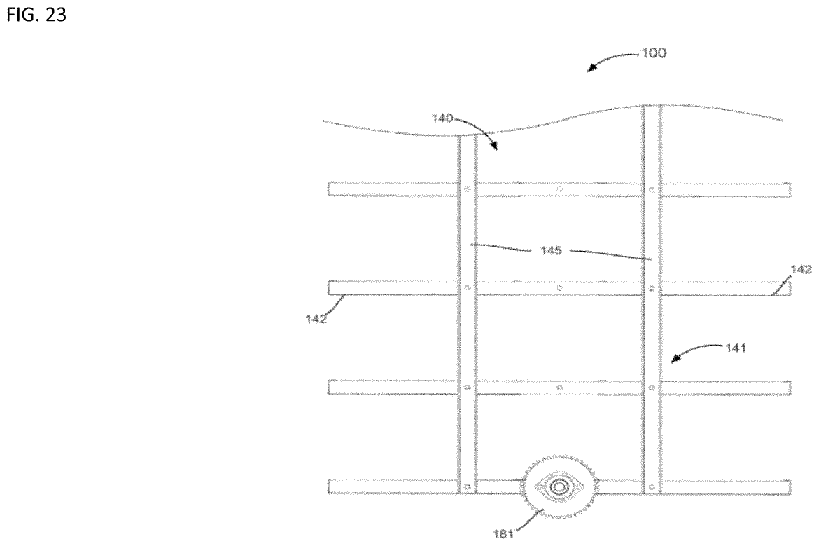

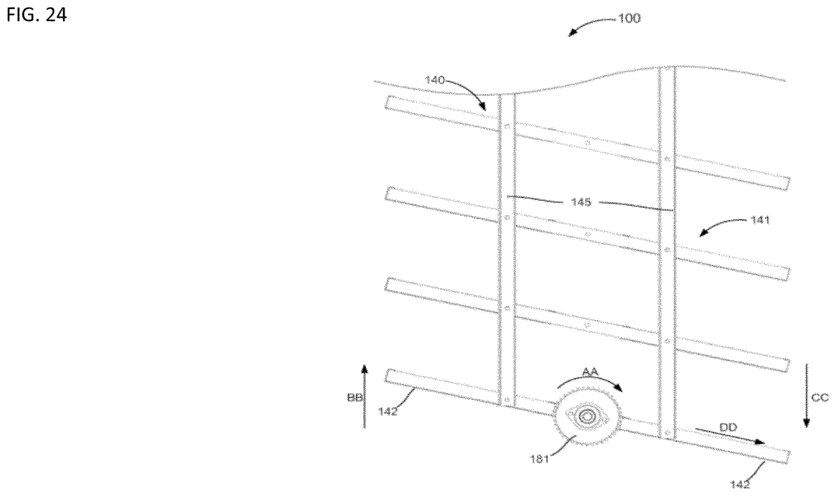

Referring now to FIG. 22, the drive assembly includes a motor 171, a drive gear 173, and a rocker assembly 180. The motor 171 can be any suitable motor defining any suitable torque and/or output speed. For example, in some embodiments, the motor 171 is a Bison 650AC. As shown in FIG. 16, the motor 171 is configured to be coupled to at least one of the uprights 111 such that a drive shaft 172 extends from the motor 171 through the drive shaft opening 113 of the upright 111 and the drive shaft opening 127 of the support member 126. The drive gear 173 is configured to be disposed about the drive shaft 172 and is housed within the volume 115 defined by the upright 111.

The rocker assembly 180 includes a rocker gear 181, a rocker shaft 182, a bearing 183, a mounting bracket 184, and a rocker bushing 186. The rocker gear 181 can be any suitable size and/or define any suitable number of teeth. Furthermore, the rocker gear 181 is disposed within the volume 115 and is operably coupled to the drive gear 173, for example via a chain (not shown). The arrangement of the drive gear 173 and the rocker gear 181 can be such that a desired gear ratio is defined between the drive gear 173 and the rocker gear 181.

The rocker shaft 182 is configured to be inserted into the rocker gear 181 and the bearing 183 and extends through the rocker shaft opening 128 of the support member 126 and the rocker shaft opening 114 of the upright 111. The bearing 183 can be used to facilitate the rotation of the rocker shaft 182 and/or to reduce wear on the rocker assembly 180. The rocker shaft 182 is further configured to be inserted through the rocker bushing 186 and is fixedly coupled (e.g., welded) to the mounting bracket 184. With the rocker shaft 182 coupled to the mounting bracket 184, the mounting bracket 184 can be coupled to the support tube 144 of a first shelf 141. Thus, with the mounting bracket 184 coupled to the first shelf 141 and the rocker shaft 182, the shelf assembly 140 is operably coupled to the motor 171.

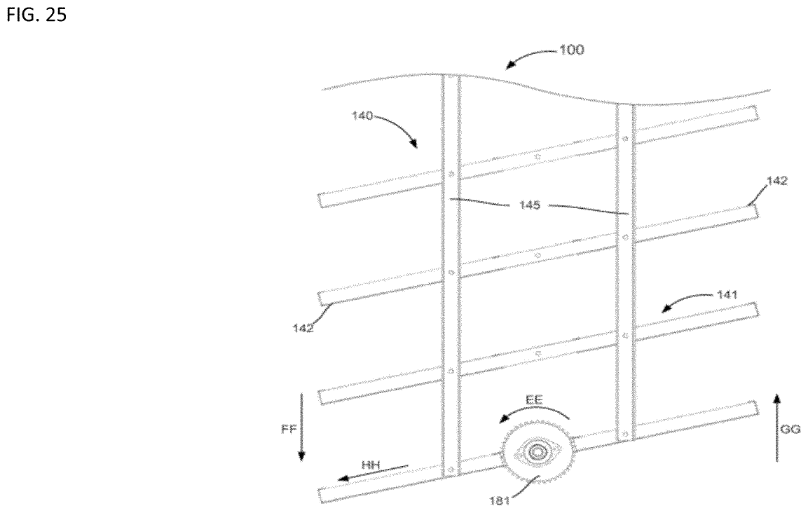

For example, FIG. 23-25 illustrate a portion of the oscillating rack 100 in a first configuration, a second configuration, and a third configuration, respectively. As seen in FIG. 23, the oscillating rack 100 can be in the first configuration such that platforms 142 of the shelves 141 are substantially parallel to a horizontal axis (e.g., the shelves 141 are parallel to the ground). In some embodiments, the shelves 141 can be substantially perpendicular to the linkages 145, while the oscillating rack 100 is in the first configuration.

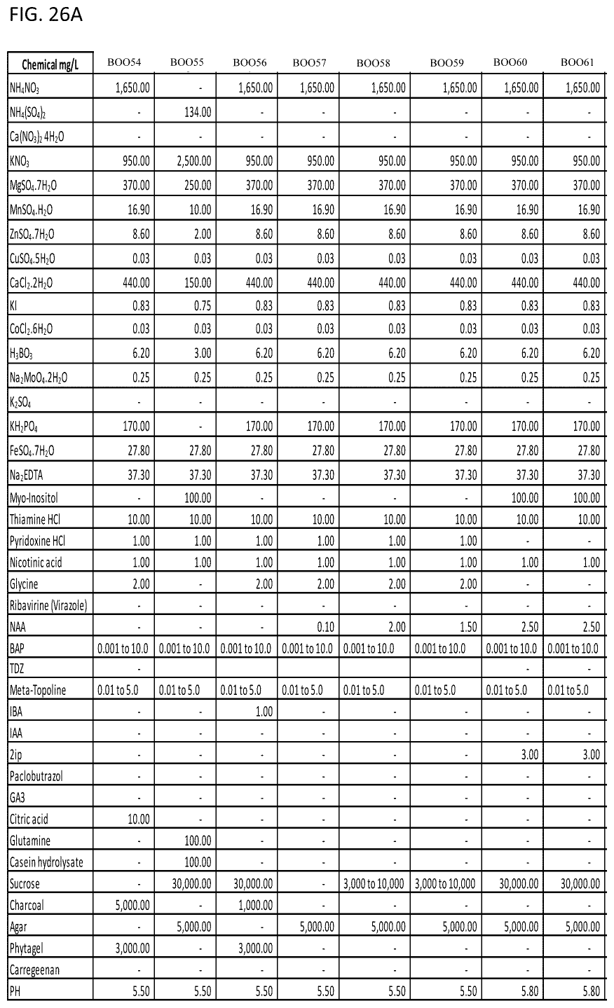

As shown in FIG. 24, the oscillating rack 100 can be moved towards the second configuration by rotating the rocker gear 181 in the direction of the arrow AA. More specifically, the motor 171 (not shown in FIG. 24) can be electrically engaged (e.g., placed in the “on” position via, for example, a control panel) such that the motor 171 rotates the drive shaft 172 and the drive gear 173. The motor 171 can be configured to rotate the drive shaft 172 at any given output speed. For example, in some embodiments, the motor 171 can be configured to rotate the drive shaft 172 at a rate between 0.5 RPM and 1 RPM.

As described above, the rocker gear 181 is operably coupled to the drive gear 173 via a chain. Thus, the chain transfers a portion of the rotational force produced by the motor 171 to the rocker gear 181 such that the rocker gear 181 rotates in the direction of the arrow AA. With the mounting bracket 184 coupled to the first shelf 141 (as described above), a portion of the rotational force, produced by the motor 171, is applied to the first shelf 141. In this manner, a first end of the first shelf 141 is urged to move in the direction of the arrow BB and a second end of the first shelf 141 is urged to move in the direction of the arrow CC. Moreover, with the linkages 145 coupled to the each of the shelves 141, the linkages 145 transfer a portion of the rotational force produced by the motor 171 to each of the shelves 141. Therefore, each shelf 141 is configured to move concurrently with the first shelf 141 in response to at least a portion of the rotational force produced by the motor 171. In addition, when in use with, for example, cultivated plant tissue, the pivoting motion of the shelves 141 can be such that a set of portions of the plant tissue, such as the roots, disposed on a surface of the platforms 142 are intermittently tilted so that the portions (e.g., roots) are alternately immersed in, and free of, a liquid nutrient contained in the vessels. Expanding further, the pivoting motion of the shelves 141 is such that the shelves 141 are placed at an angle relative to the horizontal axis, thus, the liquid nutrients flow in the direction of the arrow DD.

As shown in FIG. 25, the oscillating rack 100 can be moved from the second configuration towards the third configuration by rotating the rocker gear 181 in the direction of the arrow EE (substantially opposite the direction AA). With the rocker gear 181 being moved in the direction of the arrow EE, the first end of the first shelf 141 is urged to move in the direction of the arrow FF (substantially opposite the direction BB) and the second end portion of the first shelf 141 is urged to move in the direction of the arrow GG (substantially opposite the direction CC). Furthermore, the linkages 145 urge each of the shelves 141 of the shelf assembly 140 to move concurrently with the first shelf 141. Thus, when in use with, for example, cultivated plant tissue, the pivoting motion of the shelves 141 in the direction EE can be such that the liquid nutrient can be urged to flow in the direction of the arrow HH such that the cultured plant tissues (e.g., the roots) are alternately immersed in, and free of, the liquid nutrient contained in the vessels.

When in use, the oscillating rack 100 can be configured to oscillate between the second configuration and the third configuration. In some embodiments, the oscillating rack 100 can oscillate between the second configuration and the third configuration with a given cycle time. For example, in some embodiments, the cycle time can be 25 seconds (e.g., an oscillating time of 15 seconds and a hold time in the second configuration or the third configuration for 10 seconds before moving in the opposite direction). In other embodiments, the cycle time can be any other suitable length of time. In some embodiments, the oscillating rack 100 can include a sensor (described above). In such embodiments, the sensor, such as a magnetic sensor, can be configured to sense the position of the shelf assembly 140 relative to the frame 100. The sensor can be configured to be in electrical communication with, for example, a programmable logic controller. The programmable logic controller and the sensor can detect a system malfunction. For example, in some embodiments, the programmable logic controller can be configured to send an electrical signal to an output device to generate a suitable output if the sensor does not sense the position of the shelf assembly 140 for predetermined time period (e.g., 35 seconds). The output can be an audible alarm, a flashing light, a telephone call, an email, and/or any other suitable notification.

The components described herein can be made using any suitable manufacturing technique. For example, in some embodiments, some components can be extruded. In some embodiments, the components can be formed (e.g., bent). In such embodiments, the components can include any suitable feature such that the component defines a specific material characteristic. For example, the platforms 142 are described above as including a double return configured to increase the strength and/or rigidity of the platforms 142. In some embodiments, other components can include similar features. For example, in some embodiments, the uprights 111 can include a double return. In other embodiments, the linkages 145 can include a double return.

The components described herein can be assembled in any suitable manner. For example, in some embodiments, components can be welded. In other embodiments, at least a portion of the components can be mechanically fastened. For example, in some embodiments, portions of the components described herein can be assembled (e.g., coupled) via bolts and nuts, screws, pins, and/or the like. In some embodiments, a portion of the components can be assembled using self-clinching nuts (e.g., PEM nuts) in conjunction with bolts or screws.

While various embodiments have been described above, it should be understood that they have been presented by way of example only, and not limitation. Where schematics and/or embodiments described above indicate certain components arranged in certain orientations/or positions, the arrangement of components may be modified. Similarly, where methods and/or events described above indicate certain events and/or procedures occurring in certain order, the ordering of certain events and/or procedures may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above.

### Methods for Pistachio Bioculture

The present application provides methods for pistachio tissue culture. The methods enable a massive production of pistachio plants within a short time, at a low cost. The methods can be conducted with or without a bioreactor.

In some embodiments, the methods comprise (a) obtaining pistachio explant. Any suitable plant parts may be used. In some embodiments, single-node explants, shoot tips, basal (bottom) parts of plants with multiple buds, are used as starting material as explants. The explants can be treated to substantially reduce the chance of contamination. Any suitable methods can be used. In some embodiments, commercial bleach can be used. For example, explant can be sterilized in about 1%, 5%, 10%, 15% or more commercial bleach for about 10 minutes, 20 minutes, 30 minutes, or more depending on the condition of the explant. In some embodiments, to further reduce the chance of contamination, explant can be cut into small piece, such as about 3 mm, about 5 mm, or more in length. The small pieces can be rinsed again once, twice, or more in about 1%, 5%, 10%, or 15% commercial bleach solution and then placed on an initiation medium. The initiation medium can be any suitable medium as described herein.

This step is completed when shoot tips start breaking and forming multiple shoots from the explant. During the process, explant can be sub cultured on a fresh initiation medium every 3 to 4 weeks or any suitable period of time.

The multiple shoots initiated from the explant can be dived into small clumps, for example, clumps of 2 to 3 shoots each and transferred to a multiplication medium as described herein. This step can be conducted in a bioreactor.

Any suitable growth condition can be used. In some embodiments, the plants are grown under about 20-28° C. (e.g., about 22-24° C.), day-night cycle (e.g., 16/8 hour day/night), with a photon flux density of about 10-150 μmol/m²/s (e.g, 80-100 μmol/m²/s).

In some embodiments, a temporary immersion bioreactor is used. In some embodiments, in a single cultivation cycle, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more to fill the cultivation chamber of the bioreactor in which the plants are grown with a predetermined amount of liquid or semi-liquid medium. The medium is kept in the chamber for about 1, 2, 3, 4, 5, 6, 7, 8, 9, 10, 15, 20, 25, 30, 35, 40, 45, 50, 55, 60 minutes or more, and then drained from the chamber. In some embodiments, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes to drain the medium out of the chamber. Then optionally the chamber is dried for about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes. In some embodiments, the cultivation cycle described above takes about 1, 2, 3, 4, 5, 6, 7, 8, 9, 10, 15, 20, 25, 30, 35, 40, 45, 50, 55, 60 minutes or more.

In some embodiments, a relatively small amount of liquid medium is used, such as about 25-100 ml of liquid medium per bioreactor. Advantages of using a relatively small amount of liquid medium include, but are not limited to, better control of temporal immersion of the explants, preventing drowning of the explains, and reducing chances of contamination.

In some embodiments, the liquid medium in the bioreactors is changed with fresh one every 1 week, 2 weeks, 3 weeks, 4 weeks or more, each of which is called a growth cycle (or cycle). Advantages of using refreshed liquid medium include, but are not limited to, providing more nutrients to plants, removing accumulated detrimental chemicals in the medium due to plant metabolism, and reducing chances of contamination.

In some embodiments, an oscillating rack system is used to move liquid from one side to another. In some embodiments, the oscillation cycle is about once per two minutes, once per minute, one and a half per minute, twice per minute or more. In some embodiments, the oscillating rack system is used in the initiation and/or multiplication step. A non-limiting example of oscillating rack system is described in International Patent Application No. PCT/US2012/047622, which is incorporated herein in its entirety including any figures therein.

In some embodiments, the pistachio plant tissue biomass are multiplied for about 1.5 times, about 2 times, about 2.5 times, about 3 times, about 3.5 times, about 4 times, about 4.5 times, about 5 times, about 5.5 times, about 6.0 times, about 6.5 times, about 7 times, about 7.5 times, about 8 times, or more during each growth cycle.

Any suitable plant, plant part, plant tissue culture, or plant cell can be used as the explant. In some embodiments, the explant is pathogen-free, e.g., bacteria-free, fungi-free and/or virus-free. In some embodiments, the explant is a pistachio rhizome shoot tip. In some embodiments, the explant is taken from a plant of about 1 week old, about 2 weeks old, about 3 weeks old, about 1 month old, about 2 month old, about 3 months old, about half year old, about 1 year old, or more.

The explant can be any segment or collection of cells from apical meristems, terminal buds, axillary buds, adventitious buds, accessory buds, pseudo-terminal buds, cambium, lateral meristem, lateral bud, vegetative buds, reproductive buds, mixed buds, shoot segments, shoot apices, stem segments, immature nodal sections from stems, lateral shoots, seedlings, seeds, shoots starting to rise from the ground, immature flower buds, inflorescences, crown segments, leaf segments, roots, rhizome, or any part thereof.

The multiplied shoots are then transferred to a rooting medium as described herein. Optionally, the multiplied shoots are divided into clumps of about 3 to 6 shoots before the transfer. It usually takes about 2-4 weeks for the shoots to develop roots. Once the roots are formed, the plants can be transfer to either in vitro or in vivo conditions for further growth.

The methods described herein can be further modified and optimized, depending on the purposes, goals, and other factors that may affect pistachio tissue culture. For example, factors affecting pistachio tissue culture are disclosed in Behboodi, (Tissue culture results of all wild pistachio species and some cultivars in Iran, Acta Hort. (ISHS) 591:399-403), Wei (Master Thesis, Studies On Tissue Culture And Browning Of Pistacia Vera. L., 2008, GlobeThesis ID 2143360212988571), Can et al. (In vitro micrografting of pistachio, Pistacia vera, L. var. Siirt, on wild pistachio rootstocks, Journal of Cell and Molecular Biology 5: 25-31, 2006), Fei et 1. (Micropropagation of Pistacia vera ‘Kerman’, Agricultural Science and Technology, February 2010), each of which is incorporated herein by reference in its entirety for all purposes.

The present invention is further illustrated by the following examples that should not be construed as limiting. The contents of all references, patents, and published patent applications cited throughout this application, as well as the Figures, are incorporated herein by reference in their entirety for all purposes.

### Methods for Yam Bioculture

Bulletin OEPP/EPPO Bulletin 36, 187.

In some embodiments, this step comprises breaking field tuber dormancy to induce buds, sprouting of buds, sterilization of sprout, and subsequent cycles of cultivation in vivo or in vitro.

The tuber dormancy can be broken naturally, or by treatment with GA3, ethanol, temperature treatment, thiourea, ethylene chlorohydrins, rindite, carbon disulphide, and/or bromoethane, etc., or by methods described in Bryan, 1989 and Claassens et al., 2005, each of which is incorporated by reference in its entirety.

Any sterilization method suitable for plant can be used. In some embodiments, the sprouts are sterilized in 0.5% solution of NaDCC.

The sterilized sprouts are then cultivated in vitro (e.g., in a tube) on a solid or semi-solid medium. In some embodiments, the sprouts are first cultivated in solid medium, wherein the medium comprises MS salts, IAA, 2ip, and sucrose. In some embodiments, the concentration of IAA is about 0.1 to 1 mg/L, e.g., about 1 mg/L; the concentration of 2ip is about 1 to 10 mg/L, e.g., about 4-5 mg/L; and the concentration of sucrose is about 10 to 40 g/L, e.g., about 30 g/L. Then the sprouts are grown on a medium comprising MS salts and sucrose without any hormones. In some embodiments, the concentration of sucrose is about 10 to 40 g/L, e.g., about 20 g/L, e.g., the BOO17 medium as described herein. Any suitable growth condition can be used. In some embodiments, the sprouts are grown under about 20-28° C. (e.g., about 24° C.), day-night cycle (e.g., 16/8 hour day/night), with a photon flux density of about 50-150 μmol/m²/s (e.g., about 85-100 μmol/m²/s). In some embodiments, the cultivation takes about one to three months, e.g., about two months, or takes as long as needed until the plants are pathogen-free. As used herein, one skilled in the art would understand that the standard of “pathogen-free” varies from one pathogen species to another, and the plant can be regarded as pathogen-free as long as the population of a specific pathogen contained in the plants does not substantially affect future microtuber production.

Optionally, the step of obtaining pathogen-free yam sprouts comprises testing plants for the presence of yam pathogens, such as one or more bacteria species, fungal species, and/or virus species. In some embodiments, the virus species is selected from Dioscorea bacilliform virus (DBV, genus Badnavirus), Yam mosaic virus (YMV, genus Potyvirus), and Yam mild mosaic virus (YMMV, genus Potyvirus). In some embodiments, the testing methods comprise detecting one or more nucleotides (e.g., DNA or RNA) and/or one or more polypeptide that is specific to the pathogen, by using any suitable technologies known to one skilled in the art.

In some embodiments, the methods comprise (b) propagating the pathogen-free yam sprouts obtained in step (a) or any other sources to produce yam plants. The step is also called elongation stage in which stems of yam plants are elongated. In some embodiments, the propagation is in vitro or in vivo. In some embodiments, the propagation is done in a bioreactor of the present application or any other suitable bioreactors known to one skilled in the art, or simply in any suitable culture tubes. In some embodiments, solid, semi-solid, liquid or semi-liquid medium is used. In some embodiments, one 4-5-week-old well-developed yam plant contained multiple axillary buds is used as the starting materials. In some embodiments, such well-developed yam plant has about 3-10 axillary buds, e.g., about 4 to 7 axillary buds.

In some embodiments, the well-developed yam plant is grown either on solid medium or semi-solid medium in a culture tube, or in liquid or semi-liquid medium in a bioreactor. In some embodiments, the medium comprises MS salts and sucrose without any hormones, e.g., the propagation and multiplication media as described above. In some embodiments, the concentration of sucrose is about 10 to 40 g/L, e.g., about 20 g/L, e.g., the BOO17 medium as described herein.

Any suitable growth condition can be used. In some embodiments, the plants are grown under about 20-28° C. (e.g., about 24° C.), day-night cycle (e.g., 16/8 hour day/night), with a photon flux density of about 50-150 μmol/m²/s (e.g., about 85-100 μmol/m²/s) when a culture tuber is used, or about 10-100 μmol/m²/s (e.g., about 30-80 μmol/m²/s) when a bioreactor is used.

In some embodiments, the cultivation takes about 3-8 weeks in a solid or semi-solid medium in culture tubes, e.g., about 4-6 weeks, or about 1-4 weeks in a liquid medium in bioreactors, e.g., about 2.5-3 weeks, depending on yam variety.

In some embodiments, a temporary immersion bioreactor is used. In some embodiments, in a single cultivation cycle, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more to fill the cultivation chamber of the bioreactor in which the plants are grown with a predetermined amount of liquid or semi-liquid medium. The medium is kept in the chamber for about 1, 2, 3, 4, 5, 6, 7, 8, 9, 10, 15, 20, 25, 30, 35, 40, 45, 50, 55, 60 minutes or more, and then drained from the chamber. In some embodiments, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes to drain the medium out of the chamber. Then optionally the chamber is dried for about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes. In some embodiments, the cultivation cycle described above takes about 1, 2, 3, 4, 5, 6, 7, 8, 9, 10, 15, 20, 25, 30, 35, 40, 45, 50, 55, 60 minutes or more.

In some embodiments, the same medium is used in each cultivation cycle. In some embodiments, two or more different media are used sequentially, of which each is used in a cycle.

In some embodiments, a relatively small amount of liquid medium is used, such as about 25-100 ml of liquid medium per bioreactor. Advantages of using a relatively small amount of liquid medium include, but are not limited to, better control of temporal immersion of the explants, preventing drowning of the explains, and reducing chances of contamination.

In some embodiments, the liquid medium is the bioreactors is changed with fresh one every 1 day, 2 days, 3 days, 4 days, 5 days, 6 days, every week, every 10 days, or every two weeks. Advantages of using refreshed liquid medium include, but are not limited to, providing more nutrients to plants, removing accumulated detrimental chemicals in the medium due to plant metabolism, and reducing chances of contamination.

In some embodiments, an oscillating rack system is used to move liquid from one side to another. In some embodiments, the oscillation cycle is about once per two minutes, once per minute, one and a half per minute, twice per minute or more. In some embodiments, the oscillating rack system is used in the propagation stage (elongation stage) and/or microtuberization stages (e.g., the pre-tuberization stage and tuberization stage). A non-limiting example of oscillating rack system is described in U.S. Provisional Patent Application U.S. 61/618,344, filed on Mar. 30, 2012, which is incorporated herein in its entirety including any figures therein.

In some embodiments, the yam plants are multiplied for about 1.5 times, about 2 times, about 2.5 times, about 3 times, about 3.5 times, about 4 times, about 4.5 times, about 5 times, about 5.5 times, about 6.0 times, about 6.5 times, about 7 times, about 7.5 times, about 8 times, or more. This step results in increased shoot length and more internodes per plant.

Any suitable yam plant, plant part, plant tissue culture, or plant cell can be used as the explant for yam micropropagation. In some embodiments, the explant is pathogen-free, e.g., bacteria-free, fugi-free and/or virus-free. In some embodiments, the explant is a yam stock plant maintained by serial in vitro subculture. In some embodiments, the explant is a segment of yam seedlings. In some embodiments, the segment of yam comprises one or more axillary bud. In some embodiments, the bud is dormant or active. In some embodiments, the explant is taken from a plant of about 1 week old, about 2 weeks old, about 3 weeks old, about 1 month old, about 2 month old, about 3 months old, about half year old, about 1 year old, or more.

The explant can be any segment or collection of cells from apical meristems, terminal buds, axillary buds, adventitious buds, accessory buds, pseudo-terminal buds, cambium, lateral meristem, lateral bud, vegetative buds, reproductive buds, mixed buds, shoot segments, shoot apices, stem segments, immature nodal sections from stems, lateral shoots, seedlings, seeds, shoots starting to rise from the ground, immature flower buds, inflorescences, crown segments, leaf segments, roots, recycled microtubers, sprouts from cold-stored seed tubers, or any part thereof.

In some embodiments, the methods further comprise (c) pretreating the yam plants obtained from step (b) or any other sources to produce pretreated yam plants. This step is also called pre-tuberization stage. In some embodiments, this step was performed in a bioreactor. In some embodiments, the bioreactor is a temporary immersion bioreactor. In some embodiments, liquid or semi-liquid medium is used, e.g. the pre-tuberization media as described above.

In some embodiments, the yam plants obtained from step (b) or any other resources are cultured in a liquid medium, wherein the each plant has about 3-10 axillary buds, e.g., about 4 to 7 axillary buds. In some embodiments, the medium comprises MS salts, sucrose, at least one cytokinin, and at least one auxin, e.g., BOO18 as described herein. In some embodiments, the cytokinin is 2ip or analog thereof. In some embodiments, the auxin is IAA or analog thereof. Alternatively, the medium comprises MS salts, sucrose, and at least one growth retardant, e.g., the BOO23, BOO19, BOO20, BOO24 media described herein, or combination thereof. In some embodiment, at least one retardant is ancymidol or analog thereof. Still in some embodiments, the pre-tuberization media comprise one or more cytokinin, one or more auxin, and one or more growth retardant. In some embodiments, the concentration of 2ip is about 1 to 10 mg/L, for example, about 4-5 mg/L. In some embodiments, the concentration of IAA is about 0.1 to 10 mg/L, for example, about 1 mg/L. In some embodiments, the concentration of ancymidol is about 0.1 to 10 mg/L, for example, about 0.5 mg/L, about 1 mg/L, about 2 mg/L, or about 5 mg/L. In any case, the sucrose concentration is about 20 g/L to about 40 g/L, for example, about 30 g/L. In some embodiments, a medium comprising MS salts, sucrose, at least one cytokinin, and at least one auxin, and a medium comprising MS salts, sucrose, and at least one growth retardant are used in combination, or sequentially during the pretreatment stage in any order, in one or more culture cycles.

Any suitable growth condition can be used. In some embodiments, the plants are grown under about 20-28° C. (e.g., about 24° C.), day-night cycle (e.g., 16/8 hour day/night), with a photon flux density of about 10-100 μmol/m²/s (e.g., about 30-80 μmol/m²/s). In some embodiments, the duration of the pretreatment step is about 1-4 weeks, e.g., about 1 to 2 weeks or about 2-3 weeks.

In some embodiments, the methods further comprise (d) initiating microtubers from the pretreated yam plants obtained from step (c) or any other sources. This step is also called tuberization stage.

One or more ways to initiate tuberization of yam in vitro can be utilized in step (d). In some embodiments, the methods of present application comprise initiating tuberization in vitro by supplying relatively high concentration of sucrose. For example, the sucrose concentration in the tuberization induction media is about 5% w/v, about 6% w/v, about 7% w/v, about 8% w/v, about 9% w/v, about 10% w/v, or more.

In some embodiments, the methods of present application comprise triggering tuberization in vitro by shifting the tissue culture from long-day light conditions to short-day light conditions. For example, the photoperiod condition is changed from long-day conditions, such as about 23/1 hours (light/dark), about 22/2 hours (light/dark), about 21/3 hours (light/dark), about 20/4 hours (light/dark), about 19/5 hours (light/dark), about 18/6 hours (light/dark), about 17/7 hours (light/dark), about 16/8 hours (light/dark), about 15/9 hours (light/dark), about 14/8 hours (light/dark), or about 13/11 hours (light/dark) to short-day conditions, such as about 11/13 hours (light/dark), about 10/14 hours (light/dark), about 9/15 hours (light/dark), about 8/16 hours (light/dark), about 7/17 hours (light/dark), about 6/18 hours (light/dark), about 5/19 hours (light/dark), about 4/20 hours (light/dark), about 3/21 hours (light/dark), about 2/22 hours (light/dark), or about 1/23 hours (light/dark).

In some embodiments, the methods of present application comprise triggering tuberization in vitro by using a total darkness condition.

In some embodiments, the methods of present application comprise triggering tuberization in vitro by using cool temperature conditions. For example, the temperature during the day time and/or the night time is about 25°±1° C., 24°±1° C., 23°±1° C., 22°±1° C., 21°±1° C., 20°±1° C., 19°±1° C., 18°±1° C., 17°±1° C., 16°±1° C., 15°±1° C., 14°±1° C., or lower. In some embodiments, the day time temperature is about 20°±2° C. and night time temperature is about 18°±2° C. In some embodiments, the temperature during the night time is lower than the temperature during the day time, for example, about 0.5° C., about 1° C., about 2° C., about 3° C., about 4° C., about 5° C., or more.

In some embodiments, the methods of present application comprise triggering tuberization in vitro by using one or more phytohormones or growth regulators, such as cytokins or growth retardants. In some embodiments, the cytokin is selected from the group consisting of thidiazuron (TDZ), N⁶-benzylaminopurine (BAP, a.k.a. BA), meta-topolin (mT), zeatin, zeatin riboside, dihydrozeatin, kinetin, isopentenyladenine (ip, e.g., 2ip), adenine hemisulfate, dimethylallyladenine, N-(2-chloro-4-pyridyl)-N′-phenylurea) (4-CPPU), analog thereof, and combination thereof. In some embodiments, the growth retardants is selected from the group consisting of alar, ancymidol, chlorocholine chloride (CCC), coumarin, fluridone, tetcyclacis (TET), ancymidol, analog thereof, and combination thereof. In some embodiments, the growth retardant is a gibberellic acid (GA3) antagonist, such as ancymidol and its functional derivatives.

In some embodiments, the methods of present application comprise triggering tuberization in vitro by increased nitrate:ammonium ratio and/or increased nitrogen:carbon ratio.

In some embodiments, more than one way of triggering yam tuberization described above are simultaneously and/or sequentially used. More methods for triggering yam tuberization can be found in Donnelly et al. 2003, Seabrook et al. 1993, Gopal et al. 1998, and Gopal et al. 1997, Garner and Blake et al. 1989, Bizari et al. 1995, Nasiruddin and Blake 1994, each of which is incorporated by reference in its entirety for all purposes.

In some embodiments, step (d) was performed in a bioreactor. In some embodiments, the bioreactor is a temporary immersion bioreactor. In some embodiments, liquid or semi-liquid medium is used, e.g. the pre-tuberization media as described above. In some embodiments, the yam plants obtained from step (c) or any other resources are cultured in a liquid or semi-liquid medium. In some embodiments, the liquid medium or semi-liquid medium comprises one or more auxin, but does not comprise any cytokinin, e.g., BOO21 media described herein. Alternatively, instead of auxin, the tuberization medium comprises one or more plant retardant, such as ancymidol or analog thereof, e.g., BOO25, BOO26, BOO27, BOO22 media described herein, or combination thereof. In some embodiments, the tuberization medium comprises one or more auxin and one or more growth retardant. In some embodiments, the auxin is NAA. In some embodiments, the NAA concentration is about 0.01 to about 0.05 mg/L, for example, about 0.01 mg/L, or about 0.02 mg/L. In some embodiments, the concentration of ancymidol is about 0.1 to 10 mg/L, for example, about 0.5 mg/L, about 1 mg/L, or about 5 mg/L. In any case, the sucrose concentration in the tuberization medium is higher compared to the sucrose concentration in the pre-tuberization medium used in step (c). For examples, the sucrose concentration in the tuberization media is about 50 g/L to about 100 g/L or more, for example, about 60 g/L, about 70 g/L, or about 80 g/L. In some embodiments, the sucrose concentration is about 80 g/L. Any suitable growth condition can be used.

In some embodiments, the plants are grown under a temperature that is lower than the temperature used in step (c). For example, when the temperature used in step (c) is about 24° C., the temperature used in step (d) can be about 15-24° C. In some embodiments, the plants are cultured with continuous darkness. In some embodiments, this step lasts for about 5-6 weeks, or any period of time that is suitable for a specific yam species or a specific goal (e.g., with predetermined microtubers production number and/or size).

The methods can further comprise (e) harvesting the microtubers produced in step (d). The microtubers propagated by methods described herein can be either stored under suitable conditions for future use, or could be directly transplanted to soil without any acclimation. Optionally, this step includes washing, drying, weighing, counting and/or storing the microtubers. In some embodiments, the microtubers are stored at a temperature above 0° C. but below about 10° C., e.g., at about 4° C.

The methods described herein can be further modified and optimized, depending on the purposes and goals. For example, factors affecting yam tissue culture disclosed in Nistor et al., 2010, Rosu et al., 2004, Badoni et al., 2009, Wang et al., 1982, Abbott et al, 1986, Ewing et al., 1992, Khuri et al., 1995, Perl et al., 1991, Leclerc et al., 1994, Levyd et al., 1993, Ahmad et al., 1993, and others can be considered. More information can be found in Bajaj, 2009 (Yam Volume 3 of Biotechnology in agriculture and forestry, Springer-Verlag, 1987, ISBN 3540179666, 9783540179665); Haverkort and Anisimov, 2007 (Yam production and innovative technologies, Wageningen Academic Pub, 2007, ISBN 9086860427, 9789086860425); Raj an and Markose 2007 (Propagation of Horticultural Crops: Vol. 06. Horticulture Science Series, New India Publishing, 2007, ISBN 8189422480, 9788189422486); Leclerc 1993 (The production and utilization of yam microtubers, McGill University, 1993), Krasteva and Panayotov, 2009 (Proceedings of the fourth Balkan Symposium on Vegetables and Yams, ISHS, 2009, ISBN 9066055529, 9789066055520), each of which is incorporated herein by reference in its entirety for all purposes.

### Methods of Potato Bioculture

The present application provides methods for potato bioculture.

In another aspect of the present invention, the methods comprise (a) obtaining pathogen-free potato sprouts. Any suitable methods can be used, such as the methods described in Quazi et al. 1978; Al-Taleb 2011; and Plant Tissue Culture—Theory and Practice, a Revised Edition, Chapter 15, 1996; 2006 OEPP/EPPO, Bulletin OEPP/EPPO Bulletin 36, 187, or as described herein. In some embodiments, this step comprises breaking field tuber dormancy to induce buds, sprouting of buds, sterilization of sprout, and subsequent cycles of cultivation in vivo or in vitro.

The tuber dormancy can be broken naturally, or by treatment with GA3, ethanol, temperature, thiourea, ethylene chlorohydrins, rindite, carbon disulphide, and/or bromoethane, etc., or by methods described in Bryan, 1989 and Claassens et al., 2005, each of which is incorporated by reference in its entirety.

In some embodiments of the present invention, the induced buds are further grown into sprouts. In some further embodiments, the sprouts are further sterilized. Any sterilization method suitable for plant can be used. In some embodiments, the sprouts are sterilized in sodium dichloroisocyanurate (NaDCC), such as 0.5% solution of NaDCC. In some further embodiments, the sterilized sprouts are cultivated in vitro for one or more cycles until pathogen-free sprouts are produced.

The sterilized sprouts are then cultivated in vitro (e.g., in a tube) on a solid or semi-solid medium. In some embodiments of the present invention, sterilized sprouts are cultivated on a first medium for one or more cycle, and then cultivated on a second medium for one or more cycles until pathogen-free potato plants are produced. In some embodiments, the first medium is a solid, semi-solid, liquid, or semi-liquid medium comprising MS salts, IAA, 2ip, and sucrose. In some embodiments, the sprouts are first cultivated in solid medium, wherein the medium comprises MS salts, IAA, 2ip, and sucrose. In some embodiments, the concentration of IAA is about 0.1 to 1 mg/L, e.g., about 1 mg/L; the concentration of 2ip is about 1 to 10 mg/L, e.g., about 4-5 mg/L; and the concentration of sucrose is about 10 to 40 g/L, e.g., about 30 g/L. In some further embodiments, the first medium is a BOO18 medium.

In some embodiments of the present invention, the second medium comprises MS salts and sucrose without any hormones or growth regulators. In some embodiments, the sucrose concentration is about 10 g/L to 40 g/L, such as about 20 g/L.

Then the sprouts are grown on a medium comprising MS salts and sucrose without any hormones. In some embodiments, the concentration of sucrose is about 10 to 40 g/L, e.g., about 20 g/L, e.g., the BOO17 medium as described herein. Any suitable growth condition can be used. In some embodiments, the sterilized sprouts are grown under about 20° C. to 28° C., such as about 24° C., until pathogen-free potato plants are produced. In some embodiments of the present invention, the sterilized sprouts are grown under day/night light cycle, with a day time for about 12 hours to 20 hours, such as about 16 hours. In some embodiments, the sterilized sprouts are grown under a photon flux density of about 50 μmol/m²/s to 120 μmol/m²/s, such as about 85-100 μmol/m²/s.

In some embodiments, the sprouts are grown under about 20-28° C. (e.g., about 24° C.), day-night cycle (e.g., 16/8 hour day/night), with a photon flux density of about 50-150 μmol/m²/s (e.g., about 85-100 μmol/m²/s). In some embodiments, the cultivation takes about one to three months, e.g., about two months, or takes as long as needed until the plants are pathogen-free. As used herein, one skilled in the art would understand that the standard of “pathogen-free” varies from one pathogen species to another, and the plant can be regarded as pathogen-free as long as the population of a specific pathogen contained in the plants does not substantially affect future microtuber production.

Optionally, the step of obtaining pathogen-free potato sprouts comprises testing plants for the presence of potato pathogens, such as one or more bacteria species, fungal species, and/or virus species. In some embodiments of the present invention, in order to verify pathogen-free plants are produced, the methods further comprise testing for the presence or absence of one or more potato pathogen species after one or more cycles. In some embodiments, the virus species is selected from Potato leaf roll virus (PLRV), Potato virus A (PVA), Potato virus M (PVM), Potato virus S (PVS), Potato virus X (PVX), Potato virus Potato virus S (PVS), Potato virus X (PVX), Potato virus Y (PVY) and Potato spindle tuber viroid (PSTVd). In some embodiments, the testing methods comprise detecting one or more nucleotides (e.g., DNA or RNA) and/or one or more polypeptide that is specific to the pathogen, by using any suitable technologies known to one skilled in the art.

In some embodiments, the methods comprise (b) propagating the pathogen-free potato sprouts obtained in step (a) or any other sources to produce potato plants. The step is also called elongation stage in which stems of potato plants are elongated. In some embodiments, the propagation is in vitro or in vivo. In some embodiments, the propagation is done in a bioreactor of the present application or any other suitable bioreactors known to one skilled in the art, or simply in any suitable culture tubes. In some embodiments, solid, semi-solid, liquid or semi-liquid medium is used. In some embodiments, one 4-5-week-old well-developed potato plant contained multiple axillary buds is used as the starting materials. In some embodiments, such well-developed potato plant has about 3-10 axillary buds, e.g., about 4 to 7 axillary buds.

In some embodiments, the well-developed potato plant is grown either on solid medium or semi-solid medium in a culture tube, or in liquid or semi-liquid medium in a bioreactor. In some embodiments, the medium comprises MS salts and sucrose without any hormones, e.g., the propagation and multiplication media as described above. In some embodiments, the concentration of sucrose is about 10 to 40 g/L, e.g., about 20 g/L, e.g., the BOO17 medium as described herein.

Any suitable growth condition can be used. In some embodiments, the plants are grown under about 20-28° C. (e.g., about 24° C.), day-night cycle (e.g., 16/8 hour day/night), with a photon flux density of about 50-150 μmol/m²/s (e.g., about 85-100 μmol/m²/s) when a culture tuber is used, or about 10-100 μmol/m²/s (e.g., about 30-80 μmol/m²/s) when a bioreactor is used.

In some embodiments, the cultivation takes about 3-8 weeks in a solid or semi-solid medium in culture tubes, e.g., about 4-6 weeks, or about 1-4 weeks in a liquid medium in bioreactors, e.g., about 2.5-3 weeks, depending on potato variety.

In some embodiments, a temporary immersion bioreactor is used. In some embodiments, in a single cultivation cycle, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more to fill the cultivation chamber of the bioreactor in which the plants are grown with a predetermined amount of liquid or semi-liquid medium. The medium is kept in the chamber for about 1, 2, 3, 4, 5, 6, 7, 8, 9, 10, 15, 20, 25, 30, 35, 40, 45, 50, 55, 60 minutes or more, and then drained from the chamber. In some embodiments, it takes about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes to drain the medium out of the chamber. Then optionally the chamber is dried for about 0.1, 0.2, 0.3, 0.4, 0.5, 1, 1.5, 2, 2.5, 3, 3.5, 4, 4.5, 5, 5.5, 6, 7, 8, 9, 10, 15, 20, 25, 30 minutes or more minutes. In some embodiments, the cultivation cycle described above takes about 1, 2, 3, 4, 5, 6, 7, 8, 9, 10, 15, 20, 25, 30, 35, 40, 45, 50, 55, 60 minutes or more.

In some embodiments, the same medium is used in each cultivation cycle. In some embodiments, two or more different media are used sequentially, of which each is used in a cycle.

In some embodiments, a relatively small amount of liquid medium is used, such as about 25-100 ml of liquid medium per bioreactor. Advantages of using a relatively small amount of liquid medium include, but are not limited to, better control of temporal immersion of the explants, preventing drowning of the explains, and reducing chances of contamination.

In some embodiments, the liquid medium is the bioreactors is changed with fresh one every 1 day, 2 days, 3 days, 4 days, 5 days, 6 days, every week, every 10 days, or every two weeks. Advantages of using refreshed liquid medium include, but are not limited to, providing more nutrients to plants, removing accumulated detrimental chemicals in the medium due to plant metabolism, and reducing chances of contamination.

In some embodiments, an oscillating rack system is used to move liquid from one side to another. In some embodiments, the oscillation cycle is about once per two minutes, once per minute, one and a half per minute, twice per minute or more. In some embodiments, the oscillating rack system is used in the propagation stage (elongation stage) and/or microtuberization stages (e.g., the pre-tuberization stage and tuberization stage). A non-limiting example of oscillating rack system is described in U.S. Provisional Patent Application U.S. 61/618,344, filed on Mar. 30, 2012, which is incorporated herein in its entirety including any figures therein.

In some embodiments, the potato plants are multiplied for about 1.5 times, about 2 times, about 2.5 times, about 3 times, about 3.5 times, about 4 times, about 4.5 times, about 5 times, about 5.5 times, about 6.0 times, about 6.5 times, about 7 times, about 7.5 times, about 8 times, or more. This step results in increased shoot length and more internodes per plant.

Any suitable potato plant, plant part, plant tissue culture, or plant cell can be used as the explant for potato micropropagation. In some embodiments, the explant is pathogen-free, e.g., bacteria-free, fungi-free and/or virus-free. In some embodiments, the explant is a potato stock plant maintained by serial in vitro subculture. In some embodiments, the explant is a segment of potato seedlings. In some embodiments, the segment of potato comprises one or more axillary bud. In some embodiments, the bud is dormant or active. In some embodiments, the explant is taken from a plant of about 1 week old, about 2 weeks old, about 3 weeks old, about 1 month old, about 2 month old, about 3 months old, about half year old, about 1 year old, or more.

The explant can be any segment or collection of cells from apical meristems, terminal buds, axillary buds, adventitious buds, accessory buds, pseudo-terminal buds, cambium, lateral meristem, lateral bud, vegetative buds, reproductive buds, mixed buds, shoot segments, shoot apices, stem segments, immature nodal sections from stems, lateral shoots, seedlings, seeds, shoots starting to rise from the ground, immature flower buds, inflorescences, crown segments, leaf segments, roots, recycled microtubers, sprouts from cold-stored seed tubers, or any part thereof. In one embodiment, the explant is taken from a potato plant of about 1 week old, about 2 weeks old, about 3 weeks old, about 1 month old, about 2 month old, about 3 months old, about half year old, about 1 year old, about 2 years old plant, about 3 years old, about 5 years old, or more. The plant from which the explant is obtained can be grown in any suitable conditions, including but not limited to growing in a growth chamber, growing in a greenhouse, growing in a field, or growing in a tissue culture container (petri dish, margenta box, etc.). In some embodiments, the explant is tissue culture obtained from shoot clumps maintained as stock on growth media. In some embodiments, the explant is a nodal section having one or more axillary bud, which can be dormant or active. In some other embodiments, the explant is a seed or a part thereof.

Availability of virus-free starting material is desirable for a potato production program. Thus, in some embodiments, the virus-free micropropagated potato plants begin from an explant that is subjected to one or more antiviral treatments, such as a chemical antiviral, thermotherapy, and/or meristem-tip culture. Meristem culture is one procedure used to produce a virus-free plant. In this method, apical or axillary growing tips (0.1-0.3 mm) are dissected and allowed to grow into plantlets on special culture medium under controlled conditions. The meristem culture for virus elimination is based on the principle that many viruses are unable to infect the apical/axillary meristem of a growing plant and that a virus-free plant can be produced if a small (e.g. 0.1-0.3 mm) piece of meristemic tissue is propagated. Excision of very small meristems typically requires a high degree of expertise and the development of plants from these small meristems (mericlones) can be lengthy (i.e. 4 to 8 months). To increase the percentage of virus freedom in regenerated mericlones, meristem culture can be combined with other antiviral treatments, such as thermotherapy (high temperature treatment) or chemotherapy (treatment with antiviral compounds) to increase the production of virus-free plants.

Thus, in some embodiments, the method comprises using meristem culture, thermotherapy, chemotherapy, or any combination thereof to produce a virus-free potato plant. In one embodiment, meristem culture, thermotherapy, and chemotherapy, are used to produce a virus-free potato plant. In some embodiments, the use of an antiviral can increase the success in producing a virus-free potato plant by at least two or three times, for example from 15-25% to over 50%. In one embodiment, chemotherapy comprises using an antiviral in a medium to culture the explant. In another embodiment, thermotherapy comprises incubating an explant under a 16 h light photoperiod at 30-40 μmol/m²/s light intensity at 37° C. In some embodiments, the thermotherapy is for one week.

Accordingly, in one aspect of the present invention, a method for producing a virus-free potato plant comprises incubating a potato explant with medium, optionally comprising an antiviral; optionally, subjecting the explant of the plant culture to thermotherapy, wherein the explant grows into a plantlet; excising an apical meristem from the plantlet; and placing the apical meristem into a regeneration media; wherein a virus-free plantlet is produced. The excision is of a very small piece of meristem, and can be performed as depicted in FIG. 33.

In some embodiments, the regeneration media is selected from FIG. 32. In other embodiments, the regeneration media comprises an antiviral, such as Ribavirain (also known as Virazole). The method for producing a virus-free potato plant can also comprise culturing or subculturing, using conditions such as disclosed in PCT Publication No. WO2013016198, which is incorporated by reference in its entirety. For example, culturing or subculturing can be of the explant, apical meristem, the plantlet, or any combination thereof. The culturing or subculturing can be performed every one, two to three weeks. In one embodiment, culturing comprises incubating the explant under a 16 h light photoperiod at 80-100 μmol/m²/x light intensity at 24° C. In some embodiments, the method for producing a virus-free plant uses one or more different regeneration media, such as depicted in FIG. 32.

The plantlet produced by a method disclosed herein can be subcultured or tested for viruses. Any method known for testing for the presence of a virus can be used, such as by enzyme-linked immunosorbent assay (ELISA). The plantlet can be multiplied and subcultured, and used for further propagation. The pssent invention is applicable to a whole range of agricultural crops where a protocol for isolation and culture of meristematic cells or meristematic zones in vitro are available. Plantlets could be further induced and regenerated from the above cultures using either organogenesis or somatic embryogenesis.

In some embodiments, the methods further comprise (c) pretreating the potato plants obtained from step (b) or any other sources to produce pretreated potato plants. This step is also called pre-tuberization stage. In some embodiments, this step was performed in a bioreactor. In some embodiments, the bioreactor is a temporary immersion bioreactor. In some embodiments, liquid or semi-liquid medium is used, e.g. the pre-tuberization media as described above.

In some embodiments, the potato plants obtained from step (b) or any other resources are cultured in a liquid medium, wherein the each plant has about 3-10 axillary buds, e.g., about 4 to 7 axillary buds. In some embodiments, the medium comprises MS salts, sucrose, at least one cytokinin, and at least one auxin, e.g., BOO18 as described herein. In some embodiments, the cytokinin is 2ip or analog thereof. In some embodiments, the auxin is IAA or analog thereof. Alternatively, the medium comprises MS salts, sucrose, and at least one growth retardant, e.g., the BOO23, BOO19, BOO20, BOO24 media described herein, or combination thereof. In some embodiment, at least one retardant is ancymidol or analog thereof. Still in some embodiments, the pre-tuberization media comprise one or more cytokinin, one or more auxin, and one or more growth retardant. In some embodiments, the concentration of 2ip is about 1 to 10 mg/L, for example, about 4-5 mg/L. In some embodiments, the concentration of IAA is about 0.1 to 10 mg/L, for example, about 1 mg/L. In some embodiments, the concentration of ancymidol is about 0.1 to 10 mg/L, for example, about 0.5 mg/L, about 1 mg/L, about 2 mg/L, or about 5 mg/L. In any case, the sucrose concentration is about 20 g/L to about 40 g/L, for example, about 30 g/L. In some embodiments, a medium comprising MS salts, sucrose, at least one cytokinin, and at least one auxin, and a medium comprising MS salts, sucrose, and at least one growth retardant are used in combination, or sequentially during the pretreatment stage in any order, in one or more culture cycles.

Any suitable growth condition can be used. In some embodiments, the plants are grown under about 20-28° C. (e.g., about 24° C.), day-night cycle (e.g., 16/8 hour day/night), with a photon flux density of about 10-100 μmol/m²/s (e.g., about 30-80 μmol/m²/s). In some embodiments, the duration of the pretreatment step is about 1-4 weeks, e.g., about 1 to 2 weeks or about 2-3 weeks.

In some embodiments, the methods further comprise (d) initiating microtubers from the pretreated potato plants obtained from step (c) or any other sources. This step is also called tuberization stage.

One or more ways to initiate tuberization of potato in vitro can be utilized in step (d). In some embodiments, the methods of present application comprise initiating tuberization in vitro by supplying relatively high concentration of sucrose. For example, the sucrose concentration in the tuberization induction media is about 5% w/v, about 6% w/v, about 7% w/v, about 8% w/v, about 9% w/v, about 10% w/v, or more.

In some embodiments, the methods of present application comprise triggering tuberization in vitro by shifting the tissue culture from long-day light conditions to short-day light conditions. For example, the photoperiod condition is changed from long-day conditions, such as about 23/1 hours (light/dark), about 22/2 hours (light/dark), about 21/3 hours (light/dark), about 20/4 hours (light/dark), about 19/5 hours (light/dark), about 18/6 hours (light/dark), about 17/7 hours (light/dark), about 16/8 hours (light/dark), about 15/9 hours (light/dark), about 14/8 hours (light/dark), or about 13/11 hours (light/dark) to short-day conditions, such as about 11/13 hours (light/dark), about 10/14 hours (light/dark), about 9/15 hours (light/dark), about 8/16 hours (light/dark), about 7/17 hours (light/dark), about 6/18 hours (light/dark), about 5/19 hours (light/dark), about 4/20 hours (light/dark), about 3/21 hours (light/dark), about 2/22 hours (light/dark), or about 1/23 hours (light/dark).

In some embodiments, the methods of present application comprise triggering tuberization in vitro by using a total darkness condition.

In some embodiments, the methods of present application comprise triggering tuberization in vitro by using cool temperature conditions. For example, the temperature during the day time and/or the night time is about 25°±1° C., 24°±1° C., 23°±1° C., 22°±1° C., 21°±1° C., 20°±1° C., 19°±1° C., 18°±1° C., 17°±1° C., 16°±1° C., 15°±1° C., 14°±1° C., or lower. In some embodiments, the day time temperature is about 20°±2° C. and night time temperature is about 18°±2° C. In some embodiments, the temperature during the night time is lower than the temperature during the day time, for example, about 0.5° C., about 1° C., about 2° C., about 3° C., about 4° C., about 5° C., or more.

In some embodiments, the methods of present application comprise triggering tuberization in vitro by using one or more phytohormones or growth regulators, such as cytokins or growth retardants. In some embodiments, the cytokin is selected from the group consisting of thidiazuron (TDZ), N⁶-benzylaminopurine (BAP, a.k.a. BA), meta-topolin (mT), zeatin, zeatin riboside, dihydrozeatin, kinetin, isopentenyladenine (ip, e.g., 2ip), adenine hemisulfate, dimethylallyladenine, N-(2-chloro-4-pyridyl)-N′-phenylurea) (4-CPPU), analog thereof, and combination thereof. In some embodiments, the growth retardants is selected from the group consisting of alar, ancymidol, chlorocholine chloride (CCC), coumarin, fluridone, tetcyclacis (TET), ancymidol, analog thereof, and combination thereof. In some embodiments, the growth retardant is a gibberellic acid (GA3) antagonist, such as ancymidol and its functional derivatives.

In some embodiments, the methods of present application comprise triggering tuberization in vitro by increased nitrate:ammonium ratio and/or increased nitrogen:carbon ratio.

In some embodiments, more than one way of triggering potato tuberization described above are simultaneously and/or sequentially used. More methods for triggering potato tuberization can be found in Donnelly et al. 2003, Seabrook et al. 1993, Gopal et al. 1998, and Gopal et al. 1997, Garner and Blake et al. 1989, Bizari et al. 1995, Nasiruddin and Blake 1994, each of which is incorporated by reference in its entirety for all purposes.

In some embodiments, step (d) was performed in a bioreactor. In some embodiments, the bioreactor is a temporary immersion bioreactor. In some embodiments, liquid or semi-liquid medium is used, e.g. the pre-tuberization media as described above. In some embodiments, the potato plants obtained from step (c) or any other resources are cultured in a liquid or semi-liquid medium. In some embodiments, the liquid medium or semi-liquid medium comprises one or more auxin, but does not comprise any cytokinin, e.g., BOO21 media described herein. Alternatively, instead of auxin, the tuberization medium comprises one or more plant retardant, such as ancymidol or analog thereof, e.g., BOO25, BOO26, BOO27, BOO22 media described herein, or combination thereof. In some embodiments, the tuberization medium comprises one or more auxin and one or more growth retardant. In some embodiments, the auxin is NAA. In some embodiments, the NAA concentration is about 0.01 to about 0.05 mg/L, for example, about 0.01 mg/L, or about 0.02 mg/L. In some embodiments, the concentration of ancymidol is about 0.1 to 10 mg/L, for example, about 0.5 mg/L, about 1 mg/L, or about 5 mg/L. In any case, the sucrose concentration in the tuberization medium is higher compared to the sucrose concentration in the pre-tuberization medium used in step (c). For examples, the sucrose concentration in the tuberization media is about 50 g/L to about 100 g/L or more, for example, about 60 g/L, about 70 g/L, or about 80 g/L. In some embodiments, the sucrose concentration is about 80 g/L. Any suitable growth condition can be used.

In some embodiments, the plants are grown under a temperature that is lower than the temperature used in step (c). For example, when the temperature used in step (c) is about 24° C., the temperature used in step (d) can be about 15-24° C. In some embodiments, the plants are cultured with continuous darkness. In some embodiments, this step lasts for about 5-6 weeks, or any period of time that is suitable for a specific potato species or a specific goal (e.g., with predetermined microtubers production number and/or size).

The methods can further comprise (e) harvesting the microtubers produced in step (d). The microtubers propagated by methods described herein can be either stored under suitable conditions for future use, or could be directly transplanted to soil without any acclimation. Optionally, this step includes washing, drying, weighing, counting and/or storing the microtubers. In some embodiments, the microtubers are stored at a temperature above 0° C. but below about 10° C., e.g., at about 4° C.

The present invention also provides small microtubers (e.g., from about 2 mm to about 3 mm in length) produced using the micropropagation media, systems and methods of the present invention, wherein such microtubers produce as much or more total yield of tubers from potato plants produced from such microtubers as the total yield of tubers from potato plants produced from larger microtubers (e.g., from about 5 mm to about 7 mm in length).

The present invention also provides methods of producing potato tubers comprising obtaining pathogen-free microtubers via in vitro propagation, wherein the microtubers are produced by using any one of the methods set forth herein; planting the microtubers; and obtaining potato tubers. In some embodiments of the present invention, such microtubers are about 2 mm-about 3 mm long. In other embodiments, the microtubers are about 5 mm-about 7 mm long.

The present invention also provides methods of increasing potato tuber production, comprising obtaining pathogen-free microtubers via in vitro propagation; planting the microtubers; and obtaining potato tubers, wherein the microtubers are less than about 5 mm long, and wherein the weight of potato tuber produced by using the microtubers less than about 5 mm long is higher than the weight of potato tuber produced by using microtubers more than 5 mm long. In some embodiments of the present invention, the microtubers are about 2 mm-about 3 mm long.

The methods described herein can be further modified and optimized, depending on the purposes and goals. For example, factors affecting potato tissue culture disclosed in Nistor et al., 2010, Rosu et al., 2004, Badoni et al., 2009, Wang et al., 1982, Abbott et al, 1986, Ewing et al., 1992, Khuri et al., 1995, Perl et al., 1991, Leclerc et al., 1994, Levyd et al., 1993, Ahmad et al., 1993, and others can be considered. More information can be found in Bajaj, 2009 (Potato Volume 3 of Biotechnology in agriculture and forestry, Springer-Verlag, 1987, ISBN 3540179666, 9783540179665); Haverkort and Anisimov, 2007 (Potato production and innovative technologies, Wageningen Academic Pub, 2007, ISBN 9086860427, 9789086860425); Raj an and Markose 2007 (Propagation of Horticultural Crops: Vol. 06. Horticulture Science Series, New India Publishing, 2007, ISBN 8189422480, 9788189422486); Leclerc 1993 (The production and utilization of potato microtubers, McGill University, 1993), Krasteva and Panayotov, 2009 (Proceedings of the fourth Balkan Symposium on Vegetables and Potatoes, ISHS, 2009, ISBN 9066055529, 9789066055520), each of which is incorporated herein by reference in its entirety for all purposes.

### Bamboo Propagation/Culture

The present invention also provides methods for in vitro propagation of bamboo plants. In some embodiments, the methods comprise propagating bamboo through somatic embryogenesis.

In some embodiments, the methods start with (a) initiating embryos from a bamboo explant. In some embodiments, this step comprises culturing vegetative explants obtained from a bamboo plant on the first type of media described herein.

One or more types of explants obtained from a bamboo plant can be used. As used herein, an “explant” (a.k.a. a “mother plant”) is the source of cells to be developed during the tissue culturing process. For example, the explant can be any segment or collection of cells from apical meristems, terminal buds, axillary buds, adventitious buds, accessory buds, pseudo-terminal buds, cambium, lateral meristem, lateral bud, vegetative buds, reproductive buds, mixed buds, shoot segments, shoot apices, stem segments, immature nodal sections from stems, lateral shoots, seedlings, seeds, shoots starting to rise from the ground, immature flower buds, inflorescences, crown segments, leaf segments, or any part thereof. In some embodiments, the explants can be node segments, immature leaves, immature embryos, or mature seeds. In some embodiments, the explants can be tissue comprising meristematic cells, such as the cells located in axillary or lateral buds of a bamboo plant.

In some embodiments, the bamboo species is selected from Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, or Guadua Angustifolia, or the ones described in WO/2011/100762, which is incorporated herein by reference in its entirety.

In one embodiment, the explants are taken from a juvenile or a mature bamboo plant. In some embodiments, the explants are taken from a plant of about 1 week old, about 2 weeks old, about 3 weeks old, about 1 month old, about 2 month old, about 3 months old, about half year old, about 1 year old, about 2 years old plant, about 3 years old, about 5 years old, or more. The plant from which the explant is obtained can be grown in any suitable conditions, including but not limited to growing in a growth chamber, growing in a greenhouse, growing in a field, or growing in a tissue culture container (petri dish, margenta box, etc.). In some embodiments, the explant is tissue culture obtained from shoot clumps maintained as stock on growth media. In some embodiments, the explant is a nodal section having one or more axillary bud, which can be dormant or active. In some other embodiments, the explant is a seed or a part thereof. In some embodiments, the explants are free or substantially free of pathogens. In some embodiments, before culturing the explants on the media, the explants are sterilized. Non-limiting examples of sterilizing bamboo explants are described in WO/2011/100762, which is incorporated herein by reference in its entirety.

In some embodiments, the explants are cultured on the first type of media until one or more embryos initiate. In some embodiments, the explants are cultured on the first type of media for about 1-24 hours, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 2 months, about 2.5 months, about 3 months, about 3.5 months, about 4 months, about 4.5 months, about 5 months, about 5.5 months, about 6 months, or more.

In some embodiments, the explants are transferred from an old medium to a fresh medium of the same type after a pre-determined period of time, or to separate from contaminated tissue culture when needed.

In some embodiments, the explants are placed under a suitable temperature and a suitable light level. In some embodiments, the explants are placed under a temperature of from 65° F.-70° F. or more and a full spectrum light level of 36-54 μmole/m²/s² or more.

The embryos generated by step described above can be used for multiple purposes, or subjected to any suitable methods to produce mature embryos. In some embodiments, once an explant exhibits initiated embryos, the embryos obtained from the initiation stage can be collected and cultured in the second media as described herein to produce an embryogenic suspension. In some embodiments, the second media are liquid media.

In some embodiments, the embryos are cultured on the second media for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, about 21 hours, about 22 hours, about 23 hours, about 24 hours, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 2 months, about 2.5 months, about 3 months, about 3.5 months, about 4 months, about 4.5 months, about 5 months, about 5.5 months, about 6 months, or more. In some embodiments, the step is continued until a sufficient amount of embryogenic-like structures are obtained. In some embodiments, pulsing methods described herein are used.

In some embodiments, the embryogenic-like structures are transferred from an old medium to a fresh medium of the same type after a pre-determined period of time or to separate them from contaminated tissue culture when needed.

In some embodiments, the embryogenic-like structures are cultured in a bioreactor, such as a temporary immersion bioreactor (e.g. an ebb and flow bioreactor). Bioreactors offer a promising way of scaling-up micropropagation processes, making it possible to work in large containers with a high degree of control over the culture parameters (e.g., pH, aeration, oxygen, carbon dioxide, hormones, nutrients, etc.). Bioreactors are also compatible with the automation of micropropagation procedures, utilizing artificial intelligence, which reduces production costs. Non-limiting examples of bioreactors include those described in U.S. Pat. Nos. 3,578,431; 4,320,594; 4,669,217; 4,908,315; 4,934,096; 5,049,505; 5,088,231; 5,104,527; 5,119,588; 5,139,956; 5,171,683; 5,184,420; 5,186,895; 5,212,906; 5,225,342; 5,558,984; 5,597,731; and 6,753,178. More examples of plant micropropagation systems can be found in Etienne et al. (Bioreactors in coffee micropropagation, Braz. J. Plant Physiol., 18(1):45-54, 2006); Ziv (Bioreactor Technology for Plant Micropropagation Horticultural Reviews, Volume 24, Edited by Jules Janick ISBN 0-471-33374-3); and Paek et al. (Application of bioreactors for large-scale micropropagation systems of plants, In vitro Cell. Dev. Biol.-Plant 37:149-157, March-April 2001).

In some embodiments, the bioreactor is placed under a suitable temperature and a suitable light level. In some embodiments, the explants are placed under a temperature of from 65° F.-70° F. or more and a full spectrum light level of 36-54 μmole/m²/s² or more.

In some embodiments, during this step, the density of the embryogenic-like structures can be estimated or measured in order to determine if more cycles of culturing are needed.

The embryos suspension generated by the step described above can be used for multiple purposes, or subjected to any suitable methods to produce mature embryos. In some embodiments, once a desired embryogenic suspension is obtained, the embryos in the suspension can be transferred onto the third media as described herein. The media can be either liquid or solid. In some embodiments, the media are solid.

The embryos are further multiplied and/or induced into a maturation stage during this step. Abscisic acid in the media is helpful to induce embryo maturation. In some embodiments, charcoal (e.g., active charcoal) can be added, which surprisingly can greatly enhance embryo production and maturation. In some embodiments, the charcoal is about 0.01% to 10% of the media by weight.

In some embodiments, the embryos are cultured on the third media for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, about 21 hours, about 22 hours, about 23 hours, about 24 hours, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 2 months, about 2.5 months, about 3 months, about 3.5 months, about 4 months, about 4.5 months, about 5 months, about 5.5 months, about 6 months, or more. In some embodiments, the step is continued until enough mature embryos are obtained. In some embodiments, mature embryos can be isolated from the media while the culturing is continued to obtain more mature embryos. Embroys will establish on the third media.

In some embodiments, the embryos are transferred from an old medium to a fresh medium of the same type after a pre-determined period of time or to separate them from contaminated tissue culture when needed.

The mature embryos generated by the methods described above can be used for multiple purposes, or subjected to any suitable methods to germinate. In some embodiments, the mature embryos are germinated. In some embodiments, the mature somatic embryos are germinated on the fourth type of media of the present invention. In some embodiments, the media are solid media.

In some embodiments, the mature somatic embryos are germinated on the fourth media for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, about 21 hours, about 22 hours, about 23 hours, about 24 hours, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 2 months, about 2.5 months, about 3 months, about 3.5 months, about 4 months, about 4.5 months, about 5 months, about 5.5 months, about 6 months, or more. In some embodiments, the step is continued until germination is accomplished.

The mature embryos or the germinated bamboo plants can be used for multiple purposes. In some embodiments, the mature somatic embryos can be treated and stored under suitable conditions before germination. In some embodiments, the mature embryos or the germinated bamboo plants can be used as a stock to produce more bamboo plants through tissue culture, by using methods known to one skilled in the art, such as those described in WO/2011/100762, which is incorporated herein by reference in its entirety. In some embodiments, the germinated bamboo plants can be transferred to an in vitro or an in vivo condition to produce mature bamboo plants.

The present invention is further illustrated by the following examples that should not be construed as limiting. The contents of all references, patents, and published patent applications cited throughout this application, as well as the Figures, are incorporated herein by reference in their entirety for all purposes.

### Kits

The present invention also provides kits for plant propagation. In some embodiments, the kits include one or more media of the present invention. In some embodiments, the kits include one or more explants of a plant species.

For example, the kits can include one or more bud induction medium (BOO1), shoot elongation/maintenance media (BOO2), BOO36-i media, BOO36-ii media, BOO36-iii media, BOO36-iv media, BOO36-v media, spiked BOO36-i media, spiked BOO36-ii media, spiked BOO36-iii media, spiked BOO36-iv media, spiked BOO36-v media, reduced BOO36-i media (reduced media are described below), reduced BOO36-ii media, reduced BOO36-iii media, reduced BOO36-iv media, reduced BOO36-v media, BOO40-i media, BOO40-ii media, BOO40-iii media, BOO40-iv media, BOO40-v media, spiked BOO40-i media, spiked BOO40-ii media, spiked BOO40-iii media, spiked BOO40-iv media, spiked BOO40-v media, reduced BOO40-i media, reduced BOO40-ii media, reduced BOO40-iii media, reduced BOO40-iv media, reduced BOO40-v media, BOO37-i media, BOO37-ii media, BOO37-iii media, BOO37-iv media, BOO37-v media, spiked BOO37-i media, spiked BOO37-ii media, spiked BOO37-iii media, spiked BOO37-iv media, spiked BOO37-v media, reduced BOO37-i media, reduced BOO37-ii media, reduced BOO37-iii media, reduced BOO37-iv media, reduced BOO37-v media, BOO31-i media, BOO31-ii media, BOO31-iii media, BOO31-iv media, BOO31-v media, spiked BOO31-i media, spiked BOO31-ii media, spiked BOO31-iii media, spiked BOO31-iv media, spiked BOO31-v media, reduced BOO31-i media, reduced BOO31-ii media, reduced BOO31-iii media, reduced BOO31-iv media, reduced BOO31-v media, BOO38-i media, BOO38-ii media, BOO38-iii media, BOO38-iv media, BOO38-v media, spiked BOO38-i media, spiked BOO38-ii media, spiked BOO38-iii media, spiked BOO38-iv media, spiked BOO38-v media, reduced BOO38-i media, reduced BOO38-ii media, reduced BOO38-iii media, reduced BOO38-iv media, reduced BOO38-v media, BOO28-i media, BOO28-ii media, BOO28-iii media, BOO28-iv media, BOO28-v media, spiked BOO28-i media, spiked BOO28-ii media, spiked BOO28-iii media, spiked BOO28-iv media, spiked BOO28-v media, reduced BOO28-i media, reduced BOO28-ii media, reduced BOO28-iii media, reduced BOO28-iv media, reduced BOO28-v media, BOO29-i media, BOO29-ii media, BOO29-iii media, BOO29-iv media, BOO29-v media, spiked BOO29-i media, spiked BOO29-ii media, spiked BOO29-iii media, spiked BOO29-iv media, spiked BOO29-v media, reduced BOO29-i media, reduced BOO29-ii media, reduced BOO29-iii media, reduced BOO29-iv media, reduced BOO29-v media, BOO30-i media, BOO30-ii media, BOO30-iii media, BOO30-iv media, BOO30-v media, spiked BOO30-i media, spiked BOO30-ii media, spiked BOO30-iii media, spiked BOO30-iv media, spiked BOO30-v media, reduced BOO30-i media, reduced BOO30-ii media, reduced BOO30-iii media, reduced BOO30-iv media, reduced BOO30-v media, BOO39-i media, BOO39-ii media, BOO39-iii media, BOO39-iv media, BOO39-v media, spiked BOO39-i media, spiked BOO39-ii media, spiked BOO39-iii media, spiked BOO39-iv media, spiked BOO39-v media, reduced BOO39-i media, reduced BOO39-ii media, reduced BOO39-iii media, reduced BOO39-iv media, reduced BOO39-v media, BOO41-i media, BOO41-ii media, BOO41-iii media, BOO41-iv media, BOO41-v media, spiked BOO41-i media, spiked BOO41-ii media, spiked BOO41-iii media, spiked BOO41-iv media, spiked BOO41-v media, reduced BOO41-i media, reduced BOO41-ii media, reduced BOO41-iii media, reduced BOO41-iv media, reduced BOO41-v media, BOO42-i media, BOO42-ii media, BOO42-iii media, BOO42-iv media, BOO42-v media, spiked BOO42-i media, spiked BOO42-ii media, spiked BOO42-iii media, spiked BOO42-iv media, spiked BOO42-v media, reduced BOO42-i media, reduced BOO42-ii media, reduced BOO42-iii media, reduced BOO42-iv media, reduced BOO42-v media, BOO43-i media, BOO43-ii media, BOO43-iii media, BOO43-iv media, BOO43-v media, spiked BOO43-i media, spiked BOO43-ii media, spiked BOO43-iii media, spiked BOO43-iv media, spiked BOO43-v media, reduced BOO43-i media, reduced BOO43-ii media, reduced BOO43-iii media, reduced BOO43-iv media, reduced BOO43-v media, BOO44-i media, BOO44-ii media, BOO44-iii media, BOO44-iv media, BOO44-v media, spiked BOO44-i media, spiked BOO44-ii media, spiked BOO44-iii media, spiked BOO44-iv media, spiked BOO44-v media, reduced BOO44-i media, reduced BOO44-ii media, reduced BOO44-iii media, reduced BOO44-iv media, reduced BOO44-v media, BOO35-i media, BOO35-ii media, BOO35-iii media, BOO35-iv media, BOO35-v media, spiked BOO35-i media, spiked BOO35-ii media, spiked BOO35-iii media, spiked BOO35-iv media, spiked BOO35-v media, reduced BOO35-i media, reduced BOO35-ii media, reduced BOO35-iii media, reduced BOO35-iv media, reduced BOO35-v media, BOO38 CPPU-i media, BOO38 CPPU-ii media, BOO38 CPPU-iii media, BOO38 CPPU-iv media, BOO38 CPPU-v media, spiked BOO38 CPPU-i media, spiked BOO38 CPPU-ii media, spiked BOO38 CPPU-iii media, spiked BOO38 CPPU-iv media, spiked BOO38 CPPU-v media, reduced BOO38 CPPU-i media, reduced BOO38 CPPU-ii media, reduced BOO38 CPPU-iii media, reduced BOO38 CPPU-iv media, reduced BOO38 CPPU-v media, BOO38 DPU-i media, BOO38 DPU-ii media, BOO38 DPU-iii media, BOO38 DPU-iv media, BOO38 DPU-v media, spiked BOO38 DPU-i media, spiked BOO38 DPU-ii media, spiked BOO38 DPU-iii media, spiked BOO38 DPU-iv media, spiked BOO38 DPU-v media, reduced BOO38 DPU-i media, reduced BOO38 DPU-ii media, reduced BOO38 DPU-iii media, reduced BOO38 DPU-iv media, reduced BOO38 DPU-v media, BOO46-i media, BOO46-ii media, BOO46-iii media, BOO46-iv media, BOO46-v media, spiked BOO46-i media, spiked BOO46-ii media, spiked BOO46-iii media, spiked BOO46-iv media, spiked BOO46-v media, reduced BOO46-i media, reduced BOO46-ii media, reduced BOO46-iii media, reduced BOO46-iv media, reduced BOO46-v media, BOO45-i media, BOO45-ii media, BOO45-iii media, BOO45-iv media, BOO45-v media, spiked BOO45-i media, spiked BOO45-ii media, spiked BOO45-iii media, spiked BOO45-iv media, spiked BOO45-v media, reduced BOO45-i media, reduced BOO45-ii media, reduced BOO45-iii media, reduced BOO45-iv media, reduced BOO45-v media, BOO47-i media, BOO47-ii media, BOO47-iii media, BOO47-iv media, BOO47-v media, spiked BOO47-i media, spiked BOO47-ii media, spiked BOO47-iii media, spiked BOO47-iv media, spiked BOO47-v media, reduced BOO47-i media, reduced BOO47-ii media, reduced BOO47-iii media, reduced BOO47-iv media, and/or reduced BOO47-v media. In another embodiment, the kits can comprise one or more containers for the tissue culturing process including without limitation, tubes, jars, boxes, jugs, cups, sterile bag technology, bioreactors, temporary immersion vessels, etc. In another embodiment the kits can comprise instructions for the tissue culturing of bamboo. In another embodiment, the kits comprise combinations of the foregoing. Components of various kits can be found in the same or different containers. Additionally, when a kit is supplied, the different components of the media can be packaged in separate containers and admixed immediately before use. Such packaging of the components separately may permit long-term storage without losing the active components' functions. Alternatively, media can be provided pre-mixed.

The present invention also provides kits for bamboo plant propagation. In some embodiments, the kits include one or more media of the present invention. In some embodiments, the kits include one or more explants of a bamboo species.

In addition, also provided are kits for growing bamboo plant from somatic embryos. In some embodiments, the kits comprise one or more somatic embryos of the same bamboo species or of different bamboo species. Optionally, the kits comprise one or more media for germinating bamboo embryos. In some embodiments, the media for germinating bamboo embryos are selected from the fourth type of media as described herein.

### Propagation/Culture of Herbs and Phyto-Pharmaceutical Plants

The present invention also provides methods for in vitro propagation of herbs and phyto-pharmaceutical plants. The methods and/or media described herein may be used to culture and micropropagate any one of the following plants/plant part (Romanized Chinese nomenclature): Ai Ye, Ai Ye Tan, Ba Ji Tian, Ba Ji Tian, Bai Bian Dou (Bian Dou), Bai Bu, Bai Fan Shu Gen, Bai Fu Zi, Bai Guo (Yin Guo), Bai He, Bai Hua She She Cao, Bai Jiang Cao, Bai Mao Gen, Bai Qian, Bai Shao, Bai Tou Weng, Bai Wei, Bai Xian Pi, Bai Zhi, Bai Zhu, Bai Zi Ren, Ban Lan Gen, Ban Xia (Jiang), Ban Zhi Lian, Bei Sha Shen, Bian Xu, Bo He, Bu Gu Zhi, Cang Er Zi (Chao), Cang Zhu, Ce Bai Ye, Ce Bai Ye Tan, Cha Chi Huang, Cha Ye (Lu Cha), Chai Hu, Chang Chun Hua (Ri Ri Chun), Che Qian Cao, Che Qian Zi, Chen Pi, Chi Fu Ling, Chi Shao, Chong Wei Zi, Chuan Lian Zi, Chuan Niu Xi, Chuan Po Shi, Chuan Xin Lian, Chuan Xiong, Chun Pi (Chun Gen Pi), Ci, Shi, Ci Wu Jia (Wu Jia Shen), Da Ding, Huang, Da Fu Pi, Da Huang, Da Huang (Zhi), Da Qing Ye, Da Xiao Ji, Da Zao (Hong), Dan Dou Chi, Dan Nan Xing, Dan Shen, Dan Zhu Ye, Dang Gui, Dang Gui Wei, Dang Shen, Dao Di Wu Gong, Deng Xin Cao, Di Fu Zi, Di Gu Pi, Di Huang (Sheng), Di Yu, Di Yu Tan, Ding Shu Xiu, Ding Xiang, Dong Chong Xia Cao, Dong Gua Zi, Dou Kou (Bai Dou Kou), Du Huo, Du Zhong, E Bu Shi Cao, E Jiao, E Zhu, Er Cha, Fan Xie Ye, Fang Feng, Fang Ji (Han Fang Ji), Fen Bi Xie (Bi Xie), Feng Wei Cao, Fo Shou, Fu Hai Shi, Fu Ling, Fu Ling Pi, Fu Pen Zi, Fu Rong, Fe Shen Fu Xiao Mai, Fu Zi (Zhi), Gan Cao, Gan Jiang, Gao Ben, Gao Liang Jiang, Ge Gen, Ge Hua, Ge Qiao (Hai Ge Fen), Geng Mi, Gou Qi Zi, Gou Teng, Gu Gui Bu, Gua Lou (Gua Lou Shi), Gua Lou Pi, Gua Lou Zi, Guan Ye Jin Si Tao, Gui Zhi, Hai Jin Sha, Hai Piao Xiao, Hai Tong Pi, Hai Zao, Han Xiu Cao Gen, He Huan Hua, He Huan Pi, He Shou Wu, He Ye, He Zi, Hei Da Zao, Hei Zhi Ma, Hong Hua, Hong Jing Tian, Hou Po, Hu Huang Lian, Hu Po, Ho yao huang, Hu zhang, Hua jiao, Hua shi, Hua shi cao, Huai hua, Huang bo, huang jin gui, Huang jing, Huang lian, Huang Qi, Huang Qin, Huang shui qie, Hou man ren, huo xiang, Ji li, Ji nei jin, Ji xiang teng, Ji xue cao, Ji xue teng, Jiang huang, Jiang xiang, Jiao gu lan, Jiao zhi zi, Jie geng, Jie zi, Jin qian cao, Jin yin hua, Jin ying zi, Jing jie, Jing jie tan, Jiu cai zi, Jiu ceng ta, Ju he, Ju hong, Ju hua, Jue ming zi, Ku shen, Ku xing ren, Kun bu, Lai fu zi, Li zhi he, Lian Qiao, Lian xu, Lian zi, Lian zi xin, Liang mian zhen, Ling zhi, Liu zhi huang, Long dan, Long gu, Long yan rou, Lu gen, Lu jiao shuang, Lu lu tong, Lu rong, Lu xian cao, Luo bu ma, Luo shi teng, Ma bian cao, Ma bo, Ma chi xian, Mai dong, Mai ya, Man jing zi, Mang Xiao, Mao dong qing, Mian ma guan zhong, Mo gu xiao, Mo han lian, Mo yao, Mu dan pi, Mu gua, Mu li, Mu tong, Mu xiang, Mu zei, Nan sha shen, Niu bank Zi, Niu xi, Nu zhen, O jie, Pang da hai, Pao jiang, Pao zai cao, Pi pa ye, Po bu zi ye, Pu gong ying, Pu huang, Pu huang tan, Pu tao zi, Pu yin, Qi ye lian, Qian cao, Qian ceng ta, Qian hu, Qian nian jian, Qian shi, Qiang huo, Qin jiao, Qin pi, Qing hao, Qing pi, Qu mai, Ren dong teng, Ren shen, Rou cong rong, Rou dou kou, Rou gui, Ru xiang, San leng, San Qi, Sang bai pi, San ji sheng, Sang shen Sang ye, Sang zhi, Sha ren, Sha yuan zi, Shan dou gen, Shan yao, Shan zha, Shan zha tan, Shan zhu yu, She chuang zi, She gan, Shen jin cao, Sheng jiang, Sheng ma, Shi chiang pu, Sha di, Shi gao, Shi jian chuan, Shi jue ming, Shi liu pi, Shi wei, Shou wu teng, Shu di huang, Shu wei huang, Shui ding xiang, Shu fei ji, Si gua lou Su mu, Suan zao ren Suo yang, Tai zi shen, Tao ren, Tian dong, Tian hua fen, Tian ma, tian nan xing, Tian zhu huang, Ting li zi, Tong cao, Tou gu cao, Tu fu ling, Tu si zi, Wan dian jin, Wang bu liu xing, Wei ling xian, Wu bei zhi, Wu jia pi, Wu mei, Wu tian, Wu wei zi, Wu yao, Wu zhu yu Wu tian, Xi xian cao, Xi yang shen, Xia ku cao, Xia tian wu, Xian feng cao, Xian he cao, Xian mao, Xiang fu, Xiao hui xiang, Xiao mai, Xie bai, Xie cao, Xin yi, Xu chang qing, Xu duan, Xuan fu hua, Xuan shen, Ya she huang, Yan hu suo, Ye ju hua, Yi mu cao, Yi yi ren Yi zhi, Yi zhi xiang, Yin chen, Yin Xing ye, Yin yang huo, Yu jin, Yu li ren, Yu mi xu, Yu xing cao, Yu zhu, Yuan zhi, Zao jiao, Zao jian ci, Ze lan, Ze xie, Zhe bei mu, zhen zhu mu, Zhi cao wu, Zhi Chuan wu, Zhi gan cao, Zhi huang qi, Zhi mu, Zhi qiao, Zhi Shi, Zhi zi, Zhu ling, Zhu ru, Zi hua di ding, Zi ran tong, Zi shi ting, Zi u ye, Zi su zi, and Zi wan.

The methods and/or media described herein may be used to culture and micropropagate any one of the following plants: Artemisia argyi, brown artimisia, morinda, dolichos nut, stemona, stinking flueggea root, typhonium, ginkgo, lily, oldenlandia, thlaspi, imperata, cynanchum stauntoni, peony, pulsatilla, Cynanchum atratum, dictamnus, Angelica, Atractylodes (alba), biota seed, isatis root, pinellia, scute barbata, glehnia, polygonum aviculare, mint, psorales, xanthium fruit, atractylodes, biota, brown biota, stellaria, tea leaf, bupleurum, Madagascar periwinkle, plantago leaf, plantago seed, citrus peel, red poria, red peony, leonurus fruit, melia, cyathula, cudrania root, andrographis, ligusticum ailanthus bark, loadstone, eleuthero, euonymus, areca husk, rhubarb, isatis leaf, cirsium, red jujube, soia, Arisaema pulvis, salvia root, lophatherium, tangkuei root, tangkuei tails, codonopsis, lysimachia, juncus, kochia, lyceum root bark, rehmannia, sanguisorba, brown sanguisorba, elephantopi, Flox caryophylli, Cordyceps sinensis, benincasa, cardamom, tuhuo angelica, eucommia bark, centipede herb, zedoaria, catechu, senna leaf, siler, stephania, tokoro, pteris, citrus sarcodactylus, poria poria cortex, rubus, hibiscus root, fushen poria, levis wheat, aconite, licorice, ginger, ligusticia (kaopen), galangal, pueraria root, pueraria flower, cyclina, rice, gambir, drynaria, trichosanthes fruit, St. John's wort, cinnamon, wasabi (Wasabia japonica), lyceum fruit, lygodium spores, erythrina, ascophyllum, mimosa pudicae, albizzia flower, polygonum, lotus, chebule, black jujube, sesame seed, carthamus, rhodiola, magnolia bark, picrorrhiza, succinium, leucas, polygonum cuspidatum, zanthoxylum, orthosiphon, sophora, phellodendron bark, vanieria, polygonatum root, coptis, astragalus, scute, solanum, hemp, agastache, tribulus, paederia, gotu kola, willow, spatholobi, turmeric, dalbergia, gynostemma, brown gardenia, pratycodon, mustard, lysimachia, lonicera flower, rosa laevigata, schizonepeta, allium seed, basil, Ocimum basilicum, citrus plants, red tangerine, chrysanthemum (Chrysanthemum sp.), cassia seed, sophora flavescens, apricot, kelp, raphanus, litchi seed, forsythia, lotus stamen, lotus embryo, shiny leaf prickly ash root, Zanthoxyli nitidi, Ganoderma, solidago, gentian, longan fruit, phragmites, cornus cervi fragments, liquidambar, pyrolae, apocynum venetum, star jasmine vine, verbena, lasiosphaera, portulacae, ophiopogon, barley, vitex, mirabilitum Ilex pubescentis, aspidum, hyptix, eclipta, moutan, chaenomeles, caulis akebiae, vladimiria, equisetum, adenophora, arctium, achyranthes, ligustrum, lotus node, sterculia, physalis angulate, eriobotrya, sebastan plum cordia, dandelion, bulrush, grape seed, indian stringbush, schefflerae, rhizome rubiae, lycopodium, peucedanum, homalomena, Euryale, notopterygium, gentian macrophylla root, fraxinus Artemisia, citrus viride, dianthus, lonicera vine, ginseng, cistanche, myristica, mastic, scirpus, notoginseng, mulberry, loranthus, amomum, astragalus seed, sophora subprostrata dioscorea, crataegus, cormus, cnidium fruit, belamcanda, lycopodium, cimicifuga, acorns, kaki calyx, Chinese sage, haliotis, granatum rind, folium pyrrosiae, polygonum multifloru vine, justicia, ludwigia, milk thistle, luffa fiber, sappan wood, zizyphus, cynomorium, rehmannia, pseudostellaria, persica, asparagus, trichosanthes root, gastrodia, arisaema, bamboo, lepidium, tetrapanax, centella herba, smilax, cuscuta, ilex, vaccaria seed, clematis, gallnut, acanthopanax, mume, wutien, schizandra, lindera eyodia, siegesbeckia, American ginseng, prunella, decumbent corydalis rhizome, bidens, agrimony, curculigo, cyperus fennel, wheat, bakeri, valerian root, magnolia flower, paniculate swallow wort root, dipsacus, inula flowers, scrophularia, lippie, corydalis, chrysanthemum, leonurus, coix, alpinia fruit, veronica cinerea, capillaris, epimedium, curcuma, prunus seed, corn silk, houttuynia, polygonatum odorati, polygala, gleditsia fruit, gleditsia spine, lycopus, alisma, aconite tsao wu, astragalus, anemarrhena, aurantium fruit, aurantium immaturus, gardenia, polyporus viola, perilla, perilla seed, aster, and rhizome asteris.

In some embodiments, the list in the preceding paragraph includes common names, scientific names, plants, and plant parts. If reciting plant parts or plant products, the disclosure contemplates culturing and micropropagating the plants from which the products or parts are from.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1

### In Vitro Initiation

Fully developed pistachio plants were cut into single-node explants, shoot tips, basal (bottom) parts of plants with multiple buds, and cultured on different agar-solidified culture media under standard tissue culture conditions (16 h light photoperiod, cool white fluorescent lights at 80-100 μmol/m²/s light intensity, 24° C.).

Culture vessels (baby food jars) with ventilated lids are used in all experiments: 1-3 explants per 1 vessel, 12 vessels per each experimental medium, 40 ml of medium in each vessel. The explants are subcultured every 30 days, photographed every week, and observed daily.

The culture media differed in composition of major macronutrients (e.g., MS vs. WPM vs. DKW), individual elements (calcium, magnesium, phosphorus, zinc copper, boron, etc.), as well as in type and concentration of plant growth regulators (PGRs).

### Example 2

### In Vitro Multiplication on Solid Medium

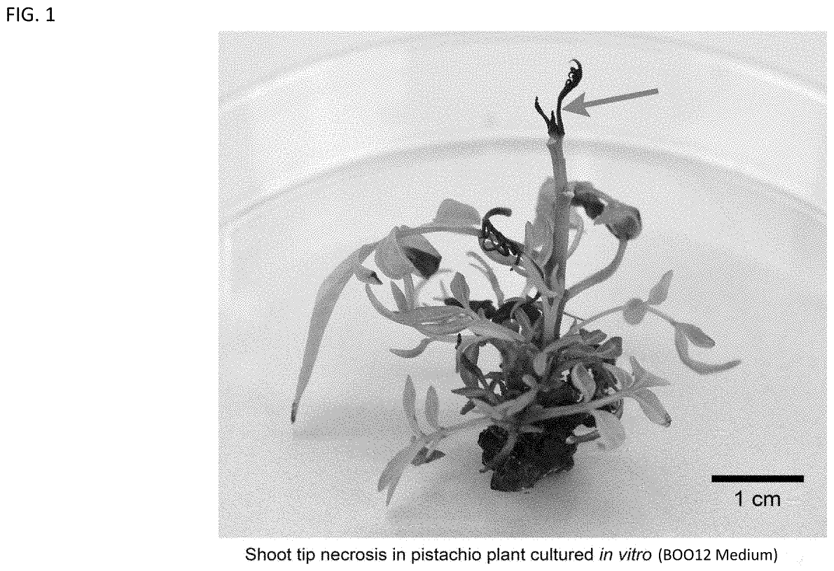

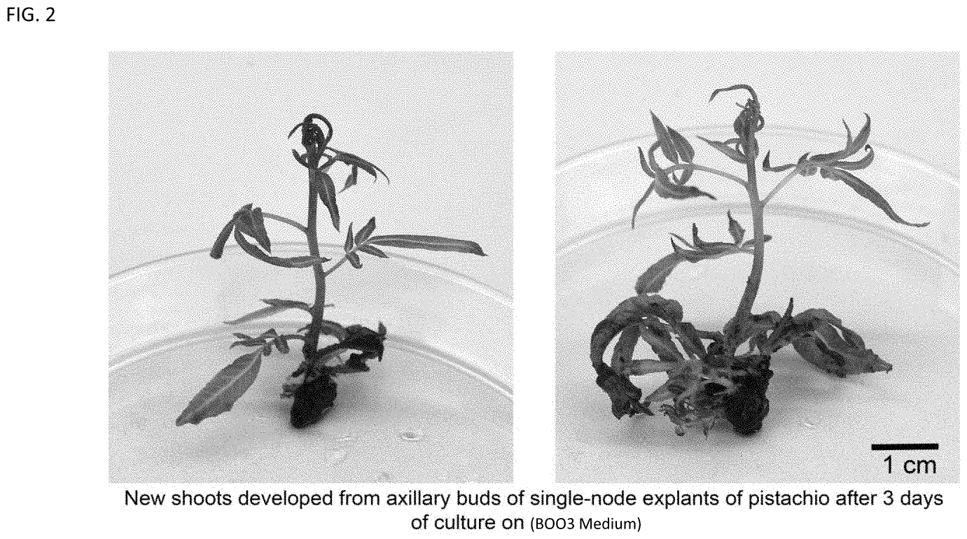

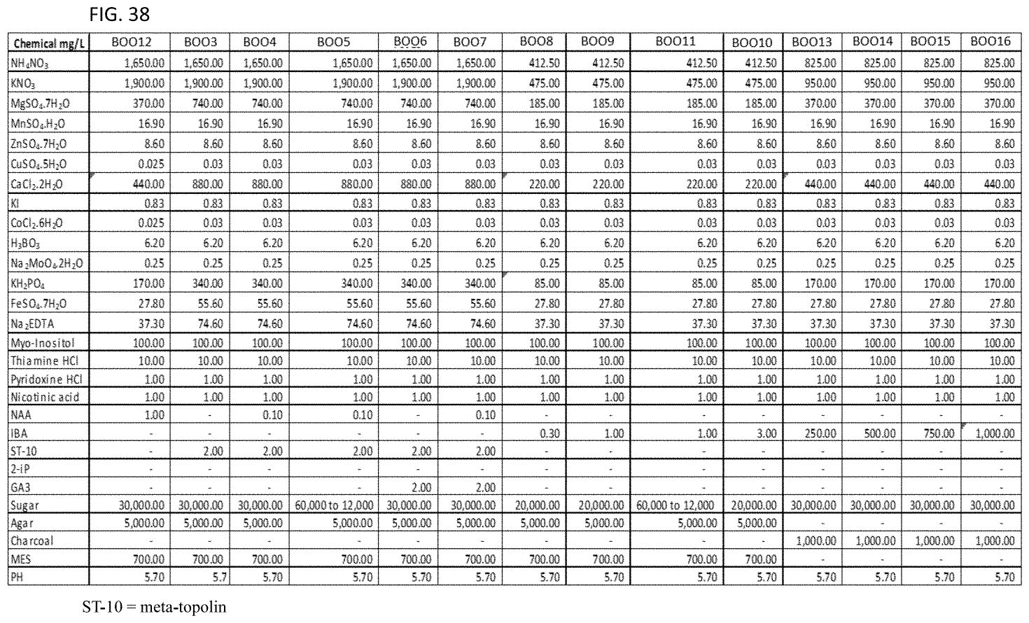

From a series of experiments examining the reactions of pistachio plants in vitro, new culture media—BOO3, BOO6, BOO4, and BOO7,—were developed that significantly increased the multiplication rate and overall quality of P. atlantica x P. intergerrina plants. Using these media in pistachio culture consistently resulted in well-developed healthy plants with minimal tissue necrosis (see FIG. 1). The compositions of these media are presented in FIG. 38, and their key advantages are based on data presented below.

Among all basal media tested, a combination of MS medium containing double concentration of meso elements (CaCl2.2H2O, MgSO4.7H2O, and KH2PO4), double iron, and Gamborg's vitamins was found to be the best for both plant growth and multiplication rate. Because the type and dosage of PGRs are usually the key factors controlling the morphogenetic response of plant cells in vitro, the main work was focused on testing various concentrations of cytokinins or cytokinin-like compounds (TDZ, 2iP, BA, meta-Topolin, ZR), alone or in combination with auxins (NAA, IAA, IBA), to induce organogenesis and high frequency multiplication in Pistacia atlantica x P. intergerrina. In the absence of PGRs (i.e., on hormone-free media), the plant growth was very poor and the culture gradually died within several weeks. In contrast, the culture media with 1-3 mg/l meta-Topolin, alone or in combination with 0.02-1 mg/l NAA or IBA, provided the highest rate of shoot organogenesis with the fastest response: each explant formed multiple meristematic buds, and the first axillary shoots appeared as early as 3 days after the explants were placed on the media. Furthermore, addition of gibberellic acid (2 mg/l of GA3) into the media induced shoot development from dormant buds of plant bases. The multiplication rate of pistachio plants cultured on BOO3, BOO6, BOO4, and BOO7 media was 2-7 times every 30 days. Without wishing to be bound by any particular theory, meta-topolin can reduce or eliminate phenolics in the tissue culture, therefore leads to increased survival rate.

### Example 3

### In Vitro Rooting on Solid Medium

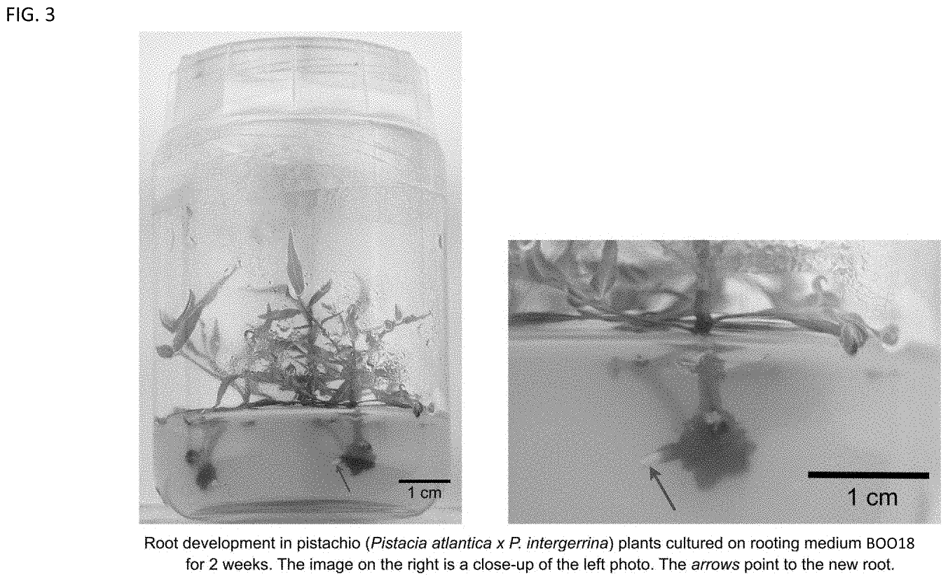

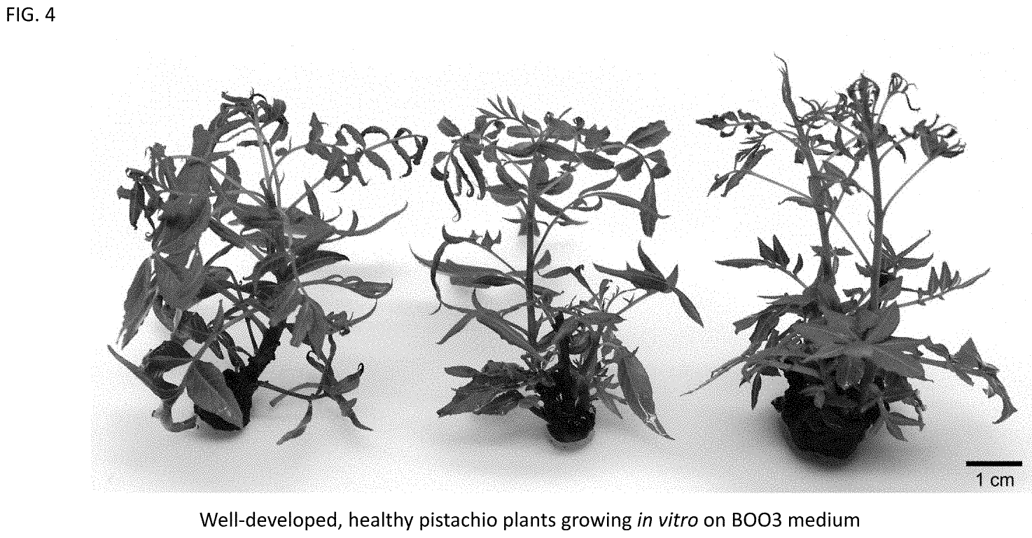

For rooting, the 3-4-week-old plants were pre-rooted on high-sucrose-containing media BOO5 or BOO11 for 1-3 weeks, then cultured on IBA-containing BOO8, BOO9, or BOO10 media (FIG. 38) for 2-4 weeks until roots developed. See, FIG. 3.

Alternatively, the pistachio plants were briefly exposed to high concentrations of IBA (BOO13, BOO14, BOO15, or BOO16 media, FIG. 38) for 1-24 hours, and then cultured on BOO8 medium for 2-4 weeks.

### Example 4

### In Vitro Multiplication in Liquid Medium

Pistachio cultures are also multiplied in liquid medium using temporary immersion bioreactor vessels. The size of bioreactor varies from 0.1 to 20 L depending on production requirements. Bioreactors are inoculated with pistachio material produced in tubes, jars, or boxes. Bioreactors is kept under standard conditions (22-24° C. and 16/8 hours day/night photoperiod). Media are refreshed every day, every two days, every three days, every four days, every five days, every six days, or every one to four weeks. After each cycle the amount of biomass increased between about 1, 2, 3, 4, 5 times or more. After several multiplication cycles the shoots were be further subjected to in vitro rooting under solid or liquid conditions.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

### Example 5

### Propagation of Stock Plants

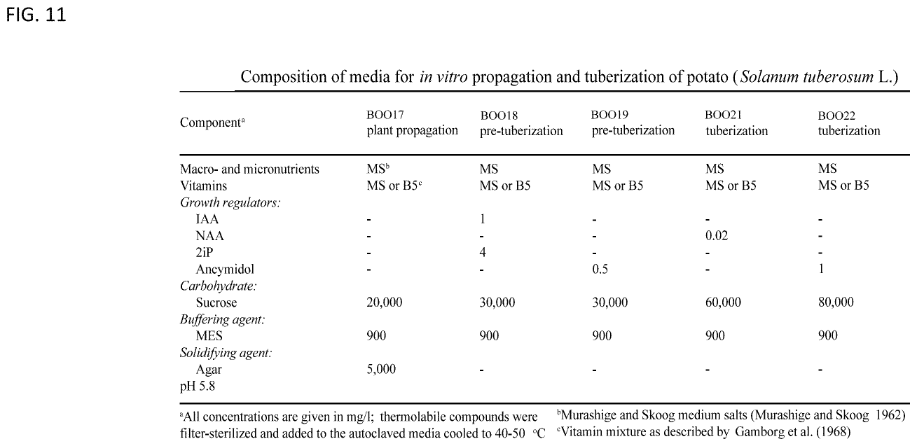

The stock plants are propagated through single-node cuttings (containing one axillary bud) or shoot-tip explants, and cultured aseptically in tubes (Sigma) on BOO17 medium without growth regulators (see FIG. 11) at 24° C. under a 16-h light period. Light is provided by cool white fluorescent tubes (Sylvania) at a photon flux density of 85-100 μmol/m²/

Alternatively, an indirect shoot organogenesis method is used to produce yam shoot explant, which is then used to produce the stock plants. Meristemic clones are grown on the solid BOO83 medium under standard tissue culture conditions, and plant shoots were regenerated from the meristemic clones after incubation.

The phrase “meristemic clones” as used herein is equivalent to the phrase “meristem derived clones”. “Meristemic clones” means that these clones were derived from a meristamatic tissue such as the apical meristem.

### Example 6

### Initiation of Sterile Yam Bioculture

This stage includes breaking field tuber dormancy (naturally, or by treatment with GA3), sprouting of buds, sterilization of sprouts in 0.5% solution of NaDCC, first cycle of cultivation in tubes on agar-solidified autoclaved BOO18 medium (FIG. 11), and subsequent cycles of cultivation on MS2 medium.

Duration: usually more than 2 months, until the plants are pathogen-free.

This stage also includes testing plants for the presence of yam viruses.

Plant growth conditions are the same as above for yam stock culture: 24° C., 16-h light period at a photon flux density of 85-100 μmol/m²/s.

### Example 7

### Propagation and Multiplication Stage

This stage is performed to multiply the number of yam plants. One 4-5-week-old well-developed yam plant contains from 4 to 7 axillary buds (embryonic shoots that lie at the junction of the stem and petiole of the plant), each of which develops into a single plant. At this stage, yam plants are grown either on solid BOO17 medium in culture tubes or in liquid BOO17 medium in small bioreactors. Duration: 4-6 weeks on solid BOO17 medium in culture tubes, or 2.5-3 weeks (depending on yam variety) in liquid medium in bioreactors.

Plant growth conditions are: 24° C., 16-h light period at a photon flux density of either 85-100 μmol/m²/s (in tubes) or 30-80 μmol/m²/s (in bioreactors).

### Example 8

### Pre-Tuberization Stage (Tuberization Stage 1)

This stage is performed in temporary immersion bioreactors. It prepares yam explants for tuberization.

Culture media: liquid BOO18 containing filter-sterilized growth regulators IAA and 2iP (FIG. 11; MS=Murashige and Skoog medium), or liquid BOO19 containing low concentration of ancymidol (anti-gibberellic substance) (see FIG. 11). In some embodiments, instead of BOO19, liquid BOO20 media is used.

BOO20 (per 1 L (1000 ml):

MS macroelements (MS=Murashige and Skoog medium)

MS Iron (II) Sulfate/EDTA chelated solution

MS microelements

MS vitamins

Sucrose—20 g

0.5 mg/l Ancymidol (equals to 2 μM) (filter-sterilized)

MES—1.95 g

pH 5.8, adjusted with 1 N KOH

Duration: 1 to 3 weeks.

Culture conditions: 24° C., 16-h light period at a photon flux density of 30-80 μmol/m²/s.

### Example 9

Tuberization stage (Tuberization Stage 2)

This stage is performed in temporary immersion bioreactors (e.g., a PV1 system, as described herein).

An exemplary PV1 System for the production of yam microtubers is provided as follows.

The PV1 vessel is a light weight polycarbonate plastic vessel with measurements of 10.5″×4″×4″, and includes a lid in the front of the vessel, and two 0.2 micron micro porous filter patches on the sides for gas exchange. The vessel and lids are autoclavable. This unit was purchased from Phytotechnology laboratories.

Setup of PV1 Vessels:

The PV1 vessels are quality inspected for defects such as cracks and washed with a mild detergent and rinsed with de-ionized water. The prepared vessels are then autoclaved at about 250 degree F. for about 40 min. The vessels are now ready to be inoculated with explants.

Inoculation of Yam Explants:

This procedure is carried out in the laminar flow hood. Nodal segments of yam plants are carefully prepared from in vitro stock yam plants maintained in test tubes or other stock maintenance containers. Each nodal segment of the prepared explants will have a minimum of one node with an active axillary bud. Approximately 50 to 100 nodal explants are carefully placed in the each of the prepared PV1 vessel with a sterile cool forceps. About 50 to about 100 mls of liquid multiplication media is added to the PV1 vessel prior to sealing the vessel with the lid. Parafilm could be used to further secure the lid of the vessel.

Culture Incubation and Media Change:

The PV1 vessels containing the yam explants are placed in a “rocker stand” that gently rocks the cultures at the rate of 2 to 5 rocking movements per minute. This movement makes sure that the yam explants are bathed in the liquid media at least two to five times every minute and also exposes the explants to drier environment thus creating an ebb and flow system. The cultures are provided with a 16/8 h photoperiod and 23±2 degree C. growth environment.

The media in the PV1 vessels are replaced once a week. This is done in the laminar flow hood. After about two to about four weeks of culture incubation, the PV1 vessels are removed from the culture room and brought to the laminar flow hoods for observation and media change to produce micro tubers. At this stage of culture, the PV1 vessels will have numerous well developed and elongated yam shoots. After carefully inspecting the PV1 vessels for optimal culture growth, the multiplication media from the PV1 vessels are replaced by micro tuber induction media.

The PV1 vessels placed on the “rocker stand” and the cultures are incubated in the dark for a period of about 6 to 8 weeks, during which time, the micro tuber induction media in the vessels is replaced with fresh media once every week.

Micro Tuber Harvest:

At the end of about 6 to 8 weeks of culture incubation, the PV1 vessels are removed from the growth room and the micro tubers are harvested using forceps. The micro tubers are dried overnight and placed in paper bags with appropriate labels and stored in the cold at about 4 degrees C. until further use.

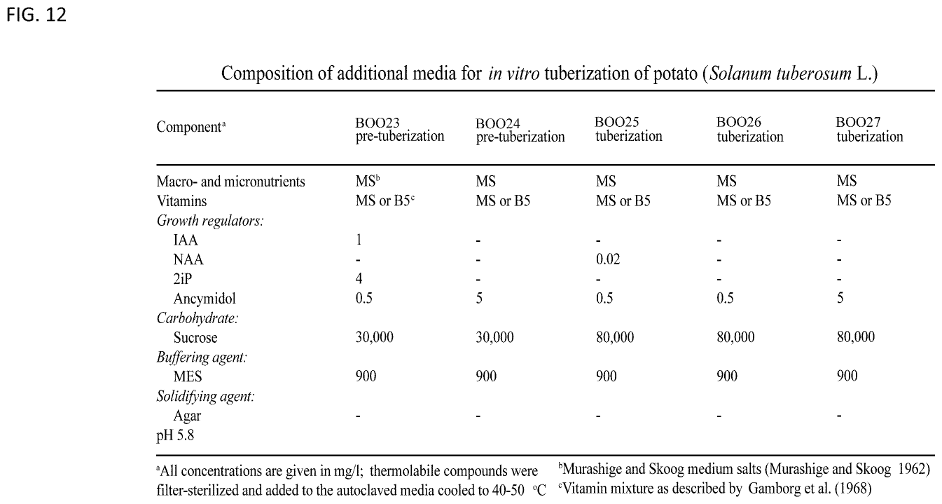

Culture media may have a high concentration of sucrose (e.g., about 60-80 g/l) and included: liquid BOO21 containing growth regulator NAA (FIG. 11), or liquid BOO26 (provided below) or BOO22 containing low concentration of ancymidol (anti-gibberellic substance) (FIG. 11). In an alternative formulation of BOO26 the concentration of MES is 0.9 g instead of 1.95 g as shown below. See, e.g., FIG. 12.

BOO26 (Per 1 L (1000 ml))

MS macro elements (MS=Murashige and Skoog medium)

MS Iron (II) Sulfate/EDTA chelated solution

MS micro elements

MS vitamins

Sucrose—80 g

0.5 mg/l Ancymidol (equals to 2 μM) (filter-sterilized)

MES—1.95 g

Duration: 6 or more weeks according to the available research publications (Kamarainen-Karppinen et al., 2010; Jimenez et al., 1999; Akita and Takayama, 1994). A longer duration of this stage may generate more microtubers (both in numbers and weight). In addition, ancymidol-containing media may be included to increase the number of microtubers and their sizes.

Culture conditions: 20-24° C., continuous darkness.

### Example 10

### Harvesting Microtubers

This step includes washing, drying, weighing, counting and storing the microtubers at 4° C.

The temporary immersion system for production of yam microtubers in vitro will result in shorter tuber development phase and a higher yield of microtubers, as compared with other available protocols (e.g., Kamarainen-Karppinen et al., 2010).

### Example 11

### Propagating New Yam Plants by Using the Harvested Microtubers

The microtubers produced in vitro are planted into soil to raise new yam plants.

### Example 12

### Planting Microtubers in Greenhouse

The microtubers produced in vitro are planted into soil to raise large yam tubers. The microtubers are grown under proper conditions and the large yam tubers can be harvested. The microtubers produced in vitro are divided into 2 groups: small size and big size with 5 microtubers per group. Each group of 5 microtubers is planted in a single 5-gallon pot, and grown under standard greenhouse conditions for 4 months. After that, the watering of plants is stopped, vegetation is allowed to dry, and all tubers are harvested, dried, weighed, and placed in cold storage at 4° C.

### Example 13

### Planting Microtubers in Field

The microtubers produced in vitro are divided into 2 groups: small size and big size. Each group comprises 5, 10, 15, 20, 25, 30, 35, 40, 45, 50 or more microtubers. Each group is planted in a horticulture field for proper time. After that, the watering of plants is stopped, vegetation is allowed to dry, and all tubers are harvested, dried, weighed, and placed in cold storage at about 4° C.

### Example 14

### Microtubers Propagation by Using the PV2 System

A PV2 system is also used for yam microtubers propagation.

The PV2 System for the production of yam microtubers is provided as follows.

The PV2 vessel is a modified 5 gallon clear polycarbonate plastic vessel, purchased from Fisher scientific. It includes two ports at the front of the vessel and a lid. One of the ports is used to fit the vessel with about a 0.2 micron air filter and the other port is used to pump in sterile media into the vessel during operation. This unit is autoclavable.

Setup of PV2 vessels:

The PV2 vessels are quality inspected for defects such as cracks and washed with a mild detergent and rinsed with de-ionized water. The washed vessels are then fitted with about a 0.2 micron air filter at one of the ports and the second port is fitted with a system of silicon tubing. The silicon tubing is approximately about one to 4 feet long. The open end of the tube is covered with a filter paper and wrapped with foil. The whole setup is autoclaved at about 250 degree F. for about 40 min. The vessels are now ready to be inoculated with yam explants.

Inoculation of Yam Explants:

This procedure is carried out in the laminar flow hood. Nodal segments of yam plants are carefully prepared from invitro stock yam plants maintained in test tubes or other stock maintenance containers. Each nodal segment of the prepared explants will have a minimum of one node with an active axillary bud. Approximately 100 to 500 nodal explants are carefully placed in the each of the prepared PV2 vessel with a sterile cool forceps. The PV2 vessel is closed with its lid and sealed with parafilm.

A 5 L bottle containing sterile yam multiplication media is attached to the free end of the silicon tube attached to the PV2 vessel.

Culture Incubation and Media Change:

The PV2 vessel setup along with the media bottle is placed in a growth room and the cultures are provided with a 16/8 h photoperiod and 23±2 degree C. growth environment. Media is pumped into the PV2 vessel using an air pump, at the rate of once every hour.

At the end of about 3 to 4 weeks of culture incubation, the multiplication media in the media bottle is replaced with micro tuber induction media. The cultures are now incubated in the dark for a period of about 6 to 8 weeks during which time; the yam plants produce micro tubers.

Micro Tuber Harvest:

At the end of about 6 to 8 weeks of culture incubation, the PV2 vessels are removed from the growth room and the micro tubers are harvested using forceps. The micro tubers are dried overnight and placed in paper bags with appropriate labels and stored in the cold at about 4 degrees C. until further use.

### Example 15

### Micropropagation of Echinacea Species

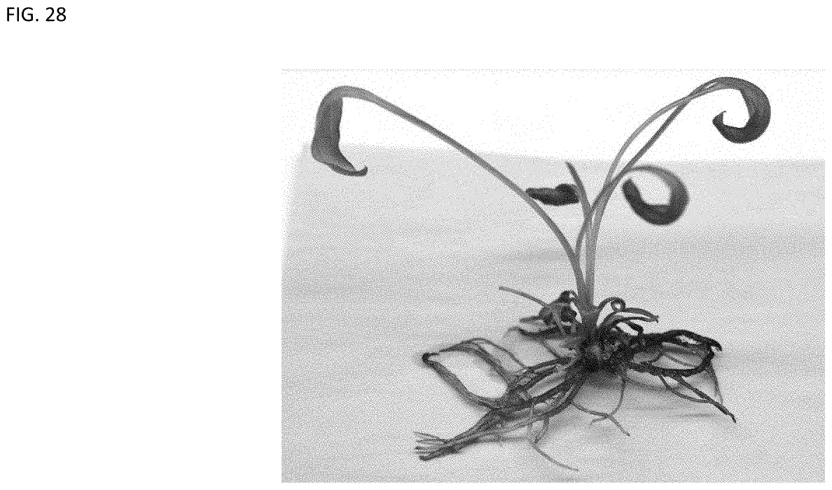

Initial explants taken from greenhouse or field grown plants are surface sterilized, using a procedure such as described in PCT Publication No. WO2013016198. In vitro cultures are initiated in culture vessels containing BOO66 solid media (FIG. 26B). After several 4-week subculture cycles, microshoots are moved into bioreactors. The initial liquid media is similar to BOO66 media but without agar. Every 7 to 10 days the initial media is replaced with Pulsing media 1 (FIG. 26B). The material is maintained in the Pulsing media 1 for 3 days. All media is refreshed every 4 weeks. The cultures in bioreactors are maintained under standard environmental conditions (16/8 hour photoperiod and 25° C.±2° C.). Microshoots are harvested at maturity (6 weeks). Singulated shoots are moved to BOO70 media (FIG. 27) for rooting. Established plants (FIG. 28) are planted in soil.

### Example 16

### Micropropagation of Hakonechloa Species

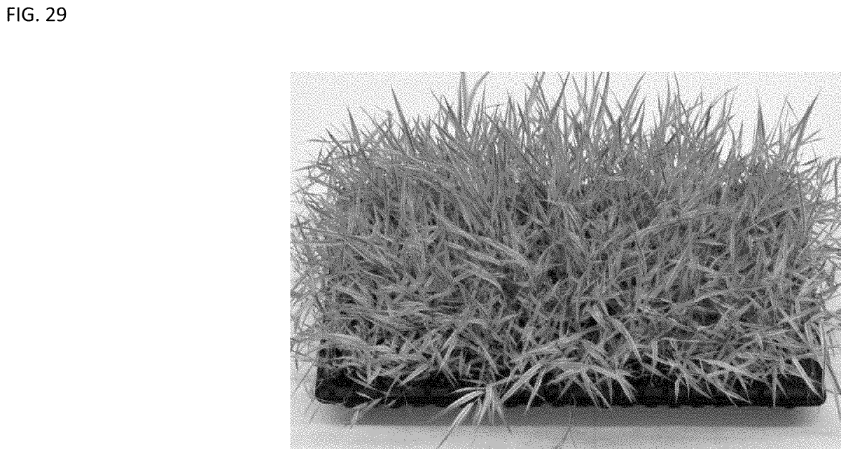

Initial explants taken from greenhouse or field grown plants are surface sterilized, using a procedure such as described in PCT Publication No. WO2013016198. In vitro cultures are initiated in culture vessels containing BOO60 solid media (FIG. 26A). After several 4-week subculture cycles, microshoots are moved into bioreactors. The initial liquid media is similar to BOO60 media but without agar. The material is maintained in BOO60 media for up to 6 weeks. The cultures are exposed to pulsing medium containing TDZ (Pulsing medium 1) FIG. 26B) followed by medium containing meta-topoline, once every 3 to 7 days. After the 6 weeks period the culture in bioreactor is pulsed weekly with Pulse 2 media (Pulsing media 2; FIG. 26B). All media is refreshed every 4 weeks. The cultures in bioreactors are maintained under standard environmental conditions (16/8 hour photoperiod and 25° C.±2° C.). Microshoots are harvested at maturity (8 to 10 weeks). Singulated shoots are moved to BOO70 media (FIG. 27) for rooting. Established plants (FIG. 29) are planted in soil.

### Example 17

### Micropropagation of Miscanthus Species

Initial explants taken from greenhouse or field grown plants are surface sterilized, using a procedure such as described in PCT Publication No. WO2013016198. In vitro cultures are initiated in culture vessels containing BOO61 solid media (FIG. 26A). After several 4-week subculture cycles, microshoots are moved into bioreactors. The initial liquid media is similar to BOO61 media but without agar. The material is maintained in BOO61 media for up to 6 weeks. The cultures are exposed to pulsing medium containing TDZ (Pulsing medium 1) (FIG. 26B) followed by medium containing meta-topoline, once every 3 to 7 days. After the 6 weeks period the culture in bioreactor is pulsed weekly with Pulse 2 media (Pulsing media 2; FIG. 26B). All media is refreshed every 4 weeks. The cultures in bioreactors are maintained under standard environmental conditions (16/8 hour photoperiod and 25° C.±2° C.). Microshoots are harvested at maturity (8 to 10 weeks). Singulated shoots are moved to BOO70 media (FIG. 27) for rooting.

### Example 18

### Micropropagation of Geranium Species

Initial explants taken from greenhouse or field grown plants are surface sterilized, using a procedure such as described in PCT Publication No. WO2013016198. In vitro cultures are initiated in culture vessels containing BOO67 solid media (FIG. 26B). After several 4-week subculture cycles, microshoots are moved into bioreactors. The initial liquid media is similar to BOO67 media but without agar. The material is maintained in BOO67 media for up to 8 weeks. The cultures are exposed to pulsing medium containing TDZ (Pulsing medium 1) (FIG. 26B) followed by medium containing meta-topoline, once every 3 to 7 days. After the 6 weeks period the media in bioreactor is changed with BOO71 media (FIG. 27). All media is refreshed every 4 weeks. The cultures in bioreactors are maintained under standard environmental conditions (16/8 hour photoperiod and 25° C.±2° C.). Microshoots are harvested at maturity (8 to 10 weeks). Singulated shoots are moved to BOO71 media (FIG. 27) for rooting.

### Example 19

Somatic Embryogenesis of Phyllostachys edulis ‘Moso’ Bamboo Using Axillary or Lateral Bud Meristem

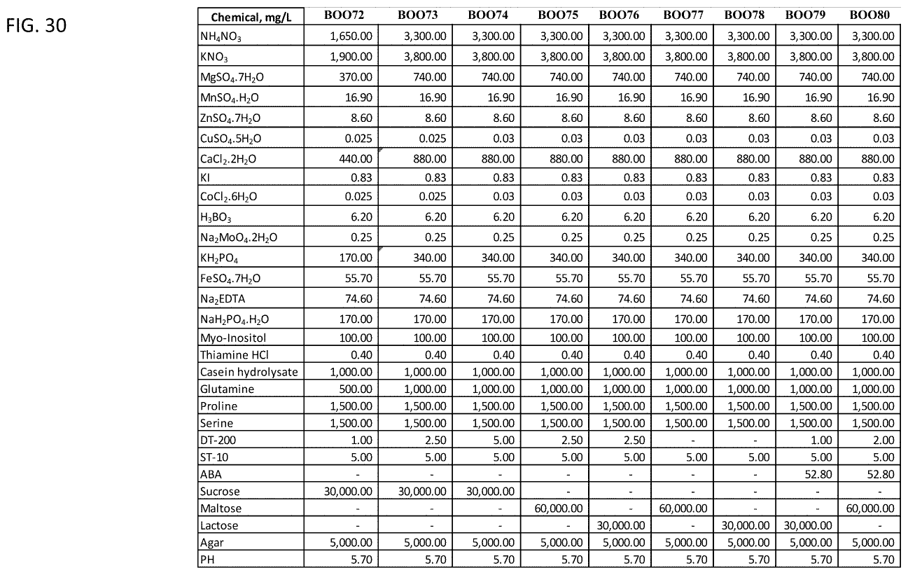

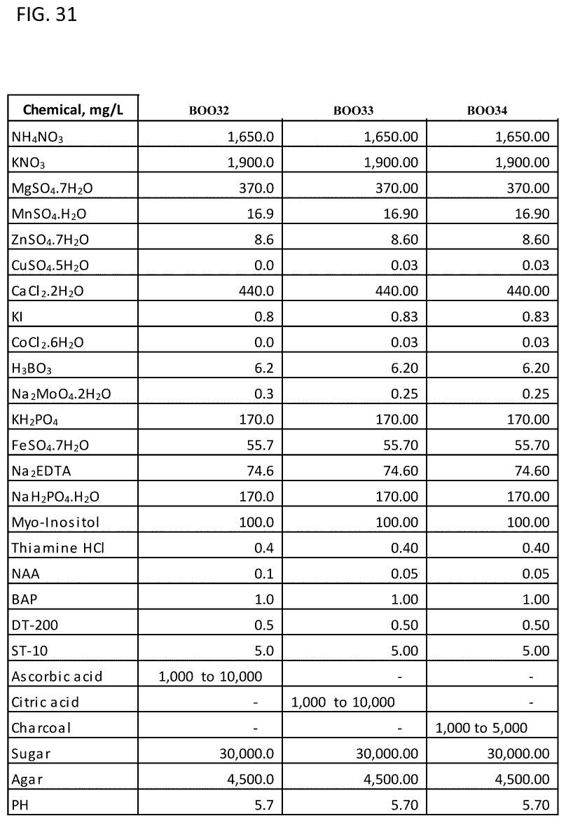

Embryogenic callus or direct somatic embryogenesis is induced from meristematic cells located into axillary or lateral buds in Moso in vitro cultures. Sterile buds are cut in segments either vertically or horizontally. The exposed meristematic cells are placed in contact with specific media. The media is described in FIG. 30.

In vitro shoot multiplication in Moso and other bamboo cultures is performed as described in PCT Publication No. WO 2013/016198, which is incorporated by reference in its entirety.

Sterile buds are removed from the in vitro shoots and cut into segments either vertically or horizontally. The exposed meristematic cells are placed in contact with BOO72, BOO73 or BOO74 media for induction of somatic embryogenesis. The cultures are incubated under standard conditions as described in PCT Publication No. WO 2013/016198.

These cultures are maintained on the same media or pulsed with media BOO75 and BOO76 for 1 to 3 days or for up to seven days. After subculturing material to fresh medium every 28 days during a period of 6 months embryo-like structures are induced. Further on clusters of embryo like structures (pro-embryos) are then transferred into establishment media BOO77 or BOO78. After desired amount of transfers, embryos are harvested and moved to media BOO79 or BOO80 for embryo maturation, for 6 to 12 weeks. The mature somatic embryos is then further desiccated or germinated on BOO72 medium containing no growth regulators.

### Example 20

### Reduction of Phenolics in Bamboo Tissue Culture

A bamboo tissue culture is prepared as described in PCT Publication No. WO 2013/016198, which is incorporated by reference in its entirety, in which the media used is BOO32, BOO33, or BOO34, and control media culture MS.

Briefly, explants are transferred to comprising MS salts supplemented with compounds known to reduce phenol production in plants. Each treatment is performed in replicates. The media is solidified and the pH is adjusted prior to autoclaving. The cultures are incubated in a growth room with a 16 h light and 8 h dark cycle at 25±2° C.

After allowing time for growth, the explants are removed and the total amounts of phenols in the culture media (for excreted phenols from explants to medium) are analyzed. Phenolic production is measured using HPLC-DAD (diode array detector) and LC-MS/MS. The phenolic production of the bamboo tissue cultures using BOO32, BOO33 and BOO34 media is lower as compared to the bamboo tissue culture using MS media.

### Example 21

### Isolation of Virus-Free Potato

In vitro potato culture were initiated using single-node cuttings of fully developed potato plants in tubes (1 single-node cutting per tube) with BOO81 medium containing 50 mg/L Ribavirin (Virazole) (see FIG. 32). The cultures were incubated under standard tissue culture conditions of 16 h light photoperiod using cool white fluorescent lights (80-100 μmol/m²/s light intensity) at 24° C.

The explants grew up to 3-5 nodes stage (about 4 to 6 cm high, 1-2-week-old culture). The cultures were then placed in an incubator at 37° C. (thermotherapy) under a 16 h photoperiod at 30-40 μmol/m²/s light intensity, for 1 week.

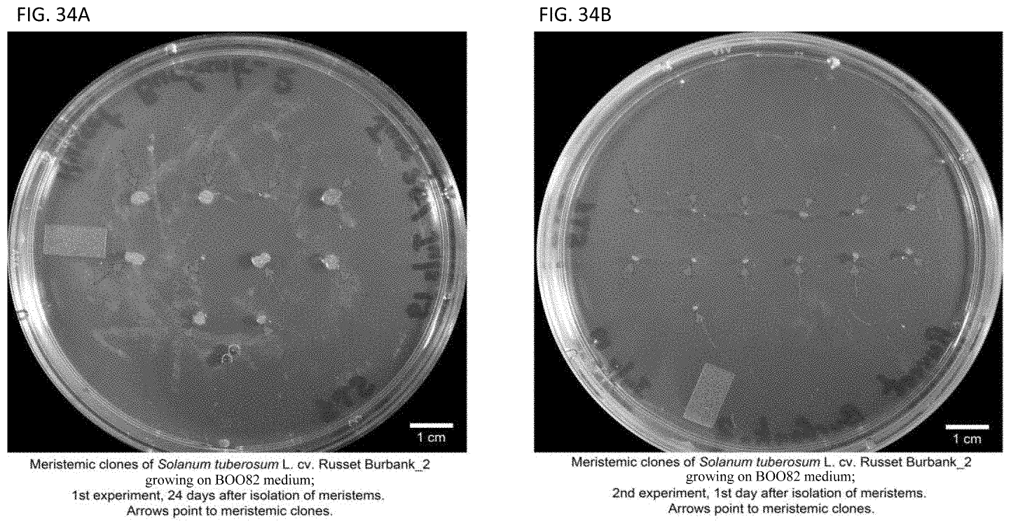

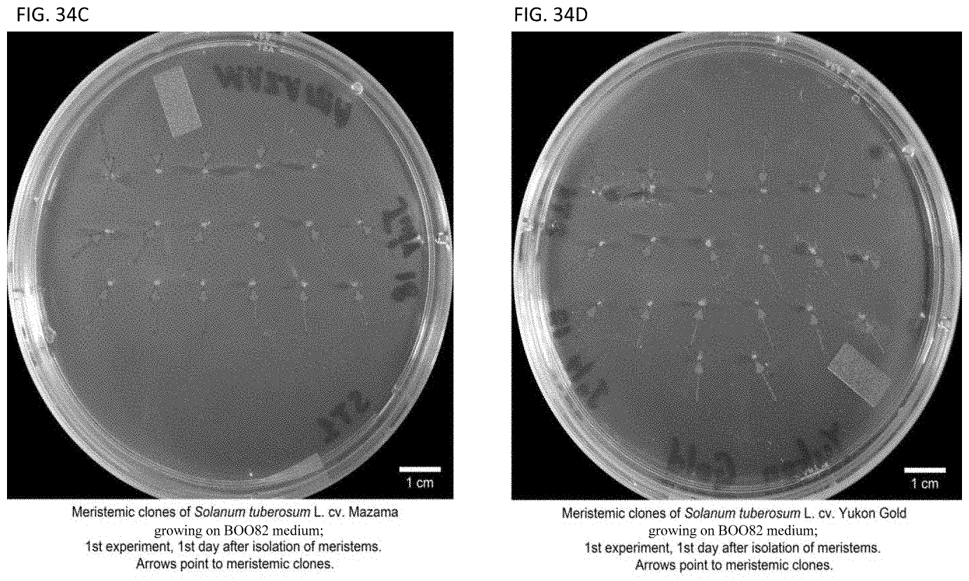

Apical meristems from thermo-treated in vitro plantlets were excised under sterile conditions (laminar flow cabinet) using a stereoscopic zoom microscope, scalpel with new blade, and forceps. A drop of sterile distilled water was used to avoid meristem desiccation during excision. In general, excision of the disc (0.2-0.3 mm thick, 0.6-1.0 mm in diameter) of apical dome is as shown in FIG. 33, without the leaf primordia. The excision is then placed carefully on agarose-solidified meristem culture medium BOO82 (see FIG. 32; callus induction medium) in a Petri dish. The isolated meristemic potato clones are depicted in FIG. 34A-34B.

The cultures were incubated at 24° C. under a 16 h photoperiod with 80-100 μmol/m²/s light intensity. Optionally, an antiviral chemical, such as Ribavirin (also known as Virazole) can be added to the BOO82 culture medium, and cultured as above.

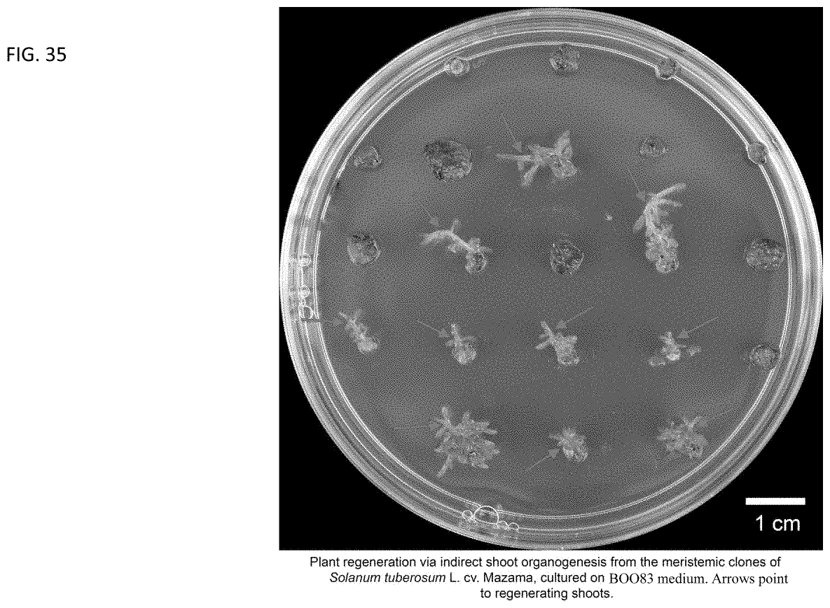

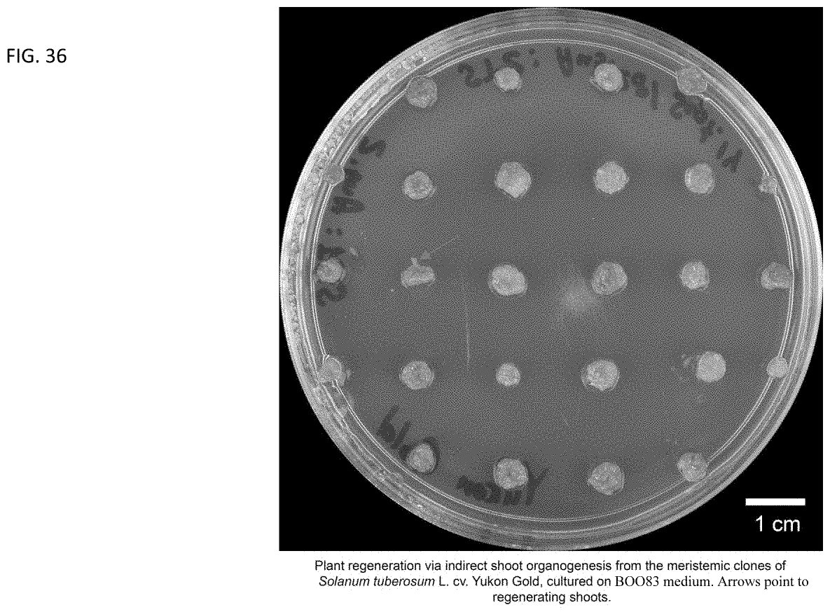

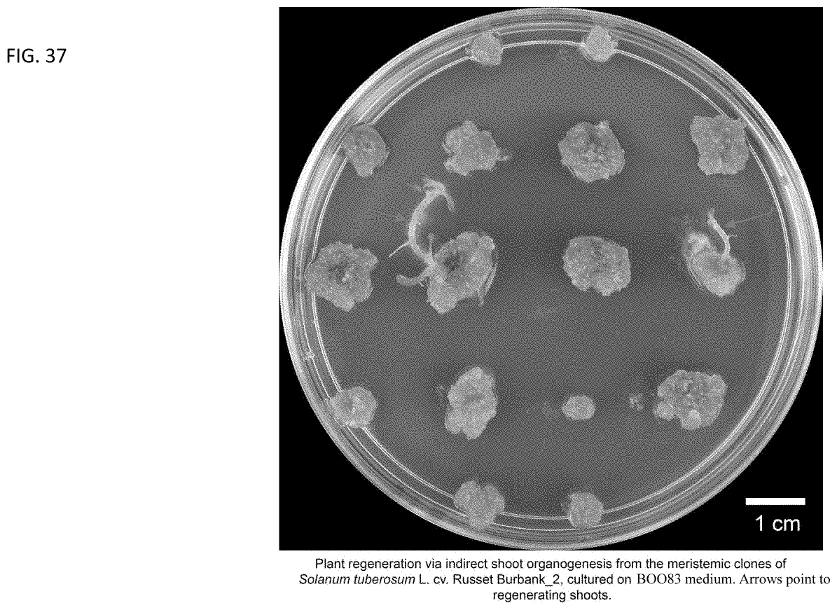

The mericlones (excised apical meristems) were subcultured to fresh BOO82 medium every 2-3 weeks. After the mericlones have reached the size of 0.5-0.7 cm in diameter (callus proliferation stage, 1-2 months of culture), they were transferred onto BOO83 medium (FIG. 32) to induce plant regeneration. Mericlones were subcultured to fresh BOO83 medium every 2-3 weeks (plant regeneration stage, 1-4 months). Plant regeneration of the mericlones are depicted in FIG. 35-37.

It takes about 5-6 months for the isolated meristems to grow into full plantlets. At this stage, the plantlets are subcultured individually to maintain their clonal identity.

The following table is an example of an experiment performed, in which the potato cultivar, number of mericlones isolated and media used to is provided, in which the first experiment depicts results in which the mericlones were established earlier than the mericlones of the second experiment, which in turn were established earlier than the mericlones in the third experiment.

TABLE 3

| | | | 3^{rd} |
|---|---|---|---|
| | 1^{st} | 2^{nd} | experiment; |
| | experiment; | experiment; | using BOO81 medium |
| Name of potato | number of mericlones | number of mericlones | number of mericlones |
| cultivar | isolated, media | isolated, media | isolated, media |
| Mazama | 23 mericlones; | 25 mericlones; | 16 mericlones; |
| | BOO82 medium | BOO82 medium | BOO82 medium |
| | BOO83 medium | BOO83 medium | |
| | 6 shoots detected | 5 shoots detected | |
| Yukon Gold | 25 mericlones; | 34 mericlones; | 38 mericlones; |
| | BOO82 medium | BOO82 medium | BOO82 medium |
| | BOO83 medium | BOO83 medium | (regeneration of |
| | | 1 shoot detected | 1 shoot was detected |
| | | | on one callus on Oct. 3rd). |
| Russet Burbank_2 | 10 mericlones; | 13 mericlones; | 64 mericlones; |
| | BOO82 medium | BOO82 medium | BOO82 medium |
| | BOO83 medium | BOO83 medium | |
| | (regeneration of 2 | 1 shoot detected | |
| | shoots was detected | | |
| | on one callus on | | |
| | Aug. 2). | | |

The meristem-derived plantlets are then tested for viruses by ELISA. The virus-negative counterparts of meristem-derived clones are multiplied and maintained by single-node culture in vitro as described above. The plants are subcultured after 4-6 weeks depending on growth response. The in vitro plants produced by this method are then used for microtuber production.

### Example 22

### Isolation of Virus-Free Cucumber

In vitro cucumber culture is initiated using single-node cuttings of fully developed cucumber in tubes (1 single-node cutting per tube) with BOO81 medium containing 50 mg/L Ribavirin (Virazole) (see FIG. 32). The cultures are incubated under standard tissue culture conditions (16/8 hours photoperiod, 25° C.±2° C.).

The explants grow up to 3-5 nodes stage (about 4 to 6 cm high, 1-2-week-old culture). The cultures are then placed in an incubator at 37° C. (thermotherapy) under a 16 h photoperiod at 30-40 μmol/m²/s light intensity, for 1 week.

Apical meristems from thermo-treated in vitro plantlets are excised under sterile conditions (laminar flow cabinet) using a stereoscopic zoom microscope, scalpel with new blade, and forceps. A drop of sterile distilled water is used to avoid meristem desiccation during excision. In general, excision of the disc (0.2-0.3 mm thick, 0.6-1.0 mm in diameter) of apical dome is as shown in FIG. 33, without the leaf primordia. The excision is then placed carefully on agarose-solidified meristem culture medium BOO85 (see FIG. 32; callus induction medium) in a Petri dish.

The cultures are incubated at 24° C. under a 16 h photoperiod with 80-100 μmol/m²/s light intensity. Optionally, an antiviral chemical, such as Ribavirin (also known as Virazole) is added to the BOO85 culture medium, and cultured as above.

The mericlones (excised apical meristems) are subcultured to fresh BOO85 medium every 2-3 weeks. After the mericlones reach the size of 0.5-0.7 cm in diameter (callus proliferation stage, 1-2 months of culture), they are transferred onto BOO86 medium (FIG. 32) to induce plant regeneration. Mericlones are subcultured to fresh BOO86 medium every 2-3 weeks (plant regeneration stage, 1-4 months).

It takes about 5-6 months for the isolated meristems to grow into full plantlets. At this stage, the plantlets are subcultured individually to maintain their clonal identity. The meristem-derived plantlets are then tested for viruses by ELISA. The virus-negative counterparts of meristem-derived clones are multiplied and maintained by single-node culture in vitro as described above. The plants are subcultured after 4-6 weeks depending on growth response. The in vitro plants produced by this method are then used for cucumber plant production.

### Example 23

Micropropagation of Cannabis species

Initial explants of Cannabis sativa taken from greenhouse or field grown plants are surface sterilized, using a procedure such as described in PCT Publication No. WO2013016198. In vitro cultures are initiated in culture vessels containing BOO57 solid media (FIG. 26A). After several 4-week subculture cycles, microshoots are moved into bioreactors. The initial liquid media is similar to BOO57 media but without agar. The material is maintained in BOO57 media for up to 6 weeks. The cultures are exposed to pulsing medium containing TDZ (Pulsing medium 1) FIG. 26B) followed by medium containing meta-topoline, once every 3 to 7 days. After the 6 weeks period the culture in bioreactor is pulsed weekly with Pulse 2 media (Pulsing media 2; FIG. 26B). All media is refreshed every 4 weeks. The cultures in bioreactors are maintained under standard environmental conditions (16/8 hour photoperiod and 25° C.±2° C.). Microshoots are harvested at maturity (8 to 10 weeks). Singulated shoots are moved to BOO70 media (FIG. 27) for rooting. Established plants (FIG. 29) are planted in soil or hydroponic applications.

### Example 24

Culture initiation of Hemp

Explants for hemp culture initiation were collected from healthy hemp stocks grown in green house and/or field. Apical and lateral buds of plants were isolated and washed thoroughly with a mild detergent and and surface sterilized under aseptic conditions. Surface sterilization can be achieved by immersing the shoot buds in solutions of bleach for a period of time ranging from about 15 min to about 2 hours. After surface sterilization, the shoots were placed in a sterile surface in the laminar flow hood, dead tissues removed using a sharp scalpel and the buds were inoculated in test tubes containing BOO3 (a.k.a. BOOS3) media, the compositions of which are presented in FIG. 38.

### Culture Multiplication

Initiated hemp cultures were multiplied in liquid media in different culture vessels varying in size and volume from about 100 mls to about 10 Gallon. Each culture cycle ranges from between about 4 to about 8 weeks based on the vessel size. The size vessels used in the trial resulted in production of a total of about 5 plants to about 15,000 plants per vessel for each culture cycle. At the end of the multiplication cycle, rooting cycle was initiated on BOO6 (a.k.a. BOOS6) media, which are presented in FIG. 38. Cultures were incubated with temperatures ranges from 25° C.+2° and a photoperiod set at 16 hrs/8 hrs of light/dark.

In view of the many possible embodiments to which the principles of the disclosed invention may be applied, it should be recognized that the illustrated embodiments are only examples and should not be taken as limiting the scope of the invention.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein.

### NUMBERED EMBODIMENTS OF THE DISCLOSURE

Notwithstanding the appended claims, the disclosure sets forth the following numbered embodiments:

1. A set of media for producing a hemp plant or plant part wherein the set of media comprises:

(a) one or more initiation medium;

(b) one or more multiplication medium; and

(c) one or more rooting medium;

wherein the initiation medium, the multiplication medium and/or the rooting medium comprises at least one cytokinin; wherein the media is selected from one of BOO1-BOO91.
2. The set of media of embodiment 1, wherein the initiation medium and the multiplication medium are the same.
3. The set of media of any one of embodiments 1 or 2, wherein each medium comprises about 20 g/L sugar, about 30 g/L sugar or about 60 to 120 g/L sugar.
4. The set of media of any one of embodiments 1-3, wherein the cytokinin in the initiation medium, the mitiplication medium and/or the rooting medium is meta-topolin (mT) or any functional derivative.
5. The set of media of embodiment 4, wherein the mT in the initiation medium, the multiplication medium and/or the rooting medium has a concentration of about 0.5 to 5.0 mg/L.
6. The set of media of any one of embodiments 1-5, wherein the rooting medium further comprises a gibberellin acid.
7. The set of media of embodiment 6, wherein the gibberellins acid is GA3.
8. The set of media of any one of embodiments 6 or 7, wherein the gibberellins acid concentration in the rooting medium is about 0.5 to 5.0 mg/L.
9. The set of media of any one of embodiments 1-8, wherein the pH of each medium is about 5.7.
10. A kit for producing a hemp plant or plant part in vitro, wherein the kit comprises a set of media of any one of embodiments 1 to 9.
11. A method for producing a hemp plant or plant part in vitro comprising:

(a) obtaining a hemp explant for hemp culture;

(b) initiating the hemp culture with the explant from step (a) on an initiation medium;

(c) multiplying the initiated hemp culture from step (b) on a multiplication medium; and

(d) transferring the multiplied hemp culture of step (c) on a rooting medium to produce a hemp plant with root, wherein the media is selected from one of BOO1-BOO91.

12. The method of embodiment 11, wherein the hemp explant is selected from apical and/or lateral buds of shoots of the hemp plants.
13. The method of any one of embodiments 11-12, wherein the explant is washed and surface sterilized.
14. The method of any one of embodiments 11-13, wherein the explant is washed with a mild detergent.
15. The method of any one of embodiments 11-12, wherein the explant is surface sterilized in solutions of bleach.
16. The method of any one of embodiments 11-15, wherein all media are solid media.
17. The method of any one of embodiments 11-15, wherein all media are liquid media.
18. The method of any one of embodiments 11-17, wherein the initiation step (b) further comprises inoculating the explant on the initiation medium in vitro.
19. The method of any one of embodiments 11-18, wherein size and volume of a culture vessel for multiplying initiated hemp culture step range from about 100 ml to about 10 gallons.
20. The method of embodiment 19, wherein a total of about 5 plants up to about 15,000 plants per the culture vessel are produced for each culture cycle.
21. The method of any one of embodiments 11-20, wherein a culture cycle for multiplication is about 4 to about 8 weeks.
22. The method of any one of embodiments 11-21, wherein the multiplied hemp culture obtained from step (c) are kept on the rooting medium until individual plant with root is developed.
23. The method of any one of embodiments 11-22, wherein the hemp culture is incubated under about 23° C. to about 27° C.
24. The method of any one of embodiments 11-23, wherein the hemp culture is incubated under 16/8 hours day/night cycle.
25. The method of any one of embodiments 11-24, wherein the initiation medium and the multiplication medium are the same.
26. The method of any one of embodiments 11-25, wherein each medium comprises about 20 g/L sugar, about 30 g/L sugar or about 60 to 120 g/L sugar.
27. The method of any one of embodiments 11-26, wherein the cytokinin in the initiation medium, the mitiplication medium and/or the rooting medium is meta-topolin (mT) or any functional derivative.
28. The method of any one of embodiments 11-27, wherein the mT in the initiation medium, the multiplication medium and/or the rooting medium has a concentration of about 0.5 to 5.0 mg/L.
29. The method of any one of embodiments 11-28, wherein the rooting medium further comprises a gibberellin acid.
30. The method of embodiment 29, wherein the gibberellins acid is GA3.
31. The method of any one of embodiments 29-30, wherein the gibberellins acid concentration in the rooting medium is about 0.5 to 5.0 mg/L.
32. The method of any one of embodiments 11-31, wherein the pH of each medium is about 5.7.
33. A method for micropropagating a plant or plant part comprising:

(a) incubating an explant of the plant on a solid media;

(b) transferring a microshoot from the explant of (a) to a bioreactor with liquid media;

(c) exposing the microshoot to a pulsing media;

(d) harvesting the microshoot at maturity; and

(e) transferring the mature microshoot to a media for rooting,

wherein the plant is a cannabis plant and the plant part is a cannabis plant part; wherein the media is selected from at least one of BOO1-BOO91.

34. The method of embodiment 33, further comprising exposing the microshoot to medium comprising meta-topoline after step (c).
35. The method of any one of embodiments 33-34, wherein the solid media is selected from FIG. 26A or 26B.
36. The method of any one of embodiments 33-34, wherein the liquid media is a media selected from FIG. 26A or 26B and without agar.
37. The method of any one of embodiments 33-36, wherein the pulsing media is selected from FIG. 26B.
38. The method of embodiment 37, wherein the pulsing media is Pulsing media 1 or Pulsing media 2 of FIG. 26B.
39. The method of any one of embodiments 33-38, wherein the pulsing media is Pulsing media 1 of FIG. 26B, wherein TDZ is substituted with a different cytokinin.
40. The method of embodiment 39, wherein the different cytokinin is BAP, Zeatin, CPPU, or DPU.
41. The method of any one of embodiments 33-40, wherein the media for rooting is selected from FIG. 27.
42. The method of any one of embodiments 33-41, wherein the media for rooting is BOO68, BOO69, BOO70, or BOO71 from FIG. 27.
43. A media for micropropagating a cannabis plant, wherein the media is selected from FIG. 26A or 26B.
44. The media of embodiment 43, wherein the media is Pulsing media 1 or Pulsing media 2 of FIG. 26B.
45. The media of any one of embodiments 43-44, wherein the media is Pulsing media 1 of FIG. 26B, wherein TDZ is substituted with a different cytokinin.
46. The media of embodiment 45, wherein the different cytokinin is BAP, Zeatin, CPPU, or DPU.
47. The media of any one of embodiments 43-46, wherein the media for rooting is selected from FIG. 27.
48. The media of embodiment 43 or 45, further comprising a BOO54, BOO55, BOO56, BOO57, BOO58, BOO59, BOO60, BOO61, BOO62, BOO63, BOO64, BOO65, BOO66, or BOO67 media from FIG. 26A or 26B.
49. A kit comprising a media of FIG. 26A or 26B.
50. The kit of embodiment 49, wherein the media is Pulsing media 1 or Pulsing media 2 of FIG. 26B.
51. The kit of embodiment 49 or embodiment 50, wherein the media is Pulsing media 1 of FIG. 26B and TDZ is substituted with a different cytokinin.
52. The kit of embodiment 51, wherein the different cytokinin is BAP, Zeatin, CPPU, or DPU.
53. The kit of any one of embodiments 49-52, further comprising a rooting media from FIG. 27.
54. The kit of embodiment 49 or 51, further comprising a BOO54, BOO55, BOO56, BOO57, BOO58, BOO59, BOO60, BOO61, BOO62, BOO63, BOO64, BOO65, BOO66, or BOO67 media from FIG. 26A or 26B.
55. A set of media for producing a pistachio plant or plant part wherein the set of media comprises:

(a) one or more initiation medium;

(b) one or more multiplication medium; and

(c) one or more rooting medium;

wherein the initiation medium and/or the multiplication medium comprises at least one cytokinin, and optionally at least one auxin, and the rooting medium comprises at least one auxin; wherein the media is selected from one of BOO1-BOO91.
56. The set of media of embodiment 55, wherein the initiation medium and the multiplication medium are the same.
57. The set of media of any one of embodiments 55-56, wherein each medium comprises about 30 g/L sugar or about 60 to 120 g/L sugar.
58. The set of media of any one of embodiments 55-57, wherein the cytokinin in the initiation medium and/or the multiplication medium is meta-topolin (mT) or any functional derivative.
59. The set of media of any one of embodiments 55-58, wherein the auxin in the initiation medium, the multiplication medium is Naphthaleneacetic acid (NAA) or any functional derivative thereof, or Indole-3-butyric acid (IBA) or any functional derivative thereof and wherein the auxin in the rooting medium is IBA or any functional derivative thereof.
60. The set of media of embodiment 58, wherein the mT in the initiation medium and/or the multiplication medium has a concentration of about 0.5 to 5.0 mg/L.
61. The set of media any one of embodiments 55-60, wherein the initiation medium and/or the multiplication medium further comprises a gibberellin acid.
62. The set of media of embodiment 61, wherein the gibberellins acid is GA3.
63. The set of media of any one of embodiments 61-62, wherein the gibberellins acid concentration in the rooting medium is about 0.5 to 5.0 mg/L.
64. The set of media of any one of embodiments 55-63, wherein the auxin concentration is about 0.01 to 5 mg/L.
65. The set of media of any one of embodiments 55-64, wherein the rooting medium does not contain any cytokinin.
66. The set of media of any one of embodiments 55-65, wherein the rooting medium comprises about 1 to about 1000 mg/L auxin.
67. The set of media of any one of embodiments 55-66, wherein the rooting medium comprises charcoal.
68. The set of media of embodiment 67, wherein the charcoal has a concentration of about 1000 mg/L.
69. The set of media of any one of embodiments 55-68, wherein the rooting medium comprises about 30 g/L sugar, or at least about 60 g/L sugar
70. The set of media of any one of embodiments 55-69, wherein the rooting medium comprises about 0.5 to 5 mg/L auxin, or at least about 30 mg/L auxin.
71. The set of media of any one of embodiments 55-70, wherein the pH of each medium is about 5.7.
72. A kit for producing pistachio plant or plant part in vitro, wherein the kit comprises a set of media of any one of embodiments 55 to 71.
73. A method for producing a pistachio plant in vitro comprising:

(a) obtaining a pistachio explant;

(b) initiating shoot from the explant obtained in step (a) on an initiation medium;

(c) multiplying the shoot initiated from step (b) on a multiplication medium; and

(d) transferring the multiplied shoots of step (c) on a rooting medium to produce a pistachio plant with root, wherein the media is selected from one of BOO1-BOO91.

74. The method of embodiment 73, wherein the pistachio explant is selected from the group consisting of single-node explants, shoot tips, basal (bottom) parts of plants with multiple buds.
75. The method of any one of embodiments 73-74, wherein culture vessels with ventilated lids are used.
76. The method of any one of embodiments 73-75, wherein the explant is washed and surface sterilized.
77. The method of embodiment 73, wherein all media are solid media.
78. The method of embodiment 73, wherein all media are liquid media.
79. The method of any one of embodiments 73-78, wherein the initiation step (b) optionally comprises sub-culturing the explant in a fresh medium every about 1 day to 1 week until first axillary shoot appears.
80. The method of any one of embodiments 73-79, wherein the step (b) lasts for about 3 days.
81. The method of any one of embodiments 73-80, wherein multiple shoots form after step (b), and the multiple shoots are divided into small clumps of about 2 or more shoots each and transferred to a multiplication medium to perform the step (c).
82. The method of any one of embodiments 73-81, wherein the shoots are kept on the multiplication medium for about 2 weeks to about 4 weeks during step (c).
83. The method of any one of embodiments 73-82, optionally the multiplied shoots obtained from step (c) are divided into smaller clumps of 2 to 3 shoots and transferred to fresh multiplication medium for further multiplication.
84. The method of embodiment 83, wherein the optional step is repeated for at least once.
85. The method of any one of embodiments 73-84, wherein the multiplied shoots obtained from step (c) are divided into clumps of 3 to 6 shoots and transferred onto a rooting medium.
86. The method of any one of embodiments 73-85, wherein the multiplied shoots are kept on the rooting medium until individual plant with root is developed.
87. The method of any one of embodiments 73-86, wherein the explant multiplies about 2 to 7 times per about every 30 days.
88. The method of any one of embodiments 73-87, wherein the multiplied shoots are kept on the rooting medium for about 1 week to about 4 weeks.
89. The method of any one of embodiments 73-88, wherein the multiplied shoots are kept on a first rooting medium having a higher sugar concentration and then transferred to a second rooting medium having a lower sugar concentration.
90. The method of embodiment 89, wherein the first rooting medium has at least about 60 g/L sugar first and second rooting medium has about 30 g/L sugar.
91. The method of any one of embodiments 89-90, wherein the first rooting medium and the second rooting medium has an auxin concentration of about 0.5 to 5 mg/L.
92. The method of any one of embodiments 73-91, wherein the multiplied shoots are kept on a first rooting medium having a higher auxin concentration and then transferred to a second rooting medium having a lower auxin concentration.
93. The method of embodiment 92, wherein the first rooting medium has at least about 30 mg/L auxin and second rooting medium has about 0.5 to 5 mg/L auxin.
94. The method of embodiment 93, wherein the first rooting medium and the second rooting medium have about the same sugar concentration.
95. The method of embodiment 94, wherein the sugar concentration is about 30 mg/L.
96. The method of any one of embodiments 73-95, wherein at least one step of the method is performed in a bioreactor.
97. The method of embodiment 96, wherein the bioreactor is a temporary immersion bioreactor.
98. The method of embodiment 97, wherein the temporary immersion bioreactor is an ebb and flow bioreactor.
99. The method of any one of embodiments 73-98, wherein the step (c) is performed in a bioreactor.
100. The method of embodiment 99, wherein the bioreactor has a predetermined size depending on production requirements.
101. The method of embodiment 96, wherein the bioreactor is placed under about 22 to about 24° C.
102. The method of embodiment 96, wherein the bioreactor is placed under 16/8 hours day/night cycle.
103. The method of embodiment 96, wherein the medium is refreshed every one to four weeks to constitute a growth cycle.
104. The method of any one of embodiments 73-103, wherein the explant is multiplied about 2 to 7 times about every 30 days.
105. The method of any one of embodiments 96-103, wherein pistachio shoots multiplied in the bioreactor are transferred to rooting medium to perform step (d).
106. The method of any one of embodiments 73-105, wherein the rooting medium is a liquid medium.
107. The method of any one of embodiments 73-106, wherein the pistachio plant with root produced is transferred to an in vitro or in vivo condition for further growth.
108. A method for inducing root from a pistachio explant in vitro comprising:

growing a pistachio explant on a first rooting medium, and then transferring the pistachio explant to a second rooting medium, wherein the first rooting medium has a higher sugar concentration compared to the second rooting medium; wherein the medium is selected from one of BOO1-BOO91.

109. A method for inducing root from a pistachio explant in vitro comprising:

growing a pistachio explant on a first rooting medium, and then transferring the pistachio explant to a second rooting medium, wherein the first rooting medium has a higher auxin concentration compared to the second rooting medium; wherein the media is selected from one of BOO1-BOO91.

110. The method of embodiment 108, wherein the first rooting medium comprises about 60 g/L to about 120 g/L sugar, and wherein the second rooting medium comprises about 30 g/L sugar.
111. The method of embodiment 108 or 110, wherein the first rooting medium comprises about the same amount of auxin.
112. The method of embodiment 108, 110, or 110, wherein the pistachio explant is pre-rooted on the first rooting medium for about 1 to about 3 weeks before being transferred to the second rooting medium.
113. The method of embodiment 108, wherein the pistachio explant is grown on the second rooting medium for about 2 to 4 weeks until roots develop.
114. The method of embodiment 109, wherein the first rooting medium comprises about 100 mg/L to 1500 mg/L IBA, and wherein the second rooting medium comprises about 0.1 to 10 mg/L IBA.
115. The method of embodiment 109 or 114, wherein the first rooting medium comprises about the same amount of sugar.
116. The method of embodiment 109, 114, or 115 wherein the pistachio explant is pre-rooted on the first rooting medium for about 1 to 24 hours before being transferred to the second rooting medium.
117. The method of embodiment 116, wherein the pistachio explant is grown on the second rooting medium for about 2 to 4 weeks until roots develop.
118. The set of media of embodiments 55-71, wherein the initiation medium and/or the multiplication medium comprise MS medium containing double concentration of meso elements (one or more of CaCl2.2H2O, MgSO4.7H2O, and KH2PO4), double iron, and one or more Gamborg's vitamins (one or more of myo-inositol, Nictotinic acid, pyridoxine salts, and thiamine salts).
119. The method of embodiments 57-108, wherein the initiation medium and/or the multiplication medium comprise MS medium containing double concentration of meso elements (one or more of CaCl2.2H2O, MgSO4.7H2O, and KH2PO4), double iron, and one or more Gamborg's vitamins (one or more of myo-inositol, Nictotinic acid, pyridoxine salts, and thiamine salts).
120. A medium for producing yam microtubers wherein said media comprises sucrose and

(1) at least one cytokinin and at least one auxin;

(2) at least one growth retardant; or

at least one cytokinin, at least one auxin, and at least one growth retardant; wherein the media is selected from at least one of BOO1-BOO91.
121. The medium of embodiment 120, wherein the sucrose has a concentration of about 25-35 g/L.
122. The medium of any one of embodiments 120-121, wherein the at least one cytokinin is 2ip.
123. The medium of embodiment 122, wherein the 2ip has a concentration of about 1 to 10 mg/L.
124. The medium of any one of embodiments 120-123, wherein the at least one auxin is IAA.
125. The medium of embodiment 124, wherein the IAA has a concentration of about 0.1 to 10 mg/L.
126. The medium of any one of embodiments 120-125, wherein the growth retardant is a gibberellins acid antagonist.
127. The medium of embodiment 126, wherein the gibberellins acid antagonist is ancymidol.
128. The medium of embodiment 127, wherein the ancymidol has a concentration of about 0.1 to 10 mg/L.
129. The medium of any one of embodiments 120-128, wherein the medium is a solid, semi-solid, liquid, or semi-liquid medium.
130. The medium of any one of embodiments 120-129, wherein the medium has a pH of about 5.5 to 6.2.
131. A medium for producing yam microtubers wherein said media comprises sucrose and

(i) at least one auxin;

(ii) at least one growth retardant; or

at least one auxin and at least one growth retardant; wherein the media is selected from at least one of BOO1-BOO91.
132. The medium of embodiment 131, wherein the sucrose has a concentration of about 50-100 g/L.
133. The medium of any one of embodiments 131-132, wherein the medium does not comprise any cytokinin.
134. The medium any one of embodiments 131-133, wherein the at least one auxin is NAA.
135. The medium of embodiment 134, wherein the NAA has a concentration of about 0.01 to about 0.1 mg/L.
136. The medium of any one of embodiments 131-135, wherein the growth retardant is a gibberellins acid antagonist.
137. The medium of embodiment 136, wherein the gibberellins acid antagonist is ancymidol.
138. The medium of embodiment 137, wherein the ancymidol has a concentration of about 0.1 to 10 mg/L.
139. The medium of any one of embodiments 131-138, wherein the medium is a solid, semi-solid, liquid, or semi-liquid medium.
140. The medium of any one of embodiments 131-139, wherein the medium has a pH of about 5.5 to 6.2.
141. A set of media for producing yam microtubers wherein the set of media comprises:

(1) one or more propagation and multiplication medium;

(2) one or more pre-tuberization medium; and

(3) one or more tuberization medium;

wherein the propagation and multiplication medium does not contain any plant hormone or plant growth regulator;
wherein the pre-tuberization medium comprises sucrose at concentration S1 and
(i) at least one cytokinin and at least one auxin;
(ii) at least one growth retardant; or
(iii) at least one cytokinin, at least one auxin, and at least one growth retardant;
   wherein the tuberization medium comprises sucrose at concentration S2 and
(i) at least one auxin;
(ii) at least one growth retardant; or
(iii) at least one auxin and at least one growth retardant;
   wherein S1 is smaller than S2; and,
   wherein the propagation and multiplication medium, the pre-tuberization medium, and the tuberization medium are used to produce yam microtubers; wherein the media is selected from at least one of BOO1-BOO91.
   142. The set of media of embodiment 141, wherein S1 is about 25-35 g/L and S2 is about 50-100 g/L.
   143. The set of media of any one of embodiments 141-142, wherein the at least one cytokinin in the pre-tuberization medium is 2ip.
   144. The set of media of embodiment 143, wherein the 2ip has a concentration of about 1 to 10 mg/L.
   145. The set of media any one of embodiments 141-144, wherein the at least in one auxin in the pre-tuberization medium is IAA.
   146. The set of media of embodiment 145, wherein the IAA has a concentration of about 0.1 to 10 mg/L.
   147. The set of media of any one of embodiments 141-146, wherein the at least one auxin in the tuberization medium is NAA.
   148. The set of media of embodiment 147, wherein the NAA has a concentration of about 0.01 to about 0.05 mg/L.
   149. The set of media of any one of embodiments 141-148, wherein the growth retardant in the pre-tuberization medium and/or the tuberization medium is a gibberellins acid antagonist.
   150. The set of media of embodiment 149, wherein the gibberellins acid antagonist is ancymidol.
   151. The set of media of any one of embodiments 150-151, wherein the ancymidol has a concentration of about 0.1 to 10 mg/L.
   152. The set of media of any one of embodiments 141-151, wherein one or more medium is a solid, semi-solid, liquid, or semi-liquid medium.
   153. The set of media of any one of embodiments 141-152, wherein one or more medium has a pH of about 5.5 to 6.2.
   154. A kit for producing yam microtubers, wherein the kit comprises a medium of any one of embodiments 120 to 140 or a set of media of any one of embodiments 141 to 153.
   155. A method for producing yam microtubers comprising:
(a) obtaining pathogen-free yam sprouts;
(b) propagating the pathogen-free yam sprouts obtained in step (a) to produce yam plants;
(c) pretreating the yam plants obtained from step (b) or any other sources to produce pretreated yam plants;
(d) initiating microtubers from the pretreated yam plants obtained from step (c); and optionally, harvesting the microtubers produced in step (d); wherein the media is selected from at least one of BOO1-BOO91.
   156. The method of embodiment 155, wherein the step (a) comprises breaking field tuber dormancy to induce buds.
   157. The method of embodiment 156, wherein field tuber dormancy was broken naturally, or by treatment with GA3, ethanol, temperature, thiourea, ethylene chlorohydrins, rindite, carbon disulphide, and/or bromoethane.
   158. The method of embodiment 156, wherein the induced buds are further grown into sprouts.
   159. The method of embodiment 158, wherein the sprouts are further sterilized.
   160. The method of embodiment 159, wherein the sprouts are sterilized in 0.5% solution of sodium dichloroisocyanurate (NaDCC).
   161. The method of embodiment 160, wherein the sterilized sprouts are further cultivated in vitro for one or more cycles until pathogen-free sprouts are produced.
   162. The method of embodiment 161, wherein the sprouts are first cultivated in a solid, semi-solid, liquid, or semi-liquid medium comprising Murashige & Skoog (MS) salts, IAA, 2ip, and sucrose.
   163. The method of embodiment 162,
   wherein the concentration of IAA is about 0.1 to 1 mg/L;
   wherein the concentration of 2ip is about 1 to 10 mg/L; and
   wherein the concentration of sucrose is about 10 to 40 g/L.
   164. The method of embodiment 162, wherein the sprouts are further cultivated in a medium comprising MS salts and sucrose without any hormones or growth regulators.
   165. The method of embodiment 164, wherein the concentration of sucrose is about 10 to 40 g/L.
   166. The method of embodiment 161, wherein the sprouts are grown under about 24° C., 16/8 hour day/night light cycle, with a photon flux density of about 85-100 μmol/m²/s.
   167. The method of embodiment 161 wherein the method further comprises testing the presence or absence of one or more yam pathogen species after one or more cycles, wherein the pathogen species is a bacteria species, fungal species, or virus species.
   168. The method of any one of embodiments 155-167, wherein the step (b) comprises propagating the pathogen free yam sprouts in a culture tube or a bioreactor.
   169. The method of embodiment 168, wherein the bioreactor is a temporary immersion bioreactor.
   170. The method of any one of embodiments 168-169, wherein each pathogen-free yam sprout has about 4-7 axillary buds.
   171. The method of embodiment 169, wherein a solid, semi-solid, liquid or semi-liquid propagation and multiplication medium is used, and wherein the propagation and multiplication medium comprises MS salts and sucrose at a concentration of about 20 g/L.
   172. The method of embodiment 171, wherein the propagation and multiplication medium does not comprise any plant hormone or growth regulator.
   173. The method of any one of embodiments 168-172, wherein the sprouts are grown under about 24° C., 16/8 hour day/night light cycle, and wherein the photon flux density is about 85-100 μmol/m²/s when the sprouts are grown in a cultivation tube, or about 30-80 μmol/m²/s when the sprouts are grown in a bioreactor.
   174. The method of any one of embodiments 168-173, wherein the yam plants are propagated with an average multiplication factor of about 5.
   175. The method of embodiment 174, wherein the yam plants are propagated for about 4-6 weeks on a solid medium, or about 2-3 weeks on a liquid medium.
   176. The method of any one of embodiments 155-160, wherein the step (c) comprising pretreating the yam plants in a bioreactor.
   177. The method of embodiment 176, wherein the bioreactor is a temporary immersion bioreactor.
   178. The method of embodiment 176, wherein the yam plants are pretreated in a medium of any one of embodiments 1-11.
   179. The method of embodiment 176, wherein the duration of the pretreatment step is about 3 weeks.
   180. The method of embodiment 176, wherein the plants are grown under about 24° C., 16/8 hour day/night light cycle, with a photon flux density of about 30-80 μmol/m²/s.
   181. The method of embodiment 155, wherein the step (d) comprising initiating yam microtubers in a bioreactor.
   182. The method of embodiment 181, wherein the bioreactor is a temporary immersion bioreactor.
   183. The method of embodiment 181, wherein the yam plants are pretreated in a medium of any one of embodiments 78-87.
   184. The method of embodiment 183, wherein the medium has a sugar concentration higher than the sucrose concentration in the medium used for step (c).
   185. The method of embodiment 183, wherein the medium has a sugar concentration of about 60-80 g/L.
   186. The method of embodiment 181, wherein the duration of step (d) is about 6 weeks.
   187. The method of embodiment 181, wherein the plants are grown under a temperature lower than the temperature used in step (c), and a photon flux density lower than the photon flux density in step (c).
   188. The method of embodiment 187, wherein the plants are grown under a temperature of about 20-24° C., with continuous darkness.
   189. A method for producing yam microtubers comprising utilizing a set of medium of any one of embodiments 141-153 or a kit of embodiment 154.
   190. A method for producing yam microtubers comprising utilizing a temporary immersion bioreactor, wherein the bioreactor comprises:

a growth vessel for incubating plant tissue in a sterile or substantially sterile environment;

a first media container having a first fluid port and a second fluid port, the first fluid port fluidically coupleable to the growth vessel;

a second media container having a first fluid port and a second fluid port, the first fluid port fluidically coupleable to the growth vessel;

a gas source fluidically coupleable to the second fluid port of the first media container and second fluid port of the second media container; and

a controller operable in a first operating mode in which pressurized gas is delivered from the gas source to the first media container to displace a first volume of liquid contained therein to the growth vessel, and a second operating mode in which pressurized gas is delivered from the gas source to the second media container to displace a second volume of liquid contained therein to the growth vessel; wherein the media is selected from at least one of BOO1-BOO91.

191. A temporary immersion bioreactor, comprising:

a growth vessel for incubating plant tissue in a sterile or substantially sterile environment;

a first media container having a first fluid port and a second fluid port, the first fluid port fluidically coupleable to the growth vessel;

a second media container having a first fluid port and a second fluid port, the first fluid port fluidically coupleable to the growth vessel;

a gas source fluidically coupleable to the second fluid port of the first media container and second fluid port of the second media container; and

a controller operable in a first operating mode in which pressurized gas is delivered from the gas source to the first media container to displace a first volume of liquid contained therein to the growth vessel, and a second operating mode in which pressurized gas is delivered from the gas source to the second media container to displace a second volume of liquid contained therein to the growth vessel; wherein the media is selected from at least one of BOO1-BOO91.

192. The temporary immersion bioreactor of embodiment 191, wherein the controller is operable in a third operating mode in which liquid contained in the growth vessel is allowed to flow from the growth vessel into at least one of the first media container and the second media container.
193. The temporary immersion bioreactor of any one of embodiments 191 or 192, wherein the controller is operable in a first incubation sequence in which the third operating mode is executed subsequent to the first operating mode.
194. The temporary immersion bioreactor of embodiment 193, wherein the controller is operable in a second incubation sequence in which the third operating mode is executed subsequent to the second operating mode.
195. The temporary immersion bioreactor of embodiment 194, wherein the controller is further operable in a plant propagation mode in which the first incubation sequence and the second incubation sequence are executed.
196. The temporary immersion bioreactor of embodiment 192, wherein the growth vessel is elevated above the first and second media containers to allow liquid to flow from the growth vessel into at least one of the first media container and the second media container in the third operating mode.
197. The temporary immersion bioreactor of any one of embodiments 191 or 196, further comprising a manifold fluidically coupleable to the growth vessel, the first media container, and the second media container,

wherein the manifold is operable to control liquid flow between the growth vessel and the first media container and between the growth vessel and the second media container.

198. The temporary immersion bioreactor of any one of embodiments 191 or 197, wherein the growth vessel includes a fluid conduit configured to siphon liquid from the growth vessel to at least one of the first media container and the second media container.
199. The temporary immersion bioreactor of any one of embodiments 191 or 198, wherein the growth vessel is an ebb and flow bioreactor.
200. The temporary immersion bioreactor of any one of embodiments 191 or 199, wherein the controller is operable to control fluid communication between the growth vessel and the first media container, between the growth vessel and the second media container, between the gas source and the first media container, and between the gas source and the second media container.
201. A system for production of yam microtubers, comprising:

a temporary immersion bioreactor of any one of embodiments 191 to 200;

a yam explant;

a pre-tuberization medium, wherein the media is any one of embodiments 120-130;

a tuberization medium, wherein the media is any one of embodiments 131-140.

202. The system of embodiment 201, wherein the yam explant is a pathogen-free yam seedling.
203. The system of of any one of embodiments 201-202, wherein the yam seeding comprises about 4 to 7 axillary buds.
204. A method for producing yam tubers comprising
(a) obtaining pathogen-free microtubers via in vitro propagation, wherein the microtubers are produced by using any one of the methods 155-190;
(b) planting the microtubers; and
(c) obtaining yam tubers.
   205. The method of embodiment 204, wherein the microtubers are about 2 mm-about 3 mm long.
   206. The method of any one of embodiments 204-205, wherein the microtubers are about 5 mm-about 7 mm long.
   207. A method of increasing yam tuber production, comprising
(a) obtaining pathogen-free microtubers via in vitro propagation;
(b) planting the microtubers; and
(c) obtaining yam tubers,
   wherein the microtubers are less than about 5 mm long, and wherein the weight of yam tuber produced by using the microtubers less than about 5 mm long is higher than the weight of yam tuber produced by using microtubers more than 5 mm long.
   208. The method of embodiment 207, wherein the microtubers are about 2 mm-about 3 mm long.
   209. A method for micropropagating a plant comprising:

(a) incubating an explant of the plant on a solid media;

(b) transferring a microshoot from the explant of (a) to a bioreactor with liquid media;

(c) exposing the microshoot to a pulsing media;

(d) harvesting the microshoot at maturity; and

(e) transferring the mature microshoot to a media for rooting,

wherein the plant is a perennial, grass, or phyto-pharmaceutical plant; wherein the media is selected from at least one of BOO1-BOO91.

210. The method of embodiment 209, further comprising exposing the microshoot to medium comprising meta-topoline after step (c).
211. The method of any one of embodiments 209-210, wherein the solid media is selected from FIG. 26A or 26B.
212. The method of any one of embodiments 209-211, wherein the liquid media is a media selected from FIG. 26A or 26B and without agar.
213. The method of any one of embodiments 209-212, wherein the pulsing media is selected from FIG. 26B.
214. The method of embodiment 213, wherein the pulsing media is Pulsing media 1 or Pulsing media 2 of FIG. 26B.
215. The method of any one of embodiments 209-214, wherein the pulsing media is Pulsing media 1 of FIG. 26B, wherein TDZ is substituted with a different cytokinin.
216. The method of embodiment 215, wherein the different cytokinin is BAP, Zeatin, CPPU, or DPU.
217. The method of any one of embodiments 209-216, wherein the media for rooting is selected from FIG. 27.
218. The method of embodiment 217, wherein the media for rooting is BOO68, BOO69, BOO70, or BOO71 from FIG. 27.
219. A media for micropropagating a plant, a perennial, grass, or phyto-pharmaceutical plant, and the media is selected from FIG. 26A or 26B.
220. A kit comprising a media of FIG. 26A or 26B.
221. The kit of embodiment 220, wherein the media is Pulsing media 1 or Pulsing media 2.
222. The kit of any one of embodiments 220-221, wherein the media is Pulsing media 1 and TDZ is substituted with a different cytokinin.
223. The kit of any one of embodiments 220-222, further comprising a rooting media from FIG. 27.
224. The kit of embodiment 220 or 222, further comprising a BOO54, BOO55, BOO56, BOO57, BOO58, BOO59, BOO60, BOO61, BOO62, BOO63, BOO64, BOO65, BOO66, or BOO67 media from FIG. 26A or 26B.
225. A method for inducing a somatic embryo from an immature lateral bud of bamboo comprising:

(a) incubating the immature lateral bud in BOO72, BOO73, or BOO74 media;

(b) subculturing material from the lateral bud from (a) to fresh media until a pro-embryo structure is induced;

(c) transferring the pro-embryo from (b) to BOO77 or BOO78 media for the pro-embryo to develop into an embryo;

(d) transferring the embryo from (c) to BOO79 or BOO80 media for embryo maturation;

226. The method of embodiment 225, further comprising desiccating or germinating the embryo from (d) on BOO72 media.
227. The method of any one of embodiments 225-226, wherein the BOO72 media in step (d) does not contain a growth regulator.
228. The method of any one of embodiments 225-227, wherein in step (a) the immature lateral bud is in BOO72, BOO73, or BOO74 media for one to three days.
229. The method of any one of embodiments 225-228, wherein in step (a) the immature lateral bud is in BOO72, BOO73, or BOO74 media for up to seven days.
230. The method of any one of embodiments 225-229, wherein in step (a) the immature lateral bud is pulsed with BOO75 and BOO76 media.
231. The method of embodiment 230, wherein the pulsing with BOO75 and BOO76 media is for one to three days.
232. The method of embodiment 230, wherein the pulsing with BOO75 and BOO76 media is for up to seven days.
233. The method of any one of embodiments 225-232, wherein in step (b), subculturing to fresh media is performed every 28 days.
234. The method of embodiment 233, wherein the subculturing performed every 28 days is for a period of about 6 months.
235. The method any one of embodiments 225-234, wherein the bamboo is Phyllostachys edulisi ‘Moso’, Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, Guadua Angustifolia, Phylostachys Nigra, Fargesia rufa, Fargesia nitida, Borinda Boliana, Fargesia murielae, Pleioblastus fortune, Fargesia robusta, or Bambusa Oldhamii.
236. A media for inducing a somatic embryo from an immature lateral bud of bamboo, wherein the media is BOO72, BOO73, BOO74, BOO75, BOO76, BOO77, BOO78, BOO79, or BOO80.
237. A kit comprising a media of embodiment 236.
238. A medium for initiating bamboo somatic embryogenesis comprising at least two cytokins.
239. The medium of embodiment 238, wherein the at least two cytokins are thidiazuron (TDZ) and meta-topolin (mT), derivatives thereof, analogs thereof, and any combinations thereof.
240. The medium of any one of embodiments 238-239, further comprising one or more carbon sources.
241. The medium of embodiment 240, wherein the carbon source is selected from the group consisting of sucrose, glucose, maltose, lactose, or a combination thereof.
242. The medium of embodiment 239, wherein the concentration of TDZ and/or mT is about 0.1 to 10 mg/L.
243. The medium of any one of embodiments 238-242, wherein the medium is a liquid medium or a solid medium.
244. The medium of any one of embodiments 238-243, wherein the solid medium comprises a gelling agent.
245. The medium of embodiment 244, wherein the gelling agent is selected from the group consisting of agar, carrageenan, gellan gum, alginic acid and its salts, agarose, and any combinations thereof.
246. The medium of any one of embodiments 238-245, wherein the medium comprises of 5 g/L agar.
247. The medium of any one of embodiments 238-246, wherein the medium comprises one or more macronutrients, one or more micronutrients, and one or more vitamins.
248. The medium of embodiment 247, wherein the macronutrients, the micronutrients, and the vitamins are selected from those in the standard MS media, or one or more components of the standard MS media with doubled amount.
249. A method for in vitro propagation of bamboo, comprising
(a) culturing an explant obtained from a bamboo plant on a first medium to produce an embryo, wherein the first medium is the medium of any one of embodiments 238-248, and optionally,
(a′) culturing the embryo of (a) is cultured on a pulsing media.
250. The method of embodiment 249, wherein the explant comprises meristematic cells.
251. The method of any one of embodiments 249-250, wherein the explant comprises meristematic cells from axillary or lateral buds of a bamboo plant.
252. The method of any one of embodiments 249-251, wherein the method further comprising (b) culturing the embryo obtained from step (a) or (a′) in a second medium to produce embryogenic-like strucutres, wherein the second medium is a liquid or solid nutrient medium, comprising at least two amino acids.
253. The method of embodiment 252, wherein the second nutrient medium further comprises one or more vitamins.
254. The method of embodiment 252, wherein the second nutrient medium further comprises one or more carbon sources.
255. The method of embodiment 254, wherein the carbon source is selected from the group consisting of sucrose, glucose, maltose, lactose, or a combination thereof.
556. The method of embodiment 255, wherein the carbon source is maltose or lactose.
257. The method of embodiment 252, wherein the method further comprises
(c) transferring and culturing the embryogenic-like structures obtained from step (b) onto a third medium to produce mature somatic embryos, wherein the third medium comprises abscisic acid (ABA).
258. The method of embodiment 257, wherein the concentration of ABA in the third medium is about 10-100 mg/L.
259. The method of embodiment 257, wherein the method further comprises
(d) germinating the mature somatic embryos obtained from step (c) to produce bamboo plants.
260. The method of embodiment 259, wherein the mature somatic embryos are germinated on a fourth medium, wherein the fourth medium does not comprise any plant growth regulator.
261. A method for reducing the production of a phenolic in bamboo comprising:

incubating a bamboo tissue culture, explant or seed in BOO32, BOO33 or BOO34 media,

wherein the bamboo tissue culture, explant, or seed produces less of the phenolic as compared to a bamboo tissue culture, explant or seed incubated in media that is not BOO32, BOO33 or BOO34.

262. The method of embodiment 261, wherein the phenolic is a polyphenol.
263. The method of embodiment 261, wherein the phenolic is a luteolin derivative, flavone, flavone glycoside or phenolic acid.
264. The method of any one of embodiments 261-263, wherein the bamboo is Phyllostachys edulisi ‘Moso’, Phyllostachys bissetti, Fargesia denudata, Pleioblastus fortunei, Sasa Veitchii, Pleioblastus viridistriatus, Thamnocalamus crassinodus, Chusquea Culeo “Cana Prieta”, Bambusa Old Hamii, Phyllostachys Moso, Phyllostachys Atrovaginata, Dendrocalamus Asper, Guadua Angustifolia, Phylostachys Nigra, Fargesia rufa, Fargesia nitida, Borinda Boliana, Fargesia murielae, Pleioblastus fortune, Fargesia robusta, or Bambusa Oldhamii.
265. A media for reducing the production of a phenolic in bamboo, wherein the media is BOO32, BOO33 or BOO34.
266. A kit comprising a media of embodiment 265.
267. A method for producing a virus-free plantlet comprising:

(a) incubating an explant in a culture with BOO81 medium;

(b) subjecting the explant of (a) to thermotherapy, wherein the explant grows into a plantlet;

(c) excising an apical meristem from the plantlet of (b);

(d) placing the apical meristem of (c) into a regeneration media;

wherein a virus-free plantlet is produced from the apical meristem of (e).

268. The method of embodiment 267, wherein in step (a), the culture is incubated under a 16 h light photoperiod at 80-100 μmol/m²/x light intensity at 24° C.
269. The method of embodiment 268, wherein the incubation is for one to two weeks.
270. The method of any one of embodiments 267-269, wherein thermotherapy comprises incubating the explant under a 16 h light photoperiod at 30-40 μmol/m²/s light intensity at 37° C.
271. The method of embodiment 270, wherein thermotherapy is for one week.
272. The method of any one of embodiments 267-271, wherein the excising of step (c) is performed as in FIG. 21.
273. The method of any one of embodiments 267-272, wherein the regeneration media of step (d) is selected from FIG. 32.
274. The method of any one of embodiments 267-273, wherein in step (d), the apical meristem in regeneration media is incubated under a 16 h light photoperiod at 80-100 μmol/m²/x light intensity at 24° C.
275. The method of any one of embodiments 267-274, wherein an antiviral is in the regeneration media of step (d).
276. The method of any one of embodiments 267-275, further comprising:

(e) subculturing the apical meristem of step (d).

277. The method of embodiment 276, wherein the subculturing is every two to three weeks.
278. The method of embodiment 277, further comprising:

(f) transferring the apical meristem to a regeneration media.

279. The method of embodiment 278, wherein the regeneration media of step (f) is different than the regeneration media of step (d).
280. The method of embodiment 278, wherein the regeneration media of step (f) is the same as the regeneration media of step (d).
281. The method of embodiment 279 or 280, further comprising:

(g) subculturing the apical meristem.

282. The method of embodiment 281, wherein the subculturing of step (g) is every two or three weeks.
283. The method of any one of embodiments 267 to 282, further comprising subculturing the plantlet.
284. The method of any one of embodiments 267 to 283, further comprising testing the plantlet for viruses.
285. The method of embodiment 284, wherein the testing is by ELISA.
286. The method of any one of embodiments 267 to 285, wherein the plantlet is an agricultural plant.
287. The method of embodiment 286, wherein the agricultural plant is potato, tomato, yam, sugar beet, cassava, cucumber or cauliflower.
288. A media for producing a virus-free plantlet, wherein the media is selected from FIG. 32.
289. A kit comprising a media of FIG. 32.
290. The kit of embodiment 289, wherein the media is BOO81.
291. The kit of any one of embodiments 289-290, further comprising a regeneration medium.
292. The kit of embodiment 291, wherein the regeneration medium is selected from FIG. 32.
293. The kit of embodiment 290, further comprising two different regeneration media.
294. The kit of embodiment 293, wherein the two different regeneration media are selected from FIG. 32.
295. A method for producing a virus-free potato plantlet comprising:

(a) incubating a potato explant in a culture with BOO81 medium;

(b) subjecting an explant of the plant culture of (a) to thermotherapy, wherein the explant grows into a plantlet;

(c) excising an apical meristem from the plantlet of (b);

(d) placing the apical meristem of (c) into a regeneration media;

wherein a virus-free plantlet is produced from the apical meristem of (e).

296. The method of embodiment 295, wherein in step (a), the culture is incubated under a 16 h light photoperiod at 80-100 μmol/m²/x light intensity at 24° C.
297. The method of any one of embodiments 295-296, wherein the incubation is for one to two weeks.
298. The method of any one of embodiments 295-297, wherein thermotherapy comprises incubating the explant under a 16 h light photoperiod at 30-40 μmol/m²/s light intensity at 37° C.
299. The method of embodiment 298, wherein thermotherapy is for one week.
300. The method of any one of embodiments 295-299, wherein the excising of step (c) is performed as in FIG. 33.
301. The method of any one of embodiments 295-300, wherein the regeneration media of step (d) is selected from FIG. 32.
302. The method of any one of embodiments 295-301, wherein in step (d), the apical meristem in regeneration media is incubated under a 16 h light photoperiod at 80-100 μmol/m²/x light intensity at 24° C.
303. The method of any one of embodiments 295-302, wherein an antiviral is in the regeneration media of step (d).
304. The method of any one of embodiments 295-303, further comprising:

(e) subculturing the apical meristem of step (d).

305. The method of embodiment 304, wherein the subculturing is every two to three weeks.
306. The method of embodiment 305, further comprising: (f) transferring the apical meristem to a regeneration media.
307. The method of embodiment 306, wherein the regeneration media of step (f) is different than the regeneration media of step (d).
308. The method of embodiment 306, wherein the regeneration media of step (f) is the same as the regeneration media of step (d).
309. The method of embodiment 307 or 308, further comprising:

(g) subculturing the apical meristem.

310. The method of embodiment 309, wherein the subculturing of step (g) is every two or three weeks.
311. The method of any one of embodiments 295 to 310, further comprising subculturing the plantlet.
312. The method of any one of embodiments 295 to 311, further comprising testing the plantlet for viruses.
313. The method of embodiment 312, wherein the testing is by ELISA.
314. A media for producing a virus-free potato plantlet, wherein the media is selected from FIG. 32.
315. A kit comprising a media of FIG. 32.
316. The kit of embodiment 315, wherein the media is BOO81.
317. The kit of any one of embodiments 315-316, further comprising a regeneration medium.
318. The kit of embodiment 317, wherein the regeneration medium is selected from FIG. 32.
319. The kit of embodiment 318, further comprising two different regeneration media.
320. The kit of embodiment 319, wherein the two different regeneration media are selected from FIG. 32.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following Claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### REFERENCES

Gamborg, O. L., et al., Nutrient requirements of suspension cultures of soybean root cells. Exp. Cell Res. 50, 151-158, (1968).
Murashige, T. and F. Skoog. 1962. A revised medium for rapid growth and bioassays with tobacco cultures. Physiol. Plant 15:473-497
Quazi et al., 1978, Pathogen-free potato plants regenerated from meristem-tip cultures, N.Z. Journal of Experimental Agriculture 6:305-307.
Al-Taleb, 2011, Production of Virus Free Potato Using Meristem Culture from Cultivars Grown under Jordanian Environment, American-Eurasian J. Agric. & Environ. Sci., 11(4):467-472.
Plant Tissue Culture—Theory and Practice, a Revised Edition, Chapter 15, Production of pathogen-free plants, Volume 5, 1996, Pages 451-482.
2006 OEPP/EPPO, Bulletin OEPP/EPPO Bulletin 36, 187, Production of pathogen-free microplants of potato.
Bryabm 1989, Breaking dormancy of potato tubers, CIP Research Guide 16
Claassens et al., 2005, Ethanol Breaks dormancy of the potato tuber apical bud, Journal of Experimental Botany, Vol. 56, No. 419, pp. 2515-2525
Kämäräinen-Karppinen et al., 2010, Novel bioreactor technology for mass propagation of potato microtubers, Plant Cell Tiss Organ Cult (2010) 101:245-249
Aremu et al. Plant Cell Tiss. Organ. Cult., DOI 10.1007/s11240-011-0007-7, 2011
Lorenzo et al., 1998, Sugarcane shoot formation in an improved temporary immersion system, Plant Cell, Tissue and Organ Culture 54: 197-200, 1998.
Escalona et al., 1999, Pineapple (Ananas comosus L. Merr) micropropagation in temporary immersion systems, Plant Cell Reports (1999) 18: 743-748
Donnelly et al. 2003, Potato Microtuber Production and Performance: A Review, Amer J of Potato Res (2003) 80:103-115
Seabrook et al. 1993, Effect of photoperiod on in vitro tuberization of potato (Solanum tuberosum L.). Plant Cell Tissue Organ Culture 34: 43-51.
Gopal et al. 1998, Effectiveness of in vitro selection for agronomic characters in potato. Euphytica 103:67-74.
Gopal et al. 1997, Microtuberization in potato (Solanum tuberosum L.) Plant Cell Reports (1998) 17: 794-798
Robert et al., A New Temporary Immersion Bioreactor System for Micropropagation, Methods in Molecular Biology, vol. 318: Plant Cell Culture Protocols, Second Edition
Garner and Blake et al. 1989, The induction and development of potato microtubers in vitro on media free of growth regulating substances. Ann. Bot., 63: 663-74
Bizari et al. 1995, Effects of activated charcoal effects on induction and development of microtubers in potato (Solanum tuberosum L). Ann Appl Biol 127:175481.
Nasiruddin and Blake 1994, Production of potato microtubers with and without growth regulators. In: P J Lmnsden, J R Nicholas, W J Davies (eds), Physiology, Growth and Development of Plants. Kluwer, Dordrecht. pp. 254-260.
Jiménez et al., 1999, Improved production of potato microtubers using a temporary immersion system, Plant Cell, Tissue and Organ Culture 59: 19-23, 1999.
AKITA and TAKAYAMA, 1994, Stimulation of potato (Solanum tuberosum L.) tuberization by semicontinuous liquid medium surface level control, Plant Cell Reports (1994) 13:184 187
NISTOR et al., Influence of potato genotypes on “in vitro” production of microtubers, Romanian Biotechnological Letters Vol. 15, No. 3, 2010
ROŞU R., CHIRU N., ROLOT J. L., 2004, Researches on genotype influence on potato microtuberization, Anale, INCDCSZ, Vol. XXXI, Tipografia Phoenix Brasov, 120-129
ANOOP BADONI and J. S. CHAUHAN, 2009, A Note on Micro Tuber Seed Production of Potato: Necessitate Step for Uttarakhand Hills, Report and Opinion, 1(5):9-11;
WANG P., and C. HU, 1982. In vitro mass tuberization and virus-free seed-potato production in Taiwan. American Potato Journal 59:33-37
ABBOTT A. J., and A. R. BELCHER, 1986. Potato tuber formation in vitro. In: L A Withers, P G Alderson (eds), Plant Tissue Culture and its Agricultural Applications. Butterworths, London, pp. 113-122
EWING E. E, and P. C. STRUIK, 1992. Tuber formation in potato: induction, initiation and growth. In: J. Janick (ed), Horticultural Review. John Wiley & Sons Inc., New York. 14:89-198;
KHURI S., and J. MOORBY, 1995. Investigations into the role of sucrose in potato cv Estima microtuber production in vitro. Annals of Botany 75:295-303;
PERL A., D. AVIV, L. WILLMITZER, and E. GALUN, 1991. In vitro tuberization in transgenic potatoes harboring B-glucuronidase linked to a patatin promoter: Effect of sucrose levels and photoperiods. Plant Science 73:87-95;
LECLERC Y., D. DONNELLY, and JEA SEABROOK, 1994. Microtuberization of layered shoots and nodal cuttings of potato: The influence of growth regulators and incubation periods. Plant Cell, Tissue Organ Culture 37:113-120
LEVY D, JEA SEABROOK, and S COLEMAN, 1993. Enhancement of tuberization of axillary shoot buds of potato (Solanum tuberosum L.) cultivars cultured in vitro. J Expt Bot 44:381-386;
AHMAD, I. and I. A. KHAN, 1993. Microtubers induction and multiplication of two potato varieties. In: I. Illahi (ed.). Advances in Plant Tissue Culture, pp. 61-5. University of Peshawar Press, Peshawar, Pakistan
Sharma et al., Micro-tuber production behaviour of some commercially important potato. (Solanum tuberosum) cultivars, Indian Journal of Agricultural Sciences 81 (11): 1008-13, November 2011
Dobranszki et al., In vitro tuberization in hormone-free systems on solidified medium and dormancy of potato microtubers, fruit, vegetable and cereal science and biotechnology, 2008, Global Science Books

1. A set of media for producing a hemp plant or plant part wherein the set of media comprises:
(a) one or more initiation medium;
(b) one or more multiplication medium; and
(c) one or more rooting medium;
wherein the initiation medium, the multiplication medium and/or the rooting medium comprises at least one cytokinin; wherein the media is selected from one of BOO1-BOO91.
